SEQUENCE LISTING (283 sequences)

SEQ ID NO: 1 - protein, 5 aa, Artificial Sequence
  Peptide linker I
GGGGS

SEQ ID NO: 2 - protein, 12 aa, Artificial Sequence
  Peptide linker II
GKSSGSGSESKS

SEQ ID NO: 3 - protein, 14 aa, Artificial Sequence
  Peptide linker III
GSTSGSGKSSEGKG

SEQ ID NO: 4 - protein, 18 aa, Artificial Sequence
  Peptide linker IV
GSTSGSGKSSEGSGSTKG

SEQ ID NO: 5 - protein, 18 aa, Artificial Sequence
  Peptide linker V
GSTSGSGKPGSGEGSTKG

SEQ ID NO: 6 - protein, 14 aa, Artificial Sequence
  Peptide linker VI
EGKSSGSGSESKEF

SEQ ID NO: 7 - protein, 6 aa, Artificial Sequence
  peptide linker
SGSGSG

SEQ ID NO: 8 - protein, 70 aa, Unknown
  DRBD peptide
FFMEELNTYRQKQGVVLKYQELPNSGPPHDRRFTFQVIIDGREFPEGEGRSKKEAKNAAAKLAVEILNKE

SEQ ID NO: 9 - protein, 4 aa, Artificial Sequence
  furin Arg-X-X-Arg
RXXR

SEQ ID NO: 10 - protein, 4 aa, Artificial Sequence
  furin Arg-X-Lys/Arg-Arg
RXXR

SEQ ID NO: 11 - protein, 11 aa, Artificial Sequence
  Peptide with cleavage site
TPLKSPPPSPR

SEQ ID NO: 12 - protein, 6 aa, Artificial Sequence
  cleavage site+C235
PQYTYA

SEQ ID NO: 13 - protein, 6 aa, Artificial Sequence
  cleavage site
VGRPRH

SEQ ID NO: 14 - protein, 6 aa, Artificial Sequence
  cleavage site
TVKGSK

SEQ ID NO: 15 - protein, 6 aa, Artificial Sequence
  cleavage site
FVFEEF

SEQ ID NO: 16 - protein, 6 aa, Artificial Sequence
  cleavage site
QNLKDA

SEQ ID NO: 17 - protein, 6 aa, Artificial Sequence
  cleavage site
VSMVDP

SEQ ID NO: 18 - protein, 6 aa, Artificial Sequence
  cleavage site
RLRAEG

SEQ ID NO: 19 - protein, 6 aa, Artificial Sequence
  cleavage site
VCGTPG

SEQ ID NO: 20 - protein, 6 aa, Artificial Sequence
  cleavage site
TENLVP

SEQ ID NO: 21 - protein, 6 aa, Artificial Sequence
  cleavage site
VVQAGI

SEQ ID NO: 22 - protein, 6 aa, Artificial Sequence
  cleavage site
QLDAIS

SEQ ID NO: 23 - protein, 6 aa, Artificial Sequence
  cleavage site
PEIRKP

SEQ ID NO: 24 - protein, 6 aa, Artificial Sequence
  cleavage site
SQTRAP

SEQ ID NO: 25 - protein, 6 aa, Artificial Sequence
  cleavage site
LNLADP

SEQ ID NO: 26 - protein, 6 aa, Artificial Sequence
  cleavage site
LLTSPD

SEQ ID NO: 27 - protein, 6 aa, Artificial Sequence
  cleavage site
ITTTPT

SEQ ID NO: 28 - protein, 6 aa, Artificial Sequence
  cleavage site
SLHSEP

SEQ ID NO: 29 - protein, 6 aa, Artificial Sequence
  cleavage site
LTEVGM

SEQ ID NO: 30 - protein, 6 aa, Artificial Sequence
  cleavage site
PLSAKP

SEQ ID NO: 31 - protein, 6 aa, Artificial Sequence
  cleavage site
ELESLP

SEQ ID NO: 32 - protein, 6 aa, Artificial Sequence
  cleavage site
TTKMAQ

SEQ ID NO: 33 - protein, 6 aa, Artificial Sequence
  cleavage site
PLEISY

SEQ ID NO: 34 - protein, 6 aa, Artificial Sequence
  cleavage site
VTTREQ

SEQ ID NO: 35 - protein, 6 aa, Artificial Sequence
  cleavage site
RLLQER

SEQ ID NO: 36 - protein, 6 aa, Artificial Sequence
  cleavage site
WIPEGE

SEQ ID NO: 37 - protein, 6 aa, Artificial Sequence
  cleavage site
STSRTP

SEQ ID NO: 38 - protein, 6 aa, Artificial Sequence
  cleavage site
SCPIKE

SEQ ID NO: 39 - protein, 6 aa, Artificial Sequence
  cleavage site
DTFLPV

SEQ ID NO: 40 - protein, 6 aa, Artificial Sequence
  cleavage site
STFDSP

SEQ ID NO: 41 - protein, 6 aa, Artificial Sequence
  cleavage site
PNGIFK

SEQ ID NO: 42 - protein, 6 aa, Artificial Sequence
  cleavage site
AIRTST

SEQ ID NO: 43 - protein, 6 aa, Artificial Sequence
  cleavage site
SINEAI

SEQ ID NO: 44 - protein, 6 aa, Artificial Sequence
  cleavage site
NVKAAW

SEQ ID NO: 45 - protein, 6 aa, Artificial Sequence
  cleavage site
EEKSAV

SEQ ID NO: 46 - protein, 6 aa, Artificial Sequence
  cleavage site
RLLRKG

SEQ ID NO: 47 - protein, 6 aa, Artificial Sequence
  cleavage site
GTKAVT

SEQ ID NO: 48 - protein, 6 aa, Artificial Sequence
  cleavage site
ATGGVK

SEQ ID NO: 49 - protein, 6 aa, Artificial Sequence
  cleavage site
PKKAQD

SEQ ID NO: 50 - protein, 6 aa, Artificial Sequence
  cleavage site
TVMNPK

SEQ ID NO: 51 - protein, 6 aa, Artificial Sequence
  cleavage site
KLKSQW

SEQ ID NO: 52 - protein, 6 aa, Artificial Sequence
  cleavage site
PLFKSA

SEQ ID NO: 53 - protein, 6 aa, Artificial Sequence
  cleavage site
ERARAK

SEQ ID NO: 54 - protein, 6 aa, Artificial Sequence
  cleavage site
WDTANN

SEQ ID NO: 55 - protein, 6 aa, Artificial Sequence
  cleavage site
PLYKEA

SEQ ID NO: 56 - protein, 6 aa, Artificial Sequence
  cleavage site
STFTNI

SEQ ID NO: 57 - protein, 6 aa, Artificial Sequence
  cleavage site
ITYRGT

SEQ ID NO: 58 - protein, 6 aa, Artificial Sequence
  cleavage site
KPRSVA

SEQ ID NO: 59 - protein, 6 aa, Artificial Sequence
  cleavage site
KPRSSP

SEQ ID NO: 60 - protein, 6 aa, Artificial Sequence
  cleavage site
VVTAEA

SEQ ID NO: 61 - protein, 6 aa, Artificial Sequence
  cleavage site
NIVTPR

SEQ ID NO: 62 - protein, 6 aa, Artificial Sequence
  cleavage site
ASASTM

SEQ ID NO: 63 - protein, 6 aa, Artificial Sequence
  cleavage site
HYGSLP

SEQ ID NO: 64 - protein, 6 aa, Artificial Sequence
  cleavage site
TPRTPP

SEQ ID NO: 65 - protein, 6 aa, Artificial Sequence
  cleavage site
VNKLIL

SEQ ID NO: 66 - protein, 6 aa, Artificial Sequence
  cleavage site
ILQLCI

SEQ ID NO: 67 - protein, 6 aa, Artificial Sequence
  cleavage site
ILKGAR

SEQ ID NO: 68 - protein, 6 aa, Artificial Sequence
  cleavage site
PLRMEE

SEQ ID NO: 69 - protein, 6 aa, Artificial Sequence
  cleavage site
PLSRTP

SEQ ID NO: 70 - protein, 6 aa, Artificial Sequence
  cleavage site
KEYFSK

SEQ ID NO: 71 - protein, 6 aa, Artificial Sequence
  cleavage site
SDTIKP

SEQ ID NO: 72 - protein, 6 aa, Artificial Sequence
  cleavage site
LQFQKN

SEQ ID NO: 73 - protein, 6 aa, Artificial Sequence
  cleavage site
LFSVPS

SEQ ID NO: 74 - protein, 6 aa, Artificial Sequence
  cleavage site
STFAQP

SEQ ID NO: 75 - protein, 6 aa, Artificial Sequence
  cleavage site
WKLLPE

SEQ ID NO: 76 - protein, 6 aa, Artificial Sequence
  cleavage site
AVHSGP

SEQ ID NO: 77 - protein, 6 aa, Artificial Sequence
  cleavage site
HLLSPW

SEQ ID NO: 78 - protein, 6 aa, Artificial Sequence
  cleavage site
KSGAAP

SEQ ID NO: 79 - protein, 6 aa, Artificial Sequence
  cleavage site
KLTVNP

SEQ ID NO: 80 - protein, 6 aa, Artificial Sequence
  cleavage site
ALHSQP

SEQ ID NO: 81 - protein, 6 aa, Artificial Sequence
  cleavage site
ENPGKE

SEQ ID NO: 82 - protein, 6 aa, Artificial Sequence
  cleavage site
PRGKFK

SEQ ID NO: 83 - protein, 6 aa, Artificial Sequence
  cleavage site
GNKVIS

SEQ ID NO: 84 - protein, 6 aa, Artificial Sequence
  cleavage site
KAKLGP

SEQ ID NO: 85 - protein, 6 aa, Artificial Sequence
  cleavage site
EDRKQP

SEQ ID NO: 86 - protein, 6 aa, Artificial Sequence
  cleavage site
KIGQGT

SEQ ID NO: 87 - protein, 6 aa, Artificial Sequence
  cleavage site
EEKTAN

SEQ ID NO: 88 - protein, 6 aa, Artificial Sequence
  cleavage site
PSSSPI

SEQ ID NO: 89 - protein, 6 aa, Artificial Sequence
  cleavage site
RCFFGA

SEQ ID NO: 90 - protein, 6 aa, Artificial Sequence
  cleavage site
GLNRIQ

SEQ ID NO: 91 - protein, 6 aa, Artificial Sequence
  cleavage site
ELRRGQ

SEQ ID NO: 92 - protein, 6 aa, Artificial Sequence
  cleavage site
MMTQPP

SEQ ID NO: 93 - protein, 6 aa, Artificial Sequence
  cleavage site
ISQTAQ

SEQ ID NO: 94 - protein, 6 aa, Artificial Sequence
  cleavage site
EVYGMM

SEQ ID NO: 95 - protein, 6 aa, Artificial Sequence
  cleavage site
KSTASW

SEQ ID NO: 96 - protein, 6 aa, Artificial Sequence
  cleavage site
VLQQQY

SEQ ID NO: 97 - protein, 6 aa, Artificial Sequence
  cleavage site
EVMEDH

SEQ ID NO: 98 - protein, 6 aa, Artificial Sequence
  cleavage site
GLKESP

SEQ ID NO: 99 - protein, 6 aa, Artificial Sequence
  cleavage site
VVRTPP

SEQ ID NO: 100 - protein, 6 aa, Artificial Sequence
  cleavage site
DLKNVK

SEQ ID NO: 101 - protein, 6 aa, Artificial Sequence
  cleavage site
NVKSKI

SEQ ID NO: 102 - protein, 6 aa, Artificial Sequence
  cleavage site
NLKHQP

SEQ ID NO: 103 - protein, 6 aa, Artificial Sequence
  cleavage site
IVYKPV

SEQ ID NO: 104 - protein, 6 aa, Artificial Sequence
  cleavage site
EVKSEK

SEQ ID NO: 105 - protein, 6 aa, Artificial Sequence
  cleavage site
IVYKSP

SEQ ID NO: 106 - protein, 6 aa, Artificial Sequence
  cleavage site
AIMSPR

SEQ ID NO: 107 - protein, 6 aa, Artificial Sequence
  cleavage site
ELDAKQ

SEQ ID NO: 108 - protein, 6 aa, Artificial Sequence
  cleavage site
RLRSSV

SEQ ID NO: 109 - protein, 6 aa, Artificial Sequence
  cleavage site
GSGTSS

SEQ ID NO: 110 - protein, 6 aa, Artificial Sequence
  cleavage site
GTSSRP

SEQ ID NO: 111 - protein, 6 aa, Artificial Sequence
  cleavage site
VTTSTR

SEQ ID NO: 112 - protein, 6 aa, Artificial Sequence
  cleavage site
RTYSLG

SEQ ID NO: 113 - protein, 6 aa, Artificial Sequence
  cleavage site
SLGSAL

SEQ ID NO: 114 - protein, 6 aa, Artificial Sequence
  cleavage site
SLYSSS

SEQ ID NO: 115 - protein, 6 aa, Artificial Sequence
  cleavage site
VTRSSA

SEQ ID NO: 116 - protein, 6 aa, Artificial Sequence
  cleavage site
LLKSHR

SEQ ID NO: 117 - protein, 6 aa, Artificial Sequence
  cleavage site
SKRSLS

SEQ ID NO: 118 - protein, 558 aa, Homo sapiens
  Full length AMF protein (1)..(558)
MAALTRDPQFQKLQQWYREHRSELNLRRLFDANKDRFNHFSLTLNTNHGHILVDYSKNLVTEDVMRMLVDLAKSRGVEAARERMFNGEKINYTEGRAVLHVALRNRSNTPILVDGKDVMPEVNKVLDKMKSFCQRVRSGDWKGYTGKTITDVINIGIGGSDLGPLMVTEALKPYSSGGPRVWYVSNIDGTHIAKTLAQLNPESSLFIIASKTFTTQETITNAETAKEWFLQAAKDPSAVAKHFVALSTNTTKVKEFGIDPQNMFEFWDWVGGRYSLWSAIGLSIALHVGFDNFEQLLSGAHWMDQHFRTTPLEKNAPVLLALLGIWYINCFGCETHAMLPYDQYLHRFAAYFQQGDMESNGKYITKSGTRVDHQTGPIVWGEPGTNGQHAFYQLIHQGTKMIPCDFLIPVQTQHPIRKGLHHKILLANFLAQTEALMRGKSTEEARKELQAAGKSPEDLERLLPHKVFEGNRPTNSIVFTKLTPFMLGALVAMYEHKIFVQGIIWDINSFDQWGVELGKQLAKKIEPELDGSAQVTSHDASTNGLINFIKQQREARVQ

SEQ ID NO: 119 - protein, 558 aa, Mus musculus
  Full length AMF protein (1)..(558)
MAALTRNPQFQKLLEWHRANSANLKLRELFEADPERFNNFSLNLNTNHGHILVDYSKNLVNKEVMQMLVELAKSRGVEAARDNMFSGSKINYTENRAVLHVALRNRSNTPIKVDGKDVMPEVNRVLDKMKSFCQRVRSGDWKGYTGKSITDIINIGIGGSDLGPLMVTEALKPYSKGGPRVWFVSNIDGTHIAKTLASLSPETSLFIIASKTFTTQETITNAETAKEWFLEAAKDPSAVAKHFVALSTNTAKVKEFGIDPQNMFEFWDWVGGRYSLWSAIGLSIALHVGFDHFEQLLSGAHWMDQHFLKTPLEKNAPVLLALLGIWYINCYGCETHALLPYDQYMHRFAAYFQQGDMESNGKYITKSGARVDHQTGPIVWGEPGTNGQHAFYQLIHQGTKMIPCDFLIPVQTQHPIRKGLHHKILLANFLAQTEALMKGKLPEEARKELQAAGKSPEDLEKLLPHKVFEGNRPTNSIVFTKLTPFILGALIAMYEHKIFVQGIMWDINSFDQWGVELGKQLAKKIEPELEGSSAVTSHDSSTNGLISFIKQQRDTKLE

SEQ ID NO: 120 - protein, 374 aa, Homo sapiens
  Human Sulfatase-modifying factor 1 (SUMF1) (1)..(374)
MAAPALGLVCGRCPELGLVLLLLLLSLLCGAAGSQEAGTGAGAGSLAGSCGCGTPQRPGAHGSSAAAHRYSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMD

SEQ ID NO: 121 - protein, 12 aa, Homo sapiens
  TfR binding peptide (1)..(12)
THRPPMWSPVWP

SEQ ID NO: 122 - protein, 9 aa, Homo sapiens
  TfR binding peptide (1)..(9)
GHKVKRPKG

SEQ ID NO: 123 - protein, 7 aa, Homo sapiens
  TfR binding peptide (1)..(7)
HAIYPRH

SEQ ID NO: 124 - protein, 262 aa, Ricinus communis
  Ricin B chain (1)..(262)
ADVCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLF

SEQ ID NO: 125 - protein, 103 aa, Vibrio cholerae
  Cholera toxin B chain (1)..(103)
TPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMAN

SEQ ID NO: 126 - protein, 69 aa, Shigella dysenteriae
  Shiga toxin subunit B (stxB) (1)..(69)
TPDCVTGKVEYTKYNDDDTFTVKVGDKELFTNRWNLQSLLLSAQITGMTVTIKTNACHNGGGFSEVIFR

SEQ ID NO: 127 - protein, 69 aa, Enterobacteria phage H19B; identical to SEQ ID NO: 126
  Stx1 B (SLT1 or VT1) subunit without the signal sequence (1)..(69)

SEQ ID NO: 128 - protein, 69 aa, Enterobacteria phage H19B; identical to SEQ ID NO: 126
  Shiga-like toxin 1 subunit B (1)..(69)

SEQ ID NO: 129 - protein, 89 aa, Escherichia coli
  Slt-Ic (Stx1c or VT1c) B subunit, putative signal peptide aa 1-19 (1)..(89)
MKKILLIAASLSFFSASVLAAPDCVTGKVEYTKYNDDDTFTVKVGDKELFTNRWNLQSLLLSAQITGMTVTIKTNACHNGGGFSEVIFR

SEQ ID NO: 130 - protein, 70 aa, Enterobacteria phage 933W
  Shiga-like toxin 2 subunit B (1)..(70)
ADCAKGKIEFSKYNEDDTFTVKVDGKEYWTSRWNLQPLLQSAQLTGMTVTIKSSTCESGSGFAEVQFNND

SEQ ID NO: 131 - protein, 89 aa, Enterobacteria phage 2851
  Slt-IIc (SltIIc or VT2c) B subunit, putative signal peptide aa 1-19 (1)..(89)
MKKMFMAVLFALVSVNAMAADCAKGKIEFSKYNENDTFTVKVAGKEYWTSRWNLQPLLQSAQLTGMTVTIKSSTCESGSGFAEVQFNND

SEQ ID NO: 132 - protein, 89 aa, Escherichia coli
  Slt-IId (SltIId or VT2d) B subunit, putative signal peptide aa 1-19 (1)..(89)
  Putative signal peptide aa 1-19 (1)..(19)
  Slt-IId (SltIId or VT2d) B subunit (1)..(89)
MKKMFMAVLFALVSVNAMAADCAKGKIEFSKYNENDTFTVKVDGKEYWTSRWNLQPLLQSAQLTGMTVTIKSSTCASGSGFAEVQFNND

SEQ ID NO: 133 - protein, 87 aa, Escherichia coli
  Putative signal peptide aa 1-19 (1)..(19)
  Slt-IIe (SltIIe or VT2e) B subunit (1)..(87)
MKKMFIAVLFALVSVNAMAADCAKGKIEFSKYNEDNTFTVKVSGREYWTNRWNLQPLLQSAQLTGMTVTIISNTCSSGSGFAQVKFN

SEQ ID NO: 134 - protein, 87 aa, Escherichia coli
  Slt-IIf (SltIIf or VT2f) B subunit (1)..(87)
  Putative signal peptide aa 1-19 (1)..(19)
MKKMIIAVLFGLFSANSMAADCAVGKIEFSKYNEDDTFTVKVSGREYWTNRWNLQPLLQSAQLTGMTVTIISNTCSSGSGFAQVKFN

SEQ ID NO: 135 - protein, 103 aa, Escherichia coli
  Heat-labile enterotoxin B chain (1)..(103)
APQSITELCSEYHNTQIYTINDKILSYTESMAGKREMVIITFKSGATFQVEVPGSQHIDSQKKAIERMKDTLRITYLTETKIDKLCVWNNKTPNSIAAISMEN

SEQ ID NO: 136 - protein, 103 aa, Escherichia coli
  Heat-labile enterotoxin B chain (LT-B, porcine), B chain without signal peptide (1)..(103)
APQTITELCSEYRNTQIYTINDKILSYTESMAGKREMVIITFKSGETFQVEVPGSQHIDSQKKAIERMKDTLRITYLTETKIDKLCVWNNKTPNSIAAISMKN

SEQ ID NO: 137 - protein, 104 aa, Escherichia coli
  Heat-labile enterotoxin IIA, B chain (LT-IIA) (1)..(104)
QVYAGVSEHFRNICNQTTADIVAGVQLKKYIADVNTNTRGIYVVSNTGGVWYIPGGRDYPDNFLSGEIRKTAMAAILSDTKVNLCAKTSSSPNHIWAMELDRES

SEQ ID NO: 138 - protein, 99 aa, Escherichia coli
  Heat-labile enterotoxin IIB, B chain (LT-IIB) (1)..(99)
GASQFFKDNCNRTTASLVEGVELTKYISDINNNTDGMYVVSSTGGVWRISRAKDYPDNVMTAEMRKIAMAAVLSGMRVNMCASPASSPNVIWAIELEAE

SEQ ID NO: 139 - protein, 267 aa, Abrus precatorius
  Abrin B chain (1)..(267)
IVEKSKICSSRYEPTVRIGGRDGMCVDVYDNGYHNGNRIIMWKCKDRLEENQLWTLKSDKTIRSNGKCLTTYGYAPGSYVMIYDCTSAVAEATYWEIWDNGTIINPKSALVLSAESSSMGGTLTVQTNEYLMRQGWRTGNNTSPFVTSISGYSDLCMQAQGSNVWMADCDSNKKEQQWALYTDGSIRSVQNTNNCLTSKDHKQGSTILLMGCSNGWASQRWVFKNDGSIYSLYDDMVMDVKGSDPSLKQIILWPYTGKPNQIWLTLF

SEQ ID NO: 140 - protein, 4 aa, Artificial Sequence
  variable peptide based on aa′s 1-4 of EDEL
XXXX

SEQ ID NO: 141 - protein, 4 aa, Homo sapiens
  EDEL motif (1)..(4)
EDEL

SEQ ID NO: 142 - protein, 4 aa, Homo sapiens
  HDEL motif (1)..(4)
HDEL

SEQ ID NO: 143 - protein, 4 aa, Homo sapiens
  HEEL motif (1)..(4)
HEEL

SEQ ID NO: 144 - protein, 4 aa, Homo sapiens
  KAEL motif (1)..(4)
KAEL

SEQ ID NO: 145 - protein, 4 aa, Homo sapiens
  KDEF motif (1)..(4)
KDEF

SEQ ID NO: 146 - protein, 4 aa, Homo sapiens
  KEDL motif (1)..(4)
KEDL

SEQ ID NO: 147 - protein, 4 aa, Homo sapiens
  KEEL motif (1)..(4)
KEEL

SEQ ID NO: 148 - protein, 4 aa, Homo sapiens
  KTEL motif (1)..(4)
KTEL

SEQ ID NO: 149 - protein, 4 aa, Homo sapiens
  KVEL motif (1)..(4)
KVEL

SEQ ID NO: 150 - protein, 4 aa, Homo sapiens
  NEDL motif (1)..(4)
NEDL

SEQ ID NO: 151 - protein, 4 aa, Homo sapiens
  PDEL motif (1)..(4)
PDEL

SEQ ID NO: 152 - protein, 4 aa, Homo sapiens
  PGEL motif (1)..(4)
PGEL

SEQ ID NO: 153 - protein, 4 aa, Homo sapiens
  QEDL motif (1)..(4)
QEDL

SEQ ID NO: 154 - protein, 4 aa, Homo sapiens
  QSEL motif (1)..(4)
QSEL

SEQ ID NO: 155 - protein, 4 aa, Homo sapiens
  REDL motif (1)..(4)
REDL

SEQ ID NO: 156 - protein, 4 aa, Homo sapiens
  RNEL motif (1)..(4)
RNEL

SEQ ID NO: 157 - protein, 4 aa, Homo sapiens
  RTDL motif (1)..(4)
RTDL

SEQ ID NO: 158 - protein, 4 aa, Homo sapiens
  RTEL motif (1)..(4)
RTEL

SEQ ID NO: 159 - protein, 6 aa, Homo sapiens
  ERSTEL motif (1)..(6)
ERSTEL

SEQ ID NO: 160 - protein, 4 aa, Homo sapiens
  KDEL motif (1)..(4)
KDEL

SEQ ID NO: 161 - protein, 5 aa, Homo sapiens
  AKDEL motif (1)..(5)
AKDEL

SEQ ID NO: 162 - protein, 4 aa, Homo sapiens
  PTEL motif (1)..(4)
PTEL

SEQ ID NO: 163 - protein, 4 aa, Homo sapiens
  STEL motif (1)..(4)
STEL

SEQ ID NO: 164 - protein, 16 aa, Artificial Sequence
  CL1 deduced C-terminal destablizing seq found in screen
ACKNWFSSLSHFVIHL

SEQ ID NO: 165 - protein, 35 aa, Artificial Sequence
  CL2 deduced C-terminal destablizing seq found in screen
SLISLPLPTRVKFSSLLLIRIMKIITMTFPKKLRS

SEQ ID NO: 166 - protein, 16 aa, Artificial Sequence
  CL6 deduced C-terminal destablizing seq found in screen
FYYPIWFARVLLVHYQ

SEQ ID NO: 167 - protein, 46 aa, Artificial Sequence
  CL9 deduced C-terminal destablizing seq found in screen
SNPFSSLFGASLLIDSVSLKSNWDTSSSSCLISFFSSVMFSSTTRS

SEQ ID NO: 168 - protein, 39 aa, Artificial Sequence
  CL10 deduced C-terminal destablizing seq found in screen
CRQRFSCHLTASYPQSTVTPFLAFLRRDFFFLRHNSSAD

SEQ ID NO: 169 - protein, 46 aa, Artificial Sequence
  CL11 deduced C-terminal destablizing seq found in screen
GAPHVVLFDFELRITNPLSHIQSVSLQITLIFCSLPSLILSKFLQV

SEQ ID NO: 170 - protein, 39 aa, Artificial Sequence
  CL12 deduced C-terminal destablizing seq found in screen
NTPLFSKSFSTTCGVAKKTLLLAQISSLFFLLLSSNIAV

SEQ ID NO: 171 - protein, 45 aa, Artificial Sequence
  CL15 deduced C-terminal destablizing seq found in screen
PTVKNSPKIFCLSSSPYLAFNLEYLSLRIFSTLSKCSNTLLTSLS

SEQ ID NO: 172 - protein, 30 aa, Artificial Sequence
  CL16 deduced C-terminal destablizing seq found in screen
SNQLKRLWLWLLEVRSFDRTLRRPWIHLPS

SEQ ID NO: 173 - protein, 50 aa, Artificial Sequence
  SL17 deduced C-terminal destablizing seq found in screen
SISFVIRSHASIRMGASNDFFHKLYFTKCLTSVILSKFLIHLLLRSTPRV

SEQ ID NO: 174 - protein, 604 aa, Homo sapiens
  1-604 COX2 protein (1)..(604)
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 175 - protein, 101 aa, Homo sapiens
  504-604 COX2 peptide (1)..(101)
FGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 176 - protein, 19 aa, Homo sapiens
  580-598 COX2 peptide (1)..(19)
NASSSRSGLDDINPTVLLK

SEQ ID NO: 177 - protein, 25 aa, Homo sapiens
  corresponding to amino acids 580 through 604 of human COX2 (1)..(25)
NASSSRSGLDDINPTVLLKERSTEL

SEQ ID NO: 178 - protein, 19 aa, Artificial Sequence
  variable peptide based on aa′s 580-598 of COX2 protein
NXSXXXXXXXXINPTXXXX

SEQ ID NO: 179 - protein, 19 aa, Mus musculus
  580-598 COX2 peptide variant (1)..(19)
NASASHSRLDDINPTVLIK

SEQ ID NO: 180 - protein, 19 aa, Felis catus
  580-598 COX2 peptide variant (1)..(19)
NASSSHSGLDDINPTVLLK

SEQ ID NO: 181 - protein, 19 aa, Artificial Sequence
  variable peptide based on aa′s 580-598 of COX2 protein
NXSSXXSXXDDINPTVLLK

SEQ ID NO: 182 - protein, 455 aa, Mus musculus
  aa 1-455 of murine IgM(mu) (1)..(455)
ESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNKDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 183 - protein, 35 aa, Mus musculus
  aa 421-455 of murine IgM(mu) (1)..(35)
ALPHLVTERTVDKSTGKPTLYNVSLIMSDTGGTCY

SEQ ID NO: 184 - protein, 20 aa, Mus musculus
  aa 436-455 of murine IgM (mu) (1)..(20)
GKPTLYNVSLIMSDTGGTCY

SEQ ID NO: 185 - protein, 20 aa, Homo sapiens
  human peptide variant of aa 436-455 of murine IgM(mu) (1)..(20)
GKPTLYNVSLVMSDTAGTCY

SEQ ID NO: 186 - protein, 20 aa, Rattus norvegicus
  rat peptide variant I of aa 436-455 of murine IgM(mu) (1)..(20)
GKPTLYQVSLIMSDTGGTCY

SEQ ID NO: 187 - protein, 20 aa, Rattus norvegicus
  rat peptide variant II of aa 436-455 of murine IgM(mu) (1)..(20)
GKPTLYQVSLIMSDTGGTSY

SEQ ID NO: 188 - protein, 453 aa, Homo sapiens
  human IgM(mu) (1)..(453)
GSLSAPTLFPLVSCENSPSDTSSVAVGCLAQDFLPDSITFSWKYKNNSDISSTRGFPSVLRGGKHAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGTTTYKVTSTLTIKESDWLSQSMFTCRVDHRGLTFQQNASSMCGPDQDTAIRVFSIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 189 - protein, 35 aa, Homo sapiens
  aa 421-455 of human IgM(mu) (1)..(35)
ALPNRVTERTVDKSTGKPTLYNVSLVMSDTAGTCY

SEQ ID NO: 190 - protein, 20 aa, Artificial Sequence
  artificial IgM(mu) peptide
GKPTLYXVSLXMSDTXGTXY

SEQ ID NO: 191 - protein, 431 aa, Mus musculus
  aa 1-431 of murine Sgk1 (1)..(431)
MTVKAEAARSTLTYSRMRGMVAILIAFMKQRRMGLNDFIQKIASNTYACKHAEVQSILKMSHPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNGTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHIFFSLINWDDLINKKITPPFNPNVSGPSDLRHFDPEFTEEPVPSSIGRSPDSILVTASVKEAAEAFLGFSYAPPVDSFL

SEQ ID NO: 192 - protein, 100 aa, Mus musculus
  aa 1-100 of murine Sgk1 (1)..(100)
MTVKAEAARSTLTYSRMRGMVAILIAFMKQRRMGLNDFIQKIASNTYACKHAEVQSILKMSHPQEPELMNANPSPPPSPSQQINLGPSSNPHAKPSDFHF

SEQ ID NO: 193 - protein, 60 aa, Mus musculus
  aa 1-60 of murine Sgk1 (1)..(60)
MTVKAEAARSTLTYSRMRGMVAILIAFMKQRRMGLNDFIQKIASNTYACKHAEVQSILKM

SEQ ID NO: 194 - protein, 33 aa, Mus musculus
  aa 1-33 of murine Sgk1 (1)..(33)
MTVKAEAARSTLTYSRMRGMVAILIAFMKQRRM

SEQ ID NO: 195 - protein, 431 aa, Homo sapiens
  human Sgk1 (1)..(431)
MTVKTEAAKGTLTYSRMRGMVAILIAFMKQRRMGLNDFIQKIANNSYACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFFSLINWDDLINKKITPPFNPNVSGPNDLRHFDPEFTEEPVPNSIGKSPDSVLVTASVKEAAEAFLGFSYAPPTDSFL

SEQ ID NO: 196 - protein, 100 aa, Homo sapiens
  aa 1-100 of human Sgk1 (1)..(100)
MTVKTEAAKGTLTYSRMRGMVAILIAFMKQRRMGLNDFIQKIANNSYACKHPEVQSILKISQPQEPELMNANPSPPPSPSQQINLGPSSNPHAKPSDFHF

SEQ ID NO: 197 - protein, 60 aa, Homo sapiens
  amino acids 1 through 60 of human Sgk1 (1)..(60)
MTVKTEAAKGTLTYSRMRGMVAILIAFMKQRRMGLNDFIQKIANNSYACKHPEVQSILKI

SEQ ID NO: 198 - protein, 33 aa, Homo sapiens
  aa 1-33 of human Sgk1 (1)..(33)
MTVKTEAAKGTLTYSRMRGMVAILIAFMKQRRM

SEQ ID NO: 199 - protein, 30 aa, Homo sapiens
  1-30 of human Sgk1 protein (1)..(30)
MTVKTEAAKGTLTYSRMRGMVAILIAFMKQ

SEQ ID NO: 200 - protein, 63 aa, Artificial Sequence
  variable peptide based on aa 1-60 of murine Sgk1
MTXXXXEXXXXXXXLTYSXXRGXVAXLXAFMKQRXMGLNDFIQKXXXNXYACKHXEVQSXLXX

SEQ ID NO: 201 - protein, 60 aa, Rattus norvegicus
  rat peptide variant of aa 1-60 of murine Sgk1 (1)..(60)
MTVKTEAARSTLTYSRMRGMVAILIAFMKQRRMGLNDFIQKLANNSYACKHPEVQSYLKI

SEQ ID NO: 202 - protein, 60 aa, Oryctolagus cuniculus
  rabbit peptide variant of aa 1-60 of murine Sgk1 (1)..(60)
MTVKTEAARGPLTYSRMRGMVAILIAFMKQRRMGLNDFIQKIANNSYACKHTEVQSILKI

SEQ ID NO: 203 - protein, 60 aa, Gallus gallus
  chicken peptide variant of aa 1-60 of murine Sgk1 (1)..(60)
MTVKAAEASGPALTYSKMRGMVAILIAFMKQRRMGLNDFIQKIATNSYACKHPEVQSILK

SEQ ID NO: 204 - protein, 60 aa, Danio rerio
  zebrafish peptide variant of aa 1-60 of murine Sgk1 (1)..(60)
MTIQTETSVSAPDLTYSKTRGLVANLSAFMKQRKMGLNDFIQKLSANSYACKHPEVQSIL

SEQ ID NO: 205 - protein, 44 aa, Mus musculus
  aa 17-60 of murine Sgk1 (1)..(44)
MRGMVAILIAFMKQRRMGLNDFIQKIASNTYACKHAEVQSILKM

SEQ ID NO: 206 - protein, 14 aa, Mus musculus
  aa 17-30 of murine Sgk1 (1)..(14)
MRGMVAILIAFMKQ

SEQ ID NO: 207 - protein, 9 aa, Mus musculus
  aa 19-27 of murine Sgk1 (1)..(9)
GMVAILIAF

SEQ ID NO: 208 - protein, 17 aa, Mus musculus
  aa 17-33 of murine Sgk1 (1)..(17)
MRGMVAILIAFMKQRRM

SEQ ID NO: 209 - protein, 7 aa, Mus musculus
  aa 19-25 of murine Sgk1 (1)..(7)
GMVAILI

SEQ ID NO: 210 - protein, 44 aa, Homo sapiens
  aa 17-60 of human Sgk1 (1)..(44)
MRGMVAILIAFMKQRRMGLNDFIQKIANNSYACKHPEVQSILKI

SEQ ID NO: 211 - protein, 210 aa, Saccharomyces cerevisiae
  aa 1-210 of MAT(alpha)2 (1)..(210)
MNKIPIKDLLNPQITDEFKSSILDINKKLFSICCNLPKLPESVTTEEEVELRDILGFLSRANKNRKISDEEKKLLQTTSQLTTTITVLLKEMRSIENDRSNYQLTQKNKSADGLVFNVVTQDMINKSTKPYRGHRFTKENVRILESWFAKNIENPYLDTKGLENLMKNTSLSRIQIKNWVSNRRRKEKTITIAPELADLLSGEPLAKKKE

SEQ ID NO: 212 - protein, 100 aa, Saccharomyces cerevisiae
  aa 1-100 of MAT(alpha)2 (1)..(100)
MNKIPIKDLLNPQITDEFKSSILDINKKLFSICCNLPKLPESVTTEEEVELRDILGFLSRANKNRKISDEEKKLLQTTSQLTTTITVLLKEMRSIENDRS

SEQ ID NO: 213 - protein, 62 aa, Saccharomyces cerevisiae
  aa 1-62 of MAT(alpha)2 (1)..(62)
MNKIPIKDLLNPQITDEFKSSILDINKKLFSICCNLPKLPESVTTEEEVELRDILGFLSRAN

SEQ ID NO: 214 - protein, 62 aa, Artificial Sequence
  variable peptide based on aa 1-62 of MAT(alpha)2
MNKIPIKDLLNPQITDEFKSSILDINKKLFSICCNLPKLPESVTTEEEVELRDILXFLSRAN

SEQ ID NO: 215 - protein, 62 aa, Artificial Sequence
  peptide variant I based on aa 1-62 of MAT(alpha)2
MNKIPIKDLLNPQITDEFKSSILDINKKLFSICCNLPKLPESVTTEEEVELRDILVFLSRAN

SEQ ID NO: 216 - protein, 62 aa, Artificial Sequence
  peptide variant II based on aa 1-62 of MAT(alpha)2
MNKIPIKDLLNPQITDEFKSSILDINKKLFSICCNLPKLPESVTTEEEVELRDILLFLSRAN

SEQ ID NO: 217 - protein, 19 aa, Saccharomyces cerevisiae
  Deg1 Determinant peptide of MAT(alpha)2 (1)..(19)
ITDEFKSSILDINKKLFSI

SEQ ID NO: 218 - protein, 30 aa, Saccharomyces cerevisiae
  Deg1 + heptad repeat peptide of MAT(alpha)2 (1)..(30)
ITDEFKSSILDINKKLFSICCNLPKLPESV

SEQ ID NO: 219 - protein, 165 aa, Saccharomyces cerevisiae
  aa 1-165 of MF(alpha)1 (1)..(165)
MRFPSIFTAVLFAASSALAAPVNTTTEDETAQIPAEAVIGYLDLEGDFDVAVLPFSNSTNNGLLFINTTIASIAAKEEGVSLDKREAEAWHWLQLKPGQPMYKREAEAEAWHWLQLKPGQPMYKREADAEAWHWLQLKPGQPMYKREADAEAWHWLQLKPGQPMY

SEQ ID NO: 220 - protein, 165 aa, Artificial Sequence
  variable peptide based on aa 1-165 of MF(alpha)1
MRFPSIFTAVLFAASSALAAPVXTTTEDETAQIPAEAVIGYLDLEGDFDVAVLPFSXSTNNGLLFIXTTIASIAAKEEGVSLDKREAEAWHWLQLKPGQPMYKREAEAEAWHWLQLKPGQPMYKREADAEAWHWLQLKPGQPMYKREADAEAWHWLQLKPGQPMY

SEQ ID NO: 221 - protein, 165 aa, Saccharomyces cerevisiae
  peptide variant I based on aa 1-165 of MF(alpha)1 (1)..(165)
MRFPSIFTAVLFAASSALAAPVQTTTEDETAQIPAEAVIGYLDLEGDFDVAVLPFSQSTNNGLLFIQTTIASIAAKEEGVSLDKREAEAWHWLQLKPGQPMYKREAEAEAWHWLQLKPGQPMYKREADAEAWHWLQLKPGQPMYKREADAEAWHWLQLKPGQPMY

SEQ ID NO: 222 - protein, 165 aa, Saccharomyces cerevisiae
  peptide variant II based on aa 1-165 of MF(alpha)1 (1)..(165)
MRFPSIFTAVLFAASSALAAPVNTTTEDETAQIPAEAVIGYLDLEGDFDVAVLPFSNSTNNGLLFIQTTIASIAAKEEGVSLDKREAEAWHWLQLKPGQPMYKREAEAEAWHWLQLKPGQPMYKREADAEAWHWLQLKPGQPMYKREADAEAWHWLQLKPGQPMY

SEQ ID NO: 223 - protein, 165 aa, Saccharomyces cerevisiae
  peptide variant III based on aa 1-165 of  MF(alpha)1 (1)..(165)
MRFPSIFTAVLFAASSALAAPVQTTTEDETAQIPAEAVIGYLDLEGDFDVAVLPFSNSTNNGLLFINTTIASIAAKEEGVSLDKREAEAWHWLQLKPGQPMYKREAEAEAWHWLQLKPGQPMYKREADAEAWHWLQLKPGQPMYKREADAEAWHWLQLKPGQPMY

SEQ ID NO: 224 - protein, 523 aa, Saccharomyces cerevisiae
  aa 1-523 of CPY (1)..(523)
MILHTYIILSLLTIFPKAIGLSLQMPMALEASYASLVEKATLAVGQEIDAIQKGIQQGWLEVETRFPTIVSQLSYSTGPKFAIKKKDATFWDFYVESQELPNYRLRVKRNNPEVLKVDFTKQYSGYLDVEADDKHFFYWFFESRNDPQNDPIILWLNGGPGCSSLTGLFFELGSSRINENLKPIFNPYSWNGNASIIYLDQPVNVGFSYSSSSVSNTVVAGEDVYAFLQLFFQHFPEYQTNDFHIAGESYAGHYIPVFADEILSQKNRNFNLTSVLIGNGLTDPLTQYRYYEPMACGEGGAPSVLPADECENMLVTQDKCLSLIQACYDSQSAFTCAPAAIYCNNAQMGPYQRTGKNVYDIRKECDGGSLCYKDLEFIDTYLNQKFVQDALGAEVDTYESCNFEINRNFLFAGDWMKPYHEHVSSLLNKGLPVLIYAGDKDFICNWLGNRAWTDVLPWVDADGFEKAEVQDWLVNGRKAGEFKNYSNFTYLRVYDAGHMAPYDQPENSHEMVNRWISGDFSFH

SEQ ID NO: 225 - protein, 28 aa, Ricinus communis
  aa 240-267 of ricin (1)..(28)
FSVYDVSILIPIIALMVYRCAPPPSSQF

SEQ ID NO: 226 - protein, 33 aa, Vibrio cholerae
  aa 180-212 of cholera toxin (1)..(33)
YGLAGFPPEHRAWREEPWIHHAPPGCGNAPRSS

SEQ ID NO: 227 - protein, 28 aa, Shigella dysenteriae
  aa 245-272+C202 of Stx1A (1)..(28)
ISFNNISAILGTVAVILNCHHQGARSVR

SEQ ID NO: 228 - protein, 28 aa, Escherichia coli
  aa 245-272 of Slt-II (1)..(28)
ISFNNISAILGTVAVILNCHHQGARSVR

SEQ ID NO: 229 - protein, 251 aa, Shigella dysenteriae
  Shiga toxin (Stx) A1 chain (1)..(251)
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAR

SEQ ID NO: 230 - protein, 251 aa, Enterobacteria phage H19B
  Shiga like toxin SLT-Ia A1 chain (1)..(251)
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAR

SEQ ID NO: 231 - protein, 28 aa, Escherichia coli
  aa 224-251 of Slt-1A1 (1)..(28)
ISFGSINAILGSVALILNCHHHASRVAR

SEQ ID NO: 232 - protein, 22 aa, Escherichia coli
  aa 224-245 of Slt-1A1 (1)..(22)
ISFGSINAILGSVALILNCHHH

SEQ ID NO: 233 - protein, 17 aa, Escherichia coli
  aa 224-240 of Slt-1A1 (1)..(17)
ISFGSINAILGSVALIL

SEQ ID NO: 234 - protein, 315 aa, Escherichia coli
  SLT-Ic A chain (1)..(315)
MKIIIFRVLTFFFVIFSVNVVAKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSVNAILGSVALILNCHHHASRVARIVPNEFPSMCPVDGRVRGITHNKILWDSSTLGAILIRRAISS

SEQ ID NO: 235 - protein, 250 aa, Enterobacteria phage 933W
  SLT-IIb A1 chain (1)..(250)
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 236 - protein, 319 aa, Escherichia coli
  SLT-IId A chain (1)..(319)
MKCILFKWVLCLLLGFSSVSYSREFTIDFSTQQSYVSSLNSIRTEISTPLEHISQGTTSVSVINHTPPGSYFAVDIRGLDVYQARFDHLRLIIEQNNLYVAGFVNTATNTFYRFSDFAHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEESQPECQITGDRPVIKINNTLWESNTAAAFLNRKSQSLYTTGE

SEQ ID NO: 237 - protein, 319 aa, Escherichia coli
  SLT-IIe A chain (1)..(319)
MKCILLKWILCLLLGFSSVSYSQEFTIDFSTQQSYVSSLNSIRTAISTPLEHISQGATSVSVINHTPPGSYISVGIRGLDVYQERFDHLRLIIERNNLYVAGFVNTTTNTFYRFSDFAHISLPGVTTISMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEAGVRVGRISFNNISAILGTVAVILNCHHQGARSVRAVNEESQPECQITGDRPVIKINNKLWESNTAAAFLNRKSQPLYTTGE

SEQ ID NO: 238 - protein, 319 aa, Escherichia coli
  SLT-IIf A chain (1)..(319)
MRHILLKLVLFFCVCLSSASYADEFTVDFSSQKSYVDSLNSIRSAISTPLGNISQGGVSVSVINHVPGGNYISLNVRGLDPYSERFNHLRLIMERNNLYVAGFINTETNTFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVRSVSQKQKTECQIVGDRAAIKVNNVLWEANTIAALLNRKPQDLTEPNQ

SEQ ID NO: 239 - protein, 240 aa, Escherichia coli
  E. coli Heat-labile enterotoxin LT A chain (human strain), A chain without signal peptide (1)..(240)
NGDKLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRTITGDTCNEETQNLSTIYLRKYQSKVKRQIFSDYQSEVDIYNRIRNEL

SEQ ID NO: 240 - protein, 240 aa, Escherichia coli
  E. coli Heat-labile enterotoxin LT A chain (porcine strain), A chain without signal peptide (1)..(240)
NGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRTITGDTCNEETQNLSTIYLREYQSKVKRQIFSDYQSEVDIYNRIRDEL

SEQ ID NO: 241 - protein, 241 aa, Escherichia coli
  E. coli Heat-labile enterotoxin LT-IIa A chain, A chain without signal peptide (1)..(241)
NDFFRADSRTPDEIRRAGGLLPRGQQEAYERGTPININLYEHARGTVTGNTRYNDGYVSTTVTLRQAHLIGQNILGSYNEYYIYVVAPAPNLFDVNGVLGRYSPYPSENEFAALGGIPLSQIIGWYRVSFGAIEGGMQRNRDYRGDLFRGLTVAPNEDGYQLAGFPSNFPAWREMPWSTFAPEQCVPNNKEFKGGVCISATNVLSKYDLMNFKKLLKRRLALTFFMSEDDFIGVHGERDEL

SEQ ID NO: 242 - protein, 31 aa, Escherichia coli
  aa 178-202 of C166LT-II (1)..(31)
YQLAGFPSNFPAWREMPWSTFAPEQCVPNNK

SEQ ID NO: 243 - protein, 243 aa, Escherichia coli
  E. coli Heat-labile enterotoxin LT-IIb A chain, A chain without signal peptide (1)..(243)
NDYFRADSRTPDEVRRSGGLIPRGQDEAYERGTPININLYDHARGTATGNTRYNDGYVSTTTTLRQAHLLGQNMLGGYNEYYIYVVAAAPNLFDVNGVLGRYSPYPSENEYAALGGIPLSQIIGWYRVSFGAIEGGMHRNRDYRRDLFRGLSAAPNEDGYRIAGFPDGFPAWEEVPWREFAPNSCLPNNKASSDTTCASLTNKLSQHDLADFKKYIKRKFTLMTLLSINNDGFFSNNGGKDEL

SEQ ID NO: 244 - protein, 24 aa, Artificial Sequence
  Cx2a module (b) + (c)
NASSSRSGLDDINPTVLLKAKDEL

SEQ ID NO: 245 - protein, 24 aa, Artificial Sequence
  IgM(mu) peptide +  KDEL peptide
GKPTLYQVSLIMSDTGGTSYKDEL

SEQ ID NO: 246 - protein, 7 aa, Simian virus 40
  nuclear localization signal peptide of SV40 Large T-antigen (1)..(7)
PKKKRKV

SEQ ID NO: 247 - protein, 16 aa, Unknown
  nuclear localization signal peptide of nucleoplasmin
KRPAATKKAGQAKKKK

SEQ ID NO: 248 - RNA, 21 nt, Artificial Sequence
  GAPDH targeted siRNA - sense strand
ccamcuuccaggagcyagamm

SEQ ID NO: 249 - RNA, 21 nt, Artificial Sequence
  GAPDH targeted siRNA - antisense strand
ucucgcuccugyaagamggdd

SEQ ID NO: 250 - protein, 14 aa, Artificial Sequence
  variable Tet1-flexible linker peptide
HLNILSTLWKYRXC

SEQ ID NO: 251 - protein, 116 aa, Unknown
  anti-EGF-R single chain antibody
QVQLVESGGGLVQPGGSLRLPCAASGSIFSLDAWGWYRQAPGKQREMVALVGSDGSTSYADSVKGRFTISRDNANNTFYLQMNSLKPEDTAVYYCYARFQSLYNSWGQGTQVTVSS

SEQ ID NO: 252 - protein, 679 aa, Homo sapiens
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 253 - protein, 71 aa, Artificial Sequence
  DRBD peptide with N-terminal Cys
CFFMEELNTYRQKQGVVLKYQELPNSGPPHDRRFTFQVIIDGREFPEGEGRSKKEAKNAAAKLAVEILNKE

SEQ ID NO: 254 - protein, 71 aa, Artificial Sequence
  DRBD peptide with C-terminal Cys
FFMEELNTYRQKQGVVLKYQELPNSGPPHDRRFTFQVIIDGREFPEGEGRSKKEAKNAAAKLAVEILNKEC

SEQ ID NO: 255 - RNA, 21 nt, Artificial Sequence
  fLuc targeted siRNA- sense strand
cuuacycugagmacuucgamm

SEQ ID NO: 256 - RNA, 21 nt, Artificial Sequence
  fLuc targeted siRNA - antisense strand
ucgaaguacucaycguaaydd

SEQ ID NO: 257 - RNA, 21 nt, Artificial Sequence
  non-silencing siRNA control - sense
ggamcuuauuucumcggagmm

SEQ ID NO: 258 - RNA, 21 nt, Artificial Sequence
  non-silencing siRNA control - antisense
cuccgaagaaamaagamccdd

SEQ ID NO: 259 - DNA, 19 bp, Unknown
  GAPDH siRNA target nucleic acid
ggtcatccatgacaacttt

SEQ ID NO: 260 - DNA, 23 bp, Artificial Sequence
  Gene Racer 5′ primer
cgactggagcacgaggacactga

SEQ ID NO: 261 - DNA, 28 bp, Artificial Sequence
  GAPDH 3′ primer
acgcctgcttcaccaccttcttgatgtc

SEQ ID NO: 262 - DNA, 26 bp, Artificial Sequence
  GAPDH 5′ nested primer
ggacactgacatggactgaaggagta

SEQ ID NO: 263 - DNA, 28 bp, Artificial Sequence
  GAPDH 3′ nested primer
aggccatgccagtgagcttcccgttcag

SEQ ID NO: 264 - RNA, 21 nt, Artificial Sequence
  VEGF targeted siRNA - sense
ggaguacccugaugagaucdd

SEQ ID NO: 265 - RNA, 21 nt, Artificial Sequence
  VEGF targeted siRNA - antisense
gaucucaucaggguacuccdd

SEQ ID NO: 266 - RNA, 21 nt, Artificial Sequence
  Bcl-xL targeted siRNA - sense
gguauuggugagucggaucdd

SEQ ID NO: 267 - RNA, 21 nt, Artificial Sequence
  Bcl-xL targeted siRNA - antisense
gauccgacucaccaauaccdd

SEQ ID NO: 268 - DNA, 21 bp, Artificial Sequence
  KDELR-1 targeted siRNA - sense
cuaccucuauaucaccaaatt

SEQ ID NO: 269 - RNA, 21 nt, Artificial Sequence
  KDELR-1 targeted siRNA - antisense
uuuggugauauagagguagaa

SEQ ID NO: 270 - DNA, 21 bp, Artificial Sequence
  KDELR-2 targeted siRNA - sense
auaggagcaggcaagguagat

SEQ ID NO: 271 - DNA, 21 bp, Artificial Sequence
  KDELR-2 targeted siRNA - antisense
cuaccuugccugcuccuautt

SEQ ID NO: 272 - RNA, 21 nt, Artificial Sequence
  KDELR-3 targeted siRNA - sense
acugauuccagauagauagag

SEQ ID NO: 273 - DNA, 21 bp, Artificial Sequence
  KDELR-3 targeted siRNA - antisense
cuaucuaucuggaaucagutt

SEQ ID NO: 274 - DNA, 21 bp, Artificial Sequence
  Sec61a targeted siRNA - sense
ggaauuugccugcuaaucatt

SEQ ID NO: 275 - RNA, 21 nt, Artificial Sequence
  Sec61a targeted siRNA - antisense
ugauuagcaggcaaauuccag

SEQ ID NO: 276 - DNA, 21 bp, Artificial Sequence
  Derlin-1 targeted siRNA - sense
gcuuagcaauggauaugcatt

SEQ ID NO: 277 - RNA, 21 nt, Artificial Sequence
  Derlin-1 targeted siRNA - antisense
ugcauauccauugcuaagcca

SEQ ID NO: 278 - DNA, 21 bp, Artificial Sequence
  PDIA2 targeted siRNA - sense
gucggaaggugauugaauatt

SEQ ID NO: 279 - DNA, 21 bp, Artificial Sequence
  PDIA2 targeted C239siRNA - antisense
uauucaaucaccuuccgacct

SEQ ID NO: 280 - DNA, 21 bp, Artificial Sequence
  Ero1L targeted siRNA - sense
ggaaugucaucuacgaagatt

SEQ ID NO: 281 - DNA, 21 bp, Artificial Sequence
  Ero1L targeted siRNA - antisense
ucuucguagaugacauuccat

SEQ ID NO: 282 - protein, 267 aa, Ricinus communis
  Ricin A chain without signal and linker peptide (1)..(266)
IFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQF

SEQ ID NO: 283 - protein, 194 aa, Vibrio cholerae
  Cholera toxin subunit A1 (1)..(194)
NDDKLYRADSRPPDEIKQSGGLMPRGQSEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSS

(19) United States
(12) Patent Application Publication
(10) Pub. No.: US 2012/0258104 A1
(43) Pub. Date: Oct. 11, 2012

(54) Delivery System and Conjugates For Compound Delivery Via Naturally Occurring Intracellular Transport Routes

(75) Inventors: Christophe J. Echeverri, Roseville, MN (US); Birte Sonnichsen, Dresden (DE); Reinhard Wähler, Berlin (DE); Mike Werner Helms, Dresden (DE)

(73) Assignee: Cenix Bioscience GMBH, Dresden (DE)

(21) Appl. No.: 13/386,347

(22) PCT Filed: Jul. 22, 2010
(86) PCT No.: PCT/EP10/04512

§ 371 Date: Jun. 25, 2012

Related U.S. Application Data
(60) Provisional application No. 61/227,669, filed on Jul. 22, 2009.

(51) Int. Cl. C07K 19/00; A61K 38/02; C12N 5/071; C12N 1/14; C12N 1/16; C12N 1/18; C12N 5/04; A61K 39/395; C12N 5/07; B82Y 5/00

(52) U.S. Cl. 424/134.1; 530/300; 530/387.3; 530/322; 514/1.1; 514/20.9; 435/375; 435/254.1; 435/256.1; 435/255.1; 435/255.2; 435/255.5; 435/348; 435/358; 435/363; 435/366; 435/410; 977/773

(57) ABSTRACT

The present invention relates to a delivery system that comprises a conjugate that facilitates the delivery of a compound such as a biologically-active macromolecule, a nucleic acid or a peptide in particular, into a cell. The present invention also relates to said conjugate for delivery of a compound, such as a biologically-active macromolecule, a nucleic acid or a peptide, into a cell. The present invention further relates to a pharmaceutical composition comprising said conjugate and to its use. The present invention also relates to a method of delivering a compound to a cell or an organism, preferably a patient. The conjugates comprise: (a) at least one module that mediates cell targeting and facilitates cellular uptake, (b) at least one module that facilitates transport to the endoplasmic reticulum (ER), (c) at least one module that mediates translocation from the ER to the cytosol, and (d) at least one compound to be delivered wherein the modules (a) to (c) and the compound (d) are linked to each other in any arrangement.

The present invention relates to a delivery system that comprises a conjugate that facilitates the delivery of a compound such as a biologically-active macromolecule, a nucleic acid or a peptide in particular, into a cell. The present invention also relates to said conjugate for delivery of a compound, such as a biologically-active macromolecule, nucleic acid or peptide, into a cell. The present invention further relates to a pharmaceutical composition comprising said conjugate and to its use. The present invention also relates to a method of delivering a compound to a cell or organism, such as a patient.

### BACKGROUND OF THE INVENTION

New therapies are under development, which seek to address diseased states at the molecular level. A major problem in the practical application of many of these new therapeutic compounds is that the compounds do not readily cross cellular membranes and, thus, cannot reach compartments within the cell where their sites of action may reside.

The inability of most large molecules to efficiently cross the plasma membrane of animal cells has typically restricted their application for research and therapeutic purposes to those involving mechanisms of action occurring outside of the cells, most often through interactions on the cell surface. However, certain types of biologically-active macromolecules, such as antisense oligonucleotides, ribozymes, RNAi-inducing nucleic acid duplexes such as siRNAs and longer nucleic acids such as plasmids, must be present within intracellular compartments such as the cytosol or the nucleus to produce their intended biological effects. Unfortunately, in addition to the problem posed by the high net charges typically carried by such molecules for getting across the hydrophobic environment of cellular membranes, their overall size also greatly exceeds the upper limits, generally estimated at around 500 Da, of what can readily diffuse across those membranes unassisted. As such, the utility of these molecules for both research and therapeutic applications is strongly dependent on the use of delivery technologies designed to facilitate their efficient accumulation at their intended site of activity.

While in vitro applications in cultured cells require this delivery process to also include the transfer of the macromolecules intact through the growth medium, in vivo applications in living animals often impose a more challenging path. This starts with introduction into the body, continues with passage through various body fluids, tissues and structures, any of which may present significant chemical or physical barriers, and ends with eventual entry into the targeted cells to reach the intended site of action. For the in vivo context, this process also implies the need to avoid or at least delay excretion out of the body long enough to allow useful amounts of uptake into targeted cells. In all contexts, the delivery solution must also minimize undesired modifications either to the introduced molecules, or to any of the tissues, fluids, structures and cells encountered along the way. For example, many lipid-based nanoparticles and liposomal formulations are significantly limited in their applicability by their restricted bio-distribution (accumulating primarily in the liver) and their inherent risks for causing cytotoxic effects [1].

In some cases, minimizing risks of undesirable secondary effects can also imply preventing unwanted interactions of the delivered macromolecules with unintended binding partners along the way. Examples of this include unspecific immune stimulation that can be unintentionally triggered by certain nucleic acid constructs. While some delivery technologies help to resolve this problem by physically shielding or encapsulating the macromolecule during transit and only releasing it or activating it at the appropriate time/location (see, for example, WO 2009/045457), others lack this functionality and rely on optimization of the molecule itself to address this issue. In the case of siRNAs and other RNAi-inducing agents, the latter has indeed been possible, both by avoiding sequence motifs known to bear higher risks of immune stimulation, and through chemical alterations to the nucleic acid backbone, which render such molecules poor substrates for unintended pathways [such as Toll Like Receptor (TLR)-based immune responses], while preserving maximal activity with the targeted machinery [such as the RNA-induced Silencing Complex (RISC)].

Ultimately, once the delivery vehicle has successfully brought its cargo to the surface of the targeted cells, it still faces one of the most formidable barriers common to all delivery paths, i.e. the targeted cell's plasma membrane, through which, as noted above, large and/or highly charged macromolecules typically cannot pass unassisted. While some delivery technologies attempt to address this by triggering cellular uptake through natural internalization processes such as endocytosis, pinocytosis or phagocytosis, all such currently-available solutions only delay the problem without actually solving it, since access to the cytosol will still require the same membrane to be crossed from within the resulting endocytic, pinocytic or phagocytic vesicles. Indeed, the successful crossing of this crucial biological membrane, whether it occurs on the cell surface or from within such intracellular vesicles, has proven to be a particularly challenging and rate-limiting step for virtually all delivery technologies tested to date.

One common approach to addressing this challenge has been to take advantage of the acidification process that virtually all cells naturally drive inside many newly-internalized vesicles of endocytic, pinocytic or phagocytic origin, typically as these get sorted towards a lysosomal fate. To this end, these delivery technologies integrate various molecules, which carry a pH-dependent ability to “force” the destabilization or permeabilization of these vesicular membranes under appropriately acidic conditions, and hopefully before the delivered molecules get damaged in the lysosome. Sometimes referred to as “endosomolytic activity”, this form of endosomal escape has been realized through several different strategies in recent years [discussed in US 2008/0200661 A1, including the inclusion of fusogenic lipids within liposomes and so-called stable nucleic acid lipid particles (SNALPs)]. Another example makes use of so-called peptide transduction domains (PTDs) derived from various proteins that have naturally evolved to mediate the transfer of macromolecules or even larger cargo such as entire viruses across cellular membranes, including some known to become activated by acidication of the endosome (US 2006/0222657 A1). A third notable example has been the use of PBAVE, an amphipathic poly(vinyl ether) whose endosomolytic activity was reversibly shielded by PEG groups linked via acid-labile maleamate bonds [2, and US 2007/0036865 A1). However, despite the variable successes noted with such technologies to date, their “forced endosomal escape” processes still represent the key rate-limiting step in most, if not all, of these solutions, thus indicating that these approaches have still not met this challenge optimally.

Finally, an important but often-overlooked issue in designing delivery solutions is the question of what happens to the delivery vehicle or construct once it has completed its mission. The possibility that these delivery molecules will fail to be metabolized and will thus accumulate within the targeted cells imposes a further requirement on the design of these molecules, especially in the context of repeated or sustained long-term treatments. In particular, the components used within the delivery vehicles or constructs should not cause any deleterious effects in this context. As a result, delivery molecules that are known to be readily and safely metabolized by targeted cells present a preferred solution, whereas those making use of artificial, non-biodegradable chemistries or molecules whose long-term effects have not been adequately characterized present increased risks.

Thus, there is an urgent need for a delivery system that can efficiently deliver compounds such as biologically-active macromolecules, nucleic acids or peptides in particular, into living cells. There is also an urgent need for a delivery system that does not cause any deleterious side effects within the cell. A delivery system that utilizes components that are readily and safely metabolized by targeted cells would also be highly desirable.

### SUMMARY OF THE INVENTION

The present invention relates to a delivery system that comprises a conjugate that facilitates the delivery of a compound such as a biologically-active macromolecule, a nucleic acid or a peptide in particular, into living cells of interest, preferably into the cytosol or nucleus of said living cells of interest. The delivery systems and conjugates of the present invention are designed to harness and/or exploit fully natural pathways for initial cell targeting and internalization, followed by retrograde transport through membranous compartments to the endoplasmic reticulum (ER) and retro-translocation from the ER to the cytosol via the ER-associated degradation pathway (ERAD). Upon reaching the cytosol, the delivery systems and conjugates of the present invention may either deliver a compound to the cytosol or continue on to deliver a compound to the nucleus.

As such, the present invention provides delivery systems and conjugates which can effectively deliver compounds such as biologically active macromolecules, nucleic acids or peptides in particular, to a targeted cytosol or nucleus by using endogenous processes that occur ubiquitously within all cells. The conjugates of the present invention maximally utilize and exploit the benefits of these endogenous processes, which are fully natural and evolutionary optimized and thus, the delivery systems and conjugates are able to deliver compounds with high efficiency, low toxicity and a broad range of application into target cells. The delivery systems and conjugates provided by the present invention allow the effective delivery of biologically active compounds into both cultured cells and living organisms, for research, therapeutic and diagnostic purposes. The conjugates provided by the present invention are designed to be degraded and therefore, not accumulate within the targeted cells. Thus, the delivery systems and the conjugates of the present invention provide at least a solution to the cytosol delivery problem in the art as well as a solution to the toxicity problems in the art that result from accumulation of non-metabolized or undegraded delivery vehicles/constructs in the targeted cell.

In a first aspect, the present invention relates to a delivery system for delivery of a compound into a cell comprising or consisting of at least one conjugate comprising, essentially consisting of or consisting of:
(a) at least one module (a) that mediates cell targeting and facilitates cellular uptake,
(b) at least one module (b) that facilitates transport to the endoplasmic reticulum (ER),
(c) at least one module (c) that mediates translocation from the ER to the cytosol, and
(d) at least one compound (d),
   wherein the modules (a), (b) and (c), and the compound (d) are linked to each other in any arrangement. The delivery systems of the present invention optionally comprise a nuclear localization signal.

In a second aspect, the present invention relates to a conjugate for delivery of a compound into a cell comprising, essentially consisting of or consisting of:
(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
   wherein the modules (a), (b) and (c), and the compound (d) are linked to each other in any arrangement. The conjugates of the present invention optionally comprise a nuclear localization signal.

In a third aspect, the present invention relates to methods of preparing a delivery system or conjugate of the invention.

In a fourth aspect, the present invention relates to the use of the delivery system or conjugate of the invention as a pharmaceutical.

In a fifth aspect, the present invention relates to a pharmaceutical composition comprising the delivery system or conjugate of the present invention and a pharmaceutically acceptable excipient, carrier, and/or diluent.

In a sixth aspect, the present invention relates to the use of a delivery system or conjugate of the invention as a diagnostic reagent.

In a seventh aspect, the present invention relates to a use of the delivery system or conjugate of the invention for the manufacture of a medicament.

In an eighth aspect, the present invention relates to a method of delivering the compound (d) to a cell using the delivery system or conjugate of the invention.

In a ninth aspect, the present invention relates to a method of delivering the compound (d) to an organism using the delivery system or conjugate of the invention.

In a tenth aspect, the present invention relates to a method of delivering the compound (d) to a patient using the delivery system or conjugate of the invention.

### BRIEF DESCRIPTION OF THE DRAWINGS

FIG. 1 (A) to (D). (A), (B), (C), and (D) contain preferred embodiments of the conjugate of the present invention. The modules, or the modules and the compound may be linked to each other either covalently, non-covalently, via an adapter molecule or via a linker molecule that optimally comprises an adapter molecule.

FIG. 2 (A and B). Detailed drawing of conjugate R-AK-CX described in Example 1. (A) illustrates a conjugate of the present invention, in which the cell targeting/uptake peptide [module (a)] is ricin toxin subunit B, the ERAD targeting/sorting peptide [module (c)] is from COX2, the ER targeting peptide [module (b)] is AKDEL, and the cargo [compound (d)] is an siRNA. The RTb is connected by a biodegradable disulfide bond to the N-terminus of the linkage peptide which carries modules (c) and (b) at the carboxy end. The siRNA cargo is linked, via the 5′-end of the sense strand containing a biodegradable (reducible) disulfide bond and an aminolinker, to the linkage peptide through an adapter derived from succinimidyl 4-formylbenzoate. The connection is made through a stable oxime bond generated by reaction of the formyl group with the aminoxy group of the branch point N-beta-aminoxyacetyl L-diaminopropionyl residue. The (SG)₃ units function as spacers to ensure that the various modules do not interfere with one another. (B) Illustrates the same molecule as described in FIG. 2 (A), but which includes a fluorescent dye at the 5′-end of the sense strand of the siRNA, to allow detection of the siRNA once it is released into the cytosol of the cell.

FIG. 3 (A) to (E). (A) illustrates a conjugate according to the present invention, wherein the modules and compound (d) are linked to each other in the following arrangement: module (a) is covalently linked to module (c) via a peptide linker molecule that comprises a cysteine side chain as branch point and a cleavage site upstream of the branch point, module (c) is covalently linked to module (b), and compound (d) is covalently linked via a disulfide-linkage to the cysteine side chain. (B) illustrates a conjugate according to the present invention, wherein the modules and compound (d) are linked to each other in the following arrangement: module (a) is covalently linked to module (c) via a first peptide linker molecule which comprises a cysteine side chain as branch point and a cleavage site upstream of the branch point, module (c) is covalently linked to module (b) via a second peptide linker molecule, and compound (d) is covalently linked via a disulfide-linkage to the cysteine side chain of the branch point. (C) illustrates another preferred embodiment, wherein compound (d) is linked via an enzymatic cleavage site instead of a disulfide-linkage to a cysteine side chain. Preferably, module (a) is cleaved off of the conjugate in the endosome or TGN, whereby making module (b) available for cellular receptors or other cellular proteins that bind to cellular receptors and then facilitate further transport to the ER. (D) illustrates a conjugate according to the present invention, wherein the at least one module (a), the at least one module (b), the at least module (c) and the at least one compound (d) are linked to each other in the following arrangements: the at least one module (a) is covalently linked to the at least one module (c) via a peptide linker molecule which comprises a cysteine side chain as a branch point and a cleavage site upstream of the branch point, the at least one module (c) is covalently linked to the at least one module (b) and the at least one compound (d) is non-covalently linked to the branch point via an ionic (electrostatic) linkage to DRBD that is covalently linked via a disulfide-linkage to the cysteine side chain. (E) illustrates a conjugate according to the present invention, wherein the modules and the compound are linked to each other in the following arrangement or combination: module (a) is covalently linked to module (c) via a peptide linker molecule which comprises a cysteine side chain as branch point and a cleavage site upstream of the branch point, module (c) is covalently linked to module (b) via a peptide linker molecule and compound (d) is non-covalently linked to the branch point via an ionic linkage to DRBD that is covalently linked via a disulfide-linkage to the cysteine side chain.

FIG. 4. Illustrates a conjugate of the present invention, in which module (a) is the non-toxic ricin toxin subunit B, RTb, the module (b) does not exist as a separate module but is part of RTb and module (c) does not exist as a separate module but is provided by part of RTb. Generally, 1-4 siRNAs as compound(s) (d) can be coupled to each RTB molecule via accessible amino groups such as those on lysine side chains plus the N-terminal amino group. The construct depicted in this Figure is referred to as DARE™ 1.01/DARE-R1/RTB-siRNA (via Lys). Briefly, the free thiol at Cys-4 is first inactivated by treatment with N-ethylmaleimide and the RTb is activated by reaction with an excess of a bifunctional crosslinker, e.g., sulfo-LC-SMPT, that contains an activated disulfide. Treatment of this intermediate with siRNA with a free thiol on the 5′-terminus of the antisense strand generates the conjugate illustrated by a simple disulfide exchange reaction. The location and number of siRNA coupling is not limited to the example shown in this Figure. Since RTB is activated with an excess of the bifunctional crosslinker sulfo-LC-SPDP (or sulfo-LC-SMPT), several molecules of siRNA per RTB monomer can be added. Separation of the entities with multiple siRNAs attached can be done by anion-exchange HPLC. The “N”s in the figure are only exemplary and do not represent actual locations of free amino side groups (except for the N-terminus).

FIG. 5. Illustrates a conjugate of the present invention, in which module (a) is the non-toxic ricin toxin subunit B, RTb, the module (b) does not exist as a separate module but is part of RTb and module (c) does not exist as a separate module but is provided as part of RTb. The cargo, compound (d), is an siRNA directly coupled via the 5′-end of the sense strand to the cysteine residue at position 4 of the RTb molecule through a biodegradable (reducible) disulfide bond. The construct depicted in this Figure is referred to as DARE™ 1.02/DARE-R2/RTB-siRNA (via Cys).

FIG. 6 (A and B). (A) illustrates a conjugate of the present invention, in which the cell targeting/uptake peptide, module (a), is ricin toxin subunit B, the ERAD targeting/sorting peptide, module (c), is from COX2, the ER targeting functionality of module (b) is provided by RTb, and the cargo, compound (d), is an siRNA. The RTb is connected by a biodegradable disulfide bond to a cysteine residue at the N-terminus of the linkage peptide which carries module (c) at the C-terminus. The siRNA cargo is linked, via the 5′-end of the sense strand containing a biodegradable (reducible) disulfide bond and an aminolinker, to the linkage peptide through an adapter derived from succinimidyl 4-formylbenzoate. The connection is made through a stable oxime bond generated by reaction of the formyl group with the aminoxy group of the branch point N-beta-aminoxyacetyl L-diaminopropionyl residue. The (SG)₃ units function as spacers to ensure that the various modules do not interfere with one another. The construct depicted in this Figure is referred to as DARE™-2.01/DARE-R-CX/RTB-Cox2-ERSTEL-siRNA (B) illustrates the same molecule as described in FIG. 6 (A) but the (SG)₃ spacers are replaced by PEG spacers. The synthesis is described in Example 2. The construct depicted in this Figure is referred to as DARE™-2.02/DARE-R-CXpeg/RTB-peg-Cox2-ERSTEL-siRNA.

FIG. 7. Illustrates a conjugate of the present invention, in which the cell targeting/uptake protein or peptide, module (a), is ricin toxin subunit B, the ERAD targeting/sorting peptide, module (c), is from COX2, the ER targeting peptide, module (b), is KDEL, and the cargo, compound (d), is an siRNA. The RTb is connected by a biodegradable disulfide bond to the N-terminus of the linkage peptide which carries modules (c) and (b) at the C-terminus. The siRNA cargo is linked via the 5′-end of the sense strand containing a biodegradable (reducible) disulfide bond and an aminolinker, to the linkage peptide through an adapter derived from succinimidyl 4-formylbenzoate. The connection is made through a stable oxime bond generated by reaction of the formyl group with the aminoxy group of the branch point N-beta-aminoxyacetyl L-diaminopropionyl residue. The (SG)₃ units function as spacers to ensure that the various modules do not interfere with one another. The construct depicted in this Figure is referred to as DARE™-2.03/DARE-R-AK-CX/RTB-Cox2-AKDEL-siRNA.

FIG. 8. Illustrates a conjugate of the present invention identical to that illustrated in FIG. 7, with the exception that module (c), the ERAD targeting peptide, is omitted. The construct depicted in this Figure is referred to as DARE™-2.04/DARE-R-AK/RTB-AKDEL-siRNA.

FIG. 9. Illustrates a conjugate of the present invention, in which the cell targeting/uptake peptide, module (a), is ricin toxin subunit B, the BRAD targeting/sorting peptide, module (c), is from Sgk1, and the ER targeting peptide, module (b), is KDEL, and the cargo, compound (d), is an siRNA. The RTb is connected by a biodegradable disulfide bond to a cysteine residue at the N-terminus of the linkage peptide which carries modules (b) and (c). The siRNA cargo is linked, via the 5′-end of the sense strand containing a biodegradable (reducible) disulfide bond and an aminolinker, to the linkage peptide through an adapter derived from succinimidyl 4-formylbenzoate. The connection is made through a stable oxime bond generated by reaction of the formyl group with the aminoxy group of the branch point N-beta-aminoxyacetyl L-diaminopropionyl residue. The (SG)₃ units function as spacers to ensure that the various modules do not interfere with one another. The construct depicted in this Figure is referred to as DARE™ 2.05/DARE-R-AK-SGK/RTB-Sgk1-AKDEL-siRNA.

FIG. 10 (A and B). (A) illustrates a conjugate of the present invention, in which module (a) is a transferrin receptor binding peptide, module (b) is KDEL and module (c) is a Cox2 peptide. All three modules are linked as a contiguous peptide. The (SG)₃ units function as spacers to ensure that the various modules do not interfere with one another. Compound (d) is an siRNA. The siRNA cargo is linked, via the 5′-end of the sense strand containing a biodegradable (reducible) disulfide bond to a cysteine residue of the peptide, located between the two (SG)₃ spacers. The construct depicted in this Figure is referred to as DARE™-3.01a/DARE-T-AK-CX_NC/TfR-Cox2-AKDEL-siRNA (N→C). (B) illustrates a conjugate of the present invention, in which the modules are the same as in FIG. 10 (A) however the construct is such that both modules (a) and (b) have their C-termini free. Module (a) is connected via its N-terminus to the branch point N-beta-aminoxyacetyl L-diaminopropionyl residue via a disulfide bond formed from 2 cysteine residues. Compound (d) is an siRNA. The siRNA cargo is linked, via the 5′-end of the sense strand containing an aminolinker, to the linkage peptide through an adapter derived from succinimidyl 4-formylbenzoate. The connection is made through a stable oxime bond generated by reaction of the formyl group of the adapter with the aminoxy group of the branch point N-beta-aminoxyacetyl L-diaminopropionyl residue. The (SG)₃ units function as spacers to ensure that the various modules do not interfere with one another. The construct depicted in this Figure is referred to as DARE™-3.01b/DARE-T-AK-CX_CC/TfR-Cox2-AKDEL-siRNA (→C; →C).

FIG. 11. Illustrates a conjugate of the present invention, in which module (a) is a transferrin receptor binding peptide, module (b) is KDEL and module (c) is an Sgk1 peptide. All three modules are linked as a contiguous peptide, with module (c) at the N-terminus and module (b) at the C-terminus. The (SG)₃ units function as spacers to ensure that the various modules do not interfere with one another. Compound (d) is an siRNA and is linked via the 5″-end of the sense strand through a biodegradable (reducible) disulfide bond to a cysteine residue of the peptide, located between the two (SG)₃ spacers. The construct depicted in this Figure is referred to as DARE™-3.02/DARE-T-AK-SGK/Sgk1-TfR-AKDEL-siRNA.

FIG. 12. Illustrates a conjugate of the present invention in which module (a) is a transferrin receptor binding peptide, module (b) is KDEL and is C-terminally linked to module (a), and module (c) is IgM(μ). Module (a) is connected via its N-terminus to the branch point N-beta-aminoxyacetyl L-diaminopropionyl residue via a disulfide bond formed from 2 cysteine residues. Compound (d) is an siRNA and is linked, via the 5′-end of the sense strand containing an aminolinker, to the linkage peptide through an adapter derived from succinimidyl 4-formylbenzoate. The connection is made through a stable oxime bond generated by reaction of the formyl group of the adapter with the aminoxy group of the branch point N-beta-aminoxyacetyl L-diaminopropionyl residue. The (SG)₃ units function as spacers to ensure that the various modules do not interfere with one another. The construct depicted in this Figure is referred to as DARE™-3.03/DARE-T-AK-IgM/TfR-AKDEL-siRNA.

FIG. 13. Illustrates a conjugate with an identical configuration to the conjugate depicted in FIG. 12 with the exception that module (b), which is the KDEL motif in this example, is now at the C-terminus of module (c), which is the IgM(μ) sequence. The construct depicted in this Figure is referred to as DARE™-3.04/DARE-T-IgM-AK/TfR-IgM(μ)-AKDEL-siRNA.

FIG. 14. Illustrates a conjugate of the present invention, whereby 2 cargo molecules, 2 compounds (d), are attached via biodegradable disulfide bonds. The cell targeting/uptake peptide, module (a), is ricin toxin subunit B, and the ERAD targeting/sorting peptide, module (c), and the ER targeting peptide, module (b), can be any module (c) and module (b) of use in a conjugate of the invention, but are located at the C-terminus of the linkage peptide. Module (a), RTb, is connected via a biodegradable (reducible) disulfide bond to a cysteine residue at the N-terminus of the linkage peptide which contains two branch point N-beta-aminoxyacetyl L-diaminopropionyl residues that are separated by a dPEG₁₂ spacer. The cargo molecules, 2 compounds (d), are siRNAs, each of which is linked via the 5″-end of the sense strand containing an aminolinker, to the linkage peptide through an adapter derived from succinimidyl 4-formylbenzoate. The connection is made through a stable oxime bond generated by reaction of the formyl group of the adapter with the aminoxy groups of the 2 branch point N-beta-aminoxyacetyl L-diaminopropionyl residues. The synthesis of an exemplary construct, in which module (c) is a Cox2 peptide and module (b) is KDEL, is described in Example 19.

FIG. 15. Illustrates the preparative anion-exchange HPLC trace of the DARE™ 3.02 construct, DARE™-T-AK-SGK with fLuc-siRNA as cargo, as described in Example 20. Separation was performed on a 1 mL Resource Q column with a linear gradient elution from 0 to 0.8 M sodium bromide in 25 mM Tris-HCl buffer, pH 7.4 containing 6 M urea during 60 min at a flow rate of 3 mL/min. The column effluent was monitored by UV at 260 and 550 nm. The x-axis is time in min and the y-axis is absorbance at 260 nm in mAU. The first peak is the desired DARE™ 3.02 construct.

FIG. 16. Illustrates the preparative anion-exchange HPLC trace of the DARE™ 3.02 construct, DARE™-T-AK-SGK with GAPDH-siRNA as cargo, as described in Example 20. Separation was performed on a 1 mL Resource Q column with a linear gradient elution from 0 to 0.8 M sodium bromide in 25 mM Tris-HCl buffer, pH 7.4 containing 6 M urea during 60 min at a flow rate of 3 mL/min. The column effluent was monitored by UV at 260 and 550 nm. The x-axis is time in min and the y-axis is absorbance at 260 nm in mAU. The first peak is the desired DARE™ 3.02 construct.

FIG. 17. Shown are PAGE analyses of the HPLC purified DARE™ 3.02 constructs with fLuc and GAPDH siRNA cargoes as described in Example 20. 15% PAGE gel, 8×6.5 cm, run for 1-1.5 h at 220 V and 25 mA with Tris-borate running buffer containing 6 M urea.

FIG. 18. MALDI-TOF mass spectrum of HPLC purified DARE™ 3.02 construct with fLuc-siRNA cargo (see Example 20). The construct is not completely stable to the MS conditions such that only a weak molecular ion with an m/z in the region of the calculated mass of 20544 Da can be observed. The observed main peak at m/z of 6830 is due to the antisense strand of the fLuc-siRNA (calculated mass 6827 Da), while the broad peak centered at m/z˜13700 is due to the sense strand conjugated to the peptide.

FIG. 19. MALDI-TOF mass spectrum of HPLC purified DARE™ 3.02 construct with GAPDH-siRNA cargo (see Example 20). The construct is not completely stable to the MS conditions such that only a weak molecular ion with an m/z in the region of the calculated mass of 20577 Da can be observed. The observed main peak at m/z of 6799 is due to the antisense strand of the GAPDH-siRNA (calculated mass 6796 Da), while the broad peak centered at m/z˜13800 is due to the sense strand conjugated to the peptide (calculated mass 13781 Da).

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention. Unless defined otherwise, all technical and scientific terms used herein generally have the same meanings as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art. Standard techniques are used for nucleic acid and peptide synthesis. The techniques and procedures are generally performed according to conventional methods in the art and various general references [e.g., 3], which are provided throughout this document. The nomenclature used herein and the laboratory procedures used in analytical chemistry and organic syntheses described below are those well known and commonly employed in the art. Standard techniques or modifications thereof are used for chemical syntheses and chemical analyses.

Preferably, the terms used herein are defined as previously described [4].

The articles “a” and “an” are used herein to refer to one or to more than one (i.e. to at least one) of the grammatical object of the article. By way of example, “an element” means one element or more than one element.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The sequences on a DNA strand which are located 5′ to a reference point on the DNA are referred to as “upstream sequences”; sequences on a DNA strand which are 3′ to a reference point on the DNA are referred to as “downstream sequences.”

A “polynucleotide” means a single strand or parallel and anti-parallel strands of a nucleic acid. Thus, a polynucleotide may be either a single-stranded or a double-stranded nucleic acid.

The term “nucleic acid” typically refers to a polynucleotide. Preferably, the nucleic acid of the conjugate of the present invention is single stranded or double stranded DNA, single stranded or double stranded RNA, siRNA, tRNA, mRNA, micro RNA (miRNA), small nuclear RNA (snRNA), small hairpin RNA (shRNA), morpholino modified iRNA (as described by Manoharan et al. in US2010/0076056 and U.S. Pat. No. 7,745,608), anti-gene RNA (agRNA), or the like.

“Homologous” as used herein, refers to the subunit sequence similarity between two polymeric molecules, e.g., between two nucleic acid molecules, e.g., two DNA molecules or two RNA molecules; or between two peptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′ATTGCC3′ and 5′TATGGC3′ share 50% homology.

As used herein, “homology” is used synonymously with “identity.” The determination of percent identity between two nucleotide or amino acid sequences can be accomplished using a mathematical algorithm. For example, a mathematical algorithm useful for comparing two sequences is the algorithm of Karlin and Altschul, 1990 [5], modified as in Karlin and Altschul, 1993 [6]. This algorithm is incorporated into the NBLAST and XBLAST programs of Altschul, et al., 1990 [7], and can be accessed, for example at the National Center for Biotechnology Information (NCBI) world wide web site having the universal resource locator “http://www.ncbi.nlm.nih.gov/BLAST/”. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997 [8]. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See http://www.ncbi.nlm.nih.gov.

The percent identity between two sequences can be determined using techniques similar to those described above, with or without allowing gaps. In calculating percent identity, typically exact matches are counted.

A “protein” according to the present invention refers to a chain of amino acid residues which may be naturally occurring or derivatives of naturally occurring amino acid residues and which are preferably linked via peptide bonds, wherein the protein consists of at least 251 amino acid residues or amino acid residue derivatives.

A “peptide” according to the present invention refers to a chain of amino acid residues which may be naturally occurring or derivatives of naturally occurring amino acid residues and which are preferably linked via peptide bonds, wherein the peptide consists of not more than 250 amino acid residues or amino acid residue derivatives. Preferably, a peptide for use in the present invention is between 10 and 250 amino acid residues or amino acid residue derivatives in length. More preferably, a peptide for use in the present invention is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 amino acids in length.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

As used herein, amino acids are represented by the full name thereof, by the three letter code corresponding thereto, or by the one-letter code corresponding thereto, as indicated in the following Table 1:

TABLE 1
Amino acids and their three letter and one letter codes.

| Full Name | Three Letter Code | One Letter Code |
|---|---|---|
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

“Amino acid analogs” refer to compounds that have the same basic chemical structure as a naturally occurring amino acid, i.e., an α carbon that is linked to a hydrogen, a carboxyl group, an amino group, and an A group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid.

“Amino acid mimetics” refer to chemical compounds that have a structure that is different from the general chemical structure of an amino acid, but that function in a manner similar to a naturally occurring amino acid.

The present invention also provides for conjugates comprising an analog of a protein or peptide as described herein. Analogs may differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. Conservative amino acid substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine.

The present invention also provides for conjugates comprising a modified protein or peptide. Modifications that do not normally alter primary sequence include in vivo or in vitro chemical derivatization of proteins and peptides, e.g., acetylation, or carboxylation. Also included in the present invention are modified proteins or peptides that are glycosylated, e.g., those made by modifying the glycosylation patterns of a protein or peptide during its synthesis and processing or in further processing steps; e.g., by exposing the protein or peptide to enzymes which affect glycosylation, e.g., mammalian glycosylating or deglycosylating enzymes. Also embraced by the present invention are proteins or peptides which have phosphorylated amino acid residues, e.g., phosphotyrosine, phosphoserine, or phosphothreonine.

It will be appreciated, of course, that the proteins and peptides of use in the conjugates of the present invention may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry that will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm), Fmoc or Boc groups. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not incorporated, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the protein or peptide of use in a conjugate of the present invention may include one or more D-amino acid residues, or may comprise amino acids which are all in the D-form. Retro-inverso forms of proteins or peptides in accordance with the present invention are also contemplated, for example, inverted peptides in which all amino acids are substituted with D-amino acid forms.

Acid addition salts of the proteins or peptides of use in a conjugate of the present invention are also contemplated as functional equivalents. Thus, a protein or peptide in accordance with the present invention that is treated with an inorganic acid such as hydrochloric, hydrobromic, sulfuric, nitric, phosphoric, hexafluorophosphoric, tetrafluoroboric, and the like, or an organic acid such as an acetic, propionic, glycolic, pyruvic, oxalic, malic, malonic, succinic, maleic, fumaric, tataric, citric, benzoic, trifluoroacetic, cinnamic, mandelic, methanesulfonic, ethanesulfonic, p-toluenesulfonic, salicyclic and the like, provides a water soluble salt of the peptide that is suitable for use in the conjugates of the present invention.

Also included are proteins and peptides that have been modified using ordinary molecular biological techniques so as to improve their resistance to proteolytic degradation or to optimize solubility properties or to render them more suitable as a therapeutic agent [e.g., when used as compound (d) in the conjugates of the invention]. Analogs of such peptides include those containing residues other than naturally occurring L-amino acids, e.g., D-amino acids or non-naturally occurring synthetic amino acids.

In addition, proteins and peptides that have been modified using ordinary molecular biological techniques so as to increase their susceptibility to proteolytic degradation [e.g., when used as modules (a), (b) and/or (c) in the conjugates of the invention] are also of use in the conjugates of the present invention. Preferably, the proteolytically susceptible protein or peptide comprises a ubiquitination site or motif. For the identification of such motifs see http://iclab.life.nctu.edu.tw/ubipred/ [9, 10]. In a preferred embodiment, a module (a), module (b), or module (c) protein or peptide of use in the conjugate of the present invention comprises a ubiquitination site or motif, whereby a polyubiquitin chain is formed on the module (a), module (b), or module (c) protein or peptide. Preferably, the polyubiquitin chain is generated at lysine 11 or lysine 48 of ubiquitin [11, 12]. Preferably, at least four ubiquitin molecules are attached to a lysine residue(s) on the proteolytically susceptible module (a), module (b), or module (c) to increase its probability of recognition and degradation by the 26S-proteasome. In addition or alternatively, the proteolytically susceptible protein or peptide has been modified to add one or more lysine residues and/or have one or more of its amino acids substituted with one or more lysine residues to create a ubiquitination site within the proteolytically susceptible protein or peptide.

It should be understood that the proteins and peptides of use in the conjugates of the invention are not limited to products of any of the specific exemplary processes listed herein.

As used herein, a “variant” of a peptide or polypeptide of use in the present invention that comprises at least one change in its amino acid sequence, wherein the at least one change is an amino acid substitution, insertion, deletion, N-terminal truncation, C-terminal truncation, or any combination of these changes. A variant of the peptide or polypeptide of use in the present invention may comprise a change at more than one of its amino acid residues. In preferred embodiments, a variant usable in the present invention exhibits a total number of up to 200 (up to 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200) changes in the amino acid sequence (i.e. substitutions, insertions, deletions, N-terminal truncations, C-terminal truncations, and/or any combination thereof). The amino acid substitutions may be conservative or non-conservative. In preferred embodiments, a variant usable in the present invention differs from the protein or domain from which it is derived by up to 1, 2, 3, 4, 5, 6, 7, 8, 9, 10, 15, 20, 25, 30, 35, 40, 45, 50, 55, 60, 65, 70, 75, 80, 85, 90, 95, or 100 amino acid substitutions, preferably conservative amino acid changes. Variants may additionally or alternatively comprise deletions of amino acids, which may be N-terminal truncations, C-terminal truncations or internal deletions or any combination of these. Such variants comprising N-terminal truncations, C-terminal truncations and/or internal deletions are referred to as “deletion variants” or “fragments” in the context of the present application. The terms “deletion variant” and “fragment” are used interchangeably herein. A deletion variant may be naturally occurring (e.g. splice variants) or it may be constructed artificially, preferably by genetic engineering means, using recombinant DNA techniques.

A “conjugate” according to the present invention refers to the physical association of the compound (d) of interest (for example, a nucleic acid molecule or a peptide) with the modules (a), (b) and (c). In some embodiments, “conjugate” refers to the non-covalent association (e.g. electrostatic interaction, hydrogen bonding interaction or hydrophobic interaction) or covalent association of the afore-mentioned components. In other embodiments, all of the components of the conjugate may be covalently attached to each other, while in other embodiments, only a subset of the components are covalently attached to each other.

“Delivery” according to the present invention refers to a process by which the compound is transported into a cell, e.g. preferably into the cytosol (cytoplasm) of a cell, or into a cell organelle, preferably the nucleus.

A “compound” in the context of the present invention refers to a biologically active compound, i.e., a compound having the potential to react with biological components. More particularly, the compounds of use in the present invention are designed to change the natural cellular processes associated with a living cell. For purposes of this specification, a natural cellular process is a process that is associated with a cell before delivery of a compound that is biologically active. In the present invention, the cellular production of, or inhibition of a material, such as a protein or an mRNA, caused by the compound of the invention that is delivered to the cell, in vivo or in vitro, is an example of a delivered compound that is biologically active. Pharmaceuticals, peptides, proteins, and nucleic acids, cytotoxic agents, radioactive agents, and other therapeutic or diagnostic moieties are examples of compounds of the present invention.

As used herein, a “biologically active compound” is a biological molecule in a form in which it exhibits a property by which it is characterized. A functional enzyme, for example, is one which exhibits the characteristic catalytic activity by which the enzyme is characterized.

In the context of the present invention, the term “linked” means that the modules and the compound are physically attached to each other or associated with each other. In some embodiments, “linked” refers to a non-covalent association (e.g., electrostatic interaction, hydrogen bonding interaction or hydrophobic interaction) or covalent association of the afore-mentioned components. In other embodiments, all of the components may be covalently attached to each other, while in other embodiments, only a subset of the components are covalently attached to each other.

The term “linked to each other in any arrangement” further means that the modules and the compound can be linked linearly and/or non-linearly with each other, and in equal or different stoichiometries to each other.

The phrase “module that mediates cell targeting and facilitates cellular uptake also referred to herein as a “cell targeting module” or “module (a)”, refers in the context of the present invention to a chemical entity, e.g. a polypeptide or oligopeptide, preferably a polypeptide, capable of (i) specifically binding to the surface of a cell of interest, wherein preferably the cell is a vertebrate cell, more preferably a mammalian cell, such as a mouse, rat, goat, sheep, dog, cat, pig, cow, horse, primate, or human cell, etc., even more preferably a human cell, and (ii) mediating entry of the module and further components of the conjugate linked thereto into an intact cell via a natural process that might be an endocytosis process, which might be a receptor-mediated uptake, pinocytosis, phagocytosis, macropinocytosis or fluid-phase endocytosis allowing access to intracellular membrane-bound organelles or vesicles. Preferably, the module that mediates cell targeting and facilitates cellular uptake is taken up by the cell by a process that results in an intracellular membrane-bound vesicle, a membrane bound tubule or a membrane bound tubular vesicular structure). The structures, which are specifically bound by the module, are preferably cell surface receptors. One of ordinary skill in the art can readily assess whether a module mediates cell targeting and facilitates cellular uptake, e.g., by (i) labelling said module, for example, with a radioactive or fluorescent marker, (ii) incubating the labelled module with intact cells, preferably mammalian cells, for example human cells, and (iii) assessing whether the labelled module can be detected inside the cells, i.e. in an intracellular membrane-bound organelle or vesicle in the cytoplasm of the intact cells, e.g. by fluorescence microscopy [see for example, 13-15].

The phrase “module that facilitates the transport to the endoplasmic reticulum (ER)”, also referred to herein as an “ER targeting module” or “module (b)”, refers in the context of the present invention to a chemical entity, e.g. polypeptide or oligopeptide, preferable an oligopeptide, capable of mediating the transport of the module and further components of the conjugate linked thereto to the ER. The transport to the ER via the Golgi apparatus is in the opposite direction to the biosynthetic-secretory transport delivering molecules destined for secretion from the ER to the Golgi apparatus and further to the plasma membrane and is, therefore, also known as retrograde transport pathway to the ER. One of ordinary skill in the art can readily assess whether a module facilitates the transport to the ER, e.g., by (i) labelling said module, for example, with a radioactive or fluorescent marker, (ii) linking said labelled module to a module that mediates cell targeting and facilitates cellular uptake [module (a)], (iii) incubating both modules with intact cells, preferably mammalian cells, for example human cells, and (iv) assessing whether said labelled module can be detected in the ER of a cell, e.g. by fluorescence microscopy or assessment of its N-glycosylation status [14, 16].

The phrase “module that mediates translocation from the ER to the cytosol”, also referred to herein as an “ERAD targeting module” or “module (c)”, refers in the context of the present invention to a chemical entity, preferably a polypeptide or oligopeptide, capable of mediating the entry of the module and further components of the conjugate linked thereto, into the cytosol from the lumen of the ER, e.g. by acting as a substrate for ER-associated degradation (ERAD). The transport out of the ER into the cytosol is also known as retro-translocation. The ERAD pathway is a cellular pathway which normally targets misfolded or mis-glycosylated proteins for ubiquitination and subsequent degradation by a protein-degrading complex, called the proteasome. By exploiting the ERAD pathway using the module that mediates translocation from the ER to the cytosol, a conjugate of the present invention is able to deliver a compound to the cytoplasm, and whereby the cell targeting, ER targeting and ERAD targeting modules of the conjugate, if still remaining, will preferably be degraded by the proteosome. One of ordinary skill in the art can readily assess whether a module mediates translocation from the ER to the cytosol, e.g., by (i) labelling said module, for example, with a radioactive or fluorescent marker, (ii) linking said labelled module to a module that mediates cell targeting and facilitates cellular uptake [module (a)] and to a module that facilitates transport to the ER [module (b)], (iii) incubating the conjugated modules with intact cells, preferably mammalian cells, for example human cells, and (iv) assessing whether said labelled module can be detected in the cytosol of a cell and is degraded over time, presumably by the proteosome, e.g. by fluorescence microscopy or western blotting [See for example, 17].

One of ordinary skill in the art can also readily assess whether the modules (a), (b) and (c) carrying the above mentioned functionalities are able to deliver a compound into a cell, by (i) labelling the modules and the compound (d), for example, with different radioactive or fluorescent markers, (ii) linking the modules (a), (b) and (c) and the compound (d) to each other, (iii) incubating the conjugated modules and compound with intact cells, preferably mammalian cells, for example human cells, and (iv) assessing whether the compound (d) and modules can be detected in the cytosol of a cell, e.g. by fluorescence microscopy.

One of ordinary skill in the art can also use co-staining of the cells to determine the intracellular sorting of the module (a); of the modules (a) and (b); of the modules (a), (b) and (c); and of the modules (a), (b) and (c) and of the compound (d), i.e. of the conjugate. For example, cells comprising a module, modules, or the conjugate can be co-stained for intracellular compartments, e.g. endosomes, lysosomes, trans-golgi network, golgi apparatus, ER, caveolae and cytoplasm using immunohistochemistry as described below in Example 7.

In a first aspect, the present invention relates to a delivery system comprising or consisting of a conjugate for delivery of a compound into a cell, wherein the conjugate comprises, essentially consisting of or consists of:
(a) at least one module (a) that mediates cell targeting and facilitates cellular uptake,
(b) at least one module (b) that facilitates transport to the endoplasmic reticulum (ER),
(c) at least one module (c) that mediates translocation from the ER to the cytosol, and
(d) at least one compound (d),
   wherein the modules (a), (b) and (c), and the compound (d) are linked to each other in any arrangement.

Preferably, a delivery system comprising or consisting of a conjugate for delivery of a compound into a cell according to the present invention comprises, essentially consisting of or consists of
(a) at least one module (a) that mediates cell targeting and facilitates cellular uptake,
(b) at least one module (b) that facilitates transport of modules (b) and (c) and compound (d) and, optionally module (a) to the endoplasmic reticulum (ER),
(c) at least one module (c) that mediates translocation of at least one compound
(d) and, optionally one or more of the modules (a), (b) or (c) from the ER to the cytosol, and
(d) at least one compound (d),
   wherein the modules (a), (b) and (c), and the compound (d) are linked to each other in any arrangement.

In a preferred embodiment, the delivery system of the present invention further comprises a nuclear localization signal.

Preferably, the delivery system according to the first aspect of the invention comprises, essentially consists or consists of a conjugate of the second aspect of the invention.

In a second aspect, the present invention relates to a conjugate for delivery of a compound into a cell comprising, essentially consisting of or consisting of:
(a) at least one module (a) that mediates cell targeting and facilitates cellular uptake,
(b) at least one module (b) that facilitates transport to the endoplasmic reticulum (ER),
(c) at least one module (c) that mediates translocation from the ER to the cytosol, and
(d) at least one compound (d),
   wherein the modules (a), (b) and (c), and the compound (d) are linked to each other in any arrangement.

Preferably, a conjugate for delivery of a compound into a cell according to the present invention comprises, essentially consisting of or consists of
(a) at least one module (a) that mediates cell targeting and facilitates cellular uptake,
(b) at least one module (b) that facilitates transport of modules (b) and (c) and compound (d) and, optionally module (a) to the endoplasmic reticulum (ER),
(c) at least one module (c) that mediates translocation of at least one compound (d) and, optionally one or more of the modules (a), (b) or (c) from the ER to the cytosol, and
(d) at least one compound (d),
   wherein the modules (a), (b) and (c), and the compound (d) are linked to each other in any arrangement.

In a preferred embodiment, the conjugate of the present invention further comprises a nuclear localization signal.

The conjugate according to the present invention comprises, essentially consists of or consists of at least one module (a), at least one module (b), at least one module (c) and at least one compound (d). The at least one module (a), the at least one module (b), the at least one module (c) and the at least one compound (d) of the conjugate of the present invention are linked to each other in any arrangement, combination, or stoichiometry.

In a preferred embodiment of the conjugate of the present invention, two or more of the modules of the conjugate may be comprised or contained within a single protein or peptide, i.e., a protein or peptide that comprises a cell targeting/uptake functionality [module (a)] and an ER transport functionality [module (b)], a protein or peptide that comprises a cell targeting/uptake functionality [module (a)] and an ER to the cytosol translocation functionality [module (c)], a protein or peptide that comprises an ER transport functionality [module (b)] and an ER to the cytosol translocation functionality [module (c)], or a protein or peptide that comprises a cell targeting/uptake functionality [module (a)], an ER transport functionality [module (b)], and an ER to the cytosol translocation functionality [module (c)]. Within these embodiments, the two or more modules are linked to each other as a contiguous protein or peptide, in any arrangement, combination, or stoichiometry.

Preferably, the modules (a), (b), (c) and the compound (d) of the conjugate of the present invention are linked to each other in one of the following arrangements or combinations: (a), (b), (c) and (d); (b), (a), (c) and (d); (b), (c), (a) and (d); (c), (b), (a) and (d); (a), (c), (b) and (d); (c), (a), (b) and (d); (c), (d), (b) and (a); (d), (c), (b) and (a); (b), (d), (c) and (a); (d), (b), (c) and (a); (b), (c), (d) and (a); (c), (b), (d) and (a); (c), (d), (a) and (b); (d), (c), (a) and (b); (a), (d), (c) and (b); (d), (a), (c) and (b); (a), (c), (d) and (b); (c), (a), (d) and (b); (b), (d), (a) and (c); (d), (b), (a) and (c); (a), (d), (b) and (c); (d), (a), (b) and (c); (a), (b), (d) and (c); or (b), (a), (d) and (c), wherein in each arrangement or combination at least one module (a), at least one module (b), at least one module (c) and at least one compound (d) is present. The respectively indicated order of the modules (a), (b) and (c) and the compound (d) signifies the links between the modules and compound, respectively. Thus, in the arrangement (a), (b), (c) and (d), (a) is linked to (b), (b) is linked to (c) and (c) is linked to (d). The term “linked” in this context has the meaning as defined above and as more specifically taught below, e.g. includes covalent linkages, non-covalent linkages and linkages via linker molecules.

It is particularly preferred that the modules (a), (b), (c) and the compound (d) of the conjugate of the present invention are linked to each other in one of the following arrangements or combinations: (a)ₓ, (b)_{y}, (c)_{z} and (d)ₙ; (b)_{y}, (a)ₓ, (b)_{z} and (d)ₙ; (b)_{y}, (c)_{z}, (a)ₓ and (d)ₙ; (c)_{z}, (b)_{y}, (a)ₓ and (d)ₙ; (a)ₓ, (c)_{z}, (b)_{y} and (d)ₙ; (c)_{z}, (a)ₓ, (b)_{y} and (d)ₙ; (c)_{z}, (d)ₙ, (b)_{y} and (a)ₓ; (d)ₙ, (c)_{z}, (b)_{y} and (a)ₓ; (b)_{y}, (d)ₙ, (c)_{z} and (a)ₓ; (d)ₙ, (b)_{y}, (c)_{z} and (a)ₓ; (b)_{y}, (c)_{z}, (d)ₙ and (a)ₓ; (c)_{z}, (b)_{y}, (d)ₙ and (a)ₓ; (c)_{z}, (d)ₙ, (a)ₓ and (b)_{y}; (d)ₙ, (c)_{z}, (a)ₓ and (b)_{y}; (a)ₓ, (d)ₙ, (c)_{z} and (b)_{y}; (d)ₙ, (a)ₓ, (c)_{z} and (b)_{y}; (a)ₓ, (c)_{z}, (d)ₙ and (b)_{y}; (c)_{z}, (a)ₓ, (d)ₙ and (b)_{y}; (b)_{y}, (d)ₙ, (a)ₓ and (c)_{z}; (d)ₙ, (b)_{y}, (a)ₓ and (c)_{z}; (a)ₓ, (d)ₙ, (b)_{y} and (c)_{z}; (d)ₙ, (a)ₓ, (b)_{y} and (c)_{z}; (a)ₓ, (b)_{y}, (d)ₙ and (c)_{z}; or (b)_{y}, (a)ₓ, (d)ₙ and (c)_{z}, wherein x is an integer of 1 to 5, i.e. 1, 2, 3, 4, or 5, preferably of 1; y is an integer of 1 to 5, i.e. 1, 2, 3, 4, or 5, preferably of 1; z is an integer of 1 to 5, i.e. 1, 2, 3, 4, or 5, preferably of 1; and n is an integer of 1 to 50, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, preferably of 2, 3, 4, 5, 6, 7, 8, 9, or 10, more preferably of 2, 3, 4, or 5.

A conjugate according to the present invention that comprises more than one compound (d) can deliver more compounds (d) into a cell, thus the efficiency of delivering a compound (d) can be increased compared to a conjugate according to the present invention that comprises modules (a), (b) and (c) and only one compound (d). Preferably, the conjugate according to the present invention comprises at least 2-50 compounds (d). More preferably, the conjugate according to the present invention comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 compounds (d). More preferably, the conjugate according to the present invention comprises at least 2, 3, 4, or 5 compounds (d). Preferably, the conjugate comprising more than one compound (d) comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 compounds (d) that are the same or different.

In a preferred embodiment, the conjugate comprising more than one compound (d) comprises at least 2 of the same compounds (d). Preferably, the at least 2 of the same compounds (d) are selected from the group consisting of 2 nucleic acids, 2 proteins, 2 peptides, 2 antigens, 2 enzymes, 2 small molecules, 2 therapeutic molecules, 2 diagnostic molecules, and 2 imaging molecules, Preferably, the at least 2 same compounds (d) comprise at least 2 of the same nucleic acids. More preferably, the at least 2 same compounds (d) comprise at least 2 of the same siRNAs.

In another preferred embodiment, the conjugate comprising more than one compound (d) comprises at least 2 different compounds (d). Preferably, the at least 2 different compounds (d) comprise a first compound (d) selected from the group consisting of a nucleic acid, a protein, a peptide, an antigen, an enzyme, a small molecule, a therapeutic molecule, a diagnostic molecule, and an imaging molecule; and a second compound (d) selected from the group consisting of a nucleic acid, a protein, a peptide, an antigen, an enzyme, a small molecule, a therapeutic molecule, a diagnostic molecule, and an imaging molecule, wherein the first compound (d) and the second compound (d) are different from each other. In a preferred embodiment, the at least 2 different compounds (d) comprise at least 2 different nucleic acids. Preferably, the at least 2 different compounds (d) comprise at least 2 different siRNAs directed to the same target. In another preferred embodiment, the at least 2 different compounds (d) comprise at least 2 different siRNAs directed to at least 2 different targets. In another preferred embodiment, the at least 2 different compounds (d) comprise at least one nucleic acid and at least one protein or peptide. Preferably, the at least one nucleic acid is an siRNA and the at least one protein or peptide is a RISC protein or peptide.

Conjugates of the present invention, wherein the module (b) or the modules (b) are positioned within the arrangement in a way that they are linked to only one other module or compound are preferred to avoid or to at least minimize steric hindrance by the other modules and/or compound(s) of the conjugate or other undesired interactions. Thus, preferred embodiments of the conjugate of the present invention are (c), (d), (a) and (b); (d), (c), (a) and (b); (a), (d), (c) and (b); (d), (a), (c) and (b); (a), (c), (d) and (b); and (c), (a), (d) and (b), wherein in each embodiment at least one module (a), at least one module (b) and at least one module (c) and at least one compound (d) is present. The presence of module (b) in the indicated position has the advantage that module (b) is free and unhindered by the other modules (a) and (c) and by compound (d) so that steric hindrance or other undesired interactions can be avoided or at least minimized. If module (b) comprises, essentially consists or consists of an oligopeptide, it is preferred that the C-terminus of such oligopeptide is free and that any linkage, be it covalent or non-covalent, to further modules, compound(s) or linker molecule occurs at or close to the N-terminus of such oligopeptide.

Particularly preferred embodiments of the conjugate of the present invention are the following arrangements (c)_{z}, (d)ₙ, (a)ₓ and (b)_{y}; (d)ₙ, (c)_{z}, (a)ₓ and (b)_{y}; (a)ₓ, (d)ₙ, (c)_{z} and (b)_{y}; (d)ₙ, (a)ₓ, (c)_{z} and (b)_{y}; (a)ₓ, (c)_{z}, (d)ₙ and (b)_{y}; and (c)_{z}, (a)ₓ, (d)ₙ and (b)_{y}, wherein x is an integer of 1 to 5, i.e. 1, 2, 3, 4, or 5, preferably of 1; y is an integer of 1 to 5, i.e. 1, 2, 3, 4, or 5, preferably of 1; z is an integer of 1 to 5, i.e. 1, 2, 3, 4, or 5; preferably of 1; and n is an integer of 1 to 10, i.e. 1, 2, 3, 4, 5, 6, 7, 8, 9 or 10, preferably of 3. Accordingly, it is particularly preferred that x is 1, y is 1, z is 1 and n is an integer of 1 to 50, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, preferably of 2, 3, 4, 5, 6, 7, 8, 9, or 10, more preferably of 2, 3, 4, or 5.

Conjugates of the present invention, wherein compound (d) or compounds (d) are positioned in second position or third position and module (b) or modules (b) are positioned within the arrangement in a way that they are linked to only one other module or compound, e.g. positioned in last position of the arrangement, i.e., wherein the C-terminus of module (b) or modules (b) is free, are preferred. Therefore, particularly preferred embodiments of the conjugate of the present invention are (c), (d), (a) and (b); (a), (d), (c) and (b); (a), (c), (d) and (b); and (c), (a), (d) and (b), wherein in each embodiment at least one module (a), at least one module (b), at least one module (c) and at least one compound (d) is present. The presence of compound (d) in second or third position has the advantage that the entrance of compound (d) into the cell and further within the cell is facilitated by avoiding steric hindrance by compound (d) for the biological action of modules (a), (b) and (c). In addition, module (b) is free and unhindered by the other modules (a) and (c) and by compound (d) so that steric hindrance and other undesired interactions can be avoided or at least minimized.

Particularly preferred embodiments of the conjugate of the present invention are (c)_{z}, (d)ₙ, (a)ₓ and (b)_{y}; (a)ₓ, (d)ₙ, (c)_{z} and (b)_{y}; (a)ₓ, (c)_{z}, (d)ₙ and (b)_{y}; and (c)_{z}, (a)ₓ, (d)ₙ and (b)_{y}, wherein x is an integer of 1 to 5, i.e. 1, 2, 3, 4, or 5, preferably of 1; y is an integer of 1 to 5, i.e. 1, 2, 3, 4, or 5, preferably of 1; z is an integer of 1 to 5, i.e. 1, 2, 3, 4, or 5; preferably of 1; and n is an integer of 1 to 50, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, preferably of 2, 3, 4, 5, 6, 7, 8, 9, or 10, more preferably of 2, 3, 4, or 5. Accordingly, it is particularly preferred that x is 1, y is 1, z is 1 and n is an integer of 1 to 50, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, preferably of 2, 3, 4, 5, 6, 7, 8, 9, or 10, more preferably of 2, 3, 4, or 5.

In the most preferred embodiments of the conjugate of the present invention, wherein module (b) is arranged terminally, preferably in last position, wherein its C-terminus is free, and compound (d) in second or third position, the arrangements of the modules (a), (b) and (c) and of the compound (d) and the number of the modules (a), (b) and (c) and of the compound (d) are as follows:
(i) (a)ₓ, (c)_{z}, (d)ₙ, and (b)_{y}, wherein x is an integer of 1, z is an integer of 1, n is an integer of 1 and y is an integer of 1,
(ii) (a)ₓ, (c)_{z}, (d)ₙ, and (b)_{y}, wherein x is an integer of 1, z is an integer of 1, n is an integer of 2 and y is an integer of 1,
(iii) (a)ₓ, (c)_{z}, (d)ₙ, and (b)_{y}, wherein x is an integer of 1, z is an integer of 1, n is an integer of 3 and y is an integer of 1,
(iv) (a)ₓ, (d)ₙ, (c)_{z} and (b)_{y}, wherein x is an integer of 1, n is an integer of 1, z is an integer of 1 and y is an integer of 1,
(v) (a)ₓ, (d)ₙ, (c)_{z} and (b)_{y}, wherein x is an integer of 1, n is an integer of 2, z is an integer of 1 and y is an integer of 1, or
(vi) (a)ₓ, (d)ₙ, (c)_{z} and (b)_{y}, wherein x is an integer of 1, n is an integer of 3, z is an integer of 1 and y is an integer of 1.

Preferably, the at least one module (a), the at least one module (b), the at least one module (c) and the at least one compound (d) of the conjugate of the present invention, which are arranged to each other in any order, combination, or stoichiometry, are linked to each other via a covalent linkage, are linked to each other via a non-covalent linkage, are linked to each other via at least one adapter molecule and/or are linked to each other via at least one linker molecule that optionally comprises at least one adapter molecule.

The term “covalent linkage” means a type of chemical linkage, wherein each atom of a bond pair contributes one electron to form a pair of electrons in a chemical bond.

The term “non-covalent linkage” means a type of chemical linkage, typically between macromolecules, that does not involve the sharing of pairs of electrons, but rather involves more dispersed variations of electromagnetic interactions.

The term “linker molecule” in the context of the present invention refers to a molecule that is able to attach or conjugate two molecules or compounds to each other. This attachment or conjugation can be achieved via a covalent linkage. Thus, any molecule having the above mentioned characteristics can be used to link the modules and the compound of the conjugate of the present invention to each other. Preferably, the linker molecule serves the purpose of spatially separating the various modules and the compound(s) to avoid steric hindrance between the modules and the compound. Such steric hindrance may inhibit access and/or interaction with the cellular structures, e.g. proteins, lipids or carbohydrate chains, to which the modules have to bind or to interact; to exert their respective function as outlined herein.

The term “adapter molecule” in the context of the present invention refers to a molecule that forms an indirect and no-covalent linkage, e.g. between a module [e.g. module (a)] and a compound (d). For example, the adapter molecule, wherein it is covalently linked to module (a), can be used to indirectly and non-covalently link module (a) to compound (d), wherein the adaptor molecule forms a non-covalent linkage to compound (d). As such, the adapter molecule also functions as a spacer to keep the compound (d) at a distance from the module (a). The indirect and non-covalent linkage is based on ionic (electrostatic) interactions or hydrophobic interactions.

The different types of linkages are exemplified in the following description for the conjugation of module (a) to compound (d). It shall be understood that this exemplification is applicable to any module-module, any module-compound (d), or any compound (d)-compound (d) conjugation. For example, module (a) of the conjugate of the present invention can be directly linked to compound (d) via a non-covalent linkage. Module (a) of the conjugate of the present invention can also be directly linked to compound (d) via a covalent linkage. Module (a) of the conjugate of the present invention can further be linked indirectly and covalently to compound (d) via a linker molecule, which forms a covalent linkage with module (a) and with compound (d). In addition, compound (d) can be linked indirectly to module (a) via an adapter molecule, wherein the adapter molecule and compound (d) are connected to each other via a non-covalent linkage and the adapter molecule is covalently linked to module (a). Further, compound (d) can be indirectly linked to module (a) via an adapter molecule and a linker molecule, wherein the adapter molecule and compound (d) are connected to each other via a non-covalent linkage, and the adapter molecule is covalently linked to a linker molecule which links module (a) and an adjacent module [e.g. module (c) or (b)].

The modules and the compound of the conjugate of the present invention can be linked via different linkage types to each other. Thus, the conjugate of the present invention does not necessarily comprise modules and a compound linked to each other via the same linkage type. For example, covalent linkages can be used with non-covalent linkages and/or with covalent linkages via linker molecules or adapter molecules. Depending upon the desired target cell delivery strategy, the conjugate can be designed with specific covalent and/or non-covalent linkages, with or without an adapter molecule and/or linker molecule. In this way, one of ordinary skill in the art can make different types of conjugates that are useful for different applications.

Preferably, the at least one module (a), the at least one module (b), the at least one module (c) and the at least one compound (d) of the conjugate according to the present invention are covalently linked to each other, preferably via a disulfide-linkage, an amide-linkage, an oxime-linkage and/or a hydrazone-linkage.

The term “disulfide-linkage” (disulfide-bond) refers to a chemical bond, which is usually derived by the coupling of two thiol groups. The linkage is also called an SS-bond or disulfide bridge. Disulfide bonds in proteins are formed between the thiol groups of cysteine residues.

The term “amide-linkage” (peptide bond) refers to a chemical bond formed between two proteins or peptides when the carboxyl group of one molecule reacts with the amine group of the other molecule, thereby releasing a molecule of water (H₂O).

The term “oxime-linkage” refers to a chemical bond, which is derived by coupling of a protein or peptide carrying aglyoxylic aldehyde functionality to a protein or peptide functionalized with an aminooxy group. The oxime linkage is obtained by reaction of an aldehyde or ketone with a hydroxylamine or aminooxy modified component. It can be used to link together all manner of molecules, i.e. small molecules, sugars, peptides, proteins, oligonucleotides, etc. These functionalities may be present in a synthesized component of a conjugate of the invention, or one or both of the functionalities may be introduced into a component of a conjugate of the invention. In a preferred method of preparing a conjugate of the present invention, an aminooxy modification is included in a synthetic peptide and a benzaldehyde function is attached to an siRNA.

The term “hydrazone-linkage” (hydrazone-bond) refers to a chemical bond, which is derived by condensing proteins or peptides with each other which are modified at their amino groups to contain an average of three to six aryl aldehyde or acyl hydrazide groups. The hydrazone linkage is obtained by reaction of an aldehyde or ketone with a hydrazine or acylhydrazine modified component. An “acylhydrazone linkage” is obtained by reaction of an aldehyde or ketone with an acylhydrazine modified component. Commercial reagent kits are available and may be used within the methods of the present invention to couple or connect two biomolecules of use in a conjugate of the present invention.

There are four commonly known types of non-covalent interactions: hydrogen bonds, ionic bonds, Van der Waals forces, and hydrophobic interactions, which may be the basis for the interaction of the modules and/or compound(s) used in the conjugates of the present invention.

Preferably, the at least one module (a), the at least one module (b), the at least one module (c) and/or the at least one compound (d) of the conjugate according to the present invention are linked to each other via non-covalent linkage, preferably an ionic (electrostatic) linkage and/or via a hydrophobic linkage.

The term “hydrophobic interaction” (hydrophobic linkage) refers to an interaction dependent from the tendency of hydrocarbons (or of lipophilic hydrocarbon-like groups in solutes) to form intermolecular aggregates in an aqueous medium.

The term “ionic (electrostatic) linkage” (ionic bond or electrostatic bond) refers to a non-covalent bond in which one atom loses an electron to form a positive ion and the other atom gains to electron to form a negative ion. In biological systems, most electrostatic bonds or interactions are between groups that are protonated and others that are deprotonated, i.e., a lysine or arginine side chain amino group interacting with either a carboxylate group of a protein or a phosphate group in a DNA or RNA molecule.

A particularly preferred linker molecule according to the present invention is a peptide, a modified peptide, an amino acid residue, a modified amino acid residue or a hydrophilic carbohydrate chain, preferably a polydiol chain with between 1 to 20 repeat units, i.e. 1, 2, 3, 4, 5, 6, 7, 8, 9, 10, 11, 12, 13, 14, 15, 16, 17, 18, 19 or 20, preferably polyethylene glycol (PEG), wherein between 1 to 20, i.e. 1, 2, 3, 4, 5, 6, 7, 8, 9, 10, 11, 12, 13, 14, 15, 16, 17, 18, 19 or 20, ethyleneglycol units are connected to each other. These linker molecules link the at least one module (a), the at least one module (b), the at least one module (c) and/or the at least one compound (d) to each other via a covalent linkage, preferably via an amide-linkage or a disulfide-linkage.

Said linker molecules can also be combined with each other, e.g. a peptide linker can be combined with a modified amino acid residue linker, or a modified amino acid residue linker can be combined with a modified peptide linker to covalently link 1) at least one module (a) to at least one module (b) or at least one module (c); 2) at least one module (b) to at least one module (a) or at least one module (c); 3) at least one module (a) to at least one module (b) and at least one module (c); or 4) at least one module (a), at least one module (b), and/or at least one module (c) to at least one compound (d). Preferably, the at least one module (a), the at least one module (b), or the at least one module (c) are covalently linked via an amide linkage. Preferably, the at least one module (a), the at least one module (b), and/or the at least one module (c) are/is covalently linked to the at least one compound (d) via a disulfide linkage.

The term “peptide linker” according to the present invention means a chain of amino acid residues which may be naturally occurring or derivatives of naturally occurring amino acid residues and which are preferably linked via peptide or disulfide bonds.

Preferably, the peptide linker of the present invention consists of between 2 and 50 or between 2 and 30 amino acid residues or amino acid residue derivatives, preferably of between 2 and 20 or between 2 and 15 amino acid residues or amino acid residue derivatives, and more preferably of between 2 and 10, between 2 and 5, or 2, 3, 4, 5, 6, 7, 8, 9 or 10 amino acid residues or amino acid residue derivatives. Preferably, the linker sequence is flexible so as not to hold the conjugate in a single rigid conformation. The peptide linker can be used to space the modules (a), (b) and (c) from each other and/or to space the modules (a), (b) and (c) from the compound (d). For example, two peptide linkers can be positioned in a conjugate of the present invention having the precise arrangement: module (a), a first peptide linker, compound (d), a second peptide linker, module (c) and module (b), such that a first peptide linker is positioned between module (a) and compound (d) and a second peptide linker is positioned between compound (d) and module (c), to provide molecular flexibility of and/or around compound (d). One of ordinary skill in the art can position the peptide linker or peptide linkers within the conjugate as necessary and specific to the modules, compound and intended use of the conjugate, and without undue experimentation. The length of the peptide linker is chosen to optimize the biological activity of the conjugate comprising the compound and can be determined empirically without undue experimentation. The linker peptide should be long enough and flexible enough to allow unhindered functionality of the modules and of the compound and to avoid steric or other undesired interactions. Examples of peptide linkers include but are not limited to GGGGS (SEQ ID NO: 1), GKSSGSGSESKS (SEQ ID NO: 2), GSTSGSGKSSEGKG (SEQ ID NO: 3), GSTSGSGKSSEGSGSTKG (SEQ ID NO: 4), GSTSGSGKPGSGEG STKG (SEQ ID NO: 5), EGKSSGSGSESKEF (SEQ ID NO: 6), and SGSGSG [(SG)₃; SEQ ID NO: 7]. Other suitable linker peptides are those as previously described in the literature [18-20] and in U.S. Pat. No. 4,751,180, U.S. Pat. No. 4,935,233, and the like.

The term “modified peptide linker” according to the present invention means a chain of amino acid residues which may be naturally occurring or a derivative of naturally occurring amino acid residues preferably linked via peptide bonds which is further chemically modified. A preferred modified peptide linker is a peptide covalently bound to polyethyleneglycol (PEG). Such a modified peptide linker can be predominantly composed of short polyethylenglycol (PEG) repeats which facilitate its synthesis. PEG is already approved for delivery and stabilization of peptide based therapeutics and is non-toxic. For example, N-Fmoc-amido-dPEG₁₂-acid can be utilized as a spacer to replace a repeat of several amino acid residues to simplify the synthesis, improve solubility, and ensure flexibility of the linker that connects the various functional domains within the synthetic peptide.

The term “amino acid residue linker” encompasses naturally occurring amino acids as well as amino acid derivatives. Preferably, the amino acids of the amino acid linker are small amino acids or hydrophobic non-aromatic amino acids. A small amino acid in the context of the present invention is preferably an amino acid having a molecular weight of less than 125 Dalton. Preferably, a small amino acid is selected from the group consisting of the amino acids glycine, alanine, serine, cysteine, threonine, valine, and derivatives thereof. A hydrophobic non-aromatic amino acid in the context of the present invention is preferably any amino acid which has a Kyte-Doolittle hydropathy index of higher than 0.5, more preferably of higher than 1.0, even more preferably of higher than 1.5 and is not aromatic. Preferably, a hydrophobic non-aromatic amino acid in the context of the present invention, is selected from the group consisting of the amino acids alanine (Kyte Doolittle hydropathy index 1.8), methionine (Kyte Doolittle hydropathy index 1.9), isoleucine (Kyte Doolittle hydropathy index 4.5), leucine (Kyte Doolittle hydropathy index 3.8), valine (Kyte Doolittle hydropathy index 4.2), and derivatives thereof having a Kyte Doolittle hydropathy index as defined above.

The term “modified amino acid residue linker” encompasses naturally occurring amino acids as well as amino acid derivatives which are chemically modified. For example, modified amino acids are prepared by reacting single amino acids with an acylating or sulfonating agent which reacts with free amino moieties present in the amino acids to form amides or sulfonamides, respectively. A preferred modified amino acid linker is an amino acid which is acetylated or sulfonated. Also preferred is the use of activated cysteine [C(NPyS)] as a modified amino acid linker.

An adapter molecule forms an indirect and non-covalent linkage, e.g. between a module [e.g. module (a), (b) or (c), preferably module (a)] and a compound (d), preferably via ionic (electrostatic) interactions or hydrophobic interactions.

In a preferred embodiment of a conjugate of the present invention, the adapter molecule indirectly and non-covalently links module (a) to compound (d) by forming a non-covalent linkage to compound (d), e.g. via hydrophobic interactions, wherein the adapter molecule is covalently linked to module (a). In addition, module (a) is covalently linked to module (c) and module (c) is covalently linked to module (b).

In another preferred embodiment of a conjugate of the present invention, an adapter molecule interacts with a compound (d) via an ionic (e.g., electrostatic) interaction or a hydrophobic interaction, wherein the adapter molecule is covalently linked to a linker molecule that connects a module (a) with a module (c). In addition, the module (c) is covalently linked to a module (b). As a result, the module (a) and the compound (d) are indirectly and non-covalently linked to each other via the adapter molecule. Thus, a conjugate of the present invention preferably comprises a linker molecule between module (a) and module (c), wherein the linker molecule is covalently linked to an adaptor molecule that is non-covalently linked to the compound (d) Preferably, the adapter molecule branches off from a side chain of the linker molecule.

Generally, one or more adapter molecules can be used to indirectly and non-covalently link, e.g. a compound (d) and a module, e.g. module (a), (b) or (c), preferably module (a), to each other. In a preferred embodiment of the conjugate of the present invention, 2, 3, 4, or 5 adapter molecules are used to indirectly and non-covalently link a compound (d) and a module, e.g. module (a), (b) or (c), preferably module (a), to each other. More preferably, 2 adapter molecules are used in the conjugate of the present invention to indirectly and non-covalently link a compound (d) and a module, e.g. module (a), (b) or (c), preferably module (a), to each other.

For example, in a preferred embodiment, a conjugate of the present invention comprises two (2) adapter molecules that each interact with a compound (d) via ionic (electrostatic) interactions and/or hydrophobic interactions, and wherein each of the two adapter molecules are covalently linked to a module (a) of the conjugate. In addition, the module (a) is covalently linked to a module (c), and the module (c) is covalently linked to a module (b). Thus, as a result, the module (a) and the compound (d) are indirectly and non-covalently linked to each other via the two adapter molecules. Preferably, the two adaptor molecules are the same. The resulting conjugate of this preferred embodiment of the invention has an increased ratio of compound (d) to delivery vehicle [i.e., modules (a), (b), and (c)].

Preferably, modules (b) and (c) are not used to covalent link to the adapter molecule to minimize the risk of interfering with their functionalities.

Preferred adapter molecules are nucleic acid binding domains of proteins such as RNA binding proteins or double stranded RNA (dsRNA) binding proteins (DRBPs), double stranded DNA (dsDNA) binding proteins (DDBPs), single chain antibodies or ligand binding domains of surface receptors. More preferred adapter molecules that may be used in the conjugates of the present invention to indirectly and non-covalently link or conjugate a module and a compound to each other are double stranded RNA binding proteins (DRBPs). The DRBP may be used in the present invention for different functions. It may function as a spacer to keep compound (d) at a distance from module(s) (a), (b), and/or (c). It may also form a stable indirect and non-covalent linkage between a compound (d) and a module, e.g. module (a), (b) or (c), preferably module (a). DRBP may also serve to neutralize or reduce the anionic charge of a compound (d) to be delivered using modules (a), (b) and (c). DRBP may further promote the uptake of a conjugate of the present invention by sufficiently reducing the anionic charge of a compound (d) such that the cationic charge of the modules (a), (b) and (c) is sufficient to enter the cell by an endocytic event.

The use of a DRBP adaptor(s) or a DDBP adaptor(s) is preferred when compound (d) is a nucleic acid. When compound (d) is a double stranded RNA (dsRNA), a conjugate of the present invention comprises a DRBP adaptor(s). When compound (d) is a double stranded DNA (dsDNA), a conjugate of the present invention comprises a DDBP adaptor(s).

Preferred dsRNA binding proteins (DRBPs) that can be employed as adapter molecules in the conjugates of the present invention and their Accession numbers in parenthesis include: PKR (AAA36409, AAA61926, Q03963), TRBP (P97473, AAA36765), PACT (AAC25672, AAA49947, NP609646), Staufen (AAD17531, AAF98119, AAD17529, P25159), NFAR1 (AF167569), NFAR2 (AF167570, AAF31446, AAC71052, AAA19960, AAA19961, AAG22859), SPNR (AAK20832, AAF59924, A57284), RHA (CAA71668, AAC05725, AAF57297), NREBP (AAK07692, AAF23120, AAF54409, T33856), kanadaptin (AAK29177, AAB88191, AAF55582, NP499172, NP198700, BAB19354), HYLL (NP563850), hyponastic leaves (CAC05659, BAB00641), ADAR1 (AAB97118, P55266, AAK16102, AAB51687, AF051275), ADAR2 P78563, P51400, AAK17102, AAF63702), ADAR3 (AAF78094, AAB41862, AAF76894), TENR (XP059592, CAA59168), RNaseIII (AAF80558, AAF59169, Z81070Q02555/S55784, P05797), and Dicer (BAA78691, AF408-401, AAF56056, S44849, AAF03534, Q9884), RDE-4 (AY071926), FLJ20399 (NP060273, BAB26260), CG1434 (AAF48360, EAA12065, CAA21662), CG13139 (XP059208, XP143416, XP110450, AAF52926, EEA14824), DGCRK6 (BAB83032, XP110167) CG1800 (AAF57175, EAA08039), FLJ20036 (AAH22270, XP134159), MRP-L45 (BAB14234, XP129893), CG2109 (AAF52025), CG12493 (NP647927), CG10630 (AAF50777), CG17686 (AAD50502), T22A3.5 (CAB03384) and accession number EAA14308. The sequences of such DRBPs are known in the art and can be obtained via their corresponding accession numbers.

A DRBP sequence for use in the present invention is FFMEELNTYRQKQGVVLKYQELP NSGPPHDRRFTFQVIIDGREFPEGEGRSKKEAKNAAAKLAVEILNKE (SEQ ID NO: 8; see also [21-22]). This preferred DRBP sequence is a dsRNA binding domain (DRBD) sequence, rather than a full DRBP sequence and is derived by truncation from PKR (Accession numbers AAA36409, AAA61926, Q03963).

More preferred adaptor molecules are variants of wild-type double stranded RNA binding proteins (DRBP variants) that have a reduced ability to bind dsRNA than the respective naturally occurring DRBPs mentioned above and are, therefore, less likely to interfere with the intended biological activity of the compound in the cell.

A DRBP variant which is more preferred in the present invention differs from the DRBP protein from which it is derived by up to 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145 or 150 amino acid changes in the amino acid sequence (i.e., substitutions, insertions, deletions, N-terminal truncations and/or C-terminal truncations). The amino acid substitutions may be conservative or non-conservative. A DRBP variant, which is preferred in the present invention can alternatively or additionally be characterised by a certain degree of sequence identity to the DRBP protein from which it is derived. Thus, the DRBP variants, which are preferred in the present invention have a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference (i.e., wild-type) DRBP.

Additionally, a DRBP variant is only regarded as a DRBP variant within the context of the present invention, if it exhibits the relevant biological activity to a degree of at least 30% of the activity of the wild-type DRBP protein. The relevant “biological activity” in the context of the present invention is the “binding activity”, i.e. the ability of the DRBP variant to bind the compound. One of ordinary skill in the art can readily assess whether a DRBP variant has a reduced dsRNA binding activity, i.e. at least 30% of the activity of the wild-type DRBP protein. Suitable assays, e.g. binding assays, for determining the “binding activity” of the DRBP variant compared to the binding activity of the wild-type DRBP are known to the person of ordinary skill in the art [22, 23].

Preferred dsDNA binding proteins (DDBPs) that can be employed as adapter molecules in the conjugates of the present invention are any protein or protein domain that comprising one of the following known DNA binding motifs: a helix-turn-helix motif, a zinc finger motif, a leucine zipper motif, a winged helix (turn helix) motif, a helix-loop-helix motif, or an HMG-box motif. In a particular embodiment, a conjugate of the present invention comprises a DDBP selected from the group consisting of HMGB1/2 (high-mobility group box 1 and 2 proteins, GeneIDs: 3146 and 3148, respectively), crp (GeneID 947867), Egr1 (GeneID 1958), Jun (GeneID 3725), FOXA1 (forkhead box A1; GeneID 3169), ETS1 (GeneID 2113), Twist1 (GeneID 22160), HIST2H2AC (histone cluster 2, GeneID 8338), and the like.

It is particularly preferred that the modules (a), (b), (c) and the compound (d) of the conjugate of the present invention have the following arrangements or combinations and comprise the following linkage types:
(i) (a)ₓ, (c)_{z}, (d)ₙ and (b)_{y}, wherein (a)ₓ is covalently linked to (c)_{z}, (c)_{z} is covalently linked to (d)ₙ, and (d)ₙ is covalently linked to (b)_{y};
(ii) (a)ₓ, (c)_{z}, (d)ₙ, and (b)_{y}, wherein (a)ₓ is covalently linked to (c)_{z}, (c)_{z} is covalently linked to (d)ₙ, and (d)ₙ is non-covalently linked to (b)_{y};
(iii) (a)ₓ, (d)ₙ, (c)_{z} and (b)_{y}, wherein (a)ₓ is covalently linked to (d)ₙ, (d)ₙ is covalently linked to (c)_{z}, and (c)_{z} is covalently linked to (b)_{y};
(iv) (a)ₓ, (d)ₙ, (c)_{z} and (b)_{y}, wherein (a)ₓ is non-covalently linked to (d)ₙ, (d)ₙ is non-covalently linked to (c)_{z}, and (c)_{z} is covalently linked to (b)_{y};
(v) (a)ₓ, (c)_{z}, (d)ₙ, and (b)_{y}, wherein (a)ₓ is covalently linked to (c)_{z} via a linker molecule, (c)_{z} is covalently linked to (d)ₙ via a linker molecule, and (d)ₙ is covalently linked to (b)_{y} via a linker molecule;
(vi) (a)ₓ, (c)_{z}, (d)ₙ, and (b)_{y}, wherein (a)ₓ is covalently linked to (c)_{z} via a linker molecule, (c)_{z} is covalently linked to (d)ₙ via a linker molecule, and (d)ₙ is non-covalently linked to (b)_{y};
(vii) (a)ₓ, (d)ₙ, (c)_{z} and (b)_{y}, wherein (a)ₓ is covalently linked to (d)ₙ via a linker molecule, (d)ₙ is covalently linked to (c)_{z} via a linker molecule and (c)_{z} is covalently linked to (b)_{y} via a linker molecule;
(viii) (a)ₓ, (d)ₙ, (c)_{z} and (b)_{y}, wherein (a)ₓ is non-covalently linked to (d)ₙ, (d)ₙ is non-covalently linked to (c)_{z}, and (c)_{z} is covalently linked to (b)_{y} via a linker molecule, or
(ix) (a)ₓ, (d)ₙ, (c)_{z} and (b)_{y}, wherein (a)ₓ is non-covalently linked to (d)ₙ via an adapter molecule that is covalently linked to (a)ₓ, (d)ₙ is non-covalently linked to (c)_{z} via an adapter molecule that is covalently linked to (c)_{z}, and (c)_{z} is covalently linked to (b)_{y} via a linker molecule, and wherein
   x is an integer of 1 to 5, preferably of 1;
   y is an integer of 1 to 5; preferably of 1;
   z is an integer of 1 to 5; preferably of 1; and
   n is an integer of 1 to 50, preferably of 2, 3, 4, 5, 6, 7, 8, 9, or 10.

It is preferred that there are no other linkages, preferably no covalent linkages, between the respective modules other than the linkages specifically indicated above or below with respect to the more preferred embodiments.

Thus, conjugates according to the present invention are particularly preferred that carry module (b) in a terminal position, preferably in last (i.e., C-terminal) position, and wherein modules (a), (b) and (c), and compound (d) are completely covalently linked to each other or partially covalently linked to each other, e.g., conjugate: (a)ₓ, (c)_{z}, (d)ₙ and (b)_{y}, wherein (a)ₓ is covalently linked to (c)_{z}, (c)_{z} is covalently linked to (d)ₙ, and (d)ₙ is covalently linked to (b)_{y}; or conjugate: (a)ₓ, (c)_{z}, (d)ₙ and (b)_{y}, wherein (a)ₓ is covalently linked to (c)_{z}, (c)_{z} is covalently linked to (d)ₙ, and (d)ₙ is non-covalently linked to (b)_{y}. In these examples, module (b) is unhindered by the other modules (a) and (c) and by the compound (d). Module (b) is also not extended by linkages of other modules. Hence, steric or other undesired interactions can be avoided or at least minimized.

For in vivo applications, it is preferred to use conjugates that comprise module (b) in the C-terminal position, and wherein modules (a), (b) and (c), and compound (d) are completely covalently linked to each other and/or covalently linked to each other via a linker molecule, e.g. conjugate: (a)ₓ, (c)_{z}, (d)ₙ and (b)_{y}, wherein (a)ₓ is covalently linked to (c)_{z}, (c)_{z} is covalently linked to (d)ₙ, and (d)ₙ is covalently linked to (b)_{y}; or conjugate: (a)ₓ, (c)_{z}, (d)ₙ, and (b)_{y}, wherein (a)ₓ is covalently linked to (c)_{z} via a linker molecule, (c)_{z} is covalently linked to (d)ₙ via a linker molecule, and (d)ₙ is covalently linked to (b)_{y} via a linker molecule; or conjugate: (a)ₓ, (d)ₙ, (c)_{z} and (b)_{y}, wherein (a)ₓ is covalently linked to (d)ₙ, (d)ₙ is covalently linked to (c)_{z}, and (c)_{z} is covalently linked to (b)_{y}. These exemplary conjugates are more stable compared to conjugates that comprise modules and compounds that are only non-covalently linked or partially non-covalently linked to each other and, thus are more preferred for in vivo applications.

For in vitro applications, e.g. in cell culture, it is preferred to use conjugates that comprise module (b) in the C-terminal position, and wherein modules (a), (b) and (c) are only partially covalently linked, e.g. conjugate: (a)ₓ, (d)ₙ, (c)_{z} and (b)_{y}, wherein (a)ₓ is non-covalently linked to (d)ₙ, (d)ₙ is non-covalently linked to (c)_{z}, and (c)_{z} is covalently linked to (b)_{y} via a linker molecule. This exemplary conjugate is less complex and easier to synthesize and, thus, more preferred for in vitro applications as predominant test systems. Nucleic acid compounds in this exemplary conjugate can also more readily be exchanged in order to test libraries of compound molecules for their biological activity in cells. Thus, the conjugates of the invention are also useful in screening assays.

Conjugates are also preferred that comprise compound (d) in second or third position, and wherein compound (d) is directly covalently linked or indirectly covalently linked via a linkage molecule to modules (a) or (c), e.g. conjugate: (a)ₓ, (d)ₙ, (c)_{z} and (b)_{y}, wherein (a)ₓ is covalently linked to (d)ₙ, (d)ₙ is covalently linked to (c)_{z}, and (c)_{z} is covalently linked to (b)_{y}; or conjugate: (a)ₓ, (d)ₙ, (c)_{z} and (b)_{y}, wherein (a)ₓ is covalently linked to (d)ₙ via a linker molecule, (d)ₙ is covalently linked to (c)_{z} via a linker molecule and (c)_{z} is covalently linked to (b)_{y} via a linker molecule. These exemplary conjugates assure flexibility of compound (d). In addition, the linker molecules connecting compound (d) with modules (a) and (c) have a spacer function, which keeps modules (a) and (c) safely away from the compound (d). Thus, steric and other undesired interactions can be avoided or at least minimized.

More preferred are conjugates according to the present invention that comprise the following arrangement:

(a)ₓ, (d)ₙ, (c)_{z} and (b)_{y}, wherein (a)ₓ is covalently linked to (d)ₙ, (d)ₙ is covalently linked to (c)_{z}, and (c)_{z} is covalently linked to (b)_{y}, and wherein x is an integer of 1, n is an integer of 2 or 3, z is an integer of 1, and y is an integer of 1, or
(a)ₓ, (d)ₙ, (c)_{z} and (b)_{y}, wherein (a)ₓ is covalently linked to (d)ₙ via a linker molecule, (d)ₙ is covalently linked to (c)_{z} via a linker molecule and (c)_{z} is covalently linked to (b)_{y} via a linker molecule, and wherein x is an integer of 1, n is an integer of 2, 3, 4, 5, 6, 7, 8, 9, or 10, z is an integer of 1 and y is an integer of 1.

It is particularly preferred that the modules (a), (b), (c) and the compound (d) of the conjugate of the present invention are linked to each other in the following arrangements, wherein
(i) (a)ₓ is covalently linked to (c)_{z}, (c)_{z} is covalently linked to (d)ₙ, and (c)_{z} is covalently linked to (b)_{y};
(ii) (a)ₓ is covalently linked to (c)_{z}, (c)_{z} is non-covalently linked to (d)ₙ, and (c)_{z} is covalently linked to (b)_{y};
(iii) (a)ₓ is covalently linked to (d)ₙ, (a)ₓ is covalently linked to (c)_{z}, and (c)_{z} is covalently linked to (b)_{y};
(iv) (a)ₓ is non-covalently linked to (d)ₙ, (a)ₓ is covalently linked to (c)_{z}, and (c)_{z} is covalently linked to (b)_{y};
(v) (a)ₓ is covalently linked to (c)_{z} via a linker molecule, (c)_{z} is covalently linked to (d)ₙ via a linker molecule, and (c)_{z} is covalently linked to (b)_{y} via a linker molecule;
(vi) (a)ₓ is covalently linked to (c)_{z} via a linker molecule, (c)_{z} is non-covalently linked to (d)ₙ via an adapter molecule that is covalently linked to (c)_{z}, and (c)_{z} is covalently linked to (b)_{y} via a linker molecule;
(vii) (a)ₓ is covalently linked to (d)ₙ via a linker molecule, (a)ₓ is covalently linked to (c)_{z} via a linker molecule and (c)_{z} is covalently linked to (b)_{y} via a linker molecule; or
(viii) (a)ₓ is non-covalently linked to (d)ₙ via an adapter molecule that is covalently linked to (a)ₓ, (a)ₓ is covalently linked to (c)_{z} via a linker molecule, and (c)_{z} is covalently linked to (b)_{y} via a linker molecule.

It is preferred that there are no other linkages, preferably no covalent linkages, between the respective modules other than the covalent linkages and non-covalent linkages, respectively, specifically indicated above.

More preferred, the modules (a), (b), (c) and the compound (d) of the conjugate of the present invention are linked to each other in the following arrangements, wherein
(i) (a)ₓ is covalently linked to (c)_{z}, (c)_{z} is covalently linked to (d)ₙ, and (c)_{z} is covalently linked to (b)_{y};
(ii) (a)ₓ is covalently linked to (c)_{z}, (c)_{z} is non-covalently linked to (d)ₙ, and (c)_{z} is covalently linked to (b)_{y};
(iii) (a)ₓ is covalently linked to (d)ₙ, (a)ₓ is covalently linked to (c)_{z}, and (c)_{z} is covalently linked to (b)_{y};
(iv) (a)ₓ is non-covalently linked to (d)ₙ, (a)ₓ is covalently linked to (c)_{z}, and (c)_{z} is covalently linked to (b)_{y};
(v) (a)ₓ is covalently linked to (c)_{z} via a linker molecule, (c)_{z} is covalently linked to (d)ₙ via a linker molecule, and (c)_{z} is covalently linked to (b)_{y} via a linker molecule;
(vi) (a)ₓ is covalently linked to (c)_{z} via a linker molecule, (c)_{z} is non-covalently linked to (d)ₙ via an adapter molecule that is covalently linked to (c)_{z}, and (c)_{z} is covalently linked to (b)_{y} via a linker molecule;
(vii) (a)ₓ is covalently linked to (d)ₙ via a linker molecule, (a)ₓ is covalently linked to (c)_{z} via a linker molecule and (c)_{z} is covalently linked to (b)_{y} via a linker molecule; or
(viii) (a)ₓ is non-covalently linked to (d)ₙ via an adapter molecule that is covalently linked to (a)ₓ, (a)ₓ is covalently linked to (c)_{z} via a linker molecule, and (c)_{z} is covalently linked to (b)_{y} via a linker molecule, and wherein
   x is an integer of 1 to 5, preferably of 1;
   y is an integer of 1 to 5; preferably of 1;
   z is an integer of 1 to 5; preferably of 1; and
   n is an integer of 1 to 50, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, preferably of 2, 3, 4, 5, 6, 7, 8, 9, or 10.

It is preferred that there are no other linkages, preferably no covalent linkages, between the respective modules other than the covalent linkages and non-covalent linkages, respectively, specifically indicated above.

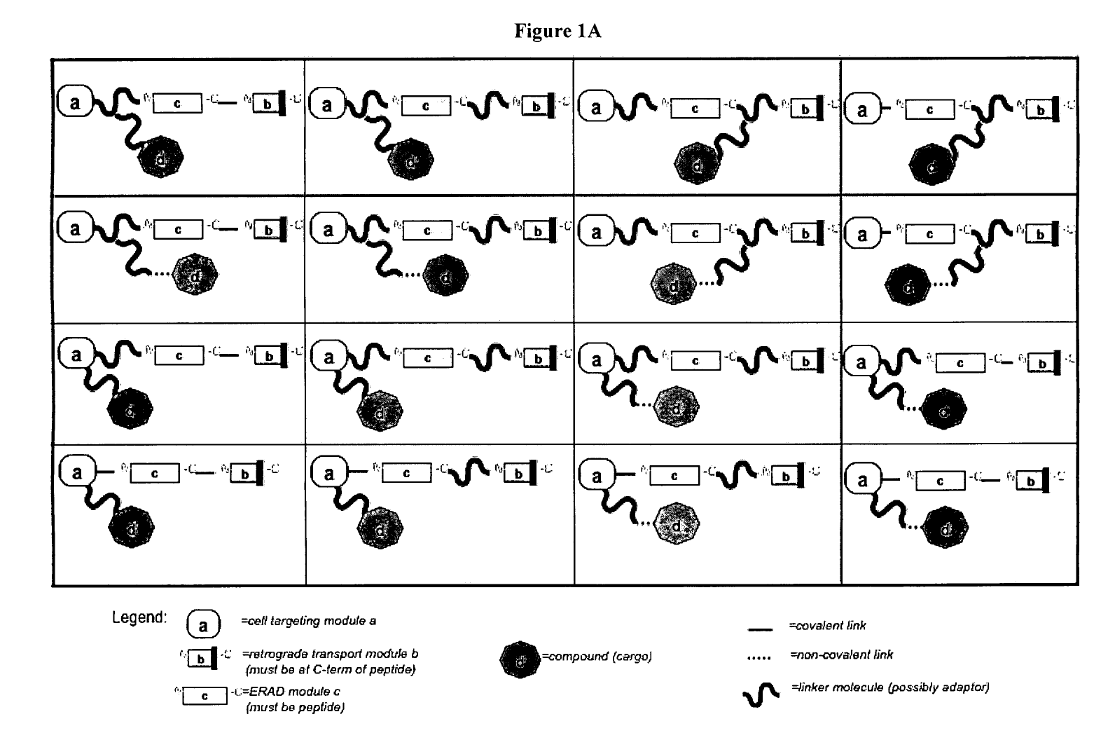

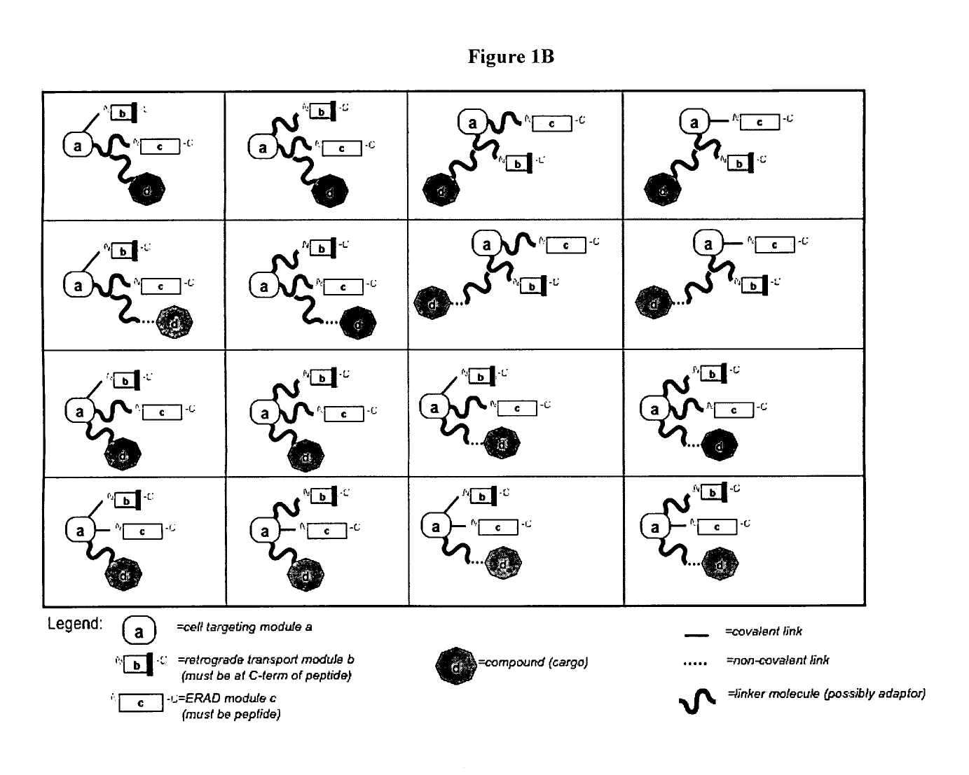

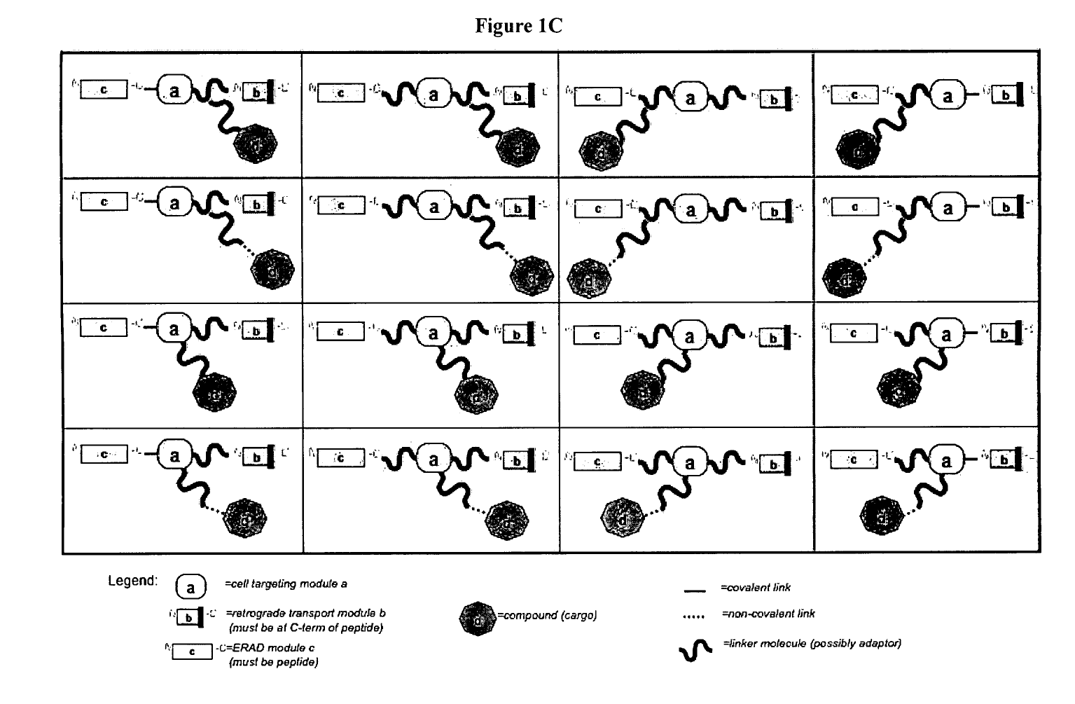

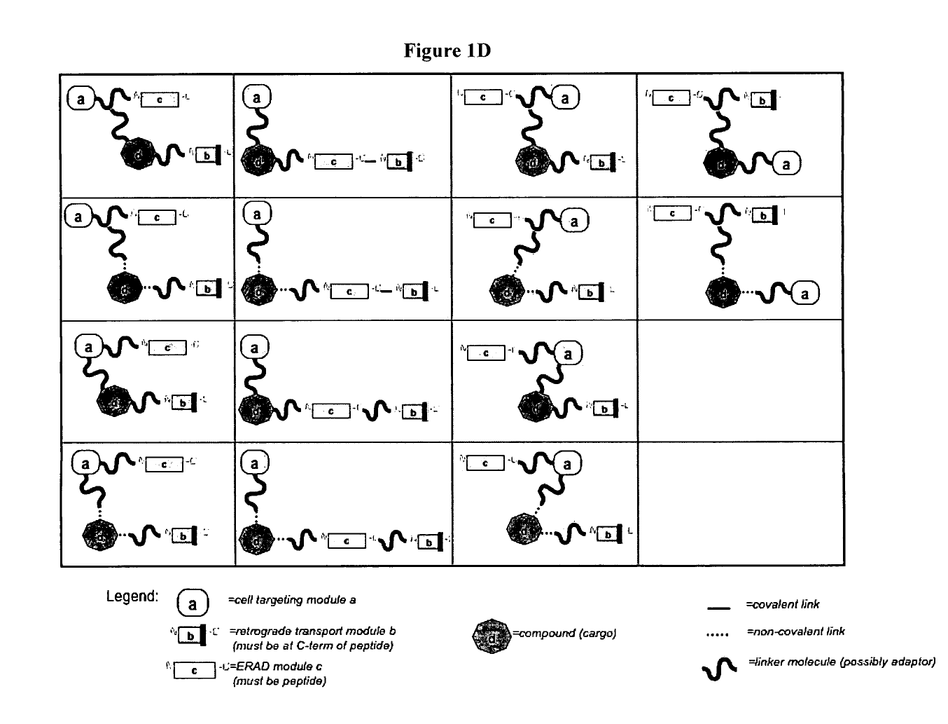

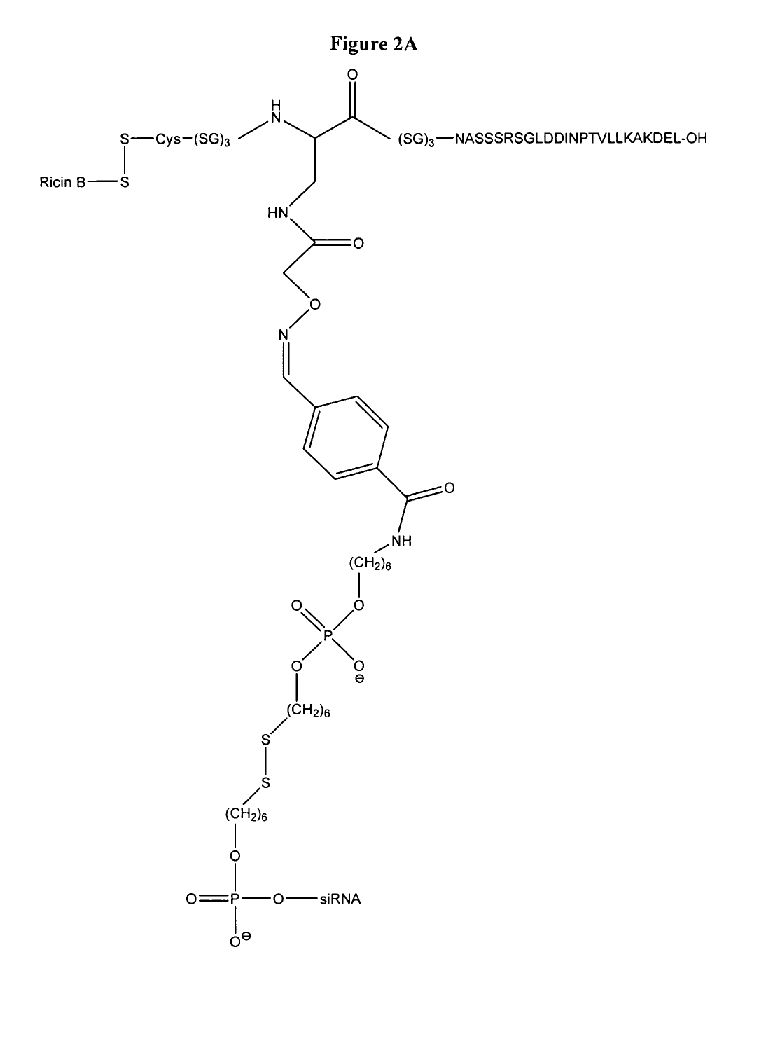

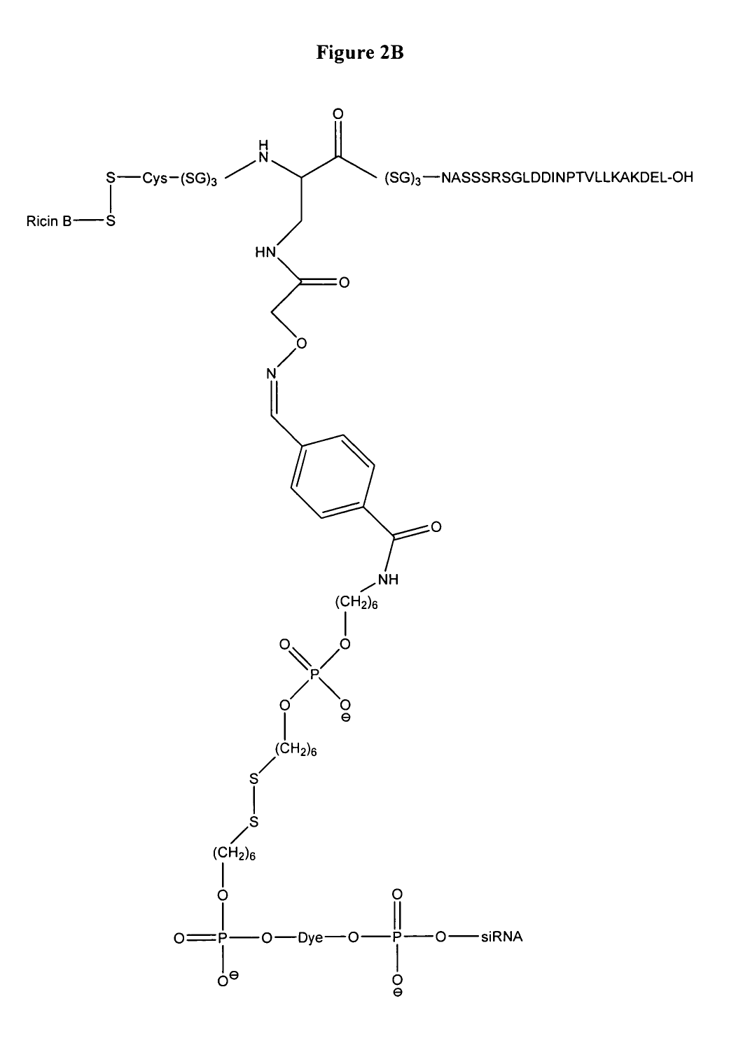

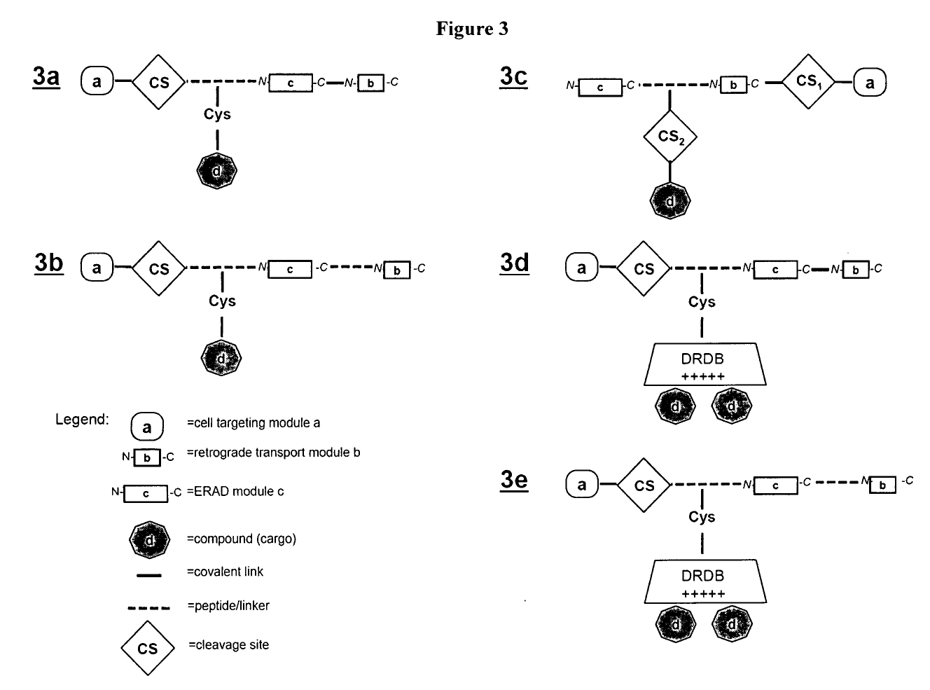

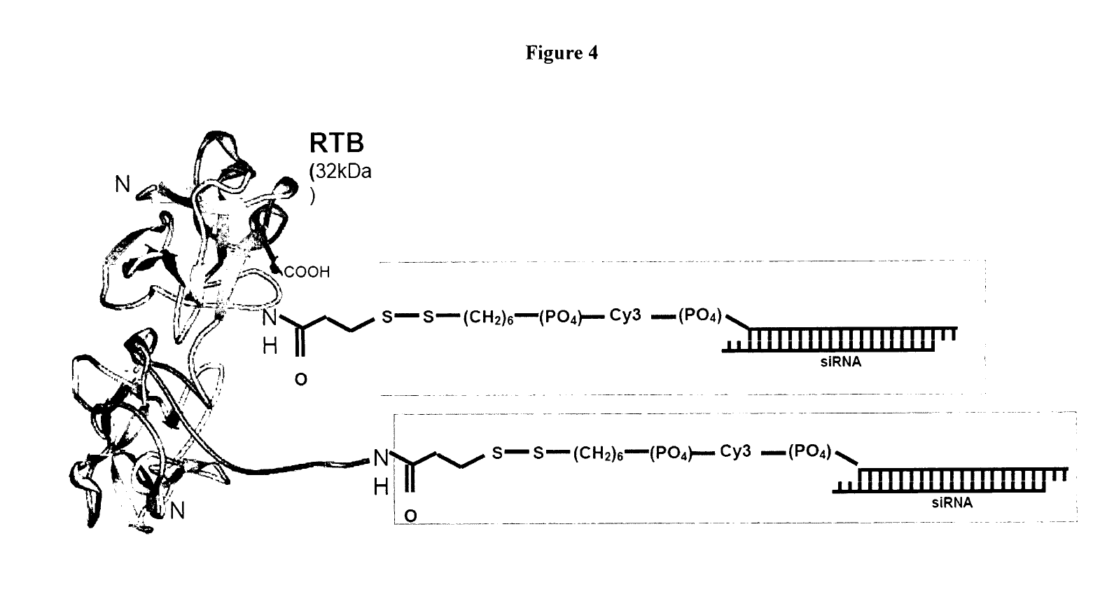

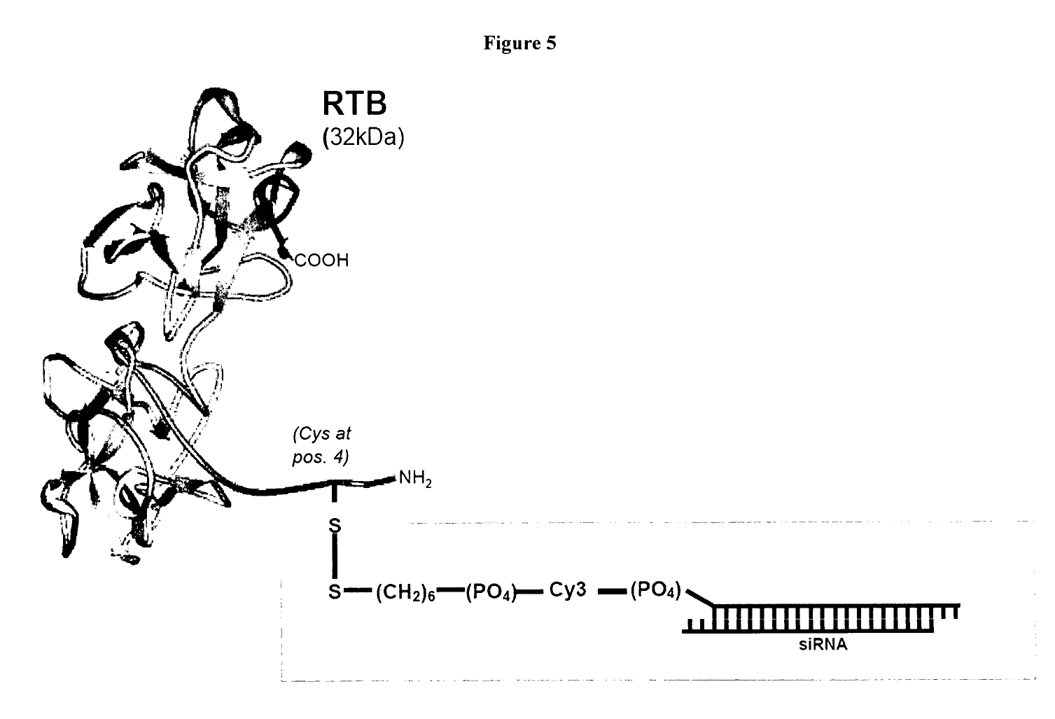

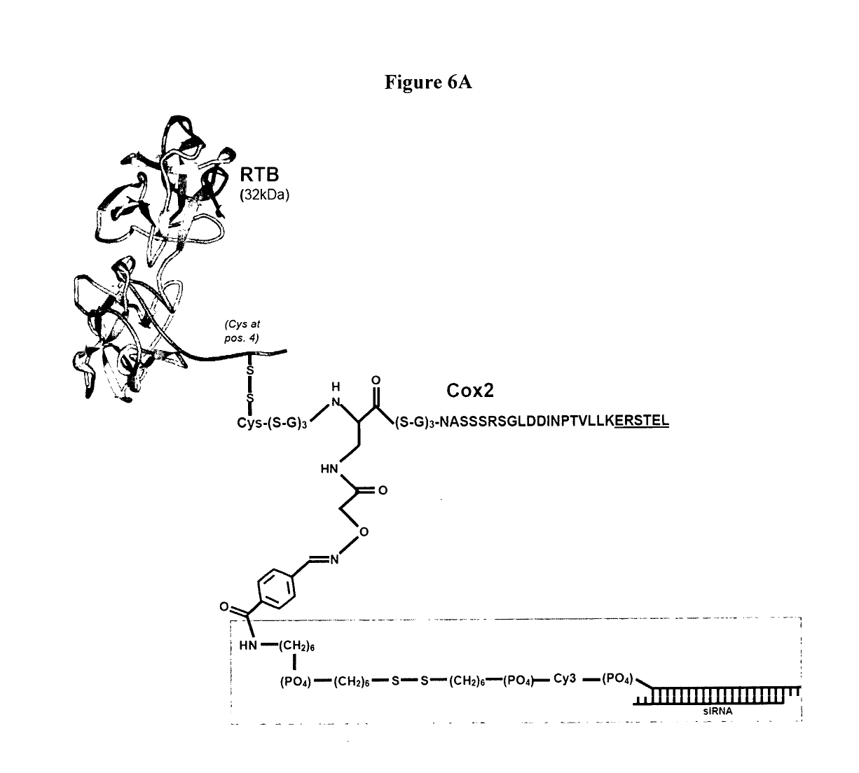

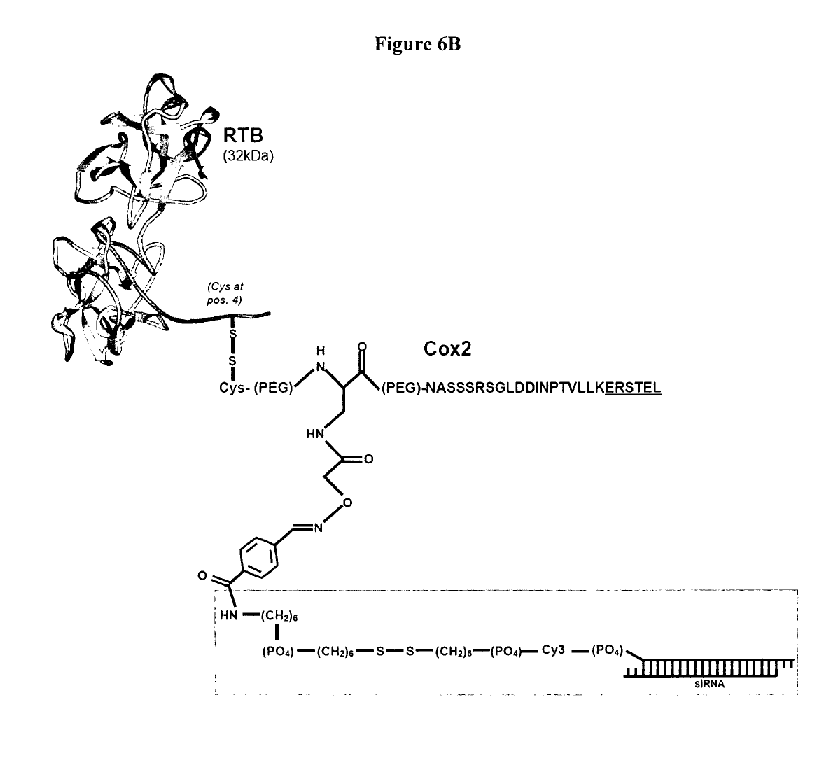

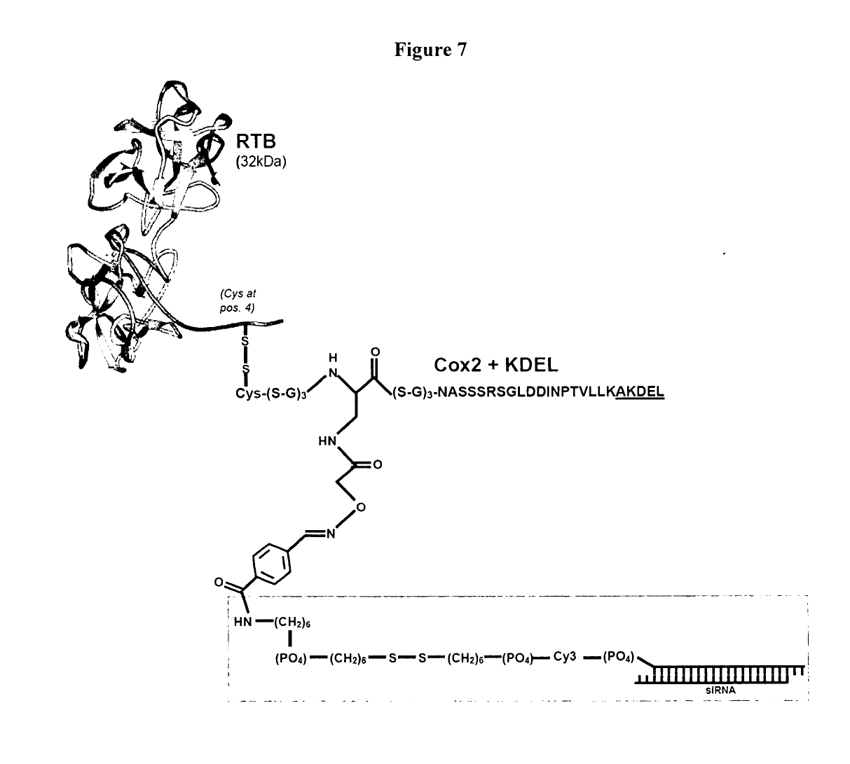

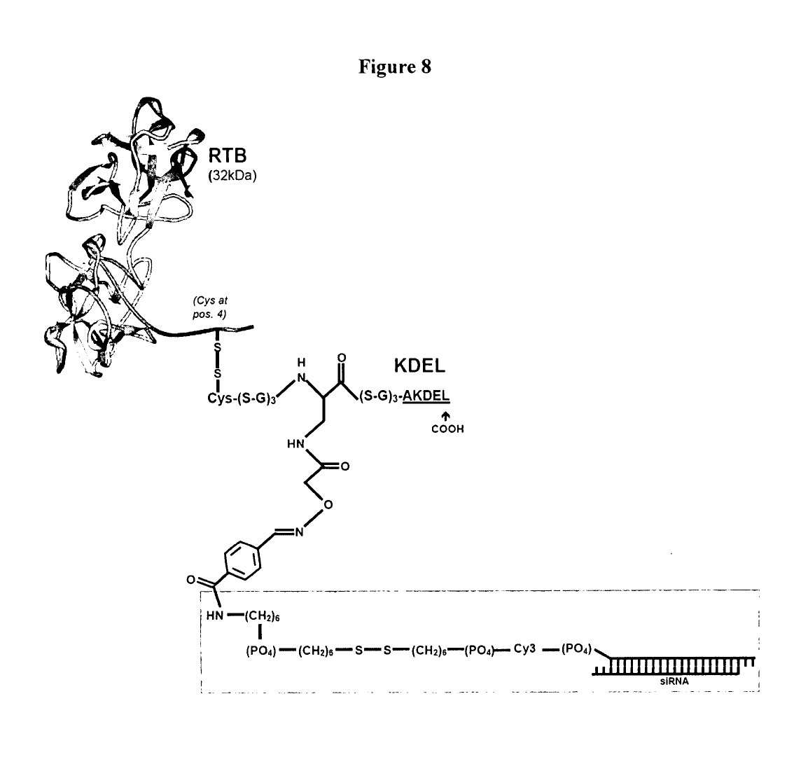

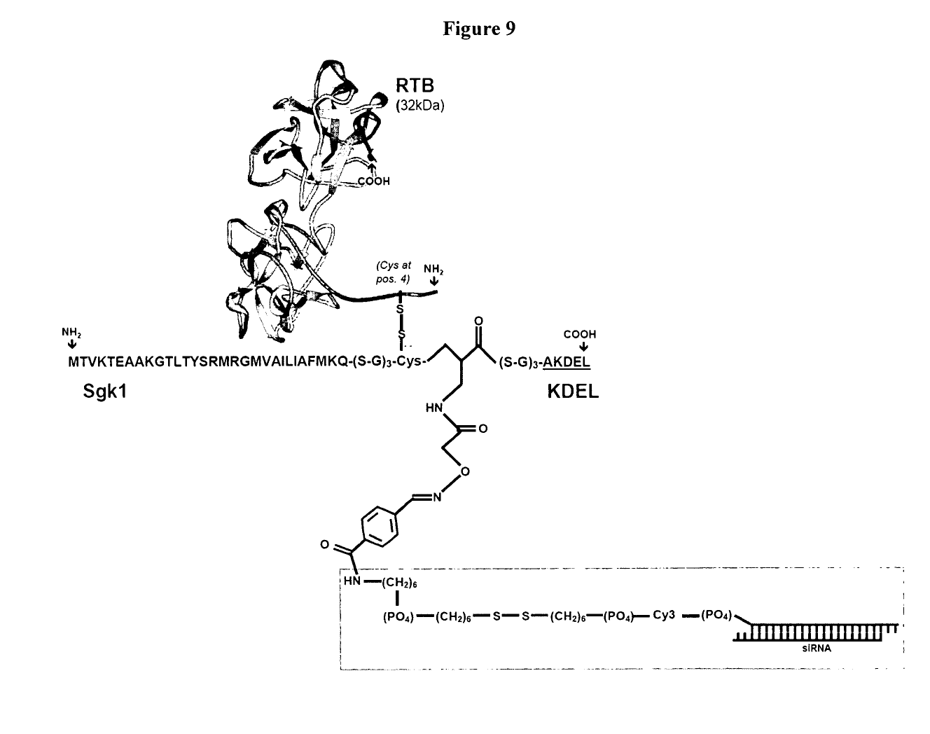

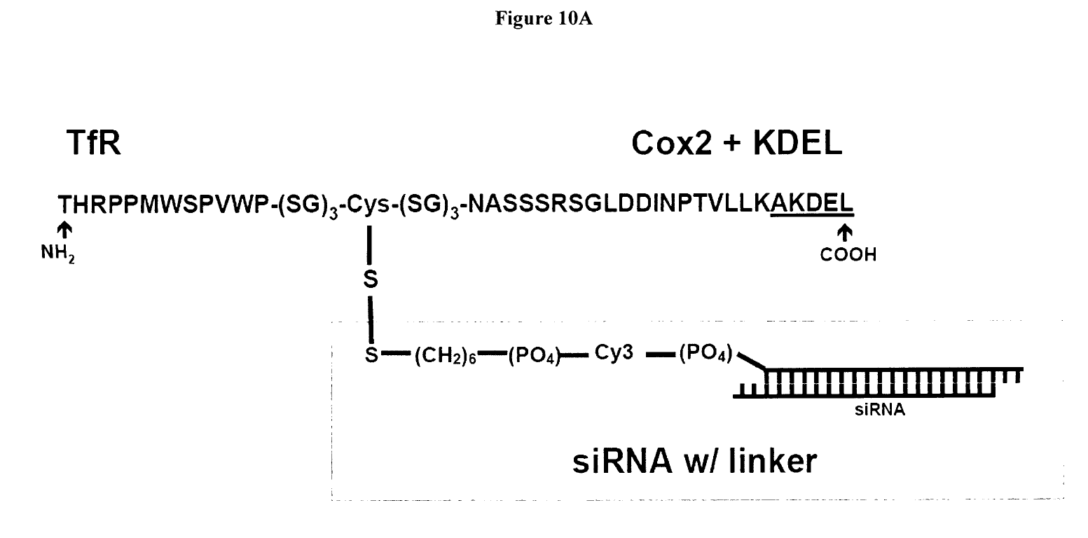

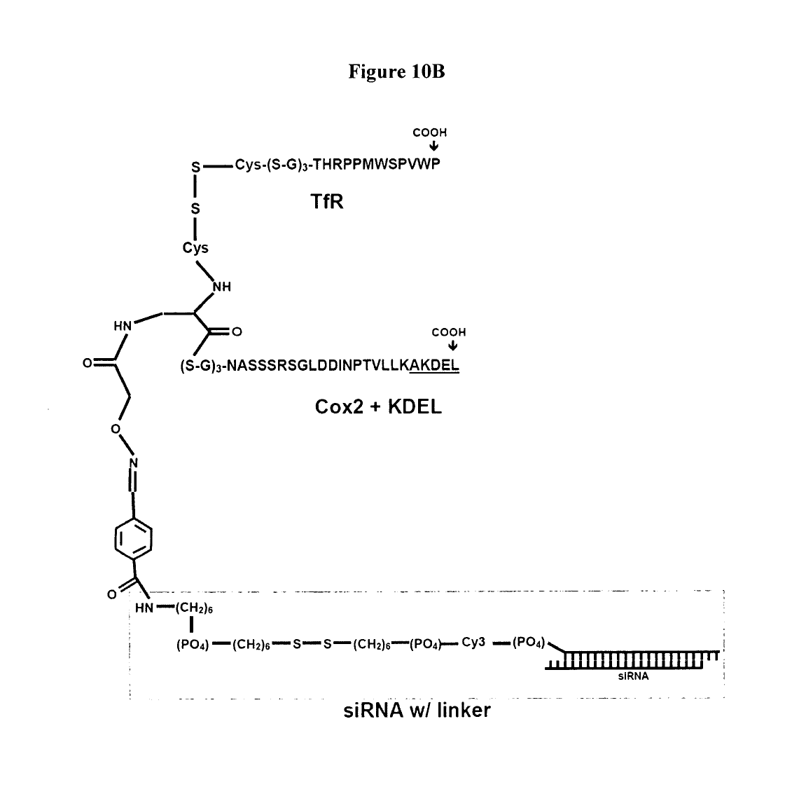

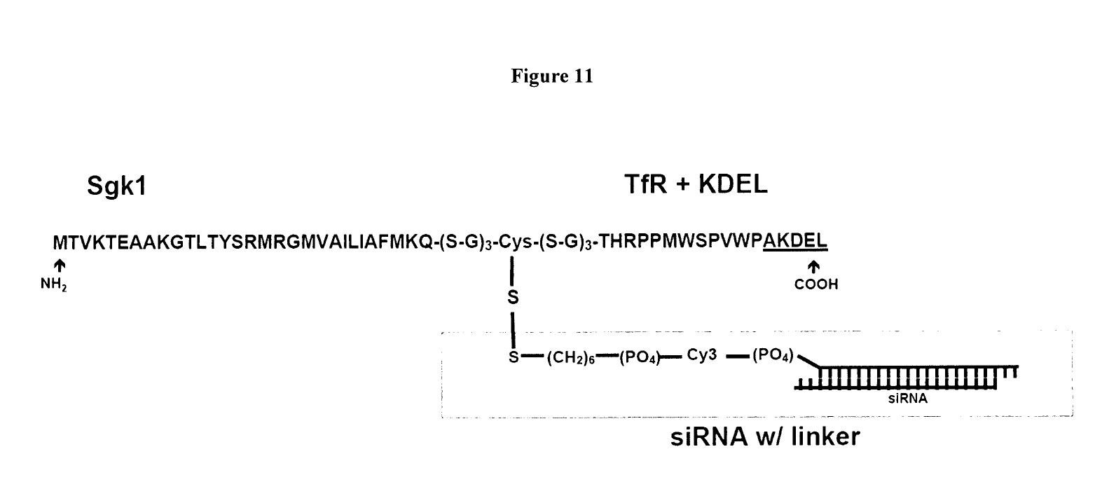

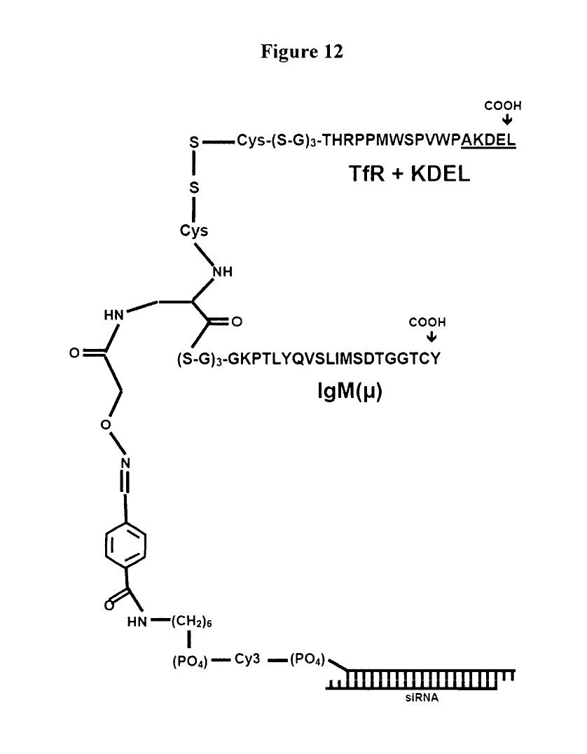

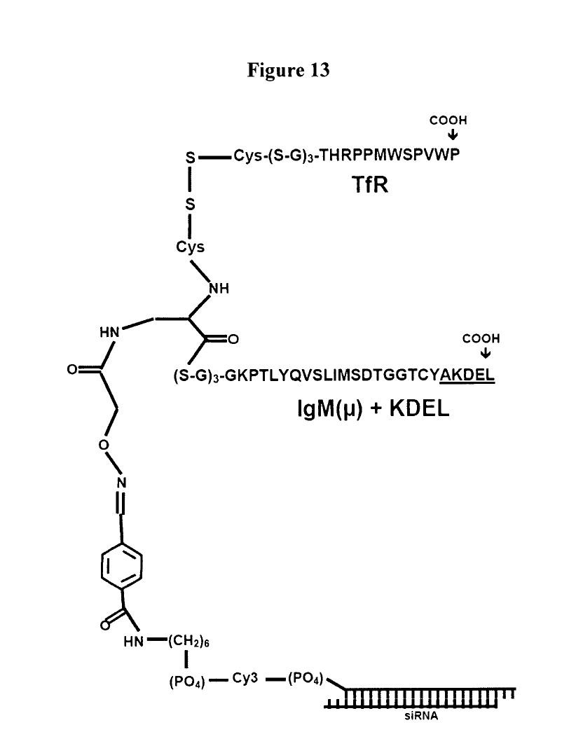

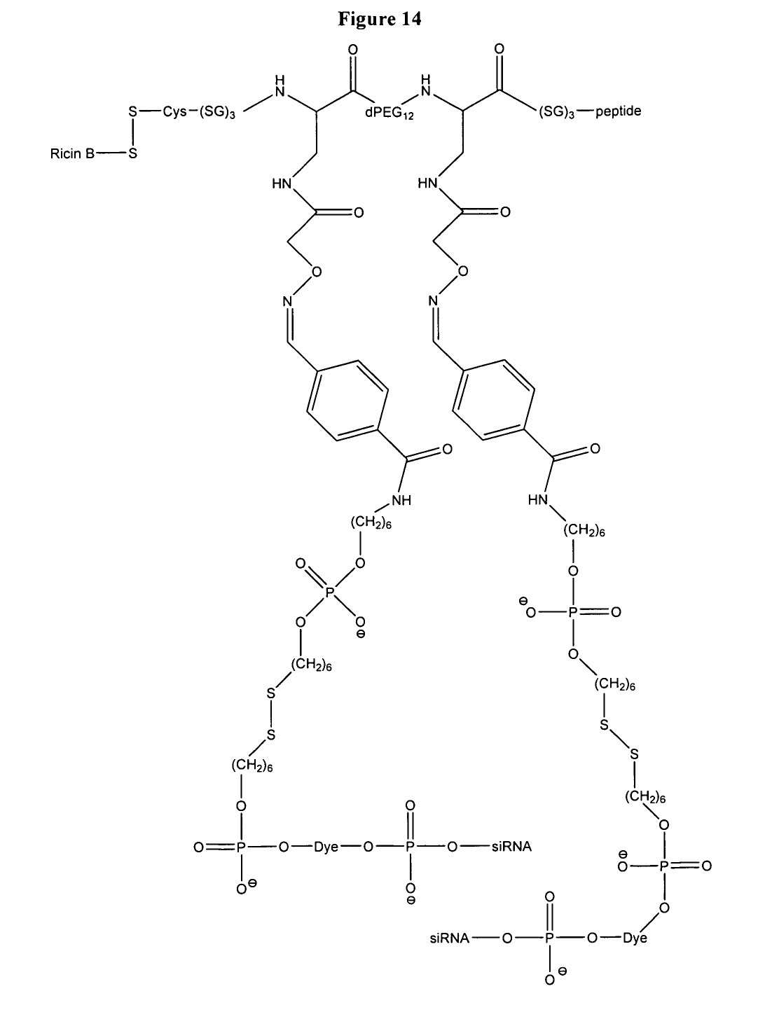

Preferred embodiments of the conjugate of the present invention are illustrated in FIGS. 1 (A) to (D), FIGS. 2 (A) and (B), FIGS. 3 (A) to (E), FIG. 4, FIG. 5, FIGS. 6 (A) and (B), FIG. 7, FIG. 8, FIG. 9, FIGS. 10 (A) and (B), FIG. 11, FIG. 12, FIG. 13, and FIG. 14. FIGS. 1 (A) to (D) illustrate preferred embodiments of the conjugate of the present invention, wherein the modules, either separately among each other, or together with the compound (d), may be linked either covalently, non-covalently, via an adapter molecule or via a linker molecule that optimally comprises an adapter molecule. FIGS. 2 (A) and (B), FIGS. 3 (A) to (E), FIG. 4, FIG. 5, FIGS. 6 (A) and (B), FIG. 7, FIG. 8, FIG. 9, FIGS. 10 (A) and (B), FIG. 11, FIG. 12, FIG. 13, and FIG. 14 illustrate additional preferred embodiments of a conjugate of the present invention as described herein and in the Examples below.

In another preferred embodiment, the linker molecule, e.g. a peptide, a modified peptide, an amino acid residue or a modified amino acid residue, of the conjugate of the present invention that covalently links the at least one module (a) and/or the at least one module (b) and/or the at least one module (c) and/or the at least one compound (d), arranged in any combination, order, or stoichiometry to each other, further comprises
(i) at least one branch point, preferably a cysteine side chain, a lysine side chain, or an unnatural amino acid containing an aminoxy moiety on the side chain, and/or
(ii) at least one cleavage site, preferably an endosomal enzyme, a trans-Golgi network enzyme, a Golgi enzyme, an ER enzyme, a cytosolic enzyme or a nuclear enzyme cleavage site.

The term “branch point” in the context of the present invention means a position in a linker molecule, e.g. in a peptide liker, preferably an amino acid side chain, to which molecules, preferably a compound or an adapter molecule, can be linked or coupled.

The term “cleavage site” in the context of the present invention means a specific amino acid sequence (e.g. a specific sequence within the amino acid sequence of the peptide linker molecule) or a specific chemical bond [e.g. a disulfide bond (S—S)] within the conjugate that is cleavable, e.g. via chemical cleavage or via cleavage by an enzyme, for example via a protease or peptidase that recognizes the specific sequence or via an enzyme which recognizes the specific chemical bond.

Wherein the linker molecule of the conjugate of the present invention comprises both a branch point and a cleavage site, it is preferred that the cleavage site is located upstream, e.g., 3′, of the branch point.

The presence of a cleavage site in the linker molecule connecting the at least one module (a), the at least one module (b), the at least one module (c), and/or the at least one compound (d) that may be arranged in any order, combination, or stoichiometry, of the conjugate of the present invention enables the separation of one or more of the modules and/or the at least one compound (d) during delivery of the compound (d) into a cell, e.g. after cellular uptake, after targeting the endoplasmic reticulum (ER), after delivery to the cytosol, or after delivery to the nucleus. Preferably, a conjugate of the present invention comprises at least 1, 2, 3, 4, 5, 6, 7, 8, 9, or 10 cleavage sites. More preferably, the conjugate comprises at least 1, 2, 3, 4, or 5 cleavage sites. Even more preferably, the conjugate comprises 1, 2, 3, 4, or 5 cleavage sites.

Preferably, a conjugate of the present invention comprises a cleavage site that is recognized by an enzyme, wherein the enzyme cleaves the conjugate at the cleavage site. The conjugate can be prepared with a cleavage site that is preferably recognized and cleaved by an enzyme that is located and active in a particular compartment or organelle of a cell or in the cell's cytosol. In a preferred embodiment, the conjugate comprises a cleavage site that is recognized and cleaved by an enzyme that is located and active in a target cell's endosome, a trans-Golgi network, Golgi, ER, cytosol, or nucleus. In another preferred embodiment, the conjugate comprises at least 2 cleavage sites, wherein each cleavage site is recognized and cleaved by at least 2 different enzymes, wherein the at least 2 different enzymes are each located and active in a different compartment, organelle or cytosol of a target cell.

In a specific embodiment, a conjugate of the present invention comprises a cleavage site that is recognized and cleaved by an endosomal enzyme, wherein the endosomal enzyme is preferably located and active in an early/recycling endosome. Preferably, the cleavage site is recognized and cleaved by furin, CHMP1A, ECE1, STAMBP, USP10, USP6, ZFYVE9, or the like.

In a specific embodiment, a conjugate of the present invention comprises a cleavage site that is recognized and cleaved by a trans-Golgi network enzyme. Preferably, the cleavage site is recognized and cleaved by furin and the like.

In a specific embodiment, a conjugate of the present invention comprises a cleavage site that is recognized and cleaved by a Golgi enzyme. Preferably, the cleavage site is recognized and cleaved by ADAM10, BACE1, CAPN8, CTSC, ECE2, MBTPS1, NCSTN, PCSK1, PCSK6, PCSK7, PSEN1, PSEN2, RHBDF1, Site-1 protease (S1P), Site-2 protease (S2P), SPPL2B, ZMPSTE24, or the like. In a particularly preferred embodiment, the cleavage site is recognized and cleaved by a Golgi-specific enzyme ECE2, PCSK7, SPPL2B, or the like.

In a specific embodiment, a conjugate of the present invention comprises a cleavage site that is recognized and cleaved by an ER enzyme. Preferably, the cleavage site is recognized and cleaved by a protein from the protein disulfide isomerase (PDI) family, BACE1, BACE2, CASP7, CTSA, CTSC, CTSH, CTSZ, cysteine protease ER-60, DPP4, ERAP2, ERMP1, HTRA2, KLK6, MBTPS1, NCLN, NCSTN, PCSK, PRSS50, RCE1, SPCS, TMPRSS3, ZMPSTE24, or the like.

In a specific embodiment, a conjugate of the present invention comprises a cleavage site that is recognized and cleaved by a cytosolic enzyme. Preferably, the cleavage site is recognized and cleaved by calpain or the like.

In a specific embodiment, a conjugate of the present invention comprises a cleavage site that is recognized and cleaved by a nuclear enzyme. Preferably, the cleavage site is recognized and cleaved by CAPN7, CASP1, CASP2, CASP3, CASP6, CASP7, CASP8, CASP14, GZMB, LONP2, PITRM1, PSMA1, PSMB1, PSMC1, PSME3, SENP1 or the like.

In a preferred embodiment, the cleavage site is positioned in the conjugate such that, when cleaved by the enzyme, the at least one module (a) of the conjugate is released from the conjugate. In this embodiment, the cleavage site is preferably positioned between module (a) and module (c) or module (b), or between module (a) and compound (d). Preferably, the cleavage site that releases module (a) from the conjugate is recognized and cleaved by an enzyme that is located and active in an endosome, the trans-Golgi network, the Golgi, the ER, the cytosol, or the nucleus of a target cell. More preferably, the cleavage site that releases module (a) from the conjugate is recognized and cleaved by an endosomal enzyme, a trans-Golgi network enzyme, a Golgi enzyme, an ER enzyme, a cytosolic enzyme, or a nuclear enzyme.

In another preferred embodiment, the cleavage site is positioned in the conjugate such that, when cleaved by the enzyme, the at least one module (b) of the conjugate is released from the conjugate. In this embodiment, the cleavage site is preferably positioned between module (b) and module (a) or module (c), or between module (b) and compound (d). Preferably, the cleavage site that releases module (b) from the conjugate is recognized and cleaved by an enzyme that is located and active in the ER, the cytosol, or the nucleus (e.g., calpain, a PDI family protein, BACE1, BACE2, CAPN7, CASP1, CASP2, CASP3, CASP6, CASP7, CASP8, CASP14, CTSA, CTSC, CTSH, CTSZ, DPP4, cysteine protease ER-60, ERAP2, ERMP1, GZMB, HTRA2, KLK6, LONP2, MBTPS1, NCLN, NCSTN, PCSK, PITRM1, PSMA1, PSMB1, PSMC1, PSME3, PRSS50, RCE1, SENP1, SPCS, TMPRSS3, ZMPSTE24, and the like). Preferably, the enzyme that is active in the ER, the cytosol, and/or the nucleus does not cleave off module (b) from the conjugate until the conjugate reaches the ER, the cytosol or the nucleus. More preferably, the cleavage site that releases module (b) from the conjugate is recognized and cleaved by an enzyme that is located and active in the ER, cytosol and/or nucleus but is not located or active in any of the cell compartments or organelles through which the conjugate of the present invention travels before reaching the ER, cytosol or nucleus. Even more preferably, the cleavage site that releases module (b) from the conjugate is recognized and cleaved by an enzyme that is located and active solely in the ER, the cytosol, and/or the nucleus.

In a specific embodiment, a conjugate of the present invention comprises a cleavage site within a peptide linker that is recognized and cleaved by an enzyme, wherein the enzyme is located and active in the ER, cytosol and/or nucleus but is not located or active in any of the cell compartments or organelles (e.g., endosomes, the Golgi, etc.) through which the conjugate of the present invention travels before reaching the ER, cytosol or nucleus (i.e., upstream of the ER, cytosol, or nucleus). Preferably, the cleavage site is recognized and cleaved by CASP7, CTSA, CTSH, CTSZ, ER-60, HTRA2, KLK6, NCLN, a PDI family protein, PRSS50, RCE1, TOR1A, and the like.

In another specific embodiment, a conjugate of the present invention comprises a cleavage site within a peptide linker that is recognized and cleaved by an enzyme, wherein the enzyme is located and active solely in the ER. Preferably, the cleavage site is recognized and cleaved by ER-60, ERMP1, a PDI family protein, SPCS1, TMPRSS3, or the like.

In another preferred embodiment, the cleavage site is positioned in the conjugate such that, when cleaved by the enzyme, the at least one compound (d) of the conjugate is released from the conjugate. In this embodiment, the cleavage site is preferably positioned between compound (d) and module (a), module (b) or module (c). When the compound (d) is desired to be delivered to the nucleus and the conjugate comprises a nuclear localization signal, the cleavage site is preferably positioned between compound (d) and the nuclear localization signal, and module (a), module (b) or module (c) such that, when cleaved by the enzyme, the at least one compound (d) and the nuclear localization signal are released from the conjugate. Preferably, the cleavage site that releases compound (d) or compound (d) and the nuclear localization signal from the conjugate is recognized and cleaved by an enzyme that is located and active in the cytosol or the nucleus.

In a preferred embodiment, the enzyme that is active in the cytosol or the nucleus does not cleave off compound (d) or compound (d) and the nuclear localization signal from the conjugate until the conjugate reaches the cytosol or the nucleus. More preferably, the cleavage site that releases compound (d) or compound (d) and the nuclear localization signal from the conjugate is recognized and cleaved by an enzyme that is located and active solely in the cytosol and/or the nucleus.

In a specific embodiment, a conjugate of the present invention comprises a cleavage site within a peptide linker that is recognized and cleaved by an enzyme, wherein the enzyme is located and active in the cytosol and/or nucleus but is not located or active in any of the cell compartments or organelles (e.g., endosomes, the trans Golgi network, the Golgi, the ER) through which the conjugate of the present invention travels before reaching the cytosol or nucleus (i.e., upstream of the cytosol or nucleus). Preferably, the cleavage site within a peptide linker is recognized and cleaved by calpain, ATG4A, CAPN10, CASP2, CASP3, CASP6, CASP9, GZMB, PREP, PREPL or the like.

In a preferred embodiment, a conjugate of the present invention comprises a cleavage site within a peptide linker that is recognized and cleaved by an enzyme, wherein the enzyme is located and active solely in the cytosol. Preferably, the cleavage site within a peptide linker is recognized and cleaved by calpain, PREPL or the like.

In another preferred embodiment, a conjugate of the present invention comprises a cleavage site within a peptide linker that is recognized and cleaved by an enzyme, wherein the enzyme is located and active solely in the nucleus. Preferably, the cleavage site within the peptide linker is recognized and cleaved by CAPN7, PITRM1, or the like.

In an alternative embodiment of the invention, the cleavage site within the conjugate is masked, such that the cleavage site is not available for cleavage until the conjugate reaches the intended compartment, organelle or cytosol in which cleavage at the cleavage site is desired. Masking of the cleavage site can be accomplished by a molecule that binds or interacts with the cleavage site within the conjugate, such that the masking molecule is released from the conjugate and the cleavage site is exposed when the conjugate reaches the intended compartment, organelle or cytosol in which cleavage of the conjugate is desired. Release of the masking molecule from the conjugate allows the cleavage enzyme to recognize and cleave the cleavage site and release the intended module, compound (d), or compound (d) and nuclear localization signal at the desired location within the cell. Alternatively, masking of a cleavage site within the conjugate of the invention may be due to the three-dimensional (3D) structure of the conjugate. In this alternative embodiment, a cleavage site is positioned within the conjugate such that it is internal (and therefore masked) within the 3D structure of the conjugate and is preferably made available for cleavage by removal of a portion of the conjugate (for example, when module (a) and/or module (b) is cleaved off from the conjugate, a cleavage site that is positioned between module (c) and compound (d) is no longer masked and is available for cleavage by its corresponding enzyme). Preferably, the masking molecule or the portion of the conjugate that is masking an internal cleavage site is released in the endosome, the TGN/Golgi Apparatus, the ER, the cytosol or the nucleus.

A preferred embodiment of the conjugate of the present invention comprises, for example, the following configuration: (a)ₓ, (d)ₙ, (c)_{z} and (b)_{y}, wherein (a)ₓ is covalently linked to (d)ₙ via a linker molecule comprising a cleavage site, (d)ₙ is covalently linked to (c)_{z} via a linker molecule comprising a different cleavage site and (c)_{z} is covalently linked to (b)_{y} and wherein x is an integer of 1, n is an integer of 1, 2, 3, 4, 5, 6, 7, 8, 9, or 10, z is an integer of 1 and y is an integer of 1. Thus, via the cleavage site between module (a) and module (d), it is possible to separate module (a) from the compound (d) and from the modules (c) and (b), e.g. after cellular uptake of the conjugate. As module (a) mediates cell targeting and facilitates cellular uptake, its function is no longer necessary after cell entry and thus, the presence of module (a) is not needed anymore. It is further possible to separate compound (d) from the modules (b) and (c) via the cleavage site between compound (d) and module (c), e.g. after transfer to the cytosol.

In a preferred embodiment of the present invention, it is preferred to add a furin cleavage site within a peptide linker molecule, preferably within a peptide linker molecule that covalently links module (a) to compound (d) and modules (c) or (b) in order to separate module (a) from the compound (d) and from modules (c) and/or (b) after uptake into the cell and/or upon reaching the Golgi apparatus. The minimal furin cleavage site is Arg-X-X-Arg (SEQ ID NO: 9). However, the furin enzyme prefers the site Arg-X-(Lys/Arg)-Arg (SEQ ID NO: 10). Furin is the major processing enzyme of the secretory pathway and is localized in the trans-golgi network. It cleaves proteins or peptides and, thus, also peptide linkers, carrying an Arg-X-X-Arg (SEQ ID NO: 9) or Arg-X-(Lys/Arg)-Arg (SEQ ID NO: 10) sequence. As a result, furin will cleave the peptide linker at the furin cleavage site between module (a) and compound (d) and modules (c) or (b), during transport of the conjugate to the ER via the TGN/Golgi Apparatus and thus, separate the module (a) from compound (d) and from the modules (c) and/or (b). It is preferred to add a calpain cleavage site within the peptide linker molecule, preferably within the peptide linker molecule that covalently links compound (d) to modules (c) or (b) in order to separate compound (d) from modules (c) and/or (b) after transfer to the cytosol. The peptide TPLKSPPPSPR (SEQ ID NO: 11) can act as a calpain cleavage site [24].

In another preferred embodiment, a conjugate of the present invention may alternatively or additionally comprise a calpain cleavage site comprising a sequence as listed in Table 2 or the like.

TABLE 2
Calpain Cleavage Sites of Use in a Conjugate
of the Present Invention.

| Substrate | Species | Cleavage Site(s) |
|---|---|---|
| ABP | Human | Pro-Gln-Tyr-Thr-Tyr-Ala |
| | | (SEQ ID NO: 12) |
| Actin | Human | Val-Gly-Arg-Pro-Arg-His |
| | | (SEQ ID NO: 13) |
| Annexin I | Bovine | Thr-Val-Lys-Gly-Ser-Lys |
| | | (SEQ ID NO: 14) |
| Arrestin | Bovine | Phe-Val-Phe-Glu-Glu-Phe |
| | | (SEQ ID NO: 15) |
| | | Gln-Asn-Leu-Lys-Asp-Ala |
| | | (SEQ ID NO: 16) |
| Calpain 30K | Chicken | Val-Ser-Met-Val-Asp-Pro |
| | | (SEQ ID NO: 17) |
| Alpain 80K | Chicken | Arg-Leu-Arg-Ala-Glu-Gly |
| | | (SEQ ID NO: 18) |
| CaMK IV | Mouse | Val-Cys-Gly-Thr-Pro-Gly |
| | | (SEQ ID NO: 19) |
| | | Thr-Glu-Asn-Leu-Val-Pro |
| | | (SEQ ID NO: 20) |
| CaM-PDE1A2 | Bovine | Val-Val-Gln-Ala-Gly-Ile |
| | | (SEQ ID NO: 21) |
| Caspase-9 | human | Gln-Leu-Asp-Ala-Ile-Ser |
| | | (SEQ ID NO: 22) |
| | | Pro-Glu-Ile-Arg-Lys-Pro |
| | | (SEQ ID NO: 23) |
| c-Fos | rat | Ser-Gln-Thr-Arg-Ala-Pro |
| | | (SEQ ID NO: 24) |
| c-Jun | rat | Leu-Asn-Leu-Ala-Asp-Pro |
| | | (SEQ ID NO: 25) |
| | | Leu-Leu-Thr-Ser-Pro-Asp |
| | | (SEQ ID NO: 26) |
| | | Ile-Thr-Thr-Thr-Pro-Thr |
| | | (SEQ ID NO: 27) |
| | | Ser-Leu-His-Ser-Glu-Pro |
| | | (SEQ ID NO: 28) |
| Connexin50 | sheep | Leu-Thr-Glu-Val-Gly-Met |
| | | (SEQ ID NO: 29) |
| | | Pro-Leu-Ser-Ala-Lys-Pro |
| | | (SEQ ID NO: 30) |
| Beta-Crystallin | bovine | Glu-Leu-Glu-Ser-Leu-Pro |
| A3 | | (SEQ ID NO: 31) |
| | | Thr-Thr-Lys-Met-Ala-Gln |
| | | (SEQ ID NO: 32) |
| dystrophin | human | Pro-Leu-Glu-Ile-Ser-Tyr |
| | | (SEQ ID NO: 33) |
| | | Val-Thr-Thr-Arg-Glu-Gln |
| | | (SEQ ID NO: 34) |
| EGFR | human | Arg-Leu-Leu-Gln-Glu-Arg |
| | | (SEQ ID NO: 35) |
| | | Trp-Ile-Pro-Glu-Gly-Glu |
| | | (SEQ ID NO: 36) |
| | | Ser-Thr-Ser-Arg-Thr-Pro |
| | | (SEQ ID NO: 37) |
| | | Ser-Cys-Pro-Ile-Lys-Glu |
| | | (SEQ ID NO: 38) |
| | | Asp-Thr-Phe-Leu-Pro-Val |
| | | (SEQ ID NO: 39) |
| | | Ser-Thr-Phe-Asp-Ser-Pro |
| | | (SEQ ID NO: 40) |
| | | Pro-Asn-Gly-Ile-Phe-Lys |
| | | (SEQ ID NO: 41) |
| GluR-1 | human | Ala-Ile-Arg-Thr-Ser-Thr |
| | | (SEQ ID NO: 42) |
| | | Ser-Ile-Asn-Glu-Ala-Ile |
| | | (SEQ ID NO: 43) |
| a-Hemoglobin | human | Asn-Val-Lys-Ala-Ala-Trp |
| | | (SEQ ID NO: 44) |
| b-Hemoglobin | human | Glu-Glu-Lys-Ser-Ala-Val |
| | | (SEQ ID NO: 45) |
| Histone H2A | bovine | Arg-Leu-Leu-Arg-Lys-Gly |
| | | (SEQ ID NO: 46) |
| Histone H2B | bovine | Gly-Thr-Lys-Ala-Val-Thr |
| | | (SEQ ID NO: 47) |
| Histone H3.2 | bovine | Ala-Thr-Gly-Gly-Val-Lys |
| | | (SEQ ID NO: 48) |
| HMG-CoA | rat | Pro-Lys-Lys-Ala-Gln-Asp |
| reductase | | (SEQ ID NO: 49) |
| Integrin beta 2 | human | Thr-Val-Met-Asn-Pro-Lys |
| | | (SEQ ID NO: 50) |
| | | Lys-Leu-Lys-Ser-Gln-Trp |
| | | (SEQ ID NO: 51) |
| | | Pro-Leu-Phe-Lys-Ser-Ala |
| | | (SEQ ID NO: 52) |
| Integrin beta 3 | human | Glu-Arg-Ala-Arg-Ala-Lys |
| | | (SEQ ID NO: 53) |
| | | Trp-Asp-Thr-Ala-Asn-Asn |
| | | (SEQ ID NO: 54) |
| | | Pro-Leu-Tyr-Lys-Glu-Ala |
| | | (SEQ ID NO: 55) |
| | | Ser-Thr-Phe-Thr-Asn-Ile |
| | | (SEQ ID NO: 56) |
| | | Ile-Thr-Tyr-Arg-Gly-Thr |
| | | (SEQ ID NO: 57) |
| Interleukin-1a | dog | Lys-Pro-Arg-Ser-Val-Ala |
| | | (SEQ ID NO: 58) |
| Interleukin-1a | human | Lys-Pro-Arg-Ser-Ser-Pro |
| | | (SEQ ID NO: 59) |
| MAP2c | rat | Val-Val-Thr-Ala-Glu-Ala |
| | | (SEQ ID NO: 60) |
| MBP | bovine | Asn-Ile-Val-Thr-Pro-Arg |
| | | (SEQ ID NO: 61) |
| | | Ala-Ser-Ala-Ser-Thr-Met |
| | | (SEQ ID NO: 62) |
| | | His-Tyr-Gly-Ser-Leu-Pro |
| | | (SEQ ID NO: 63) |
| | | Thr-Pro-Arg-Thr-Pro-Pro |
| | | (SEQ ID NO: 64) |
| Merlin | human | Val-Asn-Lys-Leu-Ile-Leu |
| | | (SEQ ID NO: 65) |
| | | Ile-Leu-Gln-Leu-Cys-Ile |
| | | (SEQ ID NO: 66) |
| MIP | rat | Ile-Leu-Lys-Gly-Ala-Arg |
| | | (SEQ ID NO: 67) |
| Myosin-V | chicken | Pro-Leu-Arg-Met-Glu-Glu |
| (brain) | | (SEQ ID NO: 68) |
| | | Pro-Leu-Ser-Arg-Thr-Pro |
| | | (SEQ ID NO: 69) |
| NKEF-B | human | Lys-Glu-Tyr-Phe-Ser-Lys |
| | | (SEQ ID NO: 70) |
| | | Ser-Asp-Thr-Ile-Lys-Pro |
| | | (SEQ ID NO: 71) |
| NMDAR 2A | rat | Leu-Gln-Phe-Gln-Lys-Asn |
| | | (SEQ ID NO: 72) |
| | | Leu-Phe-Ser-Val-Pro-Ser |
| | | (SEQ ID NO: 73) |
| p35 | mouse | Ser-Thr-Phe-Ala-Gln-Pro |
| | | (SEQ ID NO: 74) |
| p53 | human | Trp-Lys-Leu-Leu-Pro-Glu |
| | | (SEQ ID NO: 75) |
| pADPRT | bovine | Ala-Val-His-Ser-Gly-Pro |
| | | (SEQ ID NO: 76) |
| | | His-Leu-Leu-Ser-Pro-Trp |
| | | (SEQ ID NO: 77) |
| | | Lys-Ser-Gly-Ala-Ala-Pro |
| | | (SEQ ID NO: 78) |
| | | Lys-Leu-Thr-Val-Asn-Pro |
| | | (SEQ ID NO: 79) |
| Phospholipase | bovine | Ala-Leu-His-Ser-Gln-Pro |
| C-beta1 | | (SEQ ID NO: 80) |
| | | Glu-Asn-Pro-Gly-Lys-Glu |
| | | (SEQ ID NO: 81) |
| Phosphorylase | rabbit | Pro-Arg-Gly-Lys-Phe-Lys |
| kinase g | | (SEQ ID NO: 82) |
| PKC-alpha | human | Gly-Asn-Lys-Val-Ile-Ser |
| | | (SEQ ID NO: 83) |
| | | Lys-Ala-Lys-Leu-Gly-Pro |
| | | (SEQ ID NO: 84) |
| | | Glu-Asp-Arg-Lys-Gln-Pro |
| | | (SEQ ID NO: 85) |
| PKC-beta | human | Lys-Ile-Gly-Gln-Gly-Thr |
| | | (SEQ ID NO: 86) |
| | | Glu-Glu-Lys-Thr-Ala-Asn |
| | | (SEQ ID NO: 87) |
| PKC-gamma | human | Pro-Ser-Ser-Ser-Pro-Ile |
| | | (SEQ ID NO: 88) |
| | | Arg-Cys-Phe-Phe-Gly-Ala |
| | | (SEQ ID NO: 89) |
| PMCA-2 | human | Gly-Leu-Asn-Arg-Ile-Gln |
| | | (SEQ ID NO: 90) |
| | | Glu-Leu-Arg:Arg-Gly-Gln |
| | | (SEQ ID NO: 91) |
| RyR1 | rabbit | Met-Met-Thr-Gln-Pro-Pro |
| | | (SEQ ID NO: 92) |
| | | Ile-Ser-Gln-Thr-Ala-Gln |
| | | (SEQ ID NO: 93) |
| Spectrin aII | human | Glu-Val-Tyr-Gly-Met-Met |
| | | (SEQ ID NO: 94) |
| Spectrin b | human | Lys-Ser-Thr-Ala-Ser-Trp |
| | | (SEQ ID NO: 95) |
| Talin | human | Val-Leu-Gln-Gln-Gln-Tyr |
| | | (SEQ ID NO: 96) |
| Tau | human | Glu-Val-Met-Glu-Asp-His |
| | | (SEQ ID NO: 97) |
| | | Gly-Leu-Lys-Glu-Ser-Pro |
| | | (SEQ ID NO: 98) |
| | | Val-Val-Arg-Thr-Pro-Pro |
| | | (SEQ ID NO: 99) |
| | | Asp-Leu-Lys-Asn-Val-Lys |
| | | (SEQ ID NO: 100) |
| | | Asn-Val-Lys-Ser-Lys-Ile |
| | | (SEQ ID NO: 101) |
| | | Asn-Leu-Lys-His-Gln-Pro |
| | | (SEQ ID NO: 102) |
| | | Ile-Val-Tyr-Lys-Pro-Val |
| | | (SEQ ID NO: 103) |
| | | Glu-Val-Lys-Ser-Glu-Lys |
| | | (SEQ ID NO: 104) |
| | | Ile-Val-Tyr-Lys-Ser-Pro |
| | | (SEQ ID NO: 105) |
| Tyrosine 3- | bovine | Ala-Ile-Met-Ser-Pro-Arg |
| hydroxylase | | (SEQ ID NO: 106) |
| | | Glu-Leu-Asp-Ala-Lys-Gln |
| | | (SEQ ID NO: 107) |
| Vimentin | mouse | Arg-Leu-Arg-Ser-Ser-Val |
| | | (SEQ ID NO: 108) |
| | | Gly-Ser-Gly-Thr-Ser-Ser |
| | | (SEQ ID NO: 109) |
| | | Gly-Thr-Ser-Ser-Arg-Pro |
| | | (SEQ ID NO: 110) |
| | | Val-Thr-Thr-Ser-Thr-Arg |
| | | (SEQ ID NO: 111) |
| | | Arg-Thr-Tyr-Ser-Leu-Gly |
| | | (SEQ ID NO: 112) |
| | | Ser-Leu-Gly-Ser-Ala-Leu |
| | | (SEQ ID NO: 113) |
| | | Ser-Leu-Tyr-Ser-Ser-Ser |
| | | (SEQ ID NO: 114) |
| | | Val-Thr-Arg-Ser-Ser-Ala |
| | | (SEQ ID NO: 115) |
| von Willebrand | human | Leu-Leu-Lys-Ser-His-Arg |
| factor | | (SEQ ID NO: 116) |
| | | Ser-Lys-Arg-Ser-Leu-Ser |
| | | (SEQ ID NO: 117) |

One of skill in the art can easily use another cleavage site(s) in place of or in addition to the cleavage sites recited herein. Cleavage recognition sequences for other enzymes are available and accessible to anyone skilled in the art.

Preferably, the compound of a conjugate of the present invention is covalently linked to the branch point, preferably via an amide-linkage to the lysine side chain, via a disulfide-linkage to the cysteine side chain or via an unnatural amino acid containing an aminoxy moiety on the side chain.

Thus, in a preferred embodiment of a conjugate according to the present invention, the modules and the compound (d) are linked to each other in the following arrangement, wherein module (a) is covalently linked to module (c) via a peptide linker molecule which comprises a cysteine side chain as branch point and a cleavage site upstream of the branch point, module (c) is covalently linked to module (b), and compound (d) is covalently linked via a disulfide-linkage to the cysteine side chain [for example, see FIG. 3(A)].

In another preferred embodiment of the conjugate according to the present invention, the modules and the compound are linked to each other in the following arrangement, wherein module (a) is covalently linked to module (c) via a peptide linker molecule which comprises a cysteine side chain as branch point and a cleavage site upstream of the branch point, module (c) is covalently linked to module (b) via a peptide linker molecule, and compound (d) is covalently linked via a disulfide-linkage to the cysteine side chain of the branch point [for example, see FIG. 3(B)].

The cleavage site in the peptide linker molecule connecting module (a) and module (c) enables the separation of module (a), e.g., after cell entry, from the modules (c) and (b). As the cleavage site is located upstream of the branch point of the peptide linker to which the compound (d) is covalently linked, compound (d) and modules (c) and (b) can be separated from module (a).

In another preferred embodiment, compound (d) is linked via an enzymatic cleavage site instead of the disulfide-linkage to the cysteine side chain [for example, see FIG. 3(C)]. Preferably, module (a) is cleaved off of the conjugate in the endosome or TGN, whereby making module (b) available for cellular receptors or other cellular proteins that bind to cellular receptors and then facilitate further transport to the ER. In a preferred embodiment, a furin (active in the endosome and TGN) cleavage site or another proprotein convertase cleavage site may be designed in the peptide linker molecules of the present invention to cleave off a module(s) that is no longer required for further transport within the cell. Such cleavage could occur in any cell organelle (e.g. endosome, TGN, Golgi, etc.) and one of ordinary skill in the art is able to synthesize a peptide linker molecule comprising a desired cleavage site using standard methods and without undue experimentation.

Preferably, the compound (d) of a conjugate of the present invention is non-covalently linked to the branch point via an ionic linkage or via a hydrophobic linkage to DRBD or a variant thereof that is covalently linked via a disulfide linkage to the cysteine side chain.

Thus, in a preferred embodiment of a conjugate according to the present invention, the at least one module (a), the at least one module (b), the at least module (c) and the at least one compound (d) are linked to each other in the following arrangements, wherein the at least one module (a) is covalently linked to the at least one module (c) via a peptide linker molecule which comprises a cysteine side chain as a branch point and a cleavage site upstream of the branch point, the at least one module (c) is covalently linked to the at least one module (b) and the at least one compound (d) is non-covalently linked to the branch point via an ionic (electrostatic) linkage to DRBD that is covalently linked via a disulfide-linkage to the cysteine side chain [for example, see FIG. 3(D)].

In another preferred embodiment, at least two (2) compounds (d) are non-covalently linked to the branch point via an ionic linkage to the DRBD that is covalently linked via the disulfide-linkage to the cysteine side chain.

In another preferred embodiment of the conjugate according to the present invention, for example, the modules and the compound are linked to each other in the following arrangement or combination, wherein module (a) is covalently linked to module (c) via a peptide linker molecule which comprises a cysteine side chain as branch point and a cleavage site upstream of the branch point, module (c) is covalently linked to module (b) via a peptide linker molecule and compound (d) is non-covalently linked to the branch point via an ionic linkage to DRBD that is covalently linked via a disulfide-linkage to the cysteine side chain [for example, see FIG. 3(E)].

It shall be understood that the conjugates described in FIGS. 1 (A) to (D), FIGS. 2 (A) and (B), FIGS. 3 (A) to (E), FIG. 4, FIG. 5, FIGS. 6 (A) and (B), FIG. 7, FIG. 8, FIG. 9, FIGS. 10 (A) and (B), FIG. 11, FIG. 12, FIG. 13, and FIG. 14 represent only a small portion of the possible configurations of a conjugate of the present invention. One of skill in the art can make conjugates of other configurations without undue experimentation, and these conjugates are also encompassed within the scope of the present invention.

The conjugate of the present invention preferably comprises modules which are of endogenous origin in order to minimize the risk of unexpected immune reactions. Modules from exogenous sources may also be used within a conjugate of the present invention. If a module(s) from an exogenous source is used within a conjugate of the present invention, it is preferred that the exogenous module carries minimal risk of toxicity, or other unwanted activities such as immune activation, or oncogenicity.

The conjugate of the present invention comprises at least one module that mediates cell targeting and facilitates cellular uptake, designated as module (a), and is preferably of human origin.

Basically any molecule or structure that has high affinity binding to one or more than one molecule or structure on the surface of a target cell is suitable as module (a), and preferably triggers internalization into vesicular compartments capable of undergoing retrograde transport. Alternatively, module (a) can provide this target cell uptake functionality indirectly by binding to a molecule outside the target cells (i.e., in a pre-incubation before use, in the cell culture media or in an organism's blood, spinal fluid, interstitial fluid, etc., and defined herein as a “indirect targeting adapter molecule”), wherein the target cells directly recognize the indirect targeting adapter molecule, and wherein the indirect targeting adapter molecule preferably triggers internalization into vesicular compartments capable of undergoing retrograde transport.

In a preferred embodiment, a bispecific antibody (e.g., diabody or single-chain antibody) is used to bind both module (a) of the conjugate and a cell surface receptor on a desired target cell. Briefly, the bispecific antibody is pre-incubated with a conjugate comprising a module (a) that is recognized by the bispecific antibody before exposure or administration of the conjugate to a target cell. Upon exposure or administration to the target cell, the bispecific antibody-conjugate complex binds to the cell surface receptor that is recognized by the bispecific antibody. As a result of binding to the cell surface receptor, the bispecific antibody-conjugate complex preferably triggers internalization into a vesicular compartment from which retrograde transport can be initiated. In another embodiment, module (a) comprises an antibody (immunoglobulin, Ig) binding domain that is able to bind to an antibody that binds to a cell surface receptor on a desired target cell, thereby indirectly targeting the conjugate of the present invention to a cell of interest. In another preferred embodiment, module (a) comprises a biotin acceptor peptide that is able to bind to a biotinylated ligand that binds to a cell surface receptor on a desired target cell to indirectly target the conjugate of the present invention to the cell of interest.

Thus, the present invention provides a flexible platform for cell targeting since any ligand or binding particle that is able to enter a cell using endocytosis, and preferably triggers internalization into vesicular compartments capable of undergoing retrograde transport, can be exploited to target the conjugates of the present invention to a desired cell. Indeed, such targeting approaches are commonly used for targeting viral vectors and are well described in the literature (see for example, [25]). In addition, this indirect targeting approach is advantageous for the development of reagents for use with a delivery system or conjugate of the present invention, or kits comprising the same. Thus, one of skill in the art will be able to recognize and use different combinations of a module (a) and an indirect targeting adapter molecule to indirectly target conjugates encompassed by the present invention to a cell of interest, without undue experimentation.

In a particularly preferred embodiment, a conjugate of the present invention comprises a module (a) that either directly or indirectly confers a transcytosis functionality, whereby the conjugate can penetrate through or within a tissue, a tumor, an endothelial cell, and the like. Examples of molecules that may be used as module (a) for trancystosis functionality include but are not limited to albumin, orosomucoid, IgG, low density lipoprotein (LDL) cholesterol (not via LDL receptor), gonadotrophin, transferrin (not via transferrin receptor), melanotransferrin (p97; [26]), insulin, LDL, dIgA (dimeric immunoglobulin (Ig)A), vitamin B12, vitamin D, vitamin A, iron, HRP (horseradish peroxidase), ferritin, thyroglobulin, and the like (for a review, see [27]). Alternatively, one can use an antibody directed to albumin, orosomucoid, IgG, LDL cholesterol (not via LDL receptor), gonadotrophin, transferrin (not via transferrin receptor), melanotransferrin (p97), insulin, LDL, dIgA, vitamin B12, vitamin D, vitamin A, iron, HRP, ferritin, thyroglobulin, and the like, as a module (a) comprising a transcytosis functionality for use in a conjugate of the present invention.

All molecules, which are naturally taken up by any cell with high efficiency and fast kinetics can be used as module (a) or indirectly, to bind to module (a), provided that the molecule is internalized into or arrives in an intracellular membranous organelle. Such molecules preferably carry a low risk of eliciting an immune response or toxicity. Other molecules known to undergo cellular uptake, but which also carry certain secondary activities, such as an increased risk of immune stimulation may also be used as module (a).

Preferably, module (a), or the indirect targeting adapter molecule to which module (a) binds, of the conjugate of the present invention comprises a ligand of a cell surface marker that allows, causes and/or results in specific cell targeting and cellular uptake. Preferably, said ligand of a cell surface marker is a cell surface receptor ligand, an antibody, a sugar, a lipid or a nanoparticle, preferably of human origin.

It is particularly preferred that the cell surface receptor ligand is a ligand selected from the group consisting of a growth factor, a autocrine motility factor (AMF), a lipoprotein, a transferrin, a surface binding lectin, a galectin, a c-type lectin, a toxin, a fragment thereof, and a variant thereof.

Preferably, the cell surface receptor ligand is a growth factor selected from the group consisting of EGF, VEGF, BMPs, FGF, G-CSF, GM-CSF, HGF, GDFs, IGFs, NGF, TGFs, PGF, and PDGF.

In a preferred embodiment, the cell surface receptor ligand is an Autocrine Motility Factor [AMF, also known as glucose phosphate isomerse (GPI)]. AMF or other peptides, proteins, and small molecules that bind to AMF receptors and trigger its internalization are preferred cell surface receptor ligands of the present invention. Preferably, an AMF peptide of use in the conjugates of the present invention comprises an amino acid sequence comprising SEQ ID NO: 118 (full length human AMF), or a fragment or variant thereof. In another embodiment, an AMF peptide of use in the conjugates of the present invention comprises an amino acid sequence comprising SEQ ID NO: 119 (full length mouse AMF), or a fragment or variant thereof.

In another preferred embodiment, the cell surface receptor ligand is a sulfatase-modifying factor (SUMF). SUMF or other peptides, proteins, and small molecules that bind to SUMF receptors and trigger its internalization are preferred cell surface receptor ligands of the present invention. Preferably, an SUMF peptide or protein of use in the conjugates of the present invention comprises an amino acid sequence comprising human SUMF1 protein (SEQ ID NO: 120; UniProtKB/Swiss-Prot Q8NBK3 [28]), or a fragment of variant thereof.

Preferably, the cell surface ligand is a lipoprotein selected from the group consisting of a high density liproprotein (HDL) receptor/scavenger receptor family lipoprotein and a low density lipoprotein (LDL) receptor family lipoprotein.

Preferably, the cell surface ligand is a transferrin receptor (TfR) binding peptide selected from the group consisting of THRPPMWSPVWP (SEQ ID NO: 121; [29] and U.S. Pat. No. 6,743,893), GHKVKRPKG (SEQ ID NO: 122; [30] and WO2003/050238), and HAIYPRH (SEQ ID NO: 123; [29]).

Preferably, the cell surface ligand is a lectin selected from the group consisting of a soluble lectin, a collectin, and an intelectin (ITLN).

Preferably, the cell surface ligand is a galectin selected from the group consisting of LGALS1, LGALS2, LGALS3, LGALS4, LGALS5, LGALS6, LGALS7, LGALS8, LGALS9, LGALS10, LGALS11, LGALS12, and LGALS13.

Preferably, the cell surface ligand is a toxin selected from the group consisting of a bacterial toxin and a plant toxin. In a preferred embodiment, module (a) of the conjugate of the present invention comprises or consists of a toxin protein or peptide selected from the group consisting of a ricin toxin B-subunit, a cholera toxin B-subunit, a Shiga toxin (STx) B-subunit, a Shiga-like toxin (SLT) B-subunit [Verotoxin (VT) B-subunit], an E. coli heat-labile enterotoxin (LT) B-subunit, an abrin toxin B-subunit, a Pertussis toxin B-subunit, an Abrin B-subunit, a Modeccin B-subunit, a Volkensin B-subunit, Pseudomonas Exotoxin A domain I, Pseudomonas Exotoxin A domain II, and Pseudomonas Exotoxin A domain IV. Preferably, module (a) comprises a ricin toxin B-subunit peptide (SEQ ID NO: 124 or a recombinantly produced ricin toxin B-subunit as described in WO2008/157263), a cholera toxin B-subunit peptide (SEQ ID NO: 125), an Stx B-subunit peptide (SEQ ID NO: 126), an STx1 (SLT-I or VT1) B-subunit peptide (SEQ ID NO: 127), an SLT-Ib B-subunit peptide (SEQ ID NO: 128), an SLT-Ic B-subunit peptide (a VT1c peptide) (SEQ ID NO: 129), an SLT-IIb-subunit peptide (a VT2 peptide) (SEQ ID NO: 130), an SLT-IIc B-subunit peptide (a VT2c peptide) (SEQ ID NO: 131), an SLT-IId B-subunit peptide (a VT2d peptide) (SEQ ID NO: 132), an SLT-IIe B-subunit peptide (a VT2e peptide) (SEQ ID NO: 133), an SLT-IIf B-subunit peptide (a VT2f peptide) (SEQ ID NO: 134), an LT B-subunit peptide (SEQ ID NO: 135 or SEQ ID NO: 136), an LTIIa B-subunit peptide (SEQ ID NO: 137), an LTIIb B-subunit peptide (SEQ ID NO: 138), or an abrin toxin B-subunit peptide (SEQ ID NO: 139).

A growth factor, lipoprotein, transferrin, surface binding lectin, galectin, c-type lectin or toxin variant differs from the wild-type growth factor, lipoprotein, transferrin, surface binding lectin, galectin, c-type lectin or toxin protein from which it is derived by up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 200, 250, 300, 350, 400, 450, 500, 550 or 600 amino acid changes in the amino acid sequence (i.e. substitutions, insertions, deletions, N-terminal truncations and/or C-terminal truncations). Such a variant can alternatively or additionally be characterised by a certain degree of sequence identity to the wild-type protein from which it is derived. Thus, a growth factor, lipoprotein, transferrin, surface binding lectin, galectin, c-type lectin or toxin variant has a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% to the respective reference (wild-type) growth factor, lipoprotein, transferrin, surface binding lectin, galectin, c-type lectin or toxin.

A fragment (or deletion variant) of the growth factor, lipoprotein, transferrin, surface binding lectin, galectin, c-type lectin or toxin protein has preferably a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 170, 200, 250, 300, 350, 400, 450, 500, 550 or 600 amino acids at its N-terminus and/or at its C-terminus and/or internally.

Additionally, a growth factor, lipoprotein, transferrin, surface binding lectin, galectin, c-type lectin or toxin protein variant or fragment is only regarded as a growth factor, lipoprotein, transferrin, surface binding lectin, galectin, c-type lectin or toxin protein variant or fragment within the context of the present invention, if it exhibits a relevant biological activity to a degree of at least 3 to 50%, preferably at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50% of the activity of the wild-type growth factor, lipoprotein, transferrin, surface binding lectin, galectin, c-type lectin or toxin protein. In a preferred embodiment, the growth factor, lipoprotein, transferrin, surface binding lectin, galectin, c-type lectin or toxin protein variant or fragment for use in a conjugate of the present invention, exhibits its relevant biological activity to a degree of at least 4 to 50%, at least 5 to 50%, at least 10 to 50%, at least 20 to 50%, at least 30 to 50%, at least 40 to 50%, or at least 45 to 50% of the activity of the wild-type growth factor, lipoprotein, transferrin, surface binding lectin, galectin, c-type lectin or toxin protein. The relevant “biological activity” in this context is the “activity to mediate cell targeting and to facilitate cellular uptake”, i.e. the ability of the variant or fragment to contact a cell and to enter the cell. One of ordinary skill in the art can readily assess whether a growth factor, lipoprotein, transferrin, surface binding lectin, galectin, c-type lectin or toxin protein variant or fragment has the ability to mediate cell targeting and to facilitate cellular uptake, i.e. at least 3 to 50%, at least 4 to 50%, at least 5 to 50%, at least 10 to 50%, at least 20 to 50%, at least 30 to 50%, at least 40 to 50%, or at least 45 to 50%, preferably at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50% of the activity of the wild-type growth factor, lipoprotein, transferrin, surface binding lectin, galectin, c-type lectin or toxin protein. Suitable assays, e.g. in vitro tracing of fluorescently labelled variants or fragments, for determining the “activity to mediate cell targeting and to facilitate cellular uptake” of a growth factor, lipoprotein, transferrin, surface binding lectin, galectin, c-type lectin or toxin protein variant or fragment compared to the binding activity of the respective wild-type protein are known to the person of ordinary skill in the art. Examples of suitable wild-type activity standards/in vitro tracing assays of use with the present invention are well described [for example, 14, 16 and 31-34), incorporated herein in their entirety and the like].

In another embodiment of the present invention, module (a), or the indirect targeting adapter molecule to which module (a) binds, comprises an antibody. Preferably, the antibody is selected from the group consisting of an anti-TGN38/46, an anti-transferrin receptor, and an anti-growth factor receptor.

In another embodiment of the present invention, module (a), or the indirect targeting adapter molecule to which module (a) binds, comprises a sugar. Preferably, the sugar is selected from the group consisting of glucose, mannose, galactose, N-acetylglucosamine, N-acetylgalactosamine, fucose, N-acetylneuraminic acid and xylose.

In another embodiment of the present invention, module (a), or the indirect targeting adapter molecule to which module (a) binds, comprises a lipid. Preferably, the lipid is selected from the group consisting of a phospholipid, a glycolipid, a sphingolipid, and a sterol lipid.

In another embodiment of the present invention, module (a), or the indirect targeting adapter molecule to which module (a) binds, comprises a nanoparticle. Preferably, the nanoparticle is selected from the group consisting of a metal, a silicate, and a polymer. More preferably, the nanoparticle is a polymer selected from the group consisting of a poly(urethane), a poly(methyl methacrylate), a poly(vinyl alcohol), a poly(ethylene), a poly(vinyl pyrrolidone), a polylactide (PLA), a polyglycolide (PGA), a poly(lactide-co-glycolide) (PLGA), a polyanhydride and a polyorthoester.

In another embodiment of the present invention, module (a), or the indirect targeting adapter molecule to which module (a) binds, comprises a viral peptide that causes and/or results in specific cell targeting and cellular uptake. Preferably, said viral peptide is from a polyomavirus. More preferably, said viral peptide is from SV40, murine polyomavirus, BK virus, JC virus, KI virus, WU virus, and Merkel Cell polyomavirus. In the case of SV40, it has been shown to bind its cell surface receptor sialic acid on GM1 and its co-receptor MHC I, and is then transported to caveolae and from there into caveosomes; further transport brings SV40 into the smooth ER [35]. A second pathway has also been described in which SV40 avoids caveolae but exploits caveosomes to transport it from the caveosome to the ER [36]. Similar intracellular transport pathways have been described for the mouse polyomavirus (mPyV) and for other polyomaviruses [37]. Thus, a viral peptide, fragment or variant from SV40, murine polyomavirus, BK virus, JC virus, KI virus, WU virus, or Merkel Cell polyomavirus may be used as a module (a) or bound by a module (a) in the conjugates of the present invention.

The conjugate of the present invention comprises at least one module that facilitates the transport to the endoplasmic reticulum (ER), designated as module (b), and is preferably of human origin. Basically any molecule or structure that facilitates transport to the ER is suitable as module (b). Preferably, the module (b) of the conjugate of the present invention is an oligopeptide, preferably of human origin, which facilitates transport to the ER. In a conjugate of the present invention, module (b) can provide retrograde transport functionality either directly by comprising an oligopeptide that facilitates transport to the ER, or indirectly by binding to an endogenous protein, peptide or oligopeptide that facilitates transport to the ER (defined herein as an “endogenous ER transport protein, peptide or oligopeptide”).

The term “oligopeptide” in the context of the present invention means an amino acid sequence which comprises or consists of between 2 and 9 amino acid residues. Preferably, the oligopeptide of use with the conjugate of the present invention comprises between 2 and 9 amino acid residues in length. More preferably, the oligopeptide of use with the conjugate of the present invention comprises between 4 and 9 amino acid residues in length. More preferably, the oligopeptide of use with the conjugate of the present invention is 2, 3, 4, 5, 6, 7, 8 or 9 amino acid residues in length.

It is particularly preferred that the module (b), or the endogenous ER transport protein, peptide or oligopeptide to which module (b) binds, of the conjugate of the present invention comprises an oligopeptide comprising one or more of the amino acid sequence X₁X₂X₃X₄ (SEQ ID NO: 140), wherein X₁ is E, H, K, N, P, Q, R or S, preferably K or R; X₂ is D, E, A, T, V, G, S or N, preferably D or E; X₃ is E or D, preferably E; X₄ is L or F, preferably L, and wherein optionally the N-terminus and/or C-terminus comprises 1 to 3 additional amino acid residues.

More preferably, the module (b), or the endogenous ER transport protein, peptide or oligopeptide to which module (b) binds, of the conjugate of the present invention comprises an oligopeptide comprising one or more EDEL (SEQ ID NO: 141); HDEL (SEQ ID NO: 142); HEEL (SEQ ID NO: 143); KAEL (SEQ ID NO: 144); KDEF (SEQ ID NO: 145); KEDL (SEQ ID NO: 146); KEEL (SEQ ID NO: 147); KTEL (SEQ ID NO: 148); KVEL (SEQ ID NO: 149); NEDL (SEQ ID NO: 150); PDEL (SEQ ID NO: 151); PGEL (SEQ ID NO: 152); QEDL (SEQ ID NO: 153); QSEL (SEQ ID NO: 154); REDL (SEQ ID NO: 155); RNEL (SEQ ID NO: 156); RTDL (SEQ ID NO: 157); RTEL (SEQ ID NO: 158); ERSTEL (SEQ ID NO: 159); KDEL (SEQ ID NO: 160); AKDEL (SEQ ID NO: 161), PTEL (SEQ ID NO: 162); and/or STEL (SEQ ID NO: 163) motifs or variants thereof [38, 39].

The EDEL (SEQ ID NO: 141); HDEL (SEQ ID NO: 142); HEEL (SEQ ID NO: 143); KAEL (SEQ ID NO: 144); KDEF (SEQ ID NO: 145); KEDL (SEQ ID NO: 146); KEEL (SEQ ID NO: 147); KTEL (SEQ ID NO: 148); KVEL (SEQ ID NO: 149); NEDL (SEQ ID NO: 150); PDEL (SEQ ID NO: 151); PGEL (SEQ ID NO: 152); QEDL (SEQ ID NO: 153); QSEL (SEQ ID NO: 154); REDL (SEQ ID NO: 155); RNEL (SEQ ID NO: 156); RTDL (SEQ ID NO: 157); RTEL (SEQ ID NO: 158); ERSTEL (SEQ ID NO: 159); KDEL (SEQ ID NO: 160); AKDEL (SEQ ID NO: 161), PTEL (SEQ ID NO: 162); and/or STEL (SEQ ID NO: 163) motif variant differs from the respective wild-type motif from which it is derived by up to 1, 2, or 3 amino acid changes in the motif sequence (i.e. substitutions, insertions, deletions, N-terminal truncations and/or C-terminal truncations), preferably, conservative substitutions.

Additionally, said motif variant is only regarded as a motif variant within the context of the present invention, if it exhibits the relevant biological activity to a degree of at least 30%, preferably at least 50%, of the activity of the respective wild-type motif. The relevant “biological activity” in this context is the “activity to facilitate the transport to the endoplasmic reticulum (ER)”, i.e. the ability of the variant to target the conjugate to the endoplasmic reticulum (ER). The skilled person can readily assess whether an EDEL (SEQ ID NO: 141); HDEL (SEQ ID NO: 142); HEEL (SEQ ID NO: 143); KAEL (SEQ ID NO: 144); KDEF (SEQ ID NO: 145); KEDL (SEQ ID NO: 146); KEEL (SEQ ID NO: 147); KTEL (SEQ ID NO: 148); KVEL (SEQ ID NO: 149); NEDL (SEQ ID NO: 150); PDEL (SEQ ID NO: 151); PGEL (SEQ ID NO: 152); QEDL (SEQ ID NO: 153); QSEL (SEQ ID NO: 154); REDL (SEQ ID NO: 155); RNEL (SEQ ID NO: 156); RTDL (SEQ ID NO: 157); RTEL (SEQ ID NO: 158); ERSTEL (SEQ ID NO: 159); KDEL (SEQ ID NO: 160); AKDEL (SEQ ID NO: 161), PTEL (SEQ ID NO: 162); and/or STEL (SEQ ID NO: 163) motif variant has the ability to facilitate the transport to the ER, i.e. at least 30%, preferably at least 50%, of the activity of the respective wild-type motif. Suitable assays, e.g. in vitro tracing of fluorescently labelled variants, for determining the “activity to facilitate the transport to the endoplasmic reticulum (ER)” of an EDEL (SEQ ID NO: 141); HDEL (SEQ ID NO: 142); HEEL (SEQ ID NO: 143); KAEL (SEQ ID NO: 144); KDEF (SEQ ID NO: 145); KEDL (SEQ ID NO: 146); KEEL (SEQ ID NO: 147); KTEL (SEQ ID NO: 148); KVEL (SEQ ID NO: 149); NEDL (SEQ ID NO: 150); PDEL (SEQ ID NO: 151); PGEL (SEQ ID NO: 152); QEDL (SEQ ID NO: 153); QSEL (SEQ ID NO: 154); REDL (SEQ ID NO: 155); RNEL (SEQ ID NO: 156); RTDL (SEQ ID NO: 157); RTEL (SEQ ID NO: 158); ERSTEL (SEQ ID NO: 159); KDEL (SEQ ID NO: 160); AKDEL (SEQ ID NO: 161), PTEL (SEQ ID NO: 162); and/or STEL (SEQ ID NO: 163) variant compared to the binding activity of the respective wild-type motif are known to the person skilled in the art (see for example, [31]).

In another embodiment, module (b), or preferably the endogenous ER transport protein, peptide or oligopeptide to which module (b) binds, of the conjugate of the present invention is a Sortilin, SorLA, or S or CS protein, peptide or oligopeptide, or a fragment or variant thereof [40].

In another embodiment, module (b), or the endogenous ER transport protein, peptide or oligopeptide to which module (b) binds, of the conjugate of the present invention comprises a viral peptide that facilitates the transport to the ER. Preferably, said viral peptide is from a polyomavirus. More preferably, said viral peptide is from SV40, murine polyomavirus, BK virus, JC virus, KI virus, WU virus, and Merkel Cell polyomavirus. As described above, SV40 has been shown to bind its cell surface receptor sialic acid on GM1 and its co-receptor MHC I, and be transported to caveolae, then into caveosomes, and ultimately into the smooth ER [35]. SV40 has also been shown to avoid caveolae but exploit caveosomes to transport it from the caveosome to the ER [36]. Similar intracellular transport pathways have been described for the mouse polyomavirus (mPyV) and for other polyomaviruses [37]. Thus, a viral peptide, fragment or variant from SV40, murine polyomavirus, BK virus, JC virus, KI virus, WU virus, or Merkel Cell polyomavirus may be used as a module (b) or bound by module (b) in the conjugates of the present invention.

The conjugate of the present invention comprises or consists of at least one module that facilitates translocation from the endoplasmic reticulum (ER) to the cytosol (i.e., ERAD targeting), designated as module (c), and is preferably of mouse or human origin. Alternatively, module (c) can provide this ER to the cytosol translocation functionality indirectly by binding to an endogenous molecule that is capable of or is undergoing ERAD in the target cell. Examples of endogenous cellular molecule that may be bound by a module (c) of a conjugate of the present invention include but are not limited to COX2, Sgk1, null Hong Kong (NHK) variant of α1-antitrypsin (α1-AT), ASGPR H2a (a subunit of the asialoglycoprotein receptor), BACE457 [a pancreatic isoform of β-secretase (BACE)], CD38, TCRα, ΔF508 of CFTR (cystic fibrosis conductance regulator), HMG-CoA reductase (3-hydroxy-3-methyl-glutaryl-CoA reductase), Igκ LC NS (a transport-incompetent immuno-globulin light chain), KAI1 (also known as CD82), MHC (major histocompatibility complex) class I molecules, Pael-R (Pael receptor), transthyretin (TTR [41], and the like (see for example, [42]).

In a preferred embodiment, module (c) binds to a cellular molecule that has a naturally short half life due to rapid ERAD mediated degradation. Preferably, module (c) binds to an endogenous COX2 or Sgk1 protein or peptide.

Preferably, module (c) of the conjugate of the present invention comprises or consists of a peptide selected from the group consisting of Cyclooxygenase-2 (COX2), Immunoglobulin M heavy chain [IgM(μ)], Igh6 [the rat homolog to IgM (μ)], Serum/glucocorticoid regulated kinase 1 (Sgk1), MATα2, Deg1, Mating pheromone alpha-factor 1 protein (MFα1; also referred to as yeast prepro-alpha factor), yeast carboxypeptidase (CPY), a ricin toxin B-subunit, a cholera toxin B-subunit, a Shiga toxin (STx) B-subunit, a Shiga-like toxin (SLT) B-subunit [Verotoxin (VT) B-subunit], an E. coli heat-labile enterotoxin (LT) B-subunit, and an abrin toxin B-subunit, a ricin toxin A-subunit, a cholera toxin A-subunit, a Shiga toxin A1-subunit, a Shiga-like toxin A subunit (VT A-subunit), a Shiga-like toxin A1 subunit (VT A1-subunit), an E. coli heat-labile entertoxin A-subunit, an abrin A-subunit, a peptide fragment thereof, and a variant thereof.

In another embodiment, module (c) of the conjugate of the present invention is preferably selected from the group of C-terminal destabilizing oligopeptides consisting of CL1 (SEQ ID NO: 164), CL2 (SEQ ID NO: 165), CL6 (SEQ ID NO: 166), CL9 (SEQ ID NO: 167), CL10 (SEQ ID NO: 168), CL11 (SEQ ID NO: 169), CL12 (SEQ ID NO: 170), CL15 (SEQ ID NO: 171), CL16 (SEQ ID NO: 172), SL17 (SEQ ID NO: 173), a fragment thereof, and a variant thereof. Preferably, CL1 has the amino acid sequence ACKNWFSSLSHFVIHL (SEQ ID NO: 164); CL2 has the amino acid sequence SLISLPLPTRVKFSSLLLIRIMKIITM TFPKKLRS (SEQ ID NO: 165); CL6 has the amino acid sequence FYYPIWFARVLLVHYQ (SEQ ID NO: 166); CL9 has the amino acid sequence SNPFSSLFGASLLIDSVSLKSNWD TSSSSCLISFFSSVMFSSTTRS (SEQ ID NO: 167); CL10 has the amino acid sequence CRQRFSCHLTASYPQSTVTPFLAFLRRDFFFLRHNSSAD (SEQ ID NO: 168); CL11 has the amino acid sequence GAPHVVLFDFELRITNPLSHIQSVSLQITLIFCSLPSLILSKFLQV (SEQ ID NO: 169); CL12 has the amino acid sequence NTPLFSKSFSTTCGVAKKTLLLAQISSLFFLLLSSNIAV (SEQ ID NO: 170); CL15 has the amino acid sequence PTVKNSPKIFCLSSSPYLAFNLEYLSLRIFSTLSKCSNTLLTSLS (SEQ ID NO: 171); CL16 has the amino acid sequence SNQLKRLWLWLLEVRSFDRTLRRPWIHLPS (SEQ ID NO: 172); and SL17 has the amino acid sequence SISFVIRSHASIRMGASNDFFHKLYFTKCLTSVILSKFLIHLLLRSTPRV (SEQ ID NO: 173).

More preferably, the module (c) of the conjugate of the present invention comprises, essentially consists of or consists of (a) a peptide of a protein selected from the group consisting of (COX2), IgM(μ), Sgk1, MATα2, MFα1, Igh6, Deg1, CPY, Slt-I A-subunit, SLt-I B-subunit, Slt-II A-subunit, SLt-II B-subunit, Stx 1 A-subunit, Stx 1 B-subunit, ricin toxin A-subunit, ricin toxin B-subunit, cholera toxin A-subunit, cholera toxin B-subunit, LT A-subunit, LT B-subunit, LT-IIa A-subunit, LTIIa B-subunit, LTIIb A-subunit, LTIIb B-subunit, Abrin A-subunit, Abrin B-subunit, fragments thereof, and variants thereof, or
(b) a peptide comprising, essentially consisting of or consisting of the amino acid sequence CL1 (SEQ ID NO: 164), CL2 (SEQ ID NO: 165), CL6 (SEQ ID NO: 166), CL9 (SEQ ID NO: 167), CL10 (SEQ ID NO: 168), CL11 (SEQ ID NO: 169), CL12 (SEQ ID NO: 170), CL15 (SEQ ID NO: 171), CL16 (SEQ ID NO: 172), SL17 (SEQ ID NO: 173), or a fragment or variant thereof.

A COX2, IgM(μ), Sgk1, MATα2, MFα1, Igh6, Deg1, CPY, Slt-I A-subunit, SLt-I B-subunit, Slt-II A-subunit, SLt-II B-subunit, Stx 1 A-subunit, Stx1 B-subunit, ricin toxin A-subunit, ricin toxin B-subunit, cholera toxin A-subunit, cholera toxin B-subunit, LT A-subunit, LT B-subunit, LT-IIa A-subunit, LTIIa B-subunit, LTIIb A-subunit, LTIIb B-subunit, Abrin A-subunit, Abrin B-subunit variant differs from the respective wild-type COX2, IgM(μ), Sgk1, MATα2, MFα1, Igh6, Deg1, CPY, Slt-I A-subunit, SLt-I B-subunit, Slt-II A-subunit, SLt-II B-subunit, Stx 1 A-subunit, Stx1 B-subunit, ricin toxin A-subunit, ricin toxin B-subunit, cholera toxin A-subunit, cholera toxin B-subunit, LT A-subunit, LT B-subunit, LT-IIa A-subunit, LTIIa B-subunit, LTIIb A-subunit, LTIIb B-subunit, Abrin A-subunit, Abrin B-subunit peptide or protein, respectively, in that the variant comprises an amino acid sequence comprising up to 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 148, 150, 160, 170, 180, 190, 200, 220, 250, 270, 300, 331, 350, 368, 370, 371, 387, 400, 410, 415, 417, 420, 422, 424, 435, 440, 450, 470, 500, 504, 505, 510, 515, 520, 550, 560, 570, 579, 585 or 590 amino acid changes in the variant's amino acid sequence (i.e. substitutions, insertions, deletions, N-terminal truncations and/or C-terminal truncations) as compared to its corresponding wild-type protein's/peptide's amino acid sequence. Such a variant can alternatively or additionally be characterized by a certain degree of sequence identity to the wild-type protein from which it is derived. Thus, a COX2, IgM(μ), Sgk1, MATα2, MFα1, Igh6, Deg1, CPY, Slt-I A-subunit, SLt-I B-subunit, Slt-II A-subunit, SLt-II B-subunit, Stx 1 A-subunit, Stx1 B-subunit, ricin toxin A-subunit, ricin toxin B-subunit, cholera toxin A-subunit, cholera toxin B-subunit, LT A-subunit, LT B-subunit, LT-IIa A-subunit, LTIIa B-subunit, LTIIb A-subunit, LTIIb B-subunit, Abrin A-subunit, Abrin B-subunit protein variant or peptide variant has a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% to the respective reference (wild-type) COX2, IgM(μ), Sgk1, MATα2, MFα1, Igh6, Deg1, CPY, Slt-I A-subunit, SLt-I B-subunit, Slt-II A-subunit, SLt-II B-subunit, Stx 1 A-subunit, Stx1 B-subunit, ricin toxin A-subunit, ricin toxin B-subunit, cholera toxin A-subunit, cholera toxin B-subunit, LT A-subunit, LT B-subunit, LT-IIa A-subunit, LTIIa B-subunit, LTIIb A-subunit, LTIIb B-subunit, Abrin A-subunit, Abrin B-subunit amino acid sequence.

A peptide fragment (or deletion variant) of the COX2, IgM(μ), Sgk1, MATα2, MFα1, Igh6, Deg1, CPY, Slt-I A-subunit, SLt-I B-subunit, Slt-II A-subunit, SLt-II B-subunit, Stx 1 A-subunit, Stx1 B-subunit, ricin toxin A-subunit, ricin toxin B-subunit, cholera toxin A-subunit, cholera toxin B-subunit, LT A-subunit, LT B-subunit, LT-IIa A-subunit, LTIIa B-subunit, LTIIb A-subunit, LTIIb B-subunit, Abrin A-subunit, Abrin B-subunit protein or peptide preferably has a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 148, 150, 160, 170, 180, 190, 200, 220, 250, 270, 300, 331, 350, 368, 370, 371, 387, 400, 410, 415, 417, 420, 422, 424, 435, 440, 450, 470, 500, 504, 505, 510, 515, 520, 550, 560, 570, 579, 585 or 590 amino acids at its N-terminus and/or at its C-terminus and/or internally.

Additionally, a COX2, IgM(μ), Sgk1, MATα2, MFα1, Igh6, Deg1, CPY, Slt-I A-subunit, SLt-I B-subunit, Slt-II A-subunit, SLt-II B-subunit, Stx 1 A-subunit, Stx1 B-subunit, ricin toxin A-subunit, ricin toxin B-subunit, cholera toxin A-subunit, cholera toxin B-subunit, LT A-subunit, LT B-subunit, LT-IIa A-subunit, LTIIa B-subunit, LTIIb A-subunit, LTIIb B-subunit, Abrin A-subunit, Abrin B-subunit protein/peptide, protein/peptide variant or protein/peptide fragment is only regarded as a COX2, IgM(μ), Sgk1, MATalpha2, MATα2, MFα1, Igh6, Deg1, CPY, Slt-I A-subunit, SLt-I B-subunit, Slt-II A-subunit, SLt-II B-subunit, Stx 1 A-subunit, Stx1 B-subunit, ricin toxin A-subunit, ricin toxin B-subunit, cholera toxin A-subunit, cholera toxin B-subunit, LT A-subunit, LT B-subunit, LT-IIa A-subunit, LTIIa B-subunit, LTIIb A-subunit, LTIIb B-subunit, Abrin A-subunit, Abrin B-subunit protein/peptide, protein/peptide variant or protein/peptide fragment within the context of the present invention, if it exhibits the relevant biological activity to a degree of at least 30%, preferably at least 50% of the activity of the corresponding wild-type COX2, IgM(μ), Sgk1, MATα2, MFα1, Igh6, Deg1, CPY, Slt-I A-subunit, SLt-I B-subunit, Slt-II A-subunit, SLt-II B-subunit, Stx 1 A-subunit, Stx1 B-subunit, ricin toxin A-subunit, ricin toxin B-subunit, cholera toxin A-subunit, cholera toxin B-subunit, LT A-subunit, LT B-subunit, LT-IIa A-subunit, LTIIa B-subunit, LTIIb A-subunit, LTIIb B-subunit, Abrin A-subunit, Abrin B-subunit protein/peptide, respectively. The relevant “biological activity” in this context is the “activity to mediate translocation from the endoplasmic reticulum (ER) to the cytosol”, i.e. the ability of the variant or fragment to translocate from the lumen of the ER in the cytosol of a cell.

One of ordinary skill in the art can readily assess whether a COX2, IgM(μ), Sgk1, MATα2, MFα1, Igh6, Deg1, CPY, Slt-I A-subunit, SLt-I B-subunit, Slt-II A-subunit, SLt-II B-subunit, Stx 1 A-subunit, Stx1 B-subunit, ricin toxin A-subunit, ricin toxin B-subunit, cholera toxin A-subunit, cholera toxin B-subunit, LT A-subunit, LT B-subunit, LT-IIa A-subunit, LTIIa B-subunit, LTIIb A-subunit, LTIIb B-subunit, Abrin A-subunit, Abrin B-subunit protein/peptide, protein/peptide variant or protein/peptide fragment has the ability to translocate from the lumen of the ER in the cytosol, i.e. at least 30%, preferably at least 50% of the activity of the wild-type COX2, IgM(μ), Sgk1, MATα2, MFα1, Igh6, Deg1, CPY, Slt-I A-subunit, SLt-I B-subunit, Slt-II A-subunit, SLt-II B-subunit, Stx 1 A-subunit, Stx1 B-subunit, ricin toxin A-subunit, ricin toxin B-subunit, cholera toxin A-subunit, cholera toxin B-subunit, LT A-subunit, LT B-subunit, LT-IIa A-subunit, LTIIa B-subunit, LTIIb A-subunit, LTIIb B-subunit, Abrin A-subunit, Abrin B-subunit protein/peptide. Suitable assays, e.g. in vitro tracing of variants or fragments, for determining the “activity to mediate translocation from the endoplasmic reticulum (ER) to the cytosol” of a COX2, IgM(μ), Sgk1, MATα2, MFα1, Igh6, Deg1, CPY, Slt-I A-subunit, SLt-I B-subunit, Slt-II A-subunit, SLt-II B-subunit, Stx 1 A-subunit, Stx1 B-subunit, ricin toxin A-subunit, ricin toxin B-subunit, cholera toxin A-subunit, cholera toxin B-subunit, LT A-subunit, LT B-subunit, LT-IIa A-subunit, LTIIa B-subunit, LTIIb A-subunit, LTIIb B-subunit, Abrin A-subunit, Abrin B-subunit protein/peptide, protein/peptide variant or protein/peptide fragment compared to the binding activity of the respective wild-type protein/peptide are known in the art (see for example, [17]).

A peptide fragment of the COX2 protein has preferably a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 120, 150, 170, 200, 220, 250, 270, 300, 350, 370, 400, 420, 450, 470, 500, 504, 520, 550, 560, 570, 579, 585 or 590 amino acids at its N-terminus and/or at its C-terminus and/or internally, preferably at its N-terminus.

A peptide fragment of the IgM(μ) protein has preferably a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 120, 150, 170, 200, 250, 270, 300, 320, 350, 360, 370, 380, 390, 400, 410, 420, 435 or 440 amino acids at its N-terminus and/or at its C-terminus and/or internally, preferably at its N-terminus.

A peptide fragment of the Sgk1 protein has preferably a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 120, 150, 170, 200, 220, 250, 270, 300, 320, 325, 331, 350, 360, 368, 371, 380, 387, 400, 410, 415, 417, 422, or 424 amino acids at its N-terminus and/or at its C-terminus and/or internally, preferably at its C-terminus.

A peptide fragment of the MATα2 peptide has preferably a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 135, 140, 148, 150, or 160 amino acids at its N-terminus and/or at its C-terminus and/or internally, preferably at its C-terminus.

A peptide fragment of the MFα1 peptide has preferably a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 135, 140, 148, 150, or 160 amino acids at its N-terminus and/or at its C-terminus and/or internally, preferably at its C-terminus.

Preferably, module (c) of the conjugate of the present invention comprises or consists of a peptide of the human COX2 protein (UniProt P35354; SEQ ID NO: 174). It is particularly preferred that module (c) of the conjugate of the present invention comprises or consists of a

C-terminal peptide fragment of the human COX2 protein comprising or consisting of, preferably consisting of amino acids 504 through 604 (SEQ ID NO: 175) of human COX2. More preferably, module (c) of the conjugate of the present invention comprises or consists of a C-terminal peptide fragment of the human COX2 protein comprising or consisting of, preferably consisting of either amino acids 580 through 598 (SEQ ID NO: 176) or amino acids 580 through 604 (SEQ ID NO: 177) of human COX2.

In a particular preferred embodiment of the conjugate of the present invention, module (c) comprises, essentially consists or consists of a peptide comprising or consisting of the amino acid sequence NX₁SX₂X₃X₄X₅ X₆X₇X₈X₉INPTX₁₀X₁₁X₁₂X₁₃ (SEQ ID NO: 178) of COX2, wherein X₁ is A, S or V; X₂ is S, A or T; X₃ is S or V; X₄ is R, H or N; X₅ is S or T; X₆ is G, R, T or A; X₇ is L, V or M; X₈ is D, N or E; X₉ is D or N; X₁₀ is V or L; X₁₁ is L or V; X₁₂ is L or I; and X₁₃ is K or N.

In a more preferred embodiment of the conjugate of the present invention, module (c) comprises, essentially consists of or consists of a peptide comprising or consisting of the amino acid sequence NASSSRSGLDDINPTVLLK (SEQ ID NO: 176); NASASHSRLDDINPTVLIK (SEQ ID NO: 179); or NASSSHSGLDDINPTVLLK (SEQ ID NO: 180) of COX2.

In a particular preferred embodiment of the conjugate of the present invention, module (c) comprises, essentially consists of or consists of a peptide comprising or consisting of the amino acid sequence NX₁SSX₂X₃SX₄X₅DDINPTVLLK (SEQ ID NO: 181), wherein X₁ is A, G or V, X₂ is S or A, X₃ is R, H or N, X₄ is G, R or A, X₅ is L or S.

In a more particularly preferred embodiment of the conjugate of the present invention, module (c) comprises, essentially consists of or consists of a peptide comprising or consisting of the amino acid sequence NASSSRSGLDDINPTVLLKERSTEL (SEQ ID NO: 177) of human COX2.

Preferably, module (c) of the conjugate of the present invention comprises, essentially consists of or consists of a peptide of the mouse IgM(μ) protein (Accession number CAA27326; SEQ ID NO: 182). It is particularly preferred that module (c) of the conjugate of the present invention comprises or consists of a C-terminal peptide fragment of the mouse IgM(μ) protein comprising or consisting of, preferably consisting of amino acids 421 through 455 (SEQ ID NO: 183) of mouse IgM(μ). More preferably, module (c) of the conjugate of the present invention comprises or consists of a C-terminal peptide fragment of the mouse IgM(μ) protein comprising or consisting of, preferably consisting of amino acids 436 through 455 (SEQ ID NO: 184) of mouse IgM(μ).

In a more preferred embodiment of the conjugate of the present invention, module (c) comprises, essentially consists of or consists of a peptide comprising or consisting of the amino acid sequence GKPTLYNVSLIMSDTGGTCY (SEQ ID NO: 184); GKPTLYNVSLVMSDTAGTCY (SEQ ID NO: 185); GKPTLYQVSLIMSDTGGTCY (SEQ ID NO: 186); or GKPTLYQVSLIMSDTGGTSY (SEQ ID NO: 187) of IgM(μ).

Preferably, module (c) of the conjugate of the present invention comprises, essentially consists of or consists of a peptide of the human IgM(μ) protein (Accession number CAC20458; SEQ ID NO: 188). It is particularly preferred that module (c) of the conjugate of the present invention comprises or consists of a C-terminal peptide fragment of the human IgM(μ) protein comprising or consisting of, preferably consisting of amino acids 421 through 455 (SEQ ID NO: 189) of human IgM(μ). More preferably, module (c) of the conjugate of the present invention comprises or consists of a C-terminal peptide fragment of the human IgM(μ) protein comprising or consisting of, preferably consisting of amino acids 436 through 455 (SEQ ID NO: 185) of human IgM(μ).

In a particularly preferred embodiment of the conjugate of the present invention, module (c) comprises, essentially consists of or consists of a peptide comprising or consisting of the amino acid sequence GKPTLYX₁VSLX₂MSDTX₃GTX₄Y (SEQ ID NO: 190) of IgM(μ), wherein X₁ is N or Q; X₂ is I or V; X₃ is G or A; and X₄ is C or S.

Preferably, module (c) of the conjugate of the present invention comprises, essentially consists of or consists of a peptide of the mouse Sgk1 protein (UniProt Q9WVC6; SEQ ID NO: 191). It is particularly preferred that module (c) of the conjugate of the present invention comprises, essentially consists of or consists of an N-terminal peptide fragment of the mouse Sgk1 protein comprising or consisting of, preferably consisting of amino acids 1 through 100 (SEQ ID NO: 192) of mouse Sgk1. Preferably, module (c) of the conjugate of the present invention comprises, essentially consists of or consists of an N-terminal peptide fragment of the mouse Sgk1 protein comprising or consisting of, preferably consisting of amino acids 1 through 60 (SEQ ID NO: 193) of mouse Sgk1 protein. Preferably, module (c) of the conjugate of the present invention comprises, essentially consists of or consists of an N-terminal peptide fragment of the mouse Sgk1 protein comprising or consisting of, preferably consisting of amino acids 1 through 33 (SEQ ID NO: 194) of mouse Sgk1 protein.

Preferably, module (c) of the conjugate of the present invention comprises, essentially consists of or consists of a peptide of the human Sgk1 protein (UniProt accession number 00014; SEQ ID NO: 195). It is particularly preferred that module (c) of the conjugate of the present invention comprises, essentially consists of or consists of an N-terminal peptide fragment of the human Sgk1 protein comprising or consisting of, preferably consisting of amino acids 1 through 100 (SEQ ID NO: 196) of human Sgk1. Preferably, module (c) of the conjugate of the present invention comprises, essentially consists of or consists of an N-terminal peptide fragment of the human Sgk1 protein comprising or consisting of preferably, consisting of amino acids 1 through 60 (SEQ ID NO: 197) of human Sgk1 protein.

Preferably, module (c) of the conjugate of the present invention comprises, essentially consists of or consists of an N-terminal peptide fragment of the human Sgk1 protein comprising or consisting of, preferably consisting of amino acids 1 through 33 (SEQ ID NO: 198) of human Sgk1 protein. Preferably, module (c) of the conjugate of the present invention comprises, essentially consists of or consists of an N-terminal peptide fragment of the human Sgk1 protein comprising or consisting of, preferably consisting of amino acids 1 through 30 (SEQ ID NO: 199) of human Sgk1 protein.

In a particular preferred embodiment of the conjugate of the present invention, module (c) comprises, essentially consists of or consists of a peptide comprising the amino acid sequence MTX₁X₂X₃X₄EX₅X₆X₇X₈X₉X₁₀X₁₁LTYSX₁₂X₁₃RGX₁₄VAX₁₅LX₁₆AFMKQRX₁₂MGLNDFI QKX₁₈X₁₉X₂₀NX₂₁YACKHX₂₂EVQSX₂₃LX₂₄X₂₅ (SEQ ID NO: 200) of mouse Sgk1, wherein X₁ is V or I; X₂ is K or Q; X₃ is A or T; X₄ is X [X is zero (0) amino acid] or A; X₅ is A or T; X₆ is A or S; X₇ is R, K, G or V; X₈ is S, G or P; X₉ is T, P or A; X₁₀ is X or P; X₁₁ is X or D; X₁₂ is R or K; X₁₃ is M or T; X₁₄ is M or L; X₁₅ is I or N; X₁₆ is I or S; X₁₇ is R or K; X₁₈ is I or L; X₁₉ is A or S; X₂₀ is S, N, A or T; X₂₁ is T or S; X₂₂ is A, P or T; X₂₃ is I or Y; X₂₄ is K or N; and X₂₅ is M, I or L.

In a more preferred embodiment of the conjugate of the present invention, module (c) comprises, essentially consists of or consists of a peptide comprising the amino acid sequence MTVKAEAARSTLTYSRMRGMVAILIAFMKQRRMGLNDFIQKIASNTYACKHAEVQSILKM of mouse Sgk1 (SEQ ID NO: 193); MTVKTEAAKGTLTYSRMRGMVAILIAFMKQRRMGLNDFIQKIANNSYACKHPEVQSILKI (SEQ ID NO: 197) of human Sgk1; MTVKTEAAKGTLTYSRMRGMVAILIAFMKQ (SEQ ID NO: 199) of human Sgk1; MTVKTEAARSTLTYSRMRGMVAILIAFMKQRRMGLNDFIQKLANNSYACKHPEVQSYLKI (SEQ ID NO: 201) of rat Sgk1 (also referred to as Igh6; Accession number AAI05826); MTVKTEAARGPLTYSRMRGMVAILIAFMKQRRMGLNDFIQKIANNSYACKHTEVQSILKI (SEQ ID NO: 202) of rabbit Sgk1; MTVKAAEASGPALTYSKMRGMVAILIAFMKQRRMGLNDFIQKIATNSYACKHPEVQSILK (SEQ ID NO: 203) of chicken Sgk1; or MTIQTETSVSAPDLTYSKTRGLVANLSAFMKQRKMGLNDFIQKLSANSYACKHPEVQSIL (SEQ ID NO: 204) of zebrafish Sgk1.

In a more preferred embodiment of the conjugate of the present invention, module (c) comprises, essentially consists of or consists of a peptide comprising the amino acid sequence MTVKTEAAKGTLTYSRMRGMVAILIAFMKQ (SEQ ID NO: 199), MRGMVAILIAFMKQRRMGLNDFIQKIASNTYACKHAEVQSILKM (SEQ ID NO: 205); MRGMVAILIAFMKQ (SEQ ID NO: 206); GMVAILIAF (SEQ ID NO: 207); MRGMVAILIAFMKQRRM (SEQ ID NO: 208), GMVAILI (SEQ ID NO: 209), or MRGMVAILIAFMKQRRMGLNDFIQKIANNSYACKHPEVQSILKI (SEQ ID NO: 210) of Sgk1, designated as an Sgk1 peptide fragment.

Preferably, module (c) of the conjugate of the present invention comprises, essentially consists of or consists of a peptide of the MATα2 peptide from yeast (NCBI RefSeq NP 009868) (SEQ ID NO: 211). It is particularly preferred that module (c) of the conjugate of the present invention comprises or consists of an N-terminal peptide fragment of the MATα2 peptide from yeast comprising amino acids 1 through 100 (SEQ ID NO: 212). More preferably, module (c) of the conjugate of the present invention comprises, essentially consists of or consists of an N-terminal peptide fragment of the MATα2 protein from yeast comprising amino acids 1 through 62 (SEQ ID NO: 213; also referred to as Deg1 degradation signal) of MATα2.

In a particular preferred embodiment of the conjugate of the present invention, module (c) comprises, essentially consists of or consists of a peptide comprising the amino acid sequence MNKIPIKDLLNPQITDEFKSSILDINKKLFSICCNLPKLPESVTTEEEVELRDILX₁FLSRAN (SEQ ID NO: 214) of MATα2, wherein X₁ is G, V or L.

In a more preferred embodiment of the conjugate of the present invention, module (c) comprises, essentially consists of or consists of a peptide comprising the amino acid sequence MNKIPIKDLLNPQITDEFKSSILDINKKLFSICCNLPKLPESVTTEEEVELRDILGFLSRAN (SEQ ID NO: 213); MNKIPIKDLLNPQITDEFKSSILDINKKLFSICCNLPKLPESVTTEEEVELRDILVFLSRAN (SEQ ID NO: 215); or MNKIPIKDLLNPQITDEFKSSILDINKKLFSICCNLPKLPESVTTEEEVELRDILLFLSRAN (SEQ ID NO: 216) of MATα2.

In a more preferred embodiment of the conjugate of the present invention, module (c) comprises, essentially consists of or consists of a peptide comprising the amino acid sequence ITDEFKSSILDINKKLFSI (SEQ ID NO: 217); or ITDEFKSSILDINKKLFSICCNLPKLPESV (SEQ ID NO: 218) of MATα2, designated as a MATα2 peptide fragment.

Preferably, module (c) of the conjugate of the present invention comprises, essentially consists of or consists of the yeast MFα1 peptide (SEQ ID NO: 219 [9]; UniProt P01149; Accession numbers CAA25738; AAA88727).

In a particular preferred embodiment of the conjugate of the present invention, module (c) comprises, essentially consists of or consists of a peptide comprising the amino acid sequence MRFPSIFTAVLFAASSALAAPVX₁TTTEDETAQIPAEAVIGYLDLEGDFDVAVLPFSX₁STNNGLLFIX₁TTIASIAAKEEGVSLDKREAEAWHWLQLKPGQPMYKREAEAEAWHWLQLKPGQPMYKREADAEAWHWLQLKPGQPMYKREADAEAWHWLQLKPGQPMY (SEQ ID NO: 220) of MFα1, wherein X₁ is N or Q.

In a more preferred embodiment of the conjugate of the present invention, module (c) comprises, essentially consists of or consists of a peptide comprising the amino acid sequence MRFPSIFTAVLFAASSALAAPVQTTTEDETAQIPAEAVIGYLDLEGDFDVAVLPFSQSTNNGLLFIQTTIASIAAKEEGVSLDICREAEAWHWLQLKPGQPMYKREAEAEAWHWLQLKPGQPMYKREADAEAWHWLQLKPGQPMYKREADAEAWHWLQLKPGQPMY (SEQ ID NO: 221); MRFPSIFTAVLFAASSALAAPVNTTTEDETAQIPAEAVIGYLDLEGDFDVAVLPFSNSTNNGLLFINTTIASIAAKEEGVSLDKREAEAWHWLQLKPGQPMYKREAEAEAWHWLQLKPGQPMYKREADAEAWHWLQLKPGQPMYKREADAEAWHWLQLKPGQPMY (SEQ ID NO: 219); MRFPSIFTAVLFAASSALAAPVNTTTEDETAQIPAEAVIGYLDLEGDFDVAVLPFSNSTNNGLLFIQTTIASIAAKEEGVSLDKREAEAWHWLQLKPGQPMYKREAEAEAWHWLQLKPGQPMYKREADAEAWHWLQLKPGQPMYKREADAEAWHWLQLKPGQPMY (SEQ ID NO: 222); or MRFPSIFTAVLFAASSALAAPVQTTTEDETAQIPAEAVIGYLDLEGDFDVAVLPFSNSTNNGLLFINTTIASIAAKEEGVSLDKREAEAWHWLQLKPGQPMYKREAEAEAWHWLQLKPGQPMYKREADAEAWHWLQLKPGQPMYKREADAEAWHWLQLKPGQPMY (SEQ ID NO: 223) of MFα1.

Preferably, module (c) of the conjugate of the present invention comprises, essentially consists of or consists of a peptide of the yeast CPY protein (Accession number P52710; SEQ ID NO: 224).

In another preferred embodiment, a peptide fragment of the CPY protein has a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 135, 140, 148, 150, 160, 170, 180, 190, 200, 220, 250, 270, 300, 350, 370, 400, 420, 450, 470, 500, 505, 510, 515, 520 amino acids at its N-terminus, at its C-terminus, and/or internally.

Preferably, module (c) of the conjugate of the present invention comprises, essentially consists of or consists of a peptide of a toxin protein. A peptide fragment of a toxin protein preferably has a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 135, 140, 148, 150, 160, 170, 180, 190, 200, 220, 250, 251, 258, 259, 270, 300, 315, 319, 350, 370, 400, 420, 450, 470, 500, 505, 510, 515, 520, 541, amino acids at its N-terminus and/or at its C-terminus and/or internally.

In a preferred embodiment, module (c) of the conjugate of the present invention comprises, essentially consists of or consists of a peptide of a toxin protein selected from the group consisting of a ricin toxin B-subunit, a cholera toxin B-subunit, a Shiga toxin (STx) B-subunit, a Shiga-like toxin (SLT) B-subunit [Verotoxin (VT) B-subunit], an E. coli heat-labile enterotoxin (LT) B-subunit, and an abrin toxin B-subunit. Preferably, module (c) comprises a ricin toxin B-subunit peptide, a peptide from a recombinantly produced ricin toxin B-subunit (e.g., as described in WO2008/157263), a cholera toxin B-subunit peptide, an Stx B-subunit peptide, an STx1 (SLT-I or VT1) B-subunit peptide, an SLT-Ib B-subunit peptide, an SLT-Ic B-subunit peptide (a VT1c peptide), an SLT-IIb-subunit peptide (a VT2 peptide), an SLT-IIc B-subunit peptide (a VT2c peptide), an SLT-IId B-subunit peptide (a VT2d peptide), an SLT-IIe B-subunit peptide (a VT2e peptide), an SLT-IIf B-subunit peptide (a VT2f peptide), an LT B-subunit peptide, an LTIIa B-subunit peptide, an LTIIb B-subunit peptide, or an abrin toxin B-subunit peptide.

A peptide of ricin toxin B-subunit preferably comprises or consists, preferably consists of an amino acid sequence according to SEQ ID NO: 124, FSVYDVSILIPIIALMVYRCAPPPSSQF (SEQ ID NO: 225), or a fragment or variant thereof.

A peptide of cholera toxin B-subunit preferably comprises or consists, preferably consists of an amino acid sequence according to SEQ ID NO: 125, YGLAGFPPEHRAWRE EPWIHHAPPGCGNAPRSS (SEQ ID NO: 226), or a fragment or variant thereof.

A peptide of Shiga toxin (Stx) B-subunit (Stx) preferably comprises or consists, preferably consists of an amino acid sequence according to SEQ ID NO: 126, ISFNNISAI LGTVAVILNCHHQGARSVR (SEQ ID NO: 227), or a fragment or variant thereof.

A peptide of Stx1 B-subunit preferably comprises or consists, preferably consists of an amino acid sequence according to SEQ ID NO: 127, or a fragment or variant thereof.

A peptide of Slt-Ib B-subunit preferably comprises or consists, preferably consists of an amino acid sequence according to SEQ ID NO: 128, or a fragment or variant thereof.

A peptide of Slt-Ic B-subunit preferably comprises or consists, preferably consists of an amino acid sequence according to SEQ ID NO: 129, or a fragment or variant thereof.

A peptide of Slt-II B-subunit preferably comprises or consists, preferably consists of an amino acid sequence according to ISFNNISAILGTVAVILNCHHQGARSVR (SEQ ID NO: 228), or a fragment or variant thereof.

A peptide of Slt-IIb B-subunit preferably comprises or consists, preferably consists of an amino acid sequence according to SEQ ID NO: 130, or a fragment or variant thereof.

A peptide of Slt-IIc B-subunit preferably comprises or consists, preferably consists of an amino acid sequence according to SEQ ID NO: 131, or a fragment or variant thereof.

A peptide of Slt-IId B-subunit preferably comprises or consists, preferably consists of an amino acid sequence according to SEQ ID NO: 132, or a fragment or variant thereof.

A peptide of Slt-IIe B-subunit preferably comprises or consists, preferably consists of an amino acid sequence according to SEQ ID NO: 133, or a fragment or variant thereof.

A peptide of Slt-IIf B-subunit preferably comprises an amino acid sequence comprising SEQ ID NO: 134, or a fragment or variant thereof.

A peptide of LT-B B-subunit preferably comprises or consists, preferably consists of an amino acid sequence according to SEQ ID NO: 135, SEQ ID NO: 136, or a fragment or variant thereof.

A peptide of LT-IIa B-subunit preferably comprises or consists, preferably consists of an amino acid sequence according to SEQ ID NO: 137, or a fragment or variant thereof.

A peptide of LT-IIb B-subunit preferably comprises or consists, preferably consists of an amino acid sequence according to SEQ ID NO: 138, or a fragment or variant thereof.

A peptide of abrin toxin B-subunit preferably comprises or consists, preferably consists of an amino acid sequence according to SEQ ID NO: 139, or a fragment or variant thereof.

In another embodiment, a conjugate of the present invention comprises a module (c) comprising, essentially consisting of or consisting of a peptide of an A or A1 subunit of a toxin, wherein the peptide is preferably non-toxic. Preferably, module (c) comprises or consists of a toxin protein or peptide selected from the group consisting of a ricin toxin A-subunit, a cholera toxin A-subunit, a Shiga toxin (STx) A-subunit, a Shiga-like toxin (SLT) A-subunit [Verotoxin (VT) A-subunit], an E. coli heat-labile enterotoxin (LT) A-subunit, an abrin toxin A-subunit, a Pertussis toxin A-subunit, a Modeccin A-subunit, a Volkensin A-subunit, and a Pseudomonas Exotoxin A subunit, wherein the toxin protein or peptide is preferably non-toxic. Preferably, module (c) of the conjugate of the present invention comprises or consists of a non-toxic peptide of an A or A1 subunit of ricin toxin, cholera toxin, Shiga toxin (Stx), Shiga-like toxin (SLT) I (STx1, SLT-I or VT1), SLT-Ib, SLT-Ic (VT1c), SLT-IIb (Stx2 or VT2), SLT-IIc (Stx2c or VT2c), SLT-IId (Stx2d or VT2d), SLT-IIe (Stx2e or VT2e), SLT-IIf (Stx2f or VT20, LT, LTIIa, LTIIb, or abrin toxin. More preferably, module (c) of the conjugate of the present invention comprises or consists of a non-toxic peptide of an A or A1 subunit of Shiga toxin (Stx), Shiga-like toxin (SLT), or an E. coli heat labile enterotoxin (LT).

In a preferred embodiment, module (c) comprises or consists of a non-toxic peptide of ricin toxin A1-subunit (SEQ ID NO: 282; ricin toxin A).

In a preferred embodiment, module (c) comprises or consists of a non-toxic peptide of cholera toxin A1-subunit (SEQ ID NO: 283; cholera toxin A).

In a preferred embodiment, module (c) comprises or consists of a non-toxic peptide of Shiga toxin (Stx), A1-subunit (SEQ ID NO: 229; Stx A1).

In a preferred embodiment, module (c) comprises or consists of a non-toxic peptide of Shiga-like toxin I, A1-subunit (SEQ ID NO: 230; STx1 A1 (Slt-I A1 or VT1 A1) [43]). Preferably, the peptide of Slt-I A1 comprises or consists of an amino acid sequence according to ISFGSINAILGSVALILNCHHHASRVAR (SEQ ID NO: 231, amino acids 224-251 of Slt-I A 1), ISFGSINAILGSVALILNCHHH (SEQ ID NO: 232, amino acids 224-245 of Slt-I A1), ISFGSINAILGSVALIL (SEQ ID NO: 233, amino acids 224-240 of Slt-I A1), or a fragment or variant thereof.

In a preferred embodiment, module (c) comprises or consists of a non-toxic peptide of Shiga-like toxin Ic, A-subunit peptide (SEQ ID NO: 234; VT1c A).

In a preferred embodiment, module (c) comprises or consists of a non-toxic peptide of Shiga like toxin IIb A1-subunit (SEQ ID NO: 235; SLT-IIb A1, Stx2 A1, or VT2 A1).

In a preferred embodiment, module (c) comprises or consists of a non-toxic peptide of Shiga like toxin IId A-subunit (SEQ ID NO: 236; SLT-IId A, Stx2d A, or VT2d A).

In a preferred embodiment, module (c) comprises a or consists of non-toxic peptide of Shiga like toxin IIe A-subunit (SEQ ID NO: 237; SLT-IIe A, Stx2e A, or VT2e A).

In a preferred embodiment, module (c) comprises or consists of a non-toxic peptide of Shiga like toxin IIf A-subunit (SEQ ID NO: 238; SLT-IIf A, Stx2f A, or VT2f A).

In a preferred embodiment, module (c) comprises or consists of a non-toxic peptide of E. coli heat-labile entertoxin LT A-subunit [SEQ ID NO: 239 (LT A human strain) or SEQ ID NO: 240 (LT A porcine strain)].

In a preferred embodiment, module (c) comprises or consists of a non-toxic peptide of E. coli heat-labile entertoxin LT-IIa A-subunit (SEQ ID NO: 241; LT-IIa A). Preferably, module (c) of the conjugate of the present invention comprises or consists of a non-toxic peptide of LT-IIa A that comprises an amino acid sequence according to YQLAGFPSNFPAWREMPWSTFAPEQCVPNNK (SEQ ID NO: 242),

In a preferred embodiment, module (c) comprises or consists a non-toxic peptide of E. coli heat-labile entertoxin LT-IIb A-subunit (SEQ ID NO: 243; LT-IIb A).

In another embodiment, module (c) of the conjugate of the present invention comprises or consists of a viral peptide that facilitates translocation from the ER to the cytosol. Preferably, said viral peptide is from a polyomavirus. More preferably, said viral peptide is from SV40, murine polyomavirus, BK virus, JC virus, KI virus, WU virus, and Merkel Cell polyomavirus. Even more preferably, said viral peptide is from SV40 or murine polyomavirus. Polyomaviruses (e.g., mPyV and SV40) have been shown to be recognized as misfolded proteins within the ER by the ER associated degradation machinery and are subsequently transported to the cytosol by ERAD [37]. Thus, a viral peptide, fragment or variant from SV40, murine polyomavirus, BK virus, JC virus, KI virus, WU virus, or Merkel Cell polyomavirus may be used as a module (c) in the conjugates of the present invention.

One of ordinary skill in the art is well aware of methods for producing module (c) according to the present invention. For example, the module (c) may be chemically synthesized, e.g., by liquid phase or solid phase peptide synthesis, or the peptide may be genetically engineered using recombinant DNA techniques and a cellular expression system, such as bacteria, e.g., Escherichia coli, yeast cells, insect cells, mammalian cells, etc., or an in vitro expression system.

In a preferred embodiment, module (b) and module (c) are comprised in a single contiguous peptide. Preferably, the single contiguous peptide comprising module (b) and module (c) is selected from the group consisting of NASSSRSGLDDINPTVLLKERSTEL (CX1a; SEQ ID NO: 177), NASSSRSGLDDINPTVLLKAKDEL (CX2a; SEQ ID NO: 244), and GKPTLYQVSLIMSDTGGTSYKDEL (SEQ ID NO: 245).

Within the context of the present invention, the “at least one module (a), at least one module (b), and at least one module (c)” is also defined as a “delivery carrier” of the invention. Preferably, the delivery carrier comprises at least one module (a), at least one module (b), and at least one module (c), wherein the at least one module (a), the at least one module (b), and the at least one module (c) are linked to each other in any arrangement. More preferably, the delivery carrier of the present invention comprises RTB, RTB-COX2 peptide, RTB-COX2 peptide-AKDEL peptide, RTB-AKDEL peptide, RTB-Sgk1 peptide-AKDEL peptide, TfR peptide-COX2 peptide-AKDEL peptide, Sgk1 peptide-TfR peptide-AKDEL peptide, TfR peptide-AKDEL peptide-IgM(μ) peptide, or TfR peptide-IgM(μ) peptide-AKDEL peptide.

The conjugate of the present invention comprises at least one compound (d), wherein compound (d) is preferably a nucleic acid, a peptide, a protein, a pharmaceutical, a cytotoxic agent, a radioactive agent, or another therapeutic or diagnostic moiety.

In a preferred embodiment, compound (d) is a nucleic acid. Preferably, the nucleic acid is single stranded or double stranded DNA, single stranded or double stranded RNA, siRNA, tRNA, mRNA, micro RNA (miRNA), small nuclear RNA (snRNA), small hairpin RNA (shRNA), morpholino modified iRNA (for example, as described in US2010/0076056 and U.S. Pat. No. 7,745,608), anti-gene RNA (agRNA, for example [44]), or the like.

Preferably, the conjugate of the present invention is configured such that it comprises RTB-siRNA, RTB linked to an siRNA via a lysine linkage (for example, see FIG. 4), RTB linked to an siRNA via a cysteine linkage (for example, see FIG. 5), RTB-COX2 peptide-siRNA [for example, see FIGS. 6 (A) and (B)], RTB-COX2 peptide-AKDEL peptide-siRNA (for example, see FIG. 7), RTB-AKDEL peptide-siRNA (for example, see FIG. 8), RTB-Sgk 1 peptide-AKDEL peptide-siRNA (for example, see FIG. 9), TfR peptide-COX2 peptide-AKDEL peptide-siRNA [for example, see FIGS. 10(A) and (B)], Sgk1 peptide-TfR peptide-AKDEL peptide-siRNA (for example, see FIG. 11), TfR peptide-AKDEL peptide-IgM(μ) peptide-siRNA (for example, see FIG. 12), TfR peptide-IgM(μ) peptide-AKDEL peptide-siRNA (for example, see FIG. 13), or RTB-COX2 peptide-AKDEL peptide-2 siRNAs (for example, see FIG. 14).

Preferably, the conjugate of the present invention comprises a configuration as depicted in FIG. 4, FIG. 5, FIG. 6(A), FIG. 6(B), FIG. 7, FIG. 8, FIG. 9, FIG. 10(A), FIG. 10(B), FIG. 11, FIG. 12, FIG. 13, or FIG. 14.

As stated earlier, there is often a problem with delivering a nucleic acid molecule into a cell. The use of the conjugate of the present invention provides a suitable delivery system of delivering nucleic acid molecules into a cell, preferably into the cytoplasm of a cell. The nucleic acid molecules delivered by the conjugate of the present invention may be used, for example, to achieve targeted gene silencing in a wide range of experimental systems from plants to human cells. Preferably, the nucleic acid molecules delivered by the conjugate of the present invention are therapeutic nucleic acid molecules that may be used, for example, to achieve targeted gene silencing in an organism, wherein the organism is a mammal, preferably a human.

RNAi, or RNA-mediated interference, is a method of choice for achieving targeted gene silencing in a wide range of experimental systems from plants to human cells. Following introduction of siRNA or miRNA into the cell cytoplasm, these double-stranded RNA constructs can bind to a protein termed RISC. The sense strand of the siRNA or miRNA is displaced from the RISC complex providing a template within RISC that can recognize and bind mRNA with a complementary sequence to that of the bound siRNA or miRNA. Having bound the complementary mRNA, the RISC complex cleaves the mRNA and releases the cleaved strands. RNAi can provide down-regulation of specific proteins by targeting specific destruction of the corresponding mRNA that encodes for protein synthesis.

In a preferred embodiment, a conjugate of the present invention comprises a compound (d) that is an siRNA. In a more preferred embodiment, a conjugate of the present invention comprises at least 2 compounds (d) that are siRNAs. Preferably, the conjugate comprises at least 2-20 siRNAs, i.e., at least 2, 3, 4, 5, 6, 7, 8, 9, 10, 11, 12, 13, 14, 15, 16, 17, 18, 19, or 20 siRNAs. In a preferred embodiment, a conjugate of the present invention comprises at least 2-10 siRNAs. In another preferred embodiment, a conjugate of the present invention comprises 2-10, i.e., 1, 2, 3, 4, 5, 6, 7, 8, 9, or 10 siRNAs. Within certain preferred embodiments of the invention, it may be necessary to neutralize the charge of the at least 2-20 siRNAs comprised within a conjugate of the present invention using methods available in the art.

As mentioned above, a preferred conjugate of the present invention comprises at least 2 compounds (d). In a preferred embodiment, the conjugate comprises at least two compounds (d), wherein the first of the at least 2 compounds (d) is an siRNA, and the second of the at least 2 compounds (d) is a RISC component. In this preferred embodiment, co-delivery of at least one targeted siRNA and at least one RISC component as compounds (d) in a conjugate of the present invention, is useful to enhance the efficiency of RNAi in a target cell, particularly in target cells in which the RNAi machinery is limited, either endogenously or as a result of when multiple siRNAs/conjugate are delivered to the target cells.

The term “RISC component” means any protein or peptide that is a component or an associated protein of a RISC complex. Examples of RISC components for use in the conjugates of the present invention include but are not limited to Dicer (e.g., Dicer-1, Dicer-2, and the like), Argonaute family proteins (e.g., Argonaute 2, and the like), transactivating response RNA-binding protein (TRBP), double stranded RNA binding domain proteins and peptides (e.g., R2D2, R3D1, and the like), protein activator of protein kinase R(PACT), Argonaute-related proteins (e.g., Piwi and the like), helicases, and nucleases.

Antisense constructs can also inhibit mRNA translation into protein. Antisense constructs are single stranded oligonucleotides and are non-coding. These single stranded oligonucleotides have a complementary sequence to that of the target protein mRNA and can bind to the mRNA by Watson-Crick base pairing. This binding either prevents translation of the target mRNA and/or triggers RNase H degradation of the mRNA transcripts, depending upon the type of chemical modifications used in the antisense construct. Consequently, antisense oligonucleotides have tremendous potential for specificity of action (i.e., down-regulation of a specific disease-related protein). To date, these compounds have shown promise in several in vitro and in vivo models, including models of inflammatory disease, cancer, and HIV [reviewed in 45]. Antisense can also affect cellular activity by hybridizing specifically with chromosomal DNA.

Coding nucleic acid molecules can also be used. Coding nucleic acid molecules (e.g. DNA) designed to function as a substrate for relevant RNA polymerases or ribosomes to directly drive transcription or translation of encoded product contained within its sequence, typically contain an open reading frame and appropriate regulatory motifs, e.g. promoter sequences, start, stop, poly A signals, and the like.

Preferably, the nucleic acid of the conjugate of the present invention is chemically modified. Nucleic acids comprising single or multiple modifications of the phosphodiester backbone or of the backbone, the sugar, and/or the nucleobases are preferred for use in the present invention. These chemically modifications have the positive effect that they stabilize the nucleic acid and have little impact on their activity. These chemical modifications can further prevent unwanted side effects of the nucleic acid like immune reactions via TLR's and/or the interferon pathway, or expression regulation of unintended target genes [i.e., Off Target Effects (OTEs)].

Preferred modifications of the phosphodiester backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thiono-alkylphosphonates, thionoalkylphosphotriesters, phosphoroselenate, methylphosphonate, or O-alkyl phosphotriester linkages, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

Modified nucleobases include other synthetic and natural nucleobases such as 5-methylcytosine (5-Me-C or m5C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-aza uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine, and 3-deazaguanine and 3-deazaadenine.

Modified nucleic acids may also contain one or more substituted sugar moieties. For example, the invention includes nucleic acids that comprise one of the following at the 2′ position: OH; F; O-, S-, or N-alkyl, O-alkyl-O-alkyl, O-, S-, or N-alkenyl, or O-, S- or N-alkynyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)₂ON(CH₃)₂, O(CH₂)ₙNH₂, O(CH₂)nCH₃, O(CH₂)ₙONH₂, and O(CH₂)ₙON[(CH₂)ₙCH₃)]₂, where n and m are from 1 to about 10. Other preferred modified nucleic acids comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Further sugar modifications include, e.g. 2′-O-methyl, a locked nucleic acid (LNA), 2′-F, an unlocked nucleic acid (UNA), etc. Preferred backbone modifications include, e.g. peptide nucleic acid (PNA), morpholino, etc.

A “locked nucleic acid” (LNA) according to the present invention, often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation.

An “unlocked nucleic acid” (UNA) according to the present invention is comprised of monomers that are acyclic derivatives of RNA that lack the C2′-C3′-bond of the ribose ring of RNA.

A “peptide nucleic acid” (PNA) according to the present invention has a backbone composed of repeating N-(2-aminoethyl)-glycine units linked by peptide bonds.

In another preferred embodiment, compound (d) is a protein or a peptide. Proteins and peptides that may be delivered preferably include single chain antibodies, kinases, phosphatases, nucleases, inflammatory proteins, anti-infectious proteins, anti-angiogenic proteins, anti-inflammatory proteins, or any other protein or peptide or small molecule that is desired to be delivered to a cell, preferably to the cytosol of a cell.

Preferably, a compound (d) comprising a protein or peptide is coupled to modules (a), (b), and (c) via a disulfide linkage, in similar fashion as an siRNA described above and within the Examples, whereby the protein or peptide is cleaved from the delivery modules of the conjugate upon reaching the cytoplasm and is able to perform its intended function within the target cell. In an alternative preferred embodiment, an enzymatic cleavage site, as described above, is preferably present within the conjugate to enable release of compound (d) at the target cell's desired compartment, organelle or cytosol, or to separate compound (d) from the conjugate modules. In a particularly preferred embodiment, a conjugate of the present invention comprises a compound (d) comprising a protein or peptide, wherein the compound (d) is coupled to modules (a), (b), and (c) via a disulfide linkage, and wherein an enzymatic cleavage site is positioned within the conjugate, that when cleaved by an enzyme, releases compound (d) from the conjugate.

In a preferred embodiment, the compound (d) is an antigen that is desired to be delivered to the cytosol. Within this embodiment, an enzymatic cleavage site is preferably present within the conjugate to enable release of the antigen in the target cell's cytosol. Preferably, when compound (d) is an antigen, module (a) comprises a B-subunit of a toxin or a fragment or variant thereof. Preferably, the B-subunit of a toxin is ricin B-subunit (RTB) or Shiga toxin B-subunit. Such B-subunit toxin-antigen comprising conjugates of the invention are useful as vaccines to immunize an animal, preferably a mammal, more preferably a human (see for example, [46, 47].

In another preferred embodiment, module (a) comprises a non-toxic holo-toxin, wherein the non-toxic holo-toxin is preferably a non-toxic ricin holo-toxin or a non-toxic Shiga holo-toxin. Preferably, the non-toxic holo-toxin comprises an A-subunit, wherein the A-subunit comprises a mutation that eliminates or greatly reduces the toxicity of the holo-toxin. A non-toxic holo-toxin comprising a mutated A-subunit is able to provide the functionalities of modules (a), (b) and (c) of a conjugate of the invention. Preferably, the non-toxic holo-toxin is a non-toxic ricin holo-toxin, wherein ricin A-subunit comprises an R→H substitution mutation at amino acid 180 (an R180H mutation) of ricin A-subunit (SEQ ID NO: 282).

Preferably, the functionality of modules (a) and (b) are comprised within the non-toxic holo-toxin B-subunit and the functionality of module (c) is comprised within the non-toxic holo-toxin mutated A-subunit. Preferably, compound (d) is an antigen coupled to the mutated A-subunit of the non-toxic holo-toxin [module (a)+module (b)+module (c)]. Such mutated A-subunit comprising holo-toxin-antigen comprising conjugates of the invention are useful as vaccines to immunize an animal, preferably a mammal, more preferably a human (see for example, [48]).

Antigens that are contemplated to be delivered using the present invention include but are not limited to NSP4, Influenza nucleoprotein NP, LCMV glycoprotein 1, hTRT, CYFRA 21-1, p53, ras, β-catenin, CDK4, CDC27, α actinin-4, tyrosinase, TRP1/gp75, TRP2, gp100, Melan-A/MART1, gangliosides, PSMA, HER2, WT1, EphA3, EGFR, CD20, MAGE, BAGE, GAGE, NY-ESO-1, and Survivin.

In another preferred embodiment, compound (d) comprises a protein or peptide, wherein the protein or peptide has been engineered to avoid or greatly reduce the risk of degradation by the target cell's proteasome. Preferably, compound (d) comprises a protein or peptide whose site of activity is either in the cytosol or in one of the target cell's compartments or organelles through which the conjugates of the present invention travel. Within this embodiment, an enzymatic cleavage site is preferably present within the conjugate to enable release of the protein or peptide at the target cell's desired compartment, organelle or cytosol.

In another embodiment of the present invention, small molecules (i.e., drugs), therapeutic molecules, diagnostic/imaging molecules, and the like that are desired to be delivered to either the cytosol or one of the target cell's compartments or organelles through which the conjugates of the present invention travel of a particular cell. Within this embodiment, an enzymatic cleavage site, as described above, is preferably present within the conjugate to enable release of the small molecule, therapeutic molecule, diagnostic molecule, or the like at the target cell's desired compartment, organelle or cytosol.

Small molecules that are contemplated to be delivered using the present invention include but are not limited to tamoxifen, dexamethasone, taxol, paclitaxel, cisplatin, oxaliplatin, and carboplatin.

Therapeutic molecules that are contemplated to be delivered using the present invention include but are not limited to antibodies, antibody fragments, peptides, peptoids, and decoy oligonucleotides.

Diagnostic or imaging molecules that are contemplated to be delivered using the present invention include but are not limited to Herpes simplex virus thymidine kinase (HSV1-TK, i.e., for tumor cell diagnostics/imaging), fluorochromes, quantum dots, (super-)(para-) magnetic nanoparticles, labelled antibodies, labelled antibody fragments, molecular beacons, biosensors (e.g. carbonic anhydrase), oligopeptide-based probes for detection of protease activity, peptide-based fluorescent sensors of protein kinase activity, radioactively-labeled metabolites, and D2R.

Tumor suppressor proteins and peptides that may be delivered according to the present invention include but are not limited to p53, p21, p15, BRCA1, BRCA2, IRF-1, PTEN, RB, APC, DCC, NF-1, NF-2, WT-1, MEN I, MEN-II, zacl, p73, VHL, MMAC1, FCC and MCC peptides.

Various enzymes also are of interest and may be delivered using the present invention. Such enzymes include but are not limited to cytosine deaminase, adenosine deaminase, hypoxanthine-guanine phosphoribosyltransferase, galactose-1-phosphate uridyltransferase, phenylalanine hydroxylase, glucocerebrosidase, sphingomyelinase, a-L-iduronidase, glucose-6-phosphate dehydrogenase, HSV thymidine kinase and human thymidine kinase.

Another class of proteins that is contemplated to be delivered using the present invention include interleukins (IL) and cytokines. These include but are not limited to interleukin 1 (IL-1), IL-2, IL-3 IL-4, IL-5, IL-6, IL-7, IL-8, IL-9, IL-10, IL-11, IL-12, IL-13, IL-14, IL-15, P-interferon, alpha-interferon, beta-interferon, gamma-interferon, angiostatin, thrombospondin, endostatin, METH-1, METH-2, GM-CSF, G-CSF, M-CSF and tumor necrosis factor.

Cell cycle regulators may also be delivered using the present invention. Such cell cycle regulators include but are not limited to p27, p16, p21, p57, p18, p73, p19, p15, E2F-1, E2F-2, E2F-3, p107, p130 and E2F-4.

In a preferred embodiment, a conjugate of the present invention further comprises a nuclear localization signal. Use of a nuclear localization signal peptide is preferred within a conjugate of the present invention when delivery of compound (d) to the nucleus is desired. Examples of nuclear localization signals of use in the conjugates of the present invention include but are not limited to PKKKRKV of SV40 Large T-antigen (SEQ ID NO: 246) or KRPAATKKAGQAKKKK of nucleoplasmin (SEQ ID NO: 247) [49]. Preferably, a nuclear localization signal is positioned within the conjugate such that if any of the delivery carrier modules (a), (b), or (c) are released from the conjugate via enzymatic or chemical cleavage at a cleavage site within the conjugate, the nuclear localization signal remains linked to compound (d). In another preferred embodiment, a nuclear localization signal is positioned within the conjugate such that if when compound (d) is released from the conjugate via enzymatic or chemical cleavage at a cleavage site within the conjugate, the nuclear localization signal remains linked to compound (d).

In another preferred embodiment, a conjugate of the present invention can be prepared and used to deliver a compound (d) from the ER directly to the nucleus by exploiting the linked membranes of the ER and nucleus (see for example, [50]). Preferably, the conjugate comprises a compound (d) that comprises a DNA molecule, a transcription factor or a small molecule that modulates transcription. In a particularly preferred embodiment, the conjugate comprises at least 2 compounds (d), wherein the first compound (d) is a DNA molecule and the second compound (d) is a transcription factor or a small molecule that modulates transcription.

In a third aspect, the present invention relates to methods of preparing a delivery system or conjugate of the invention. In a preferred embodiment, the method of preparing a conjugate of the invention comprises coupling (i.e., covalently or non-covalently linking, synthesizing, producing recombinantly, and the like) at least one module (a) that mediates cell targeting and facilitates cellular uptake, at least one module (b) that facilitates transport to the endoplasmic reticulum (ER), at least one module (c) that mediates translocation from the ER to the cytosol, and at least one compound (d), wherein the modules (a), (b) and (c) and the compound (d) are linked to each other in any arrangement and in any stoichiometry. The present invention also provides kits comprising at least one component of a conjugate of the invention. Preferably, a kit of the present invention comprises a module (a), a module (b), a module (c), and/or a compound (d). The kit optionally includes a peptide linker and/or a peptide comprising a cleavage site.

In a fourth aspect, the present invention relates to the use of the delivery system or conjugate of the present invention as a pharmaceutical.

In a fifth aspect, the present invention relates to a pharmaceutical composition comprising the conjugate of the present invention or a pharmaceutically acceptable salt thereof and a pharmaceutically acceptable excipient, carrier, and/or diluent. Preferably, the pharmaceutical composition comprises a pharmaceutically acceptable excipient, carrier and/or diluent and a conjugate of the present invention comprising at least one module (a), at least one module (b), at least one module (c) and at least one compound (d), wherein the modules (a), (b) and (c), and the compound (d) are linked to each other in any arrangement.

Any conjugate of the present invention may be admixed with a pharmaceutically acceptable excipient, carrier, or diluent, or a mixture thereof. Even though the conjugates of the present invention (including their pharmaceutically acceptable salts, esters and pharmaceutically acceptable solvates) can be administered alone, they will generally be administered in admixture with a pharmaceutical buffer, diluent, or excipient, particularly for human therapy.

The term “excipient” when used herein is intended to indicate all substances in a pharmaceutical formulation which are not active ingredients such as, e.g., binders, lubricants, thickeners, surface active agents, preservatives, emulsifiers, buffers, decharging agents, flavoring agents, or colorants. Examples of such suitable excipients for the various different forms of pharmaceutical compositions described herein have been previously described [51]. Preferably, to neutralize the high negative charge of the nucleic acids within a conjugate of the present invention, human protamine, spermine, spermidine or other polycations, can be added to the conjugate or a formulation of the conjugate of the present invention.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

As used herein, “pharmaceutically acceptable carrier” includes any material, which when combined with the conjugate retains the activity of the conjugate activity and is non-reactive with the subject's immune system. Examples include, but are not limited to, any of the standard pharmaceutical carriers such as a phosphate buffered saline solution, water, emulsions such as oil/water emulsion, glycerol, ethanol, and various types of wetting agents. Other carriers may also include sterile solutions, tablets including coated tablets and capsules. Typically such carriers contain excipients such as starch, milk, sugar, glucose, lactose, certain types of clay, gelatin, stearic acid or salts thereof, methyl cellulose, magnesium stearate, mannitol, sorbitol, magnesium or calcium stearate, talc, vegetable fats or oils, gums, glycols, or other known excipients. Such carriers may also include flavor and color additives or other ingredients. Compositions comprising such carriers are formulated by well known conventional methods.

The term “pharmaceutically acceptable salt” refers to a salt of the conjugate of the present invention. Suitable pharmaceutically acceptable salts include acid addition salts which may, for example, be formed by mixing a solution of the conjugate of the present invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Illustrative examples of pharmaceutically acceptable salts include, but are not limited to, acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, sodium, stearate, sulfate, subacetate, succinate, tannate, tartrate, teoclate, tosylate, triethiodide, undecanoate, valerate, and the like [see, for example, 52]. When compound (d) of a conjugate of the present invention is a nucleic acid, the pharmaceutically acceptable salt is preferably a sodium salt.

Pharmaceutical compositions of the invention are suitable for use in a variety of drug delivery systems. Suitable formulations for use in the present invention, including acceptable carrier or diluents for therapeutic use are well known in the pharmaceutical art, and methods for drug delivery are described (see for example [53 and 54].

The pharmaceutical compositions may be formulated for any appropriate manner of administration to an organism, preferably a mammal, and even more preferably a human. As used herein, “administering” includes topical, transdermal, intradermal, oral, nasal, inhalation, transmucosal, intravenous, intra-arterial, intravascular, intracardiac, intraosseous, intrathecal, intracranial, epidural, intracerebral, intracerebroventricular, intracisternal, intraperitoneal, intralesional, intravesical, intravitreal, intracaverous, intravaginal, vaginal, intrauterine, rectal, subcutaneous or intramuscular administration and the means or the implantation of a slow-release device e.g., an osmotic pump, to the subject. The concentration of a conjugate of the present invention in the pharmaceutical composition will vary upon the particular application, the nature of the disease, the frequency of administration, or the like.

Commonly, the pharmaceutical compositions are administered parenterally, e.g., intravenously. Thus, the invention provides pharmaceutical compositions for parenteral administration that comprise the conjugate of the present invention dissolved or suspended in an acceptable carrier, preferably an aqueous carrier, e.g., water, buffered water, saline, PBS, alcohol, and the like. The pharmaceutical compositions may further comprise pharmaceutically acceptable auxiliary substances as required to approximate physiological conditions, such as pH adjusting and buffering agents, tonicity adjusting agents, wetting agents, detergents and the like.

These pharmaceutical compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9 and most preferably from 7 and 8.

In some embodiments, the conjugates of the invention can be incorporated into liposomes formed from standard vesicle-forming lipids. A variety of methods are available for preparing liposomes, as described in, e.g., [55-57]; U.S. Pat. Nos. 4,235,871, 4,501,728 and 4,837,028. The targeting of liposomes using a variety of targeting agents is well known in the art (see, e.g., U.S. Pat. Nos. 4,957,773 and 4,603,044). Standard methods for coupling targeting agents to liposomes can be used. These methods generally involve incorporation into liposomes of lipid components, such as phosphatidylethanolamine, which can be activated for attachment of targeting agents, or derivatized lipophilic compounds, such as lipid-derivatized peptides of the invention. Targeting mechanisms generally require that the targeting agents be positioned on the surface of the liposome in such a manner that the target moieties are available for interaction with the target, for example, a cell surface receptor. Commonly used lipid delivery methods that are used to deliver siRNAs have been previously described and may be of use with the conjugates of the present invention [58-61].

In a preferred embodiment, a conjugate of the present invention, particularly wherein the conjugate comprises an siRNA as compound (d), is administered in vivo using a method currently used for therapeutic siRNAs. Such methods include but are not limited to cholesterol conjugation to the conjugate, the use of polycation nanoparticles to deliver the conjugate to a target cell via a cell surface ligand that binds to a receptor on the target cell, encapsulation of the conjugate into a cationic or neutral lipid bilayer using SNALPs (stable nucleic acid lipid particles) that are coated with diffusible PEG-lipid conjugates, masked endosomolytic agent (MEA)-dynamic polyconjugates (DPCs) comprising a ligand to target the conjugate to a specific cell, the use of protamine-tagged (or any other positive charged molecule-tagged) specific antibody to target the conjugate to a specific cell for receptor-mediated uptake, the use of RNA aptamers to target the conjugate to a specific cell, the use of immunoliposomes, Trans-IT TKO, LF2000, and the like [62-64].

The dosage ranges for the administration of the conjugates of the invention are those large enough to produce the desired effect in which the symptoms of the disease or condition to be treated show some degree of amelioration. The dosage should not be so large as to cause adverse side effects. Generally, the dosage will vary with the age, condition, sex and extent of the disease in a subject or patient and can be determined by one of skill in the art. Dosage regimens are adjusted to provide the optimum desired response (e.g., a therapeutic response). For example, a single bolus may be administered, several divided doses may be administered over time or the dose may be proportionally reduced or increased as necessitated by the therapeutic situation. It is especially advantageous to formulate parenteral compositions in dosage unit form for ease of administration and uniformity of dosage.

Preferably, the conjugates of the present invention are administered intravenously at a dose ranging from about 1 to about 4000 nmol/kg, from about 1 to about 3000 nmol/kg, from about 1 to about 2000 nmol/kg, from about 1 to about 1000 nmol/kg, from about 100 to about 4000 nmol/kg, from about 100 to about 3000 nmol/kg, from about 100 to about 2000 nmol/kg, from about 100 to about 1000 nmol/kg, from about 200 to about 4000 nmol/kg, from about 200 to about 3000 nmol/kg, from about 200 to about 2000 nmol/kg, from about 200 to about 1000 nmol/kg, from about 300 to about 4000 nmol/kg, from about 300 to about 3000 nmol/kg, from about 300 to about 2000 nmol/kg, from about 300 to about 1000 nmol/kg, from about 500 to about 4000 nmol/kg, from about 500 to about 3000 nmol/kg, from about 500 to about 2000 nmol/kg, from about 500 to about 1000 nmol/kg, from about 1000 to about 4000 nmol/kg, from about 1000 to about 3000 nmol/kg, from about 1000 to about 2000 nmol/kg, from about 2000 to about 4000 nmol/kg, from about 2000 to about 3000 nmol/kg, from about 3000 to about 4000 nmol/kg, from about 1 to about 500 nmol/kg, from about 1 to about 400 nmol/kg, from about 1 to about 300 nmol/kg, from about 1 to about 200 nmol/kg, from about 1 to about 100 nmol/kg, from about 10 to about 500 nmol/kg, from about 10 to about 400 nmol/kg, from about 10 to about 300 nmol/kg, from about 10 to about 200 nmol/kg, from about 10 to about 100 nmol/kg, from about 100 to about 500 nmol/kg, from about 100 to about 400 nmol/kg, from about 100 to about 300 nmol/kg, from about 100 to about 200 nmol/kg, from about 200 to about 500 nmol/kg, from about 200 to about 400 nmol/kg, from about 200 to about 300 nmol/kg, from about 300 to about 500 nmol/kg, from about 300 to about 400 nmol/kg, from about 400 to about 500 nmol/kg, from about 1 to about 50 nmol/kg, from about 1 to about 40 nmol/kg, from about 1 to about 30 nmol/kg, from about 1 to about 20 nmol/kg, from about 1 to about 10 nmol/kg, from about 1 to about 5 nmol/kg, from about 1 to about 4 nmol/kg, from about 1 to about 3 nmol/kg, from about 1 to about 2 nmol/kg, from about 2 to about 5 nmol/kg, from about 2 to about 4 nmol/kg, from about 2 to about 3 nmol/kg, from about 3 to about 5 nmol/kg, or from about 3 to about 4 nmol/kg.

Preferably, the conjugates of the present invention are administered intracranially or via an osmotic pump at a dose ranging from about 0.001 to about 10 nmol, from about 0.001 to about 5 nmol, from about 0.001 to about 3 nmol, from about 0.001 to about 2 nmol, from about 0.001 to about 1 nmol, from about 0.001 to about 0.5 nmol, from about 0.001 to about 0.3 nmol, from about 0.001 to about 0.2 nmol, from about 0.001 to about 0.1 nmol, from about 0.001 to about 0.05 nmol, from about 0.001 to about 0.03 nmol, from about 0.001 to about 0.02 nmol, from about 0.001 to about 0.01 nmol, from about 0.001 to about 0.005 nmol, from about 0.001 to about 0.003 nmol, from about 0.001 to about 0.002 nmol, from about 0.002 to about 10 nmol, from about 0.002 to about 5 nmol, from about 0.002 to about 3 nmol, from about 0.002 to about 2 nmol, from about 0.002 to about 1 nmol, 0.002 to about 0.5 nmol, from about 0.002 to about 0.3 nmol, from about 0.002 to about 0.2 nmol, from about 0.002 to about 0.1 nmol, from about 0.002 to about 0.05 nmol, from about 0.002 to about 0.03 nmol, from about 0.002 to about 0.02 nmol, from about 0.002 to about 0.01 nmol, from about 0.002 to about 0.005 nmol, from about 0.002 to about 0.003 nmol, from about 0.003 to about 10 nmol, from about 0.003 to about 5 nmol, from about 0.003 to about 3 nmol, from about 0.003 to about 2 nmol, from about 0.003 to about 1 nmol, 0.003 to about 0.5 nmol, from about 0.003 to about 0.3 nmol, from about 0.003 to about 0.2 nmol, from about 0.003 to about 0.1 nmol, from about 0.003 to about 0.05 nmol, from about 0.003 to about 0.03 nmol, from about 0.003 to about 0.02 nmol, from about 0.003 to about 0.01 nmol, from about 0.003 to about 0.005 nmol, from about 0.005 to about 10 nmol, from about 0.005 to about 5 nmol, from about 0.005 to about 3 nmol, from about 0.005 to about 2 nmol, from about 0.005 to about 1 nmol, 0.005 to about 0.5 nmol, from about 0.005 to about 0.3 nmol, from about 0.005 to about 0.2 nmol, from about 0.005 to about 0.1 nmol, from about 0.005 to about 0.05 nmol, from about 0.005 to about 0.03 nmol, from about 0.005 to about 0.02 nmol, from about 0.005 to about 0.01 nmol, from about 0.01 to about 10 nmol, from about 0.01 to about 5 nmol, from about 0.01 to about 3 nmol, from about 0.01 to about 2 nmol, from about 0.01 to about 1 nmol, from about 0.01 to about 0.5 nmol, from about 0.01 to about 0.3 nmol, from about 0.01 to about 0.2 nmol, from about 0.01 to about 0.1 nmol, from about 0.01 to about 0.05 nmol, from about 0.01 to about 0.03 nmol, from about 0.01 to about 0.02 nmol, from about 0.02 to about 10 nmol, from about 0.02 to about 5 nmol, from about 0.02 to about 3 nmol, from about 0.02 to about 2 nmol, from about 0.02 to about 1 nmol, from about 0.02 to about 0.5 nmol, from about 0.02 to about 0.3 nmol, from about 0.02 to about 0.2 nmol, from about 0.02 to about 0.1 nmol, from about 0.02 to about 0.05 nmol, from about 0.02 to about 0.03 nmol, from about 0.03 to about 10 nmol, from about 0.03 to about 5 nmol, from about 0.03 to about 3 nmol, from about 0.03 to about 2 nmol, from about 0.03 to about 1 nmol, from about 0.03 to about 0.5 nmol, from about 0.03 to about 0.3 nmol, from about 0.03 to about 0.2 nmol, from about 0.03 to about 0.1 nmol, from about 0.03 to about 0.05 nmol, from about 0.05 to about 10 nmol, from about 0.05 to about 5 nmol, from about 0.05 to about 3 nmol, from about 0.05 to about 2 nmol, from about 0.05 to about 1 nmol, from about 0.05 to about 0.5 nmol, from about 0.05 to about 0.3 nmol, from about 0.05 to about 0.2 nmol, from about 0.05 to about 0.1 nmol, from about 0.1 to about 10 nmol, from about 0.1 to about 5 nmol, from about 0.1 to about 3 nmol, from about 0.1 to about 2 nmol, from about 0.1 to about 1 nmol, from about 0.1 to about 0.5 nmol, from about 0.1 to about 0.3 nmol, from about 0.1 to about 0.2 nmol, from about 0.2 to about 10 nmol, from about 0.2 to about 5 nmol, from about 0.2 to about 3 nmol, from about 0.2 to about 2 nmol, from about 0.2 to about 1 nmol, from about 0.2 to about 0.5 nmol, from about 0.2 to about 0.3 nmol, from about 0.3 to about 10 nmol, from about 0.3 to about 5 nmol, from about 0.3 to about 3 nmol, from about 0.3 to about 2 nmol, from about 0.3 to about 1 nmol, from about 0.3 to about 0.5 nmol, from about 0.5 to about 10 nmol, from about 0.5 to about 5 nmol, from about 0.5 to about 3 nmol, from about 0.5 to about 2 nmol, from about 0.5 to about 1 nmol, from about 1 to about 10 nmol, from about 1 to about 5 nmol, from about 1 to about 3 nmol, from about 1 to about 2 nmol, from about 2 to about 10 nmol, from about 2 to about 5 nmol, from about 2 to about 3 nmol, from about 3 to about 10 nmol, from about 3 to about 5 nmol, or from about 5 to about 10 nmol.

More preferably, the conjugates of the invention, when administered via an osmotic pump, are administered at a daily dose of about 3 nmol.

Additional pharmaceutical methods may be employed to control the duration of action. Controlled release preparations may be achieved by the use of polymers to conjugate, complex or adsorb the conjugates of the present invention. The controlled delivery may be exercised by selecting appropriate macromolecules (for example, polyesters, polyamino carboxymethylcellulose, and protamine sulfate) and the concentration of macromolecules as well as the methods of incorporation in order to control release. Another possible method to control the duration of action by controlled release preparations is to incorporate the conjugate into particles of a polymeric material such as polyesters, polyamino acids, hydrogels, poly(lactic acid) or ethylene vinylacetate copolymers.

In order to protect the conjugates of the present invention, and the peptides or proteins comprised within said conjugates, from binding with plasma proteins, it is preferred that the conjugates be entrapped in microcapsules prepared, for example, by coacervation techniques or by interfacial polymerization, for example, hydroxymethylcellulose or gelatin-microcapsules and poly(methymethacrylate) microcapsules, respectively, or in colloidal drug delivery systems, for example, liposomes, albumin microspheres, microemulsions, nanoparticles, and nanocapsules or in macroemulsions. Such teachings have been previously described [53].

The conjugates of the invention are well suited for use in targetable drug delivery systems such as synthetic or natural polymers in the form of macromolecular complexes, nanocapsules, microspheres, or beads, meso-particles, and lipid-based systems including oil-in-water emulsions, micelles, mixed micelles, liposomes, and resealed erythrocytes. These systems are known collectively as colloidal drug delivery systems. Typically, such colloidal particles containing the dispersed conjugates are about 50 nm-2 μm in diameter. The size of the colloidal particles allows them to be administered intravenously such as by injection, or as an aerosol. Materials used in the preparation of colloidal systems are typically sterilizable via filter sterilization, nontoxic, and biodegradable, for example albumin, ethylcellulose, casein, gelatin, lecithin, phospholipids, and soybean oil. Polymeric colloidal systems are prepared by a process similar to the coacervation of microencapsulation. The targeted delivery system-encapsulated conjugate may be provided in a formulation comprising other compounds as appropriate and an aqueous physiologically acceptable medium, for example, saline, phosphate buffered saline, or the like.

In an exemplary embodiment, the conjugates of the present invention are components of a liposome, used as a targeted delivery system. When phospholipids are gently dispersed in aqueous media, they swell, hydrate, and spontaneously form multilamellar concentric bilayer vesicles with layers of aqueous media separating the lipid bilayer. Such systems are usually referred to as multilamellar liposomes or multilamellar vesicles (MLVs) and have diameters ranging from about 100 nm to about 4 μm. When MLVs are sonicated, small unilamellar vesicles (SUVS) with diameters in the range of from about 20 nm to about 50 nm are formed, which contain an aqueous solution in the core of the SUV.

Examples of lipids useful in liposome production include phosphatidyl compounds, such as phosphatidylglycerol, phosphatidylcholine, phosphatidylserine, and phosphatidylethanolamine. Particularly useful are diacylphosphatidylglycerols, wherein the lipid moiety comprises from 14-18 carbon atoms, particularly from 16-18 carbon atoms, and are saturated. Illustrative phospholipids include egg phosphatidylcholine, dipalmitoylphosphatidylcholine, and distearoylphosphatidylcholine.

In a sixth aspect, the conjugates of the present invention may be of use as diagnostic reagents. For example, labeled compounds can be used to locate areas of inflammation or tumor metastasis in a patient suspected of having an inflammation. For this use, the compounds can be labeled with ¹²⁵I, ¹⁴C, or tritium.

In a seventh aspect, the present invention relates to the use of the delivery system or conjugate of the invention for the manufacture of a medicament (i.e., a pharmaceutical composition). The pharmaceutical compositions may be used to treat humans or animals, in human and veterinary medicine respectively.

In an eighth aspect, the present invention relates to a method of delivering the compound (d) to a cell, which comprises the steps:
(a) providing a cell,
(b) contacting a conjugate of the present invention with said cell,
   under the conditions that allow the conjugate to be internalized by the cell, thereby delivering compound (d) to the cell. In one embodiment, the cell is an isolated cell or cultured cell.

Preferably, the cell is a eukaryotic cell, an invertebrate cell, a vertebrate cell, a nematode cell, a fungal cell, an Aspergillus cell, a yeast cell, a Sacchromyces cell, a Pichia cell, an insect cell, an Sf9 cell, an animal cell, a non-human animal cell, a Chinese hamster ovary (CHO) cell, a mammalian cell, a non-human mammalian cell, a primate cell, a non-human primate cell, a human cell, or a plant cell. In a preferred embodiment, the method of delivering a compound (d) to a cell results in increased or decreased gene expression and/or protein production in the cell.

In a particularly preferred embodiment, the method of delivering a compound (d) to a cell comprises the steps:
(a) providing a cell,
(b) contacting a conjugate of the present invention with said cell,
   under the conditions that allow the conjugate to be internalized by the cell, thereby delivering compound (d), and whereby gene expression of said cell is modified (i.e., increased or decreased) and/or protein production in said cell is modified (i.e., increased or decreased). Thus, methods of modifying gene expression and/or protein production in a cell using the delivery system or conjugate of the present invention are also provided. Preferably, the cell is an isolated cell or a cultured cell. More preferably, the cell is an isolated cell or cultured cell used for recombinant gene expression, protein production, and/or drug, small molecule, or biological molecule screening. Preferably, the isolated cell or cultured cell is a eukaryotic cell, an invertebrate cell, a vertebrate cell, a nematode cell, a fungal cell, an Aspergillus cell, a yeast cell, a Sacchromyces cell, a Pichia cell, an insect cell, an insect cell, an animal cell, a non-human animal cell, a CHO cell, a mammalian cell, a non-human mammalian cell, a primate cell, a non-human primate cell, a human cell, or a plant cell.

In a ninth aspect, the present invention relates to a method of delivering a compound (d) to an organism comprising the step of:
(a) administering a sufficient amount of a conjugate of the present invention to a patient, thereby delivering the compound (d) to the organism.

Preferably, the organism is an animal, a mammal, a human, or a plant. In a preferred embodiment, the method of delivering a compound (d) to an organism results in increased or decreased gene expression and/or protein production in a cell of the organism. In another preferred embodiment, the method of delivering a compound (d) to an organism results in increased immunity or an increased immune response in the organism.

In a tenth aspect, the present invention relates to a method of delivering a compound (d) to a patient comprising the step of:
(a) administering a sufficient amount of a conjugate of the present invention to a patient, thereby delivering the compound (d) to the patient.

As used herein, a “patient” refers to an organism suffering from and/or undergoing treatment for a disorder, disease or condition. The patient can be any animal but is preferably a mammal, such as a cow, horse, mouse, rat, cat, dog, pig, goat, sheep, chicken, or a primate. In a preferred embodiment, the patient is a human. Preferably, the patient is an animal, a non-human animal, a mammal, a non-human mammal, or a human. More preferably, the patient is a human suffering from and/or undergoing treatment for a disorder, disease or condition mediated by increased, decreased, insufficient, aberrant or unwanted target gene expression or protein production. In an another embodiment, the patient is suffering from and/or undergoing treatment for a disorder, disease or condition mediated by decreased, insufficient, or lack of immunity.

In a preferred embodiment, a method of delivering a compound (d) to a patient comprises the step of administering to a patient a sufficient amount of a conjugate comprising, essentially consisting of or consisting of:
(a) at least one module (a) that mediates cell targeting and facilitates cellular uptake,
(b) at least one module (b) that facilitates transport to the endoplasmic reticulum (ER),
(c) at least one module (c) that mediates translocation from the ER to the cytosol, and
(d) at least one compound (d),
wherein the modules (a), (b) and (c), and the compound (d) are linked to each other in any arrangement, and wherein the conjugate optionally comprises a nuclear localization signal, and
   thereby delivering the compound (d) to the patient.

Preferably, the compound (d) to be delivered to a patient using a method according to the invention is an siRNA.

In a further aspect, the present invention relates to the conjugates of the present invention for use in therapy and prevention of disease, which can be prevented or treated by the delivery of at least one compound (d).

A “disease” is a state of health of an organism, wherein the organism cannot maintain homeostasis, and wherein if the disease is not ameliorated then the organism's health begins or continues to deteriorate.

Because RNAi mediated silencing is expected to persist for several days after administering a conjugate according to the invention comprising an siRNA as compound (d), in many instances, it is possible to administer the conjugates of the present invention with a frequency of less than once per day, or, for some instances, only once for the entire therapeutic regimen. For example, treatment of some cancer cells may be mediated by a single bolus administration, whereas a chronic viral infection may require regular administration, e.g., once per week or once per month.

The present invention provides conjugates which can effectively deliver compounds such as biologically active macromolecules, nucleic acids or peptides in particular, to a cell, either in culture or within an organism by using endogenous processes that occur ubiquitously within all cells.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be encompassed by the present invention.

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### EXAMPLES

Abbreviations used herein include: kilogram (kg), milligram (mg), milliliter (mL), microliter (μL), molar (M), millimolar (mM), micromolar (μM), micromoles (μmol), nanomoles (nmol), hour (h), kiloDalton (kDa), degrees Celsius (° C.), minute (min), millimeter (mm), micron (μm), nanometer (nm), amino acid (aa), wild-type (wt), gravity (g), and intraperitoneal (i.p.).

### Example (1)

### Synthesis of DARE™ delivery system delivery modules and preparation of the modules-siRNA conjugate DARE™-R-CX (FIG. 2, DARE™ 2.01) and DARE™-R-AK-CX (DARE™ Delivery Vehicle Design 2.03)

(i) Synthesis of the Linkage Molecules Containing Delivery Modules (b) and (c):

Two [“module (b)+module (c)”+linker] molecules: H₂N—C(NPyS)(S-G)₃(DprAoa)(S-G)₃ NASSSRSGLDDINPTVLLKERSTEL-OH [“module (b)+module (c)” functionalities are provided by a human COX2 peptide comprising an amino acid sequence comprising SEQ ID NO: 177; CX1] and H₂N—C(NPyS)(S-G)₃(DprAoa)(S-G)₃NASSSRSGLDDINPTVLLK AKDEL-OH [“module (b)+module (c)” comprise SEQ ID NO: 244; CX2a] are synthesized commercially by standard solid-phase Fmoc peptide chemistry, deprotected in the standard fashion and purified by reversed phase High Performance Liquid Chromatography (HPLC) to a purity of >95%. The activated cysteine residue is introduced using Boc-Cys(NPys)-OH (Bachem product no. A-2825) as a building block. Fmoc-Dpr(Boc-Aoa)-OH (Novabiochem product no. 04-12-1185) is used to introduce the N-β-aminoxyacetyl L-diaminopropionyl residue. Quality control (QC) of the purified peptide is done by amino acid analysis, electrospray mass spectroscopy (ESMS) and analytical reversed phase HPLC.

(ii) Synthesis of the Delivery Carrier Comprising Modules (a), (b) and (c) and the Linker:

To prepare module (a), recombinant Ricin toxin B subunit [(Ricin B comprising SEQ ID NO: 124, which is obtained commercially from Vector Laboratories, Inc., catalog no. L-1290 or prepared recombinantly] and supplied or prepared as a 1 mg/mL solution in 10 mM aqueous sodium phosphate, 0.15 M NaCl, pH 7.5, containing 0.08% sodium azide and 50 mM 2-mercaptoethanol, is supplemented with fresh 50 mM 2-mercaptoethanol and incubated for 1 h at room temperature (RT) to ensure that the Cys residue at position 4 from the C-terminus is completely in the fully reduced form. The solution is then dialyzed against degassed 10 mM sodium phosphate buffer, 150 mM NaCl, pH 7.5 in a Slide-A-Lyzer dialysis cassette with molecular weight cut off of 10 kDa, volume 0.5-3 mL (Pierce no. 66380). Two dialyses are run for 2 h each at RT, followed by a final dialysis overnight at 4° C. The solution containing Ricin B is reacted overnight at RT under nitrogen with a phosphate buffered saline (PBS) solution containing 1.1 mole equivalents of either of the linkage molecules containing modules (b) and (c) from Example 1(i) above. The desired carrier [modules (a)+(b)+(c)] is then purified by preparative gel filtration [Size Exclusion Chromatography (SEC)] using a HiLoad 16/60 Superdex 75 prep grade column (GE Healthcare, part no. 17-1068-01) eluted with 50 mM sodium dihydrogen phosphate buffer, 100 mM NaCl, 2 μM EDTA, pH 5.0 at a flow rate of 1 mL/min. Identification of the desired carrier peak is enabled by having calibrated the SEC column with Ricin B and with the linker-peptide entity from Example 1(i). The product is analyzed by ESMS and by native gel electrophoresis and compared to Ricin B and the linker-peptide.

(iii) Preparation of Cargo Compound (d) [an siRNA]:

A double stranded RNA molecule comprised of two 21 mer strands, with a double stranded region of 19 nucleotides in length and 2 nucleotides overhanging at the 3′ end of each strand, and targeting glyceraldehyde 3-phosphate dehydrogenase (GAPDH), wherein the sense strand comprises CCAuCUUCCAGGAGCgAGAuu (SEQ ID NO: 248), wherein lowercase u or g represents a 2′-O-Me-modified nucleotide; and the antisense strand comprises UCUCGCUCCUGgAAGAuGGdTdG (SEQ ID NO: 249), wherein lowercase u or g represents a 2′-O-Me-modified nucleotide and wherein the antisense strand has a 5′-phosphate and deoxynucleotides at its 3′ end (dNdN), is synthesized such that the 5′-terminus of the sense strand is modified with 5′-(C6 aminolinker)-phosphate-(C6-SS—C6 spacer)-phosphate-Cy3. The Cy3 dye is for tracking purposes by fluorescence and the disulfide bond ensures that the cargo can finally be released within the reducing environment of the cell. The single strands were analyzed by ESMS and analytical HPLC for QC prior to annealing. The desalted lyophilized siRNA is dissolved in sterile sodium tetraborate buffer pH 8.5 and reacted with 10 molar equivalents of the adaptor molecule SFB (succinimidyl 4-formylbenzoate, Thermo Scientific, catalog no. 22419) dissolved in 10% by volume of DMSO for 3 h at RT. The siRNA bearing a benzaldehyde function is isolated by dialysis against 50 mM sodium phosphate, 100 mM NaCl, 2 μM EDTA, pH 5 using a Slide-A-Lyzer dialysis cassette with a molecular weight cut-off of 3.5 kDa, volume 0.5-3 mL (Pierce no. 66330). Two dialyses are performed for 2 h each at RT followed by a third dialysis overnight at 4° C. The final solution is concentrated to a final volume of approximately 1 mL using a small ultrafiltration cell. QC of the adapter modified siRNA is done by ESMS and analytical HPLC. A small aliquot of the sample is analyzed for the presence of the aldehyde moiety by reaction with an excess of Cascade Blue hydrazide (Molecular Probes, catalog no. C-687) in buffer at pH 5, desalted by ethanol precipitation and analyzed by native anion-exchange HPLC on a MonoQ column (GE Healthcare) using multiwavelength detection (260 nm for the RNA, 399 nm for the Cascade Blue and 550 nm for the Cy3).

(iv) Coupling of the Cargo [Compound (d)] to the Delivery Carrier [Modules (a)+(B)+(C) and a Linker]

The carrier from Example 1(ii) above is mixed with an approximately equimolar amount of the adapter-siRNA component (cargo) from Example 1(iii) above and kept for several hours at RT. The desired conjugate is purified by preparative SEC on a HiLoad 16/60 Superdex 75 prep grade column (GE Healthcare, part no. 17-1068-01) eluted at 1 mL/min with sterile PBS, pH 7.4. The column effluent is monitored at 260 nm and 550 nm. Calibration of the column is carried out prior to the preparative purification using the individual reaction components. Those fractions containing the conjugate are combined and concentrated by ultrafiltration (Amicon device) and the final concentrate is stored at 4° C. QC is performed by native gel electrophoresis and analytical SEC on Superdex 75 10/300 GL column (GE Healthcare, part no. 17-5174-01).

Since the DARE™ constructs comprise several components linked together covalently (in most cases by 2 disulfide bonds), and comprise polypeptides as well as a cargo molecule, it may be difficult to characterize them as single entities by molecular weight using standard MS techniques such as MALDI-TOF or ESMS. While characterization by PAGE or gel filtration certainly gives a general indication of their homogeneity, to be sure that the molecule isolated comprises all the expected component parts, it is preferred to incubate the DARE™ construct with a reducing agent such as dithiothreitol (DTT) or tris(2-carboxyethyl)phosphine (TCEP) to cleave all accessible disulfide bonds. This will generate 3 molecules, in the case of 2 disulfide (S—S) bonds, that can be separated by ion-pair reversed phase HPLC (HPLC) and characterized by ESMS. If necessary, the individual components may also be sequenced by MS-MS, however in most cases, it should suffice that the measured masses of the components match the expected (calculated) masses.

Additionally, a small aliquot of the product is treated with dithiothreitol (DTT) to reduce the two accessible disulfide bonds to generate 3 reaction products (i.e., ricin B, linker-peptide construct plus adapter and HS—(CH₂)₆—OP(O₂)—O-Cy3-siRNA) that are analyzed by ESMS and analytical SEC using a Superdex 75 10/300 GL column eluted with PBS.

It will be apparent to one of skill in the art that the approach described within this Example may be used to attach other cargoes, e.g. a double stranded DNA, a single stranded miRNA antagonist (antagomir), an antisense oligonucleotide, and the like to a delivery carrier (i.e., [module (a)+module (b)+module (c)] of the present invention. It may be advantageous to attach the single stranded cargoes via their 3′-termini. The 3′-modified single strands are made by procedures that are standard to those skilled in the art.

A detailed drawing of conjugate DARE™-R-AK-CX as described in Example 1 is shown in FIG. 2 (A) as DARE™-R-AK-CX/2.03 without the Cy3 and in FIG. 2(B) as DARE™-R-AK-CX/2.03 with the Cy3.

### Example (2)

### Synthesis of DARE™ Delivery Modules and Preparation of a Delivery siRNA Conjugate DARE™-R-CXpeg, (a DARE™ Delivery Vehicle Design 2.0)

(i) Synthesis of the Linkage Molecule Containing Modules (b) and (c):

The module (b)+module (c)+linker peptide H₂N—C(NPyS)(dPEG12)(DprAoa)(dPEG12) NASSSRSGLDDINPTVLLKERSTEL-OH [“module (b)+module (c)” functionalities are provided by a human COX2 peptide comprising an amino acid sequence comprising SEQ ID NO: 177; CXpeg] is synthesized commercially by standard solid-phase Fmoc peptide chemistry, deprotected in the standard fashion and purified by reversed phase HPLC to a purity of >95%. The activated cysteine residue is introduced using Boc-Cys(NPys)-OH (Bachem product no. A-2825) as a building block. Fmoc-Dpr(Boc-Aoa)-OH (Novabiochem product no. 04-12-1185) is used to introduce the N-β-aminoxyacetyl L-diaminopropionyl residue. dPEG12 is introduced using Fmoc-dPEG₁₂-acid (Quanta BioDesign, product no. 10283). QC of the purified peptide is done by amino acid analysis, ESMS and analytical reversed phase HPLC.

(ii) Synthesis of the Delivery Carrier [Linker Plus Modules (a), (b) and (c)]:

The synthesis of the delivery carrier from ricin B and the linker-peptide from Example 2(i) above is described in Example 1(ii) above. Briefly, a ricin B [module (a)] is prepared as described in Example 1(ii), then reacted overnight at RT under nitrogen with a PBS solution containing 1.1 mole equivalent of the [linker-module (c)-module (b)] product of Example 2(i). The delivery carrier [modules (a), (b), and (c) and the linker] is purified and analyzed as described above in Example 1(ii).

(iii) Preparation of the Cargo siRNA [Compound (d)]:

The cargo siRNA [compound (d)] is prepared as described in Example 1, section (iii) above.

(iv) Coupling of Compound (d) to the Carrier Module:

The components from Example 2(ii) and (iii) above are combined and the DARE™-R-CXpeg conjugate is isolated and analyzed as described in Example 1(iv) above.

### Example (3)

### Synthesis of a DARE™ Delivery Vehicle Design 3.1 with a Teti Peptide as Module (a) for Delivering an siRNA Cargo

This Example describes the preparation of a conjugate comprising a neuronal cell targeting peptide Teti [65, 66] as module (a). Teti protein targets neurons and has the same binding characteristics as tetanus toxin [65, 66].

(i.) Synthesis of a Teti Peptide Based Module (a):

A Teti peptide HLNILSTLWKYR-(flexible linker)-C (SEQ ID NO: 250), wherein the flexible linker is either GGG, SGSG, or SGSGSG, is synthesized by standard solid-phase Fmoc peptide chemistry, deprotected in the standard fashion and purified by reversed phase HPLC to a purity of >95%. QC of the purified peptide is done by amino acid analysis, ESMS and analytical reversed phase HPLC.

(ii.) Synthesis of the Linkage Molecule Containing Modules (b) and (c):

The [module (b)+module (c)+linker] peptide H₂N—C(NPyS)(dPEG12)(DprAoa)(dPEG12) NASSSRSGLDDINPTVLLKAKDEL-OH [the peptide comprising “module (b)+module (c)” comprises an amino acid sequence comprising SEQ ID NO: 244] is synthesized by standard solid-phase Fmoc peptide chemistry, deprotected in the standard fashion and purified by reversed phase HPLC to a purity of >95%. The activated cysteine residue is introduced using Boc-Cys(NPys)-OH (Bachem product no. A-2825) as a building block. Fmoc-Dpr(Boc-Aoa)-OH (Novabiochem product no. 04-12-1185) is used to introduce the N-β-aminoxyacetyl L-diaminopropionyl residue. dPEG12 is introduced using Fmoc-dPEG₁₂-acid (Quanta BioDesign, product no. 10283). QC of the purified peptide is done by amino acid analysis, ESMS and analytical reversed phase HPLC.

(iii.) Synthesis of the Delivery Carrier Comprising Modules (a), (b) and (c) and the Linker:

A solution containing module (a) from Example 3(i) above in 100 mM sodium phosphate, 150 mM NaCl, 2 mM EDTA, pH 7.5 is reacted overnight at RT under nitrogen with a solution containing 1.1 mole equivalents of the linkage molecule containing modules (b) and (c) from Example 3(ii) above in the same buffer. The desired carrier is then purified by preparative gel filtration (SEC) using a HiLoad 16/60 Superdex 75 prep grade column (GE Healthcare, part no. 17-1068-01) eluted with 100 mM sodium dihydrogen phosphate buffer, 100 mM NaCl, 2 μM EDTA, pH 5.0 at a flow rate of 1 mL/min. Identification of the desired carrier peak is enabled by having calibrated the SEC column with the 2 individual starting materials. The product is analyzed by ESMS, native gel electrophoresis and analytical reversed phase HPLC.

(iv.) Preparation of the Cargo siRNA [Compound (d)]:

A Tuschl-style siRNA targeting GAPDH is synthesized, purified and analyzed as described in Example 1(iii) with the 5′-terminus of the sense strand modified with 5′-(C6 aminolinker)-phosphate-(C6-SS-C6 spacer)-phosphate-Cy3.

(v.) Coupling Compound (d) to the Delivery Carrier:

The delivery carrier from Example 3(iii) above is mixed with an approximately equimolar amount of the adapter-siRNA component (cargo) from Example 3(iv) above and kept for several hours at RT. The desired conjugate is purified by preparative SEC on a HiLoad 16/60 Superdex 75 prep grade column (GE Healthcare, part no. 17-1068-01) eluted at 1 mL/min with sterile PBS, pH 7.4. The column effluent is monitored at 260 nm and 550 nm. Calibration of the column is carried out prior to the preparative purification using the individual reaction components. Those fractions containing the conjugate are combined and concentrated by ultrafiltration (Vivaspin device) and the final concentrate is stored at 4° C. QC is performed by native gel electrophoresis and analytical SEC on a Superdex 75 10/300 GL column (GE Healthcare, part no. 17-5174-01). Additionally, a small aliquot of the product is treated with DTT to reduce the two accessible disulfide bonds to generate 3 reaction products (i.e., module (a), linker-peptide construct plus adapter and HS—(CH₂)₆—OP(O₂)—O-Cy3-siRNA) that are analyzed by ESMS, analytical SEC using a Superdex 75 10/300 GL column eluted with PBS, and by analytical reversed phase HPLC.

### Example (4)

### Synthesis of a DARE™ Delivery Vehicle Design 3.2 with a Single Chain Antibody as Module (a) and an siRNA Cargo

(i) Synthesis of Module (a):

An anti-EGFR single chain antibody (SEQ ID NO: 251) is synthesized with an additional cysteine at the C-terminus using solid-phase Fmoc chemistry, deprotected in the standard fashion and purified by reversed phase HPLC to a purity of >95%. QC of the purified peptide is performed using amino acid analysis, ESMS and analytical reversed phase HPLC.

(ii) Synthesis of the Linkage Molecule Containing Modules (b) and (c):

The [module (b)+module (c)+linker] peptide N-acetyl-C(NPyS)(dPEG12)(DprAoa) (dPEG12)NASSSRSGLDDINPTVLLKAKDEL-OH [the peptide comprising “module (b)+module (c)” comprises an amino acid sequence comprising SEQ ID NO: 244] is synthesized by standard solid-phase Fmoc peptide chemistry, deprotected in the standard fashion and purified by reversed phase HPLC to a purity of >95%. The activated cysteine residue is introduced using Boc-Cys(NPys)-OH (Bachem product no. A-2825) as a building block. Fmoc-Dpr(Boc-Aoa)-OH (Novabiochem product no. 04-12-1185) is used to introduce the N-β-aminoxyacetyl L-diaminopropionyl residue. dPEG12 is introduced using Fmoc-dPEG₁₂-acid (Quanta BioDesign, product no. 10283). QC of the purified peptide is done by amino acid analysis, ESMS and analytical reversed phase HPLC.

(iii) Synthesis of the Delivery Carrier Comprising Modules (a), (b), (c) and the Linker:

A solution containing module (a) from Example 4(i) above in 100 mM sodium phosphate, 150 mM NaCl, 2 mM EDTA, pH 7.5 is reacted overnight at RT under nitrogen with a solution containing 1.1 mole equivalents of the linkage molecule containing modules (b) and (c) from Example 4(ii) above in the same buffer and containing enough N,N-dimethylformamide (DMF) to ensure solubility of both components. The desired carrier is then purified by preparative gel filtration (SEC) using a HiLoad 16/60 Superdex 75 prep grade column (GE Healthcare, part no. 17-1068-01) eluted with 100 mM citrate buffer, 2 μM EDTA, pH 6.0 at a flow rate of 1 mL/min. Identification of the desired carrier peak is enabled by having calibrated the SEC column with the two individual starting materials. The product is analyzed by ESMS, native gel electrophoresis and analytical reversed phase HPLC.

(iv) Preparation of the Cargo siRNA [Compound (d)]:

A Tuschl-style siRNA targeting GAPDH is synthesized, purified, and analyzed as in Example 1(iii) with the 5′-terminus of the sense strand modified with 5′-(C6 aminolinker)-phosphate-(C6-SS-C6 spacer)-phosphate-Cy3.

(v) Coupling Compound (d) to the Delivery Carrier:

The carrier from Example 4(iii) above is mixed with an approximately equimolar amount of the adapter-siRNA (cargo) from Example 4(iv) above and kept overnight at RT. The desired conjugate is purified and analyzed as described in Example 3(v) above. A small aliquot of the product is treated with DTT to reduce the two accessible disulfide bonds to generate 3 reaction products, viz. module (a), linker-peptide construct plus adapter and HS—(CH₂)₆—OP(O₂)—O-Cy3-siRNA that are analyzed by ESMS, analytical SEC using a Superdex 75 10/300 GL column eluted with PBS, and by analytical reversed phase HPLC.

### Example (5)

### Synthesis of a DARE™ Delivery Vehicle Design 3.3a to Deliver a Non-Covalently Linked siRNA Cargo

(i) Construction of an Aldehyde Modified Transferrin as Module (a)

Human serum transferrin (SEQ ID NO: 252; Sigma, Invitrogen is reacted under mild conditions with sodium periodate to generate reactive aldehyde functionalities on the carbohydrate moieties using the published protocol of d'Alessandro et al. [67]. It has previously been shown that conjugation of peroxidase hydrazide to an aldehyde modified transferrin yields a bioconjugate that is fully recognizable by both anti-transferrin and anti-peroxidase antibodies [67].

(ii) Synthesis of a Linkage Molecule Comprising a Branched Peptide Moiety Containing Modules (b) and (c)

The PEG containing [module (b)+module (c)+linker] peptide construct 12-(aminooxy)dodecanoyl-(dPEG12)-bLys-(dPEG12)NASSSRSGLDDINPTVLLKAKDEL-OH [the peptide comprising “module (b)+module (c)” comprises an amino acid sequence comprising SEQ ID NO: 244], whereby the side chain amine of the branching lysine (bLys) residue in addition carries the sequence (dPEG12)Cys(NPys), is synthesized commercially by standard solid-phase Fmoc peptide chemistry, deprotected in the standard fashion and purified by reversed phase HPLC to a purity of >95%. The N-terminal 12-(aminooxy)dodecanoyl moiety is introduced using 12-(Boc-aminooxy)-dodecanoic acid (Bachem, catalog no. A-4720). dPEG12 is introduced using Fmoc-dPEG₁₂-acid (Quanta BioDesign, product no. 10283). The branch point lysine residue is introduced using the Fmoc-Lys(ivDde)-OH (Merck Novabiochem, product no. 04-121193) building block. QC of the purified peptide is done by amino acid analysis, ESMS and analytical reversed phase HPLC.

(iii) Production of a Genetically Engineered DRBD Carrying an N-Terminal Cysteine:

A double stranded RNA binding domain (DRBD): FFMEELNTYRQKQGVVLKYQELPNSGPPHDRRFTFQVIIDGREFPEGEGRSKKEAKNAAAKLAVEILNKE (SEQ ID NO: 8) is produced genetically by recombinant engineering with an N-terminal Cys residue CFFMEELNTYRQKQGVVLKYQELPNSGPPHDRRFTFQVIIDGREFPEGEGRSKKEAKNAAAKLAVEILNKE (SEQ ID NO: 253), or alternatively, synthesized with an additional cysteine at the C-terminus FFMEELNTYRQKQGVVLKYQELPNSGPPHDRRFTFQVIIDGREFPEGEGRSKKEAKNAAAKLAVEILNKEC (SEQ ID NO: 254) using solid-phase Fmoc chemistry, deprotected in the standard fashion and purified by reversed phase HPLC to a purity of >95%. QC of the purified peptide is done by amino acid analysis, ESMS and analytical reversed phase HPLC.

(iv) Preparation of the siRNA Cargo Binding Construct Comprising the Targeting Module (a) Linked to the Sorting Modules [(b) and (c)] and the DRBD Adaptor:

The aldehyde modified transferrin from Example 5(i) above is first reacted with 2 mole equivalents of the aminoxy bearing linkage molecule containing modules (b) and (c) from Example 5(ii) above in degassed 100 mM citrate buffer at pH 6 and kept overnight at 4° C. The desired intermediate is purified by preparative SEC on a HiLoad 16/60 Superdex 75 prep grade column (GE Healthcare, part no. 17-1068-01) eluted at 1 mL/min with sterile PBS, pH 7.4. This intermediate is then conjugated to the N-terminal cysteine containing DRBD from Example 5(iii) above via disulfide exchange with the Cys(NPys) residue in an overnight reaction in PBS at 4° C. The desired cargo binding modality is purified by preparative SEC on a HiLoad 16/60 Superdex 200 prep grade column (GE Healthcare, part no. 17-1069-01) eluted at 1 mL/min with sterile PBS, pH 7.4. Final QC analysis is performed by gel electrophoresis and ESMS, plus cleavage of the construct by DTT and analysis of the two components.

### Example (6)

### Synthesis of a DARE™ Delivery Vehicle Design 3.3b to Deliver a Non-Covalently Linked dsDNA Cargo

(i) Construction of an Aldehyde Modified Transferrin as Module (a)

Human serum transferrin (SEQ ID NO: 252; Sigma, Invitrogen) is reacted under mild conditions with sodium periodate to generate reactive aldehyde functionalities on the carbohydrate moieties using the published protocol of d'Alessandro et al. [67]. It has previously been shown that conjugation of peroxidase hydrazide to an aldehyde modified transferrin yields a bioconjugate that is fully recognizable by both anti-transferrin and anti-peroxidase antibodies [67].

(ii) Synthesis of a Linkage Molecule Comprising a Branched Peptide Moiety Containing Modules (b) and (c)

The PEG containing [module (b)+module (c)+linker] peptide construct 12-(aminooxy)dodecanoyl-(dPEG12)-bLys-(dPEG12)NASSSRSGLDDINPTVLLKAKDEL-OH [the peptide comprising “module (b)+module (c)” comprises an amino acid sequence comprising SEQ ID NO: 244], whereby the side chain amine of the branching lysine (bLys) residue in addition carries the sequence (dPEG12), is synthesized by standard solid-phase Fmoc peptide chemistry, deprotected in the standard fashion and purified by reversed phase HPLC to a purity of >95%. The N-terminal 12-(aminooxy)dodecanoyl moiety is introduced using 12-(Boc-aminooxy)-dodecanoic acid (Bachem, catalog no. A-4720). dPEG12 is introduced using Fmoc-dPEG₁₂-acid (Quanta BioDesign, product no. 10283). The branch point lysine residue is introduced using the Fmoc-Lys(ivDde)-OH (Merck Novabiochem, product no. 04-121193) building block. QC of the purified peptide is done by amino acid analysis, ESMS and analytical reversed phase HPLC.

(iii) Preparation of the Arylhydrazine Containing Construct Comprising the Targeting Module (a) Linked to the Sorting Modules [(b) and (c)] and Linker:

The aldehyde modified transferrin from Example 6(i) above is first reacted with 2 mole equivalents of the aminoxy bearing linkage molecule containing modules (b) and (c) from Example 6(ii) above in degassed 100 mM citrate buffer at pH 6 and kept overnight at 4° C. The desired intermediate is purified by preparative SEC on a HiLoad 16/60 Superdex 75 prep grade column (GE Healthcare, part no. 17-1068-01) eluted at 1 mL/min with sterile PBS, pH 7.4. The primary amino group on the dPEG12 of this intermediate is then reacted with 4 mole equivalents of sulfosuccinimidyl 6-hydrazinonicotinate acetone hydrazone (sulfo-S-HyNic, sulfo-SANH, SoluLink product no. S-1011-010) in 100 mM HEPES, 150 mM NaCl pH 8.0 for 2 h at RT to introduce an arylhydrazine functionality protected as the acetone hydrazone. The activated construct is then desalted using a Vivaspin 2 polyethersulfone (PES) ultrafiltration spin column (molecular weight cut-off 5 kDa, Sartorius Stedim Biotech, part no. VS0211) and buffer exchanged into 100 mM citrate buffer pH 6.0.

(iv) Synthesis of an Aromatic Aldehyde Modified Adapter Molecule Derived from Human High-Mobility Group Protein HMGB2 (a DDBP) Carrying the SV40 NLS at its N-Terminus.

SV40_{NLS}-HMGB2₁₈₆ is expressed in Escherichia coli using the published protocol of Sloots et al. [68], which is incorporated herein in its entirety by reference. The purified protein is reacted with 2 mole equivalents of MTFB (SoluLink product no. S-1035) in 100 mM citrate buffer pH 6.0 for 2 h at RT, which functionalizes a cysteine thiol with a 4-formylbenzamide moiety via a (PEG)₃ spacer. The desired activated construct is then desalted using a Vivaspin 2 polyethersulfone (PES) ultrafiltration spin column (molecular weight cut-off 5 kDa, Sartorius Stedim Biotech, part no. VS0211), using 100 mM citrate buffer pH 6.0 for washing.

### (v) Synthesis of the dsDNA Cargo Binding Delivery Construct Comprising Module (a) Linked to the Sorting Modules (b) and (c) and the NLS Tagged DDBP Adapter

The arylhydrazine modified targeting and sorting construct from Example 6(iii) above is mixed with an equimolar amount of the aldehyde modified adapter construct from Example 6(iv) above in 100 mM citrate buffer pH 6.0 and incubated overnight at RT to connect the two components via a stable bis-arylhydrazone bond. The desired dsDNA cargo binding delivery construct is purified by preparative SEC on a HiLoad 16/60 Superdex 200 prep grade column (GE Healthcare, part no. 17-1069-01) eluted at 1 mL/min with sterile PBS, pH 7.4. Final QC analysis is performed by gel electrophoresis.

(vi) Loading with dsDNA Cargo Binding Delivery Construct with a dsDNA Cargo.

The dsDNA cargo binding delivery construct from Example 6(v) above is mixed with a dsDNA (for instance a transcription factor decoy) in PBS pH 7.4 and incubated at RT for 30 min. The amount of dsDNA that can be bound will depend on the sequence length and is able to be determined by titration experiments and monitoring of the reaction by PAGE. The final DARE™ construct is purified on a preparative gel or by ion-exchange HPLC.

An optional biodegradable disulfide bond may also be included in the hydrazone linker fragment that covalently connects the targeting or sorting component to the DDBP adapter by using for example, S-SS-4FB (SoluLink product no. S-1037-010) as an aromatic aldehyde containing entity for modifying a primary amine.

### Example (7)

### Use of a Targeted Delivery Carrier-Cargo Conjugate of the Invention to Elicit siRNA-Induced Silencing in Cultured Mammalian Cells

(i) Fluorescent Labeling of Protein Modules

In order to monitor the intracellular trafficking of module (a) alone, and module (a) with modules (b) and (c) by microscopy, the peptide or protein modules (a) can be labeled with a fluorescent dye. By way of example, ricin B is labeled with Cy3 Maleimide Monoreactive dye (GE Healthcare, PA23031) according to the manufacturer's protocol. Briefly, 1 mg/mL of full length ricin B-subunit (Vector Laboratories) is dialyzed against PBS supplemented with 1 mM EDTA. The terminal sulfhydryl group on the ricin B is made available by reduction with 100× molar excess of TCEP. The vial is flushed with nitrogen gas and closed. Sample is mixed thoroughly and incubated for 10 min at RT. An aliquot of Cy3 maleimide monofunctional dye, sufficient for the labeling of 1 mg of protein is dissolved in anhydrous dimethylformamide. The vial is flushed with nitrogen gas and closed. The sample is mixed thoroughly, incubated for 2 h at RT and mixed every 30 min. The reaction is left at 4° C. overnight. Separation of ricin B from the free dye is done by multistep dialysis against PBS. Absorbance of the sample at 552 nm and 280 nm is read in a spectrophotometer and the final dye/protein or dye/peptide ratio is calculated.

(ii) Preparation of a Dye Labeled-Module (a)+Module (b) Construct

Ricin B subunit [SEQ ID NO: 124; module (a)] is labeled with Cy3 NHS ester and then linked through a disulfide bond to a module (b) comprising a KDEL peptide (SEQ ID NO: 160) with a free C-terminus. Briefly, 0.5 mL of 1 mg/mL full length ricin B subunit (Vector Laboratories) in PBS containing 50 mM 2-mercaptoethanol (2-ME) is desalted and then buffer exchanged against sterile 100 mM sodium tetraborate buffer, pH 8.5 containing 5 mM lactose using a Vivaspin 2 polyethersulfone (PES) ultrafiltration spin column (molecular weight cut-off of 5 kDa, Sartorius Stedim Biotech, part no. VS0211) and then stirred in air to dimerize it, to prevent the thiol from potentially reacting with the Cy3 NHS ester in the subsequent reaction. The ricin B dimer is then fluorescently labeled by reaction with 4 molar equivalents (relative to ricin B monomer) of Cy3 NHS ester (GE Healthcare, catalog no. PA13101) dissolved in 25 μL of pure DMSO for 3 h at 10° C. The solution is then desalted on a Vivaspin 2 PES 5 kDa molecular weight cut-off spin column and transferred into PBS containing 5 mM lactose and 1 mM EDTA at pH 7. The Cy3-labeled ricin B dimer is reduced with fresh 50 mM 2-ME and incubated for 1 h at RT. The Cy3-labeled ricin B is recovered using a Vivaspin 2 PES, 5 kDa molecular weight cut-off spin column and buffer exchanged into degassed PBS containing 5 mM lactose and 1 mM EDTA, pH 7 and then reacted overnight at 10° C. under an argon atmosphere with 1.1 mole equivalents of the module (b) peptide, H₂N-Cys(NPys)-(SG)-3-KDEL-OH, prepared by standard solid-phase Fmoc peptide chemistry. The dye-labeled module (a)+module (b) construct is purified by gel electrophoresis.

(iii.) Monitoring Intracellular Sorting of DARE™ Modules in Cultured Cells

The following modules and conjugates are monitored:
Ricin B [module (a)], fluorescently labeled with Cy3 as described under Example 7(i) above
Ricin B [module (a)], including a C-terminally attached KDEL sequence [SEQ ID NO: 160; module (b)], prepared and fluorescently labeled with Cy3 as described under Example 7(ii) above
Ricin B [module (a)], including modules (b) and (c) as described in Example 1(i) and (ii), fluorescently labeled with Cy3 as described under Example 7(ii) above
Ricin B [module (a)], including modules (b) and (c), conjugated to an siRNA molecule as described in Example 1, wherein the siRNA is
   Specific and targeting GAPDH, or
   Non-specific, comprising a firefly luciferase fLuc:
      sense: 5′-CUUACgCUGAGuACUUCGAuu-3′ (SEQ ID NO: 255), and antisense: 5′-UCGAAGUACUCAgCGUAAgdTdG-3′ (SEQ ID NO: 256), wherein the lowercase u or g represents a 2′-O-Me-modified nucleotide, and wherein the antisense strand has a 5′-phosphate and two deoxynucleotides at its 3′ end (dTdT).

HeLa (human), U2-OS (human) and NIH-3T3 (murine) cells are each grown on collagen coated 384-well plates suitable for microscopy (Aurora Biotechnologies) using Dulbecco's Modified Eagle Media (DMEM) supplemented with 4 mM glutamine (Invitrogen) and 10% fetal bovine serum (Invitrogen) under standard conditions. In order to monitor internalization and intracellular transport of DARE™ modules and conjugates, cells are treated with a range of 1-100 μg/mL of fluorescently labeled module/conjugate for 30 min on ice, followed by 2-3 washing steps with cold medium, before warming up to 37° C. for different time periods ranging from 30 min to several hours (e.g. 0.5, 1, 2, 4, 6, 8, 16) and up to several days (e.g., 1, 2, 3, 4, 5, 6, 7). Alternatively, cells are incubated with the same amount of module/conjugate at 37° C. for the indicated time periods without a preceding binding and washing step on ice. At the indicated time points, cells are washed five (5) times with PBS, and fixed with 4% paraformaldehyde for 45 min. The cell membranes are permeabilized by incubation with 0.1-0.2% Triton X-100, and 0.01 to 0.02% Saponin in PBS for up to 30 min at RT. Non-specific binding sites are blocked by incubation with 10% fetal calf serum (Invitrogen) in PBS for 30 min. This step can optionally be combined with the permeabilization. The permeabilized cells are incubated with primary antibodies as listed below. Antibody incubations are performed in blocking buffer at 4° C. for up to 16 h. The cells are then washed with PBS and incubated with the appropriate fluorescently labeled (preferably with FITC or Alexa 488) standard secondary antibodies directed to the primary antibody at RT for 2 h, and then washed with PBS. Intracellular sorting of the modules/conjugates is determined by co-staining of the cells for intracellular compartments:

Endosomes:

Early and recycling endosomal compartments are identified through co-internalization of fluorescently labeled transferrin (Invitrogen, Alexa-633 conjugate, Catalog No. T-23362) at 10-100 μg/mL using the same experimental conditions as described for the modules and conjugates.

Late endosomal compartments are identified through co-internalization of fluorescently labeled LDL particles (LDL-DiI, bti inc. Stoughton Mass., USA) at 5-20 μg/mL, using the same experimental conditions as described for the modules and conjugates.

Lysosomes:

Lysosomes are identified by antibody staining using a rat monoclonal antibody (1D4B; ABCAM, Cambridge UK) to murine LAMP 1 (lysosomal-associated membrane protein 1) at 0.1-0.5 μg/mL. Human LAMP1 can be detected by staining using a rabbit polyclonal antibody at 1:500 (Abeam, ab24170).

Trans-Golgi-Network:

The trans-Golgi-network (TGN) is identified by antibody staining using a mouse monoclonal antibody (2F7.1; ABCAM, Cambridge UK) to TGN46 (trans golgi network protein of 46 kDa) at a dilution of 1:100 to 1:500.

Golgi Apparatus:

The Golgi Apparatus are identified by antibody staining using an antibody to mannosidase II (ab12277; ABCAM, Cambridge UK) at a dilution of 1:100 to 1:1000 in mouse cells. In human cells, the Golgi Apparatus can be detected by staining using a mouse monoclonal antibody against Golgin-97 (Invitrogen A-21270) at approximately 1 μg/mL.

Endoplasmic Reticulum (ER):

The ER is identified by antibody staining using a chicken polyclonal antibody to Calreticulin (ABCAM, Cambridge UK, ab14234) at a dilution of 1:500. Alternatively, ER exit sites can be stained by using a rabbit polyclonal antibody against Derlin-1 (Sigma, D4443) at a dilution of 1:200.

Caveolae:

Caveolae are identified by antibody staining using a rabbit monoclonal antibody to Caveolin-1 (New England Biolabs, D46G3) at a dilution of 1:500. Alternatively, caveolar internalization can be visualized by co-internalization with fluorescently labeled AMF (alias GPI, GeneID: 100008744). AMF labelling is done with a Fluorescein-EX labelling kit (Invitrogen). Cells are incubated with labelled AMF at 50 μg/mL [69, 70].

Cytoplasm

Delivery of the siRNA [compound (d)] to the cytoplasm is followed by microscopy via the fluorescent dye attached to the 5′-end of the sense strand of the siRNA. Preferably the fluorescent dye is Cy3 or Cy5.

Images are acquired using an automated microscope (ImageExpress, Molecular Devices) or an LSM510 confocal microscope (Zeiss), and co-localization between the modules/conjugates and different cellular organelles/compartments is determined by automated image analysis (Cellenger, Definiens).

Alternatively, a multiparametric approach is used to detect colocalization of the conjugate and/or the modules and/or compound (d) of the invention and involves three different analysis techniques. In addition to the basic qualitative approach to identifying colocalization, two statistical methods are employed to quantitate colocalization using a Definiens Enterprise image analysis software.

For qualitative analysis of colocalization, captured channels are pseudo-colored using an appropriate color look-up table provided with the image analysis software, to convert greyscale into color, where x shade of grey equals y color. The Definiens system, for example, can convert a greyscale image into red, green, blue, yellow, violet or turquoise. Thus, if the pixels are co-stained with red and green, then yellow colored pixels indicate colocalization.

Quantitative statistical analyses using intensity correlation coefficient-based techniques are also performed, using two approaches, the Manders' coefficient, which is a modified version of the Pearson's coefficient, and Li's approach. Prior to calculation of coefficients, background is first excluded using a fluorescence intensity threshold, thereby identifying regions of interest. This background threshold is set manually for each assay. The Manders' coefficients, m₁ and m₂, are then calculated for all remaining pixels in each image:

m₁=Σᵢ^{S1u,coloc}/Σᵢ^{S1i}

m₂Σᵢ^{S2i,coloc}Σᵢ^{S2i}

Where, S1i,coloc is the sum of the intensities of channel 1 that colocalise with channel 2 and S1i is the sum of the intensities in channel 1. Similarly, S2i,coloc is the sum of the intensities of channel 2 that colocalise with channel 1 and S2i is the sum of the intensities in channel 2. When calculated, a Manders' coefficient of 1 indicates complete colocalisation and a coefficient of 0 indicates complete exclusion.

In contrast, Li's approach assumes that for two sets of random staining intensities of N number of pixels, the sum of the product of their differences will tend towards zero:

Σ_{N}(Aᵢ−a)(Bᵢ−b)˜0

Where a or b is the mean intensity of the distribution with N number of values of Aᵢ or Bᵢ, the intensity of each individual pixel. Intensity counts for each pixel in each image are therefore normalized to give a value between 0 and 1 and are plotted on a graph against the product of (Aᵢ−a)(Bᵢ−b) for each pixel, which varies between minus 1 and plus 1. In these graphs, pixels to the right of x=0 indicate colocalization, while pixels to the left of x=0 indicate complete exclusion.

A positive result using all three methods described above provides a very good assessment of colocalization [71-74].

(iv.) Testing for Degradation of the Delivery Carrier Modules

Cells are treated with a series of titrations of the modules/conjugates described in Example 7(iii) above, for different time periods ranging from 1 to 7 days. At the indicated times (1 h, 8 h, 1, 2, 3, 4, 5, 6, or 7 days), cells are lysed, and equal amounts of total protein are separated by SDS-PAGE. Degradation of the delivery carrier modules is monitored by western blotting and probing with an antibody directed against ricin B (obtained from ABCAM, Cambridge, UK, ab48415, used at a dilution of 1:100 to 1:1000).

(v.) Functional Testing of DARE™ Delivery

Cells are treated with a series of titrations of the modules/conjugates described in Example 7(iii) above, for different time periods ranging from 1 to 7 days. For comparison, cells are transfected with equimolar amounts of the targeting siRNA and the non-targeting control using commercially available transfection reagents, e.g. Dharmafect (ThermoFisher) or RNAiMax (Invitrogen). After the indicated time periods (1, 2, 3, 4, 5, 6, or 7 days), cells are lysed and tested for silencing of the target gene by quantitative RT-PCR (qRT-PCR), which is performed on a SDS7900 Thermocycler (Applied Biosystems) with gene specific validated TaqMan probes (Applied Biosystems), or gene specific primers and the SyBr-Green method, according to the manufacturers' recommendations. Gene expression is normalized to a housekeeping gene (e.g. 18S ribosomal RNA, RPL13A, or a specifically selected set of housekeeping genes if necessary [75]).

(vi.) Testing for Interferon Response Caused by DARE™ Delivery

Activation of the interferon pathway is monitored by determining expression levels of OAS1, OAS2, STAT1, IFNB1, and IFIT2 in treated cells compared to untreated cells by qRT-PCR as described above in Example 7(v.). Primer sequences of use to detect an interferon response by qRT-PCR of OAS1, OAS2, STAT1, IFN-beta and IFIT2 include commercially available Human TaqMan probes: OAS1 (Hs00973637_m1), OAS2 (Hs00942643_m1), STAT1 (Hs01014002_m1), IFN-beta (Hs00277188_s1), IFIT1 (Hs01911452_s1), and IFIT2 (Hs00533665_m1), and Mouse TaqMan probes: OAS1 (Mm00449297_m1), OAS2 (Mm00460961_m1), STAT1 (Mm00439518_m1), IFN-beta (Mm00439552_s1), IFIT1 (Mm00515153_m1), and IFIT2 (Mm00492606_m1) (Applied Biosystems/LifeTechnologies, Inc.).

While this Example illustrates the preparation, use and characterization of a ricin B [module (a)] targeted conjugate of the invention, the teachings of this Example are applicable to any conjugate of the invention. One of ordinary skill in the art will know how to modify the teachings of the Example accordingly and without undue experimentation.

### Example (8)

### Synthesis of a DARE™ Delivery Construct with Target siRNA as Compound (d) but without the Cell Targeting/Uptake Module (A)

(i) Synthesis of the Linkage Molecule Comprising Modules (b) and (c)

The [module (b)+module (c)+linker] peptide H₂N-(SG)₃-C-(SG)₃-NASSSRSGLDDINPTV LLKAKDEL-OH [“module (b)+module (c)” comprise SEQ ID NO: 244] is synthesized by standard solid-phase Fmoc peptide chemistry, deprotected in the standard fashion and purified by reversed phase HPLC to a purity of >95%. QC of the peptide is done by amino acid analysis, mass spectroscopy and analytical reversed phase HPLC. Activation of the free thiol of the purified peptide is done by reaction in pyridine with 1.5 mole equivalents of 2,2′-dithiobis(5-nitropyridine) (DTNP, Sigma-Aldrich product no. 158194) to give H₂N-(SG)₃-C(pNpys)-(SG)₃-NASSSRSGLDDINPTVLLKAKDEL-OH, which is purified by preparative reversed phase HPLC to >95% purity and analyzed as noted above.

(ii) Preparation of the Cargo siRNA [Compound (d)]

A Tuschl-style siRNA targeting GAPDH is synthesized, purified and analyzed as described in Example 1(iii) except that the 5′-terminus of the sense strand is modified with a 5′-(C₆-SS-C₆)-phosphate-Cy3 entity.

(iii) Preparation of the [Module (b)+Module (c)+Module (d)] Construct in which (d) is siRNA

The cargo siRNA from Example 8(ii) above is treated with 100 mM DTT in PBS containing 1 mM EDTA for 1 h at 37° C. to cleave the disulfide bond. The free thiol containing siRNA is then desalted on a Vivaspin 2 polyethersulfone 3 kDa molecular weight cut-off ultrafiltration spin column (Sartorius Stedim Biotech, part no. VS0292) using degassed PBS containing 1 mM EDTA pH 7 as eluent. The thiol-siRNA is subsequently reacted overnight under argon with 1.1 mole equivalents of the linkage molecule containing modules (b) and (c) from Example 8(i) above in PBS containing 1 mM EDTA pH 7. The desired module (b)+module (c)+module (d) construct is purified by reversed phase HPLC. The product is analyzed by ESMS, native gel electrophoresis and analytical HPLC. Further analysis is done using DTT cleavage to obtain two fragments, the molecule comprising modules (b) and (c), and the HS—(CH₂)₆—OP(O₂)—O-Cy3-siRNA, that can each be separately identified by MS.

Although this specific example describes the use of a COX2 peptide as the ERAD targeting module (c), an AKDEL peptide (SEQ ID NO: 161) as the ER translocation module (b), and the attachment of the siRNA through a disulfide bond to a cysteine residue, one of skill in the art is able to envision and make conjugates comprising other peptide(s) and using a different attachment and/or a different configuration without undue experimentation that are also embodiments of the present invention. For example, (SG)₃ (SEQ ID NO: 7) can be replaced by dPEG12 and the siRNA may be attached via an oxime bond using the aminooxy group on a DprAoa residue (i.e., instead of the cysteine in this Example).

### Example (9)

### Pharmacodynamics of a DARE™ Delivery Conjugate

To evaluate the in vivo activity of a DARE™ delivery conjugate of the present invention, the pharmacodynamics are tested after systemic application. A DARE™ delivery construct of the present invention is administered intravenously in mice via the tail vein (or alternatively intraperitoneally). Bio-distribution is determined in two different mouse models. In one model, an endogenously expressed gene (GAPDH) is targeted; in the second model, an exogenously introduced reporter transgene (firefly luciferase, fLuc) is targeted. A non-silencing siRNA conjugate and a non-targeting [i.e., lacking module (a)] conjugate are also prepared as controls.

(i). Synthesis of the Conjugates

All conjugates are prepared as described in Examples 1 and 6, and the following siRNA sequences are preferably used:

### GAPDH:

sense: SEQ ID NO: 248 and
antisense: SEQ ID NO: 249
fLuc:
sense: SEQ ID NO: 255, and
antisense: SEQ ID NO: 256,
Non-silencing control (targeting NP number 2, a nucleoprotein of influenza virus):
sense: 5′-GGAuCUUAUUUCUuCGGAGuu-3′ (SEQ ID NO: 257), and
antisense: 5′-CUCCGAAGAAAuAAGAuCCdTdT-3′ (SEQ ID NO: 258), wherein “u” and “g” represents 2′-O-Me-modified nucleotides and all antisense strands have a 5″-phosphate.

(ii) In Vivo Testing

The GAPDH targeting conjugate is tested for GAPDH specific knockdown in Balb/c mice [available from Jackson Laboratories (www.jax.org), Charles River (www.criver.com), Taconic (www.taconic.com), or Harlan (www.harlan.com)], while luciferase knockdown is evaluated in a mouse strain that is transgenic for firefly luciferase (Promega pGL3) and expresses high levels of the enzyme in virtually all tissues [76]. Gender matched mice that are 6-10 weeks of age are used.

A dose escalation of the DARE™ delivery construct is performed, for example using a range of 100 to 2000 nmol/kg. The DARE™ delivery construct dose is then injected in a volume of 100-300 μL PBS (or other physiological buffer). As described within this Example, the dose at which the highest knock down of fLuc is achieved, while avoiding lethality, is determined. This dose is preferably used subsequently for all other systemic applications.

Each experiment consists of the following groups with n=10 mice/group.
1. DARE™ delivery construct with target siRNA (directed to either GAPDH or luciferase and corresponding to the in vivo model used) as compound (d), prepared as described in Example 1
2. DARE™ delivery construct with non-target siRNA as compound (d), prepared as described in Example 1
3. DARE™ delivery construct with target siRNA as compound (d) but without a cell targeting/uptake module (a), prepared as described in Example 8 above.

Mice are euthanized at 24-72 h post DARE™ delivery construct dose injection and tissues of interest (e.g. brain, lung, heart, liver, kidney, spleen, muscle, ovaries, uterus, mammary glands, pancreas, lymph nodes, bone, and any other tissue of interest) are sampled and analyzed as described below.

### Luciferase Measurements:

For luciferase protein measurement, tissues are homogenized using a tissue lyser/mixer mill (Qiagen), metal beads and luciferase cell culture lysis reagent (e.g. Promega PR-E1531), and then centrifuged for 5 min at maximum speed (13,000 g) in a table top centrifuge before the supernatant is transferred to a new reaction tube. The supernatant is either stored at −80° C. or used immediately to measure luciferase protein levels in a luminometer, using a luciferase assay system (e.g. Promega) according to the manufacturer's instructions.

### RNA Isolation:

Tissue samples are stored in RNAlater (Qiagen) for subsequent qRT-PCR and 5′-RACE analysis or frozen in liquid nitrogen for subsequent luciferase and tissue protein (to normalize for luciferase activity per mg protein) quantification. After euthanasia, the tissues/organs of interest are removed and immediately frozen in liquid nitrogen. RNA is isolated from the tissue samples with the RNeasy kit (Qiagen) according to the manufacturer's instructions and RNA quality is determined with an Agilent 2100 Bioanalyzer using the RNA 6000 Nano kit (Agilent) according to the manufacturers' instructions.

### 5′ RACE-PCR:

5′ RACE is performed to detect RNAi specific RNA degradation products. The detection is performed by a modified GeneRacer PCR (Invitrogen, Calsbad, Calif.) as described before [77-79]. Briefly, a 44mer RNA-oligo, which is a pre-designed kit component (GeneRacer™ RNA Oligo) is ligated to 5′-uncapped, degraded RNA before reverse transcription. Following this, a PCR is performed with a primer set consisting of a gene-specific primer 3′ of the siRNA recognition site and a complementary primer binding to the 44mer RNA-Oligo sequence:

For GAPDH (human and mouse) the sequences are as follows:

GAPDH siRNA target sequence:
(SEQ ID NO: 259)
5′-GGTCATCCATGACAACTTT-3′;
GeneRacer 5′ Primer:
(SEQ ID NO: 260)
5′-CGACTGGAGCACGAGGACACTGA-3′;
GAPDH 3′ Primer:
(SEQ ID NO: 261)
5′- ACGCCTGCTTCACCACCTTCTTGATGTC-3′;
GeneRacer 5′ Nested Primer:
(SEQ ID NO: 262)
5′- GGACACTGACATGGACTGAAGGAGTA-3′;
and
GAPDH 3′ Nested Primer:
(SEQ ID NO: 263)
5′- AGGCCATGCCAGTGAGCTTCCCGTTCAG-3′.

Agarose gel analysis and sequencing of the amplified DNA is then used to identify the resulting DNA fragment as an RNAi specific degradation product of the gene of interest. In case of low abundant degradation products, a nested PCR is carried out after the primary PCR.

### RT-qPCR:

RT-qPCR is performed on a SDS7900 Thermocycler (Applied Biosystems) with gene specific validated TaqMan probes (Applied Biosystems) according to the manufacturer's recommendations. Gene expression is normalized to a pool of housekeeping genes (e.g. 18S rRNA, RPLPO, Hmbs, Ppib, and/or Pgk1) selected for gene expression analysis in mouse tissue to normalize for natural expression variation in vivo [75].

### GAPDH ELISA and Western Blots:

GAPDH protein expression is determined with a standard GAPDH specific ELISA assay (e.g. from BIOO Scientific). Tissue is lysed by the addition of RIPA (Radio-immunoprecipitation assay; Sigma Aldrich) buffer and total protein concentration is measured by BCA assay (Bicinchoninic acid; Perbio) prior to analysis by ELISA according to the manufacturer's instructions or by western blot analysis according to standard procedures.

### RNA In Situ Hybridization:

In situ RNA detection is performed according to established procedures including Proteinase K digestion and acetic anhydride pre-treatment. The tissue sample is fixed in 4% PFA for 24-30 h after extraction before soaking in 30% sucrose for 24-30 h. It is then cooled to −70° C. in isopentane and 5 μm thick sections are cut in a cryostat-microtome. A GAPDH-specific digoxygenin labeled probe is prepared from a GAPDH cDNA containing plasmid with and SP6 or T7 RNA polymerase with the DIG RNA labeling Kit (Roche Applied Science) according to the manufacturer's recommendations and as described earlier [80]. The probe is incubated on the tissue sections in a humidified chamber at 65° C. overnight. The DIG labeled probe is detected with a sheep anti-DIG antibody conjugated to alkaline phosphatase (AP; Roche). The sections are then developed by the addition of BM purple (Roche) or another AP substrate.

### Immunohistochemistry and Histology:

For distribution analysis of the fluorescently (e.g.Cy3) labeled DARE™ delivery construct and analysis of target protein expression by immunohistochemistry, tissues are fixed in 4% paraformaldehyde, 0.05% glutaraldehyde in PBS for 24 h and then soaked in 30% sucrose for 36 h. The tissues are then frozen at −80° C. for storage, and 7 μm sections are cut at −20° C. and placed on slides. Microscopy analysis is performed as described above in Example 7.

For antibody staining and histology, tissue is fixed overnight in 10% buffered formalin before paraffin embedding and sectioning on a microtome. GAPDH protein expression is detected using a GAPDH specific antibody (rabbit mAB 14C10, Cell Signaling, or similar). Antigen detection is performed according to the manufacturer's recommendations following microwave assisted antigen retrieval using citrate buffer. Detection of primary antibody is done with an anti-rabbit HRP or fluorophore labeled secondary antibody (Abeam) before microscopy analysis using standard protocols or, in the case of a fluorophore labeled secondary antibody, as described above in Example 7.

### Example (10)

### Pharmacokinetics of a DARE™ Delivery Construct

To determine the knock down effect over time, a blood clotting factor, Factor VII (FVII) is targeted in the liver using a DARE™ delivery construct according to the present invention. Published siRNA sequences against FVII [81] or previously in vitro optimized siRNAs against FVII are used as compound (d) in a DARE™ delivery construct and made as described in the Examples above. The optimal knock down dose of the resulting DARE™-FVII conjugate is determined in liver in experiments as described in Example 9. The DARE™-FVII conjugate is then tested in vivo at this optimal knock down dose.

All procedures are done in normal C57BL/6 or Balb/c mice (gender and age matched, 6-10 weeks of age, obtained from Charles River). The optimal knock-down dose of DARE™-FVII is administered intravenously to mice via tail vein injection. Control mice are injected via the tail vein with the same DARE™ delivery construct as DARE™-FVII except that the control DARE™ construct comprises a non-targeting control siRNA as compound (d) instead of the siRNA against FVII. Blood samples are taken retro-orbitally from the DARE™-FVII treated and control treated mice repeatedly, on a twice weekly basis, until 40 days post injection and serum levels of FVII protein are measured using an activity-based chromogenic assay (Biophen FVII; Aniara, Mason, Ohio) [81] to determine the length of time that FVII protein levels remain knocked-down below that of the control mice. Based upon the length of time it takes for the circulating FVII protein levels of the DARE™-FVII treated mice to reach the circulating FVII protein levels of the control treated mice (i.e., baseline FVII levels), repeated administration times can be calculated. For example, if the circulating FVII protein levels of the DARE™-FVII treated mice reach the baseline FVII levels at 30 days post injection, repeated injections of the DARE™-FVII dose will be made every 30 days and retro-orbital blood samples will obtained and analyzed twice weekly. If the circulating FVII levels decrease and increase in similar fashion after a second and third injection of DARE™-FVII, then this indicates that there is no strong immune response against DARE™-FVII.

### Example (11)

### Testing for Immunostimulatory Effects of a DARE™ Delivery Conjugate

SiRNA molecules have been shown to stimulate the immune system via interaction with the toll-like receptors TLR3, TLR7 and/or TLR8 [82]. The immune responses to TLR7/8 can be overcome or at least minimized by chemically modifying the siRNAs. Immunological responses resulting from such interactions can be examined in human PBMCs (peripheral blood monocytes) as described [83, 84]. Briefly, buffy coats are obtained from the blood of human donors. PBMCs are purified from the buffy coats by Ficoll density centrifugation. The purified PBMCs are then seeded in 96 well plates at 2×10⁵ cells/well or a different previously optimized density. The cells are then incubated at 37° C. with the siRNA, which is complexed with a transfection reagent or coupled to other molecules enabling transfection, i.e. a DARE™ delivery conjugate (final concentration: up to 1 μM). At different time points (e.g. 4 h and 24 h post transfection), supernatant is removed and the TNFα and/or IFNα concentration is determined via ELISA and compared to untreated PBMCs. The ELISAs are performed using commercially available ELISA kits [TNFα Elisa Jumbo Kit, # IM 11121, Beckman Coulter; and Human IFNα ELISA (multi species), #3169016, Thermo Fisher Scientific].

TLR7 and TLR8 mediate an inflammatory response caused by activation of the innate immune response [82]. TLR8, which is an important mediator of nonspecific siRNA immune effects in human cells, is not fully functional in mice [83]. Consequently, effects related to TLR8 are not relevant to mouse studies. To evaluate possible TLR8 mediated effects, human PBMCs can be used as described above. These cells will produce TNFα, even if the oligonucleotide only stimulates TLR8 but not TLR7 [83]. Thus, incubating human PBMCs with the DARE™ construct (at up to 1 μM final concentration), followed by a TNFα ELISA will be sufficient to evaluate a TLR7 and a TLR8 mediated response.

In addition, immune responses could also result from the DARE™ module(s) that transports the siRNA. Regarding an immediate immune response, the same assays as described above for siRNA will be sufficient for their characterization. If a delayed immune response occurs, e.g. mediated by antibodies, it will be detected when the DARE™ conjugate is administered a second time after approximately 30 days in an animal experiment, and the knock down effect is significantly reduced (see Examples 9 and 10 re: in vivo knock down).

In addition to the above and to further ensure that the effects observed with a DARE™ delivery conjugate of the present invention are sequence specifically mediated by the DARE™-delivery conjugate siRNA [compound (d)] and not by target-unrelated reactions to the siRNA or the DARE™ modules or delivery conjugate, knockout (k.o.) mice of the relevant TLR3 and TLR7 receptors can be used (TLR3 k.o. mice: B6; 129S1-tlr3^{tm1Flv}/J, http://jaxmice.jax.org/strain/005217.html and TLR7 k.o. mice: B6.129S1-Tlr7^{tm1Flv}/J, http://jaxmice.jax.org/strain/008380.html, both from Jackson Laboratories). Specific effects via the DARE™ delivery conjugate siRNA will be the same in wt mice and in k.o. mice for the TLRs of the same strain (C57BL/6 is the wt strain corresponding with the above k.o. strains, available from Jackson Laboratories www.jax.org, Charles River www.criver.com, Taconic www.taconic.com, or Harlan www.harlan.com). For all experiments, gender and age matched mice (6-10 weeks of age) are used. These animal experiments are helpful to differentiate between the effects attributed to the siRNA [compound (d)] and the effects that may be produced by the immune system or an anti-angiogenic effect.

Specifically, GAPDH (or another endogenous gene) is targeted with an siRNA [compound (d)] of a DARE™ delivery construct according to the present invention. K.o. mice or cells as described above are used to evaluate the effects mediated by TLRs. The mice or cell experiments are analyzed as described in the Examples above by qRT-PCR, 5′RACE, Western blot and/or an enzymatic assay (e.g. KDalert™ GAPDH Assay Kit from Invitrogen/Life Technologies) for GAPDH expression.

Different versions of the modular DARE™ conjugate of the present invention are prepared according to Example 1 and delivered systemically via tail vein injection into mice. Each experimental group consists of 10 animals. Each experiment includes the following groups:
1. DARE™ delivery construct with a non-target siRNA as compound (d)
2. DARE™ delivery construct with a target siRNA as compound (d)
3. DARE™ delivery construct without an siRNA [i.e., lacking compound (d)]
4. Naked target siRNA (i.e., compound (d) only).

The optimal DARE™ dose as determined above in Example 9 is used here to determine whether any of the observed effects of the DARE™ constructs of the present invention are mediated by TLRs. The mice (or cells) are maintained for 2-60 days, depending on when the siRNA mediated effects are expected to occur. If GAPDH is used, the mice are analyzed after 48 h, at which time, the mice are euthanized and tissue samples are collected from the major organs (i.e., liver, spleen, kidney, brain, heart). When a tumor model is used, the mice are observed for up to 60 days. At each time point, animals are euthanized and tissues of interest as well as tumor samples are collected. The collected tissues and tumor samples are processed and analyzed for knock down expression of the targeted gene (i.e. GAPDH) by qRT-PCR, 5′RACE and western blot analysis as described above in Example 9.

### Example (12)

### Analysis of DARE™ Delivery Conjugate Toxicity

The potential toxicity of a DARE™ delivery conjugate of the present invention is assessed by measuring serum levels of liver enzymes and cytokines repeatedly up to 48 h post injection. A DARE™ construct with a non-targeting siRNA as compound (d) and a DARE™ construct without an siRNA [i.e., lacking a compound (d)] will be compared against PBS injection. The DARE™ delivery constructs are injected via tail vein injections as described in Example 9 above. Blood samples are collected retro-orbitally from the mice repeatedly up to 48 h post-injection and serum is obtained. Serum levels of the mouse cytokines TNF-alpha and IL-6 are measured by sandwich ELISA with reagents according to the manufacturer's instructions (R&D Systems, Minneapolis, Minn.). Serum levels of mouse IFN-alpha are measured by using a sandwich ELISA kit according to the manufacturer's instructions (PBL Biomedical, Piscataway, N.J.). Serum levels of alanine aminotransferase (ALT) and aspartate aminotransferase (AST) are measured by using automated systems at a veterinary diagnostic laboratory. If any statistically significant increases in liver enzymes and/or cytokines are detected, then further investigations should be conducted to determine the full toxicological impact of the conjugate.

### Example (13)

### Preparation and Administration of a DARE™ Delivery Conjugate Having a VEGF-Specific siRNA as Compound (d) In Vivo: Xenograft Model for Oncology

To demonstrate efficacy of a DARE™ delivery construct of the present invention in a tumor model, a well-established xenograft tumor model is used to study the knockdown of tumor relevant targets.

In this Example, the expression of VEGF (Vascular endothelial growth factor) is knocked down and the effect of this knockdown on tumor vascularization and growth is evaluated [85-92]. The experiments are carried out in two independent tumor models in gender and age matched (6-10 weeks) immunoincompetent mice (preferably athymic nude mice, Harlan-Winkelmann). PC-3 prostate adenocarcinoma cells (ATCC CRL 1435) are injected subcutaneously at 3×10⁶ in 0.1 mL of serum-free F-12K medium (Invitrogen) into the dorsal flank region of the mouse. After the tumors are clearly established and reach a volume of 50-100 mm³, the control siRNA and DARE™ delivery conjugate formulations are delivered systemically by tail vein injections or intratumorally in independent experiments.

The following constructs and conjugates are prepared following the teachings of Examples 1 and 5. Each experiment consists of 5 groups, with n=14 mice/group:
1. DARE™ delivery construct without siRNA [i.e., lacking compound (d)]
2. Naked VEGF Target siRNA Sequence comprising a sense strand comprising 5′-GGAGUACCCUGAUGAGAUCdTdT-3′ (SEQ ID NO: 264), and an antisense strand comprising 5′-GAUCUCAUCAGGGUACUCCdTdT-3′ (SEQ ID NO: 265).
3. Naked non-target (Luciferase) siRNA comprising a sense strand comprising SEQ ID NO: 255, and an antisense strand comprising SEQ ID NO: 256.
4. DARE™ delivery construct with a compound (d) comprising a VEGF siRNA comprising a sense strand comprising SEQ ID NO: 264, and an antisense strand comprising SEQ ID NO: 265.
5. DARE™ delivery construct with a compound (d) comprising a non-target siRNA comprising a sense strand comprising SEQ ID NO: 257, and an antisense strand comprising SEQ ID NO: 258.
   siRNA sequences targeting VEGF are selected based on published sequences [92, 93]. Doses range from 100 to 2000 nmol/kg in 100 μL for systemic delivery and 0.05 to 5 nmol in 25 μL for local intratumoral delivery.

To minimize an immunogenic effect on vascularization as previously reported [94], chemically modified siRNA sequences including selective introduction of 2′-O-Me nucleosides into the antisense strand are used [95, 96]. Non-targeting siRNA controls are optimized for this system to match the immunostimulatory effect of the VEGF targeted siRNA [97]. To assess immunostimulatory capacity of the siRNAs, a panel of cytokines and cytokine triggered mRNA is measured from mouse serum and target tissue, respectively. The immuno markers include, but are not limited to, interferon-α (IFNα), IL-6, IFNγ, tumor necrosis factor-α (TNFα), IL-12 and interferon induced tetratricopeptide repeat protein 1 (IFIT-1 or p56) mRNA [98, 99]. Mouse serum is analyzed for cytokines using commercially available ELISA assays, following standard procedures at 1-48 h after siRNA injections. IFIT mRNA levels are assessed at 1-48 h after siRNA injections by RT-qPCR with commercially available TaqMan probes as described in Example 9.

In the first part of this study, 6 animals are used per group for molecular analyses. Animals are euthanized 2 days post treatment. In the second part of this study, 8 animals are used per group to analyze tumor growth/remission and vascularization. Animals are observed for up to 3 months or until moribund. Molecular analyses are carried out as follows or as described in

### Example 9

### RNA Isolation

After euthanasia, tumors are removed and immediately frozen in liquid nitrogen. RNA is isolated from tumor tissue with the RNeasy kit (Qiagen) according to the manufacturer's manual and RNA quality is determined with an Agilent 2100 Bioanalyzer using the RNA 6000 Nano kit (Agilent) according to the manufacturer's instructions.

### 5′ RACE-PCR:

5′ RACE-PCR is performed on individual tumor samples as described above in Example (9) using VEGF specific 5′ and 3′ primers and nested primers.

### RT-qPCR:

RT-qPCR is performed on individual tumor samples using an SDS7900 Thermocycler (Applied Biosystems) with gene specific validated VEGF TaqMan probes (Hs00900055 ml, Applied Biosystems) according to the manufacturer's recommendations. Gene expression is normalized to a pool of housekeeping genes (e.g. 18S rRNA, RPLPO, Hmbs, Ppib, and/or Pgk1) selected for gene expression analysis in PC3 tumors to normalize for natural expression variation in vivo as previously described [75].

### VEGF ELISA:

VEGF protein expression is determined for individual tumor samples using a standard ELISA assay. Tumor tissue is lysed by the addition of RIPA buffer (Sigma Aldrich) and concentration measured by BCA assay (Perbio) according to the manufacturer's instructions. VEGF ELISA is performed with a commercial Quantikine human VEGF Immunoassay kit (R&D systems) according to the manufacturer's instructions.

### RNA In Situ Hybridization:

For RNA in situ hybridization, tumors are removed and immediately frozen in liquid nitrogen. Ten (10) μm Microtome sections are placed on microscope slides and fixed with 4% PFA. Detection is performed according to established procedures including Proteinase K digestion and acetic anhydride pre-treatment. A VEGF-specific DIG labeled probe is prepared from a VEGF cDNA containing plasmid with the DIG RNA labeling Kit (Roche Applied Science) according to the manufacturer's recommendations as published before [80]). The probe is incubated on the tissue sections in a humidified chamber at 65° C. overnight. The DIG labeled probe is detected with a sheep anti-DIG antibody conjugated to alkaline phosphatase (AP; Roche). The sections are then developed by the addition of BM purple (Roche) or another AP substrate.

### Efficacy Studies:

To determine the efficacy of the DARE™ delivery conjugate comprising a VEGF siRNA as compound (d), tumor size and the extent of tumor vascularization following treatment are determined. All control groups are similarly monitored for comparison.

### Tumor Growth/Remission:

Tumor size is measured every other day with a calliper, beginning on the date of treatment.

### Tumor Vascularization:

After termination of the experiment to assess tumor growth in response to DARE™-siRNA treatment, the extent of tumor vascularization is assessed as described before [86, 100]. Tumors are fixed in 10% buffered formalin before they are paraffin embedded and cut on a Microtome to obtain 5-15 μm sections. Hematoxylin and eosin (H&E) staining and immunohistochemistry for CD31 (to visualize blood vessels) expression is performed. Tumor tissue sections are pretreated with 0.1% trypsin for 10-15 min at 37° C. before incubation with rat anti-mouse CD31 (mAb MEC13.3, PharMingen, San Diego, Calif.) at a 1:500 dilution overnight at 4° C. Immunoreactivities are preferably visualized with the avidin-biotin complex technique using Vectastain Elite ABC kit (Vector Laboratories, Burlingame, Calif.) with diaminobenzidine as chromogen, or alternatively, by immunofluorescence. For comparison of vascularization, intratumoral CD31 positive vessels are counted per field of view.

### Example (14)

### Preparation and Administration of a DARE™ Delivery Conjugate Having a Bcl-xL Specific siRNA as Compound (d) In Vivo: Xenograft Model for Oncology

In this Example, the expression of the anti-apoptotic protein Bcl-xL is knocked down in a well established xenograft tumor model and its effect on tumor growth and apoptosis is determined [101, 102]. The experiments are carried out in gender and age matched, immuno-incompetent mice using PC-3 prostate adenocarcinoma cells (ATCC CRL 1435) as described above in Example 13.

The constructs and conjugates are prepared following the teachings of Examples 1 and 5. Each experiment consists of 5 groups, with n=14 mice/group:
1. DARE™ delivery construct without siRNA [i.e., lacking compound (d)]
2. Naked target Bcl-xL siRNA comprising a sense strand comprising 5′-GGUAUUGGUGAGUCGGAUCdTdT-3′(SEQ ID NO: 266), and an antisense strand comprising 5′-GAUCCGACUCACCAAUACCdTdT-3′ (SEQ ID NO: 267).
3. Naked non-target (Luciferase) siRNA comprising a sense strand comprising SEQ ID NO: 255, and an antisense strand comprising SEQ ID NO: 256.
4. DARE™ delivery construct with a compound (d) comprising target Bcl-xL siRNA comprising a sense strand comprising SEQ ID NO: 266, and an antisense strand comprising SEQ ID NO: 267.
5. DARE™ delivery construct with a compound (d) comprising a non-target siRNA comprising a sense strand comprising SEQ ID NO: 257, and an antisense strand comprising SEQ ID NO: 258.

Doses range from 100 to 2000 nmol/kg in 100 μL for systemic delivery and 0.05 to 5 nmol in 25 μL for local intratumoral delivery.

In the first part of this study, 6 animals are used per group for molecular knock-down analyses and animals are euthanized 2 days post treatment. In the second part of this study, 8 animals are used per group to analyze tumor growth/remission and apoptosis. Animals are observed at least twice weekly for up to 3 months or until moribund. Molecular analyses are carried out as follows or as described in Example 9 and Example 13.

### RNA Isolation:

After euthanasia, tumors are removed and immediately frozen in liquid nitrogen. RNA is isolated from tumor tissue with the RNeasy kit (Qiagen) according to the manufacturer's manual and RNA quality is determined with an Agilent 2100 Bioanalyzer using the RNA 6000 Nano kit (Agilent) according to the manufacturer's instructions.

### 5′ RACE-PCR:

5′ RACE-PCR is performed on individual tumor samples as described above in Example 9 using Bcl-xL specific 5′ and 3′ primers and nested primers.

### RT-qPCR:

RT-qPCR is performed on individual tumor samples using an SDS7900 Thermocycler (Applied Biosystems) with gene specific validated Bcl-xL TaqMan probes (Hs00236329_m1, Applied Biosystems) according to the manufacturer's recommendations. Gene expression is normalized to a pool of housekeeping genes (e.g. 18S rRNA, RPLPO, Hmbs, Ppib, and/or Pgk1) selected for gene expression analysis in PC-3 tumors to normalize for natural expression variation in vivo as previously described [75].

### Bcl-xL ELISA:

Bcl-xL protein expression is determined for individual tumor samples using a standard ELISA assay. Tumor tissue is lysed by the addition of RIPA buffer (Sigma-Aldrich) and concentration measured by BCA assay (Perbio) according to the manufacturer's instructions. Bcl-xL protein levels in the tumors are determined using a commercially available human Total Bcl-xL DuoSet ELISA kit (R&D Systems) according to the manufacturer's instructions.

### RNA In Situ Hybridization:

For RNA in situ hybridization, tumors are removed and immediately frozen in liquid nitrogen. Ten (10) μm Microtome sections are placed on microscope slides and fixed with 4% PFA. Detection is performed according to established procedures including Proteinase K digestion and acetic anhydride pre-treatment. A Bcl-xL-specific DIG labeled probe is prepared from a plasmid containing Bcl-xL cDNA. This is done with a DIG RNA labeling Kit (Roche Applied Science) according to the manufacturer's recommendations as previously described [80]. The probe is incubated on the tissue sections in a humidified chamber at 65° C. overnight. The DIG labeled probe is detected with a sheep anti-DIG antibody conjugated to alkaline phosphatase (AP: Roche). The sections are then developed by the addition of BM purple (Roche) or another AP substrate.

### Efficacy Studies:

To determine the efficacy of the DARE™ delivery conjugate comprising a Bcl-xL siRNA as compound (d), tumor size and the extent of tumor cell apoptosis following treatment are determined. All control groups are similarly monitored for comparison.

### Tumor Growth/Remission:

Tumor size is measured every other day with calipers, beginning on the date of treatment.

### Tumor Cell Apoptosis:

After termination of the experiment to assess tumor growth in response to DARE™-siRNA treatment, tumor cell apoptosis is analyzed using a TUNEL assay (Terminal deoxynucleotidyl transferase-mediated dUTP nick-end labelling) as previously described [102, 103]. For this purpose, tumors are immediately frozen after extraction. Sections of 4 μm are cut with a cryostat and fixed in acetone before the TUNEL stain is performed. Total cell numbers are determined by DAPI (Invitrogen) nuclei staining and images of the sections are acquired by fluorescence microscopy. Fractions of apoptotic (TUNEL positive) cells are calculated by automated analysis with Definiens enterprise software (Definiens).

### Example (15)

### Administration of a DARE™ Delivery Conjugate to Deliver Compound (d) In Vivo: Syngeneic Model for Oncology

In addition to the xenograft models in Examples 13 and 14, the DARE™ delivery conjugate of the present invention is examined in a syngeneic tumor model to assess its activity and distribution in an immunocompetent mouse model with more natural vascularization compared to a xenograft model. For this purpose, FVB/N mice are inoculated with firefly luciferase expressing DB7 tumor cells. DB7 tumor cells were originally derived from FVB/NTg(MMTV-PyVmT Y315F/Y322F) mice and have been previously described [104]. To increase tumor take, the cells were passaged through FVB/N mice before implantation. For imaging purposes, DB7 cells were transduced with a retroviral vector [105] expressing a dual function reporter gene (L2G) comprised of firefly luciferase (fLuc) and green fluorescent protein (GFP) driven by a hybrid promoter consisting of the β-actin promoter and the cytomegalovirus enhancer (CAGS). Transduced cells were screened for fLuc expression with an IVIS 50 system (Caliper LifeSciences, Hopkinton, Mass.) and 25 positive clones selected and combined to obtain a population representative of the parental population (DB7luc+).

To study the tumor penetration and efficacy of DARE™-siRNA delivery conjugates of the present invention, gender and age matched mice (6-10 weeks of age) are injected with 2.5×10⁶ DB7luc+ cells subcutaneously. Tumors are allowed to establish for 2 weeks before the conjugates are injected.

The siRNA sequence for luciferase is optimized in vitro or an already described sequence [76] is used. siRNAs are controlled for immunostimulatory effects as described in Example 11.

siRNA and DARE™ construct formulations are prepared as described in Examples 1 and 5 are delivered systemically by tail vein injections or intratumorally in independent experiments. Each experiment consists of 5 groups with n=5 mice/group:
1. DARE™ delivery construct without siRNA[i.e., lacking compound (d)];
2. Naked fLuc siRNA comprising a sense strand comprising SEQ ID NO: 255, and an antisense strand comprising SEQ ID NO: 256;
3. Naked non-target siRNA comprising a sense strand comprising SEQ ID NO: 257, and an antisense strand comprising SEQ ID NO: 258;
4. DARE™ delivery construct with fLuc siRNA comprising a sense strand comprising SEQ ID NO: 255, and an antisense strand comprising SEQ ID NO: 256 as compound (d); and
5. DARE™ delivery construct with a non-target siRNA comprising a sense strand comprising SEQ ID NO: 257, and an antisense strand comprising SEQ ID NO: 258 as compound (d).

Doses used range from 100 to 2000 nmol/kg in 100 μL for systemic delivery and 0.05 to 5 nmol in 25 μL for local intratumoral delivery. Mice are euthanized at several time points post DARE™ injection (ranging from 1-7 days) and the tumors removed for molecular analysis as follows or as described above in Example 9. Tumors are stored in RNAlater (Qiagen) for subsequent analysis of fLuc mRNA levels by qRT-PCR and RNAi specific degradation of fLuc mRNA by 5′-RACE using fLuc specific 5′ and 3′ primers and nested primers. For quantification of luciferase and total tissue protein levels (to obtain the amount of luciferase per protein tissue), the tumors are frozen in liquid nitrogen. For luciferase enzyme activity measurement, the tumor is homogenized, using a tissue lyser/mixer mill (Qiagen), metal beads and luciferase cell culture lysis reagent (Promega PR-E1531), centrifuged for 5 min at maximum speed in a table top centrifuge (13,000 g) before the supernatant is transferred to a new reaction tube. The supernatant is either stored at −80° C. or used immediately to measure luciferase in a luminometer, using a luciferase assay system (Promega) according to the manufacturer's instructions.

### Example (16)

### Demonstration of DARE™ Conjugate Delivery In Vivo: Local Delivery to the Central Nervous System (CNS)

Different versions of a modular DARE™ delivery conjugate of the present invention are delivered to the brain in a mouse model.

The following siRNA sequences are preferably used:

### GAPDH:

sense: SEQ ID NO: 248;
antisense: SEQ ID NO: 249;
Non-silencing control:
sense: SEQ ID NO: 257, and
antisense: SEQ ID NO: 258.

The constructs and conjugates are prepared as described in Example 1. GAPDH specific knockdown is tested in Balb/c mice. Gender and age matched mice (6-10 weeks of age) are used. Single injections and long-term infusions are performed. Each experiment includes the following groups with n=10 animals/group:
1. DARE™ delivery construct without siRNA [i.e., lacking compound (d)]
2. Naked GAPDH siRNA comprising a sense strand comprising SEQ ID NO: 248, and an antisense strand comprising SEQ ID NO: 249;
3. Naked non-target siRNA comprising a sense strand comprising SEQ ID NO: 257, and an antisense strand comprising SEQ ID NO: 258;
4. DARE™ delivery construct with GAPDH siRNA comprising a sense strand comprising SEQ ID NO: 248, and an antisense strand comprising SEQ ID NO: 249 as compound (d); and
5. DARE™ delivery construct with a non-target siRNA comprising a sense strand comprising SEQ ID NO: 257, and an antisense strand comprising SEQ ID NO: 258 as compound (d).

For local delivery to the caudate putamen, single injections of 1 μL of DARE™ (total doses ranging from 0.05 to 5 nmol) in PBS are injected. Before the injection, animals are anaesthetized preferably by i.p. injection of 3.6% chloral hydrate (10 mL/kg) in H₂O, which is reapplied at half dose in the case where an animal begins to wake up. In preparation for the injection, the animal is then positioned in a stereotaxic apparatus (Axel Semrau, Sprockhoevel, Germany). After opening the skin by a scalpel incision, the skull is cleaned and opened with a fine drill (0.5 mm diameter) in preparation for the injection with a Hamilton syringe. Drilling and injections are performed according to the stereotaxic coordinates previously described [106, 107]. For injections into the caudate putamen, the coordinates for the tip of the syringe are (from bregma): Lateral—1.6 mm, Dorso-Ventral—3.8 mm, Anterior-Posterior—0.5 mm.

For long-term delivery, a DARE™ conjugate of the present invention is delivered via an osmotic pump (Alzet brain infusion kit) into the third ventricle at AP: −0.5 mm; ML: 0 mm, DV: −3 mm, relative to Bregma) as previously described [108, 109]. Briefly, the animals are prepared as above for single injections before a cannula ending at the appropriate coordinates is implanted and fixed to the skull. The osmotic pump is filled with a DARE™ conjugate of the present invention to achieve a delivery rate of 0.01 to 0.5 nmol per day in a daily volume of 5 μL for an infusion period of 2 weeks. The pump is implanted subcutaneously in the neck of the animals and connected to the cannula via silicone tubing.

Following the single injections, the animals are euthanized at 1-7 days post-injection. In the case of the infusions, the animals are euthanized immediately after the 2 weeks of infusion. The brain of each animal is immediately removed and processed for analysis of DARE™ distribution and efficacy as follows or as described above in Example 7 and Example 9. For RNA and protein analysis, the brains are dissected immediately following death of the animal and tissue is collected from different areas of interest and immediately frozen in liquid nitrogen. RNA is isolated with the Qiagen RNeasy Lipid tissue kit according to the manufacturer's manual. RT-PCR and 5′-RACE are performed as described in Example 9 above.

### Immunohistochemistry:

For distribution analysis of the Cy3 labeled DARE™ construct and analysis of protein expression by immunohistochemistry, the brain of each animal is fixed in 4% PFA, 0.05% glutaraldehyde in PBS for 24 h before being soaked in 30% sucrose for 36 h. The brain tissue is then frozen at −80° C. for storage, and 7 μM sections are cut at −20° C. and placed on slides for microscopy analysis. GAPDH protein expression is detected using a GAPDH specific antibody (rabbit mAB 14C10, Cell Signaling, or similar). Antigen detection is performed according to the manufacturer's recommendations following microwave assisted antigen retrieval using citrate buffer. Detection of the primary antibody is done with an anti-rabbit horseradish peroxidise (HRP- or fluorophore-labeled secondary antibody (Abeam) and then analyzed by microscopy using standard protocols or, in the case of a fluorophore labeled secondary antibody, as described above in Example 7.

### RNA In Situ Hybridization:

In situ RNA detection is performed according to established procedures including Proteinase K digestion and acetic anhydride pre-treatment. Brain tissue is fixed in 4% PFA for 24-30 h after extraction before soaking in 30% sucrose for 24-30 h. It is then cooled to −70° C. in isopentane and 5 μm thick sections are cut in a cryostat-microtome. A target-specific digoxygenin labeled probe is prepared from a GAPDH cDNA containing plasmid with and SP6 or T7 RNA polymerase with the DIG RNA labeling Kit (Roche Applied Science) according to the manufacturer's recommendations and as described earlier [110]. The probe is incubated on the tissue sections in a humidified chamber at 65° C. overnight. The DIG labeled probe is detected with a sheep anti-DIG antibody conjugated to alkaline phosphatase (AP; Roche). The sections are then developed by the addition of BM purple (Roche) or another AP substrate.

While this Example illustrates the preparation, use and characterization of a specific, ricin B-[i.e., module (a)] targeted conjugate of the invention to deliver a GAPDH targeted siRNA as compound (d), the teachings of this Example are applicable to any conjugate of the invention. In particular, one of skill in the art may replace the GAPDH targeted siRNA with another siRNA directed against a target in which CNS gene expression knockdown is desired. In addition, one of skill in the art can replace the GAPDH targeted siRNA of the conjugate described in this Example with another compound (d) that is desired to be delivered to a cell in the CNS. As described above, modules (a), (b) and (c) can also be modified accordingly by one of skill in the art to suit the intended purpose and target cell within the CNS. These embodiments may be prepared without undue experimentation and are encompassed within the scope of the present invention.

### Example (17)

### Use of Chemical Inhibitors of the Retrograde Pathway to Monitor DARE™ Conjugate Delivery Via Retrograde Transport

To monitor DARE™ conjugate delivery via retrograde transport, one can use chemical inhibitors or drugs that interfere in these pathways. These drugs have been commonly used in the literature and include brefeldin A (disrupts Golgi) and monensin (modulates transport to the Golgi, e.g. low concentrations increase ricin toxicity while higher concentrations protect against it) [111]. Thus, one can follow the DARE™ conjugates through the cell via co-stainings for the different organelles.

Retrograde pathway inhibitors are expected to prevent the transport from the endosome to the Golgi. If the inhibitor does indeed inhibit the transport of a conjugate of the present invention, indicated by a reduced RNAi effect and/or by confocal microscopy (i.e., wherein a fluorescently labeled DARE™ construct is no longer able to reach the ER), then this result indicates that the retrograde pathway is used by the DARE™ conjugate to deliver its compound (d) to the cytosol. Thus, if a DARE™ conjugate according to the present invention trafficks through the retrograde pathway to reach the ER, then pre-treatment of the cells with a retrograde pathway inhibitor before DARE™ conjugate addition should result in a reduction in fluorescently labeled DARE™ conjugates in the ER of the cells. Further, if inhibitor pre-treatment results in a reduced RNAi effect, then the DARE™ conjugate most likely uses the retrograde pathway to deliver its compound (d) (i.e., the siRNA cargo) to the cytosol.

Brefeldin A (BFA; Sigma-Aldrich, product no. B5936) is added to the cells with a final concentration of 5 μg/mL. This concentration results in rapid fusion of the Golgi with the ER within 30 min [111, 112]. However, a lower concentration of BFA of 0.5-1 μg/mL is sufficient in some cell lines to inhibit retrograde transport while enhancing cell survival for 1-3 days [111,112]. BFA also causes the fusion of early endosomes and the TGN.

Alternatively, nordihydroguaiaretic acid (NDGA; Sigma-Aldrich, product no. 74540), a lipoxygenase inhibitor, is added to the cells (in serum free medium) with a final concentration of 25 μM. This concentration results in rapid fusion of the Golgi with the ER within 30 min [113-115].

Alternatively, cyclofenil diphenol (CFD; Sigma-Aldrich, product no. C3490-10MG), a non-steroidal estrogen, is added to the cells with a final concentration of 25 μM. This concentration results in rapid fusion of the Golgi with the ER within 30 min.

Alternatively, Retro-1 or Retro-2 (Chembridge, www.chembridge.com) added to the cells with a final concentration of 25 μM. These latter two inhibitors do not cause fusion of cell organelles but specifically inhibit toxins (ricin, shiga, and the like) from being transported from the endosome to the TGN [new 116].

As a further alternative to the above inhibitors, Golgicide A (Sigma-Aldrich, product no. G0923-5MG, [117]) or other inhibitors of retrograde transport can be used.

The inhibitor of retrograde transport is added 30 min prior to the addition of the DARE™-siRNA construct. Knock down of the target mRNA and the target protein (e.g. GAPDH or luciferase) is evaluated after 6, 24 and 48 h using RT-qPCR and the appropriate protein assays, e.g. standard GAPDH enzyme activity assay or luciferase activity assay, as described in Example 9. Incubation with the inhibitor may be stopped by changing the medium before the incubation period is over if the inhibitor shows excessive cell toxicity; e.g. the inhibitor is removed after 6 h (or earlier) by changing the medium but the RT-qPCR and the protein assays are still performed after 24 and 48 h.

In addition or as an alternative to the RNAi experiments described above, retrograde transport can also be demonstrated via immunohistochemical analysis. NIH-3T3, HeLa or other appropriate cell lines are incubated with the DARE™-siRNA construct, which carries a fluorophore such as Cy3, for 15-60 min, followed by a medium change. At several time points thereafter (e.g. 30 min, 1, 2, 4, 6 and 24 h), the cells are fixed, stained with antibodies for different cell organelles and examined by confocal microscopy. During the incubation with the DARE™-siRNA, the inhibitor is added to half of the wells to demonstrate the use of the retrograde pathway for the transport of the DARE™-siRNA. For organelle markers, the following are used: Transferrin conjugated to a fluorophore to stain the early and recycling endosome (added to the cells when the DARE™-siRNA is added); LAMP 1 antibody to stain lysosomes; Mannosidase II antibody to stain the Golgi Apparatus; Calreticulin, Calnexin (or Derlin-1) antibody to stain the ER; and nuclei can be stained with Hoechst dye (Invitrogen).

### Example (18)

### siRNAs Against Key Genes of the Retrograde Pathway

Knock down of key components of the retrograde pathway and ERAD via siRNA(s) that target these key components can also be used to track the pathway of conjugates of the invention. As an alternative to Example 17's use of chemical inhibitors of the retrograde pathway, key proteins for the retrograde transport of the DARE™-siRNA can also be knocked down with an siRNA. The analyses are identical to those described above in Example 17, i.e. reduced knock down by DARE™-siRNA and inhibited retrograde transport of the DARE™ siRNA. One to two days prior to the addition of DARE™-siRNA to the cells, the cells are transfected with an siRNA against one or several of the following genes: KDELR-1 (Accession number 10945), KDELR-2 (Accession number 11014), KDELR-3 (Accession number 11015), Sec61a1 (Accession number 29927), Derlin-1 (also referred to as DERL-1, Accession number 79139), PDIA2 (Accession number 64714), and Ero1L (Accession number 30001), comprising one of the following siRNA sequences or an siRNA sequence as prepared by one of skill in the art:

| KDELR-1: | |
|---|---|
| (SEQ ID NO: 268) | |
| sense: | 5′-CUACCUCUAUAUCACCAAATT-3′, |
| (SEQ ID NO: 269) | |
| antisense: | 5′-UUUGGUGAUAUAGAGGUAGAA-3′, |
| KDELR-2: | |
| (SEQ ID NO: 270) | |
| sense: | 5′-AUAGGAGCAGGCAAGGUAGAT-3′, |
| (SEQ ID NO: 271) | |
| antisense: | 5′-CUACCUUGCCUGCUCCUAUTT-3′, |
| KDELR-3: | |
| (SEQ ID NO: 272) | |
| sense: | 5′-ACUGAUUCCAGAUAGAUAGAG-3′, |
| (SEQ ID NO: 273) | |
| antisense: | 5′-CUAUCUAUCUGGAAUCAGUTT-3′, |
| Sec61a: | |
| (SEQ ID NO: 274) | |
| sense: | 5′-GGAAUUUGCCUGCUAAUCATT-3′, |
| (SEQ ID NO: 275) | |
| antisense: | 5′-UGAUUAGCAGGCAAAUUCCAG-3′, |
| Derlin-1: | |
| (SEQ ID NO: 276) | |
| sense: | 5′-GCUUAGCAAUGGAUAUGCATT-3′, |
| (SEQ ID NO: 277) | |
| antisense: | 5′-UGCAUAUCCAUUGCUAAGCCA-3′, |
| PDIA2: | |
| (SEQ ID NO: 278) | |
| sense: | 5′-GUCGGAAGGUGAUUGAAUATT-3′, |
| (SEQ ID NO: 279) | |
| antisense: | 5′-UAUUCAAUCACCUUCCGACCT-3′, |
| Ero1L: | |
| (SEQ ID NO: 280) | |
| sense: | 5′-GGAAUGUCAUCUACGAAGATT-3′, |
| and | |
| (SEQ ID NO: 281) | |
| antisense: | 5′-UCUUCGUAGAUGACAUUCCAT-3′. |

### Example (19)

### DARE™ Conjugates Comprising at Least Two Compound (d) Molecules Per Conjugate

This Example describes the preparation of a conjugate comprising 2 compounds (d), wherein the compounds (d) are two of the same target siRNA (see FIG. 14). One of skill in the art can appreciate that by increasing the number of compound (d) molecules conjugated to the conjugate of the present invention, one can increase the potency of the conjugate and thus, the delivery system of the present invention. In the case where the at least 2 compounds (d) are siRNAs, a positively charged molecule (i.e., spermine, spermidine or a positively charged peptide) may need to be added to the formulation, or may need to be used at a higher concentration in the formulation than required for the single siRNA-conjugate of the present invention, to compensate for the increased negative charge due to multiple siRNAs.

(i) Synthesis of the Linkage Molecule Comprising Modules (b) and (c):

The [module (b)+module (c)+2 linkers] peptide H₂N—C(NPys)-(SG)-3-(DprAoa)(dPEG12) (DprAoa)-(SG)₃-NASSSRSGLDDINPTVLLKAKDEL-OH [the peptide comprising “module (b)+module (c)” comprises an amino acid sequence comprising SEQ ID NO: 244] is synthesized commercially by standard solid-phase Fmoc peptide chemistry, deprotected in the standard fashion and purified by reversed phase HPLC to a purity of >95%. QC of the peptide is done by amino acid analysis, mass spectroscopy and analytical reversed phase HPLC. The activated cysteine residue is introduced using Boc-Cys(NPys)-OH (Bachem product no. A-2825) as a building block. Fmoc-Dpr(Boc-Aoa)-OH (Novabiochem product no. 04-12-1185) is used to introduce the N-β-aminoxyacetyl L-diaminopropionyl residue. dPEG12 is introduced using Fmoc-dPEG₁₂-acid (Quanta BioDesign, product no. 10283). QC of the purified peptide is done by ESMS and analytical reversed phase HPLC.

(ii) Synthesis of the Delivery Carrier Comprising Modules (a), (b) and (c) and 2 Linkers:

To prepare module (a), recombinant ricin toxin B subunit (SEQ ID NO: 124; Vector Laboratories, Inc., catalog no. L-1290) and supplied as a 1 mg/mL solution in 10 mM aqueous sodium phosphate, 0.15 M NaCl, pH 7.5, containing 0.08% sodium azide and 50 mM 2-ME is supplemented with fresh 50 mM 2-ME and incubated for 1 h at RT to ensure that the Cys residue at position 4 is fully reduced. The sample is desalted using a Vivaspin 2 polyethersulfone (PES) ultrafiltration spin column (molecular weight cut-off of 5 kDa, Sartorius Stedim Biotech, part no. VS0211) and the buffer exchanged to degassed 10 mM phosphate buffer, 150 mM NaCl, 1 mM EDTA pH 7. The resulting ricin B solution is reacted overnight at 10° C. under argon with 1.1 mole equivalents of the linkage molecule containing modules (b) and (c) from Example 19(i) above. The desired delivery carrier is then purified by preparative gel filtration using a HiLoad 16/60 Superdex 75 prep grade column (GE Healthcare, part no. 17-1068-01), eluted with 50 mM sodium dihydrogen phosphate buffer, 100 mM NaCl, 2 μM EDTA pH 5.0 at a flow rate of 1 mL/min. Identification of the desired carrier peak is enabled by having pre-calibrated the SEC column with ricin B and the linker-peptide entity from Example 19(i). The product is analyzed by native gel electrophoresis and by DTT cleavage into 2 components, each of which are individually analyzed.

(iii) Preparation of the Cargo siRNA [Compound (d)]:

A Tuschl-style siRNA targeting GAPDH is synthesized, purified and analyzed exactly as described in Example 1(iii), wherein the 5′-terminus of the sense strand is modified with a 5′-(C₆-aminolinker)-phosphate-(C₆-SS-C₆)-phosphate-Cy3 entity. The primary amine is further reacted with the adapter molecule SFB following the procedure in Example 1(iii) and desalted and buffer exchanged.

(iv) Coupling of a Double siRNA Cargo [2 Compounds (d)] to the Delivery Carrier [Modules (a)+(b)+(c) and 2 Linkers]:

The delivery carrier from Example 19(ii) above is reacted overnight at 10° C. with 3 mole equivalents of the adapter-siRNA cargo from Example 19(iii) above in phosphate buffer pH 5. The desired module (a)+module (b)+module (c)+compounds (d) conjugate is purified by preparative SEC on a HiLoad 16/60 Superdex 75 prep grade column (GE Healthcare, part no. 17-1068-01), eluted at 1 mL/min with sterile PBS, pH 7.4. QC is performed by native gel electrophoresis and analytical SEC on a Superdex 75 10/300 GL column (GE Healthcare, part no. 17-5174-01). Further analysis is done by incubating the product with DTT or TCEP to cleave the two accessible disulfide bonds and give three molecules, each of which can be isolated by HPLC, individually characterized by ESMS and, if necessary, sequenced.

It will be apparent to one of skill in the art that the approach described within this Example may be used to attach other cargos, e.g. a nucleic acid, a protein, a peptide, a therapeutic moiety, and the like, to a delivery carrier (i.e., [module (a)+module (b)+module (c)] of the present invention.

### Example (20)

### Synthesis of DARE™ 3.02 constructs (DARE™-T-AK-SGK), Sgk1-TfR-AKDEL-siRNA (see FIG. 11), carrying fLuc and GAPDH targeted siRNAs respectively

(i) Synthesis of the Linkage Molecule Containing Modules (a), (b) and (c), viz. Sgk1-TfR-AKDEL

The [module (a) (SEQ ID NO: 121)+module (b) (SEQ ID NO: 161)+module (c) (SEQ ID NO: 199)+linker peptide (SEQ ID NO: 7)] of sequence MTVKTEAAKGTLTYSRMRGMVAILIAFMKQ-(S-G)₃-Cys-(S-G)₃-THRPPMWSPVWPA KDEL was synthesized by standard solid-phase Fmoc chemistry, deprotected in the standard fashion and purified twice by preparative reversed phase HPLC. The purity was estimated at 57-84% (due to shoulders on the back and front of the peak) by analytical reversed phase HPLC on a Vydac 218TP54 column using a gradient from 0.1% aqueous TFA to 0.1% TFA in 60% acetonitrile during 40 min, eluted at 1 mL/min. The mass measured by matrix assisted laser desorption ionization mass spectroscopy (MALDI-MS) in positive ion mode was 6346.81 Da for M+H⁺; the calculated mass of C₂₇₅H₄₄₂N₇₈O₈₂S₆ is 6345.41 Da. The cysteine thiol was then activated by reaction of the purified peptide (50 mg, ca. 7 μmol) with 5-nitro-2-[(5-nitropyridin-2-yl)disulfanyl]pyridine (6.2 mg, 20 μmol; from Sigma-Aldrich, catalog #43765) in pyridine (5 mL) for 2 h at room temperature with stirring, to give 11 mg of the desired MTVKTEAAKGTLTYSRMRGMVAILIAFMKQ-(S-G)₃-Cys(pNPys)-(S-G)₃-THRP PMWSPVWPAKDEL after two preparative RP-HPLC purifications. The purity of the activated peptide was 78.8% by reversed phase HPLC. MALDI-TOF MS showed the correct M+H⁺ ion at m/z 6500.64; the calculated mass for C₂₈₀H₄₄₄N₈₀O₈₄S₇ is 6499.56 Da.

(ii) Preparation of the siRNA Cargo Compounds (d)

A double stranded RNA molecule comprised of two 21mer strands, with a double stranded region of 19 nucleotides in length and 2 nucleotides overhanging at the 3′-end of each strand, and targeting glyceraldehyde 3-phosphate dehydrogenase (GAPDH), wherein the sense strand comprises 5′-CCAuCUUCCAGGAGCgAGAuu (SEQ ID NO: 248), wherein lowercase u or g represents a 2′-O-methylribonucleotide; and the antisense strand comprises 5′-UCUCGCUCCUGgAAGAuGGdTdT (SEQ ID NO: 249), wherein lowercase u or g represents a 2′-O-methylribonucleotide and wherein the antisense strand has a 5′-phosphate and deoxynucleotides at its 3′-end (dNdN), was synthesized such that the 5′-terminus of the sense strand was modified with a 5-(C6-SS-C6 spacer)-phosphate-Cy3 moiety. In addition, a double stranded RNA molecule comprised of two 21 mer strands, with a double stranded region of 19 nucleotides in length and 2 nucleotides overhanging at the 3′-end of each strand, and targeting firefly luciferase (fLuc), wherein the sense strand comprises 5″-CUUACgCUGAGuACUUCGAuu (SEQ ID NO: 255), wherein lowercase u or g represents a 2′-O-methylribonucleotide; and the antisense strand comprises 5′-UCGAAGUACUC AgCGUAAgdTdG (SEQ ID NO: 256), wherein lowercase g represents a 2′-β-methylribonucleotide and wherein the antisense strand has a 5″-phosphate and deoxynucleotides at its 3′-end (dNdN), was synthesized such that the 5′-terminus of the sense strand was modified with a 5-(C6-SS-C6 spacer)-phosphate-Cy3 moiety. The four HPLC-purified individual single strands were all analyzed by HPLC and MALDI-TOF MS. In order to prepare the two duplexes for the disulfide exchange reaction with the activated linkage molecule containing modules (a), (b) and (c), 50 A₂₆₀ units of each duplex was dissolved in 0.5 mL of sterile 0.2 M aqueous sodium acetate, pH 6 containing 100 mM dithiothreitol (DTT) and kept at 37° C. for 2 h to cleave the disulfide bond. The solutions were then desalted using degassed water as eluent and lyophilized.

(iii) Coupling of the siRNA Cargo [Compound (d)] to the Deliver Carrier [Modules (a)+(b)+(c) and Linker]

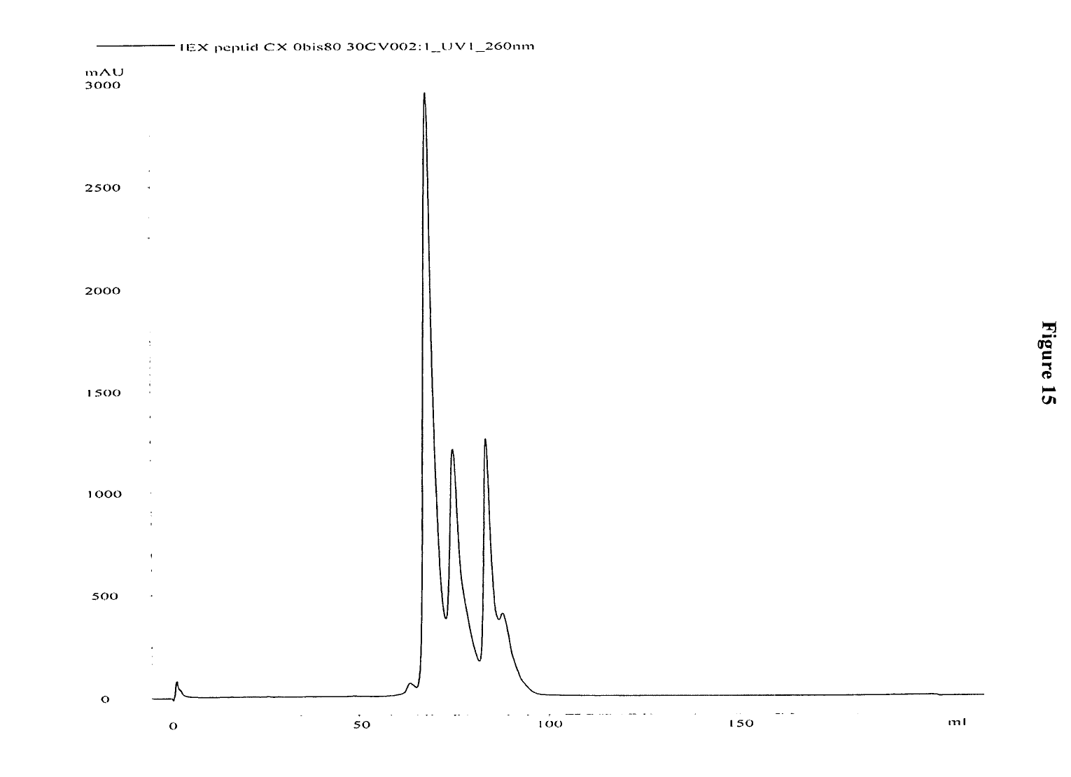

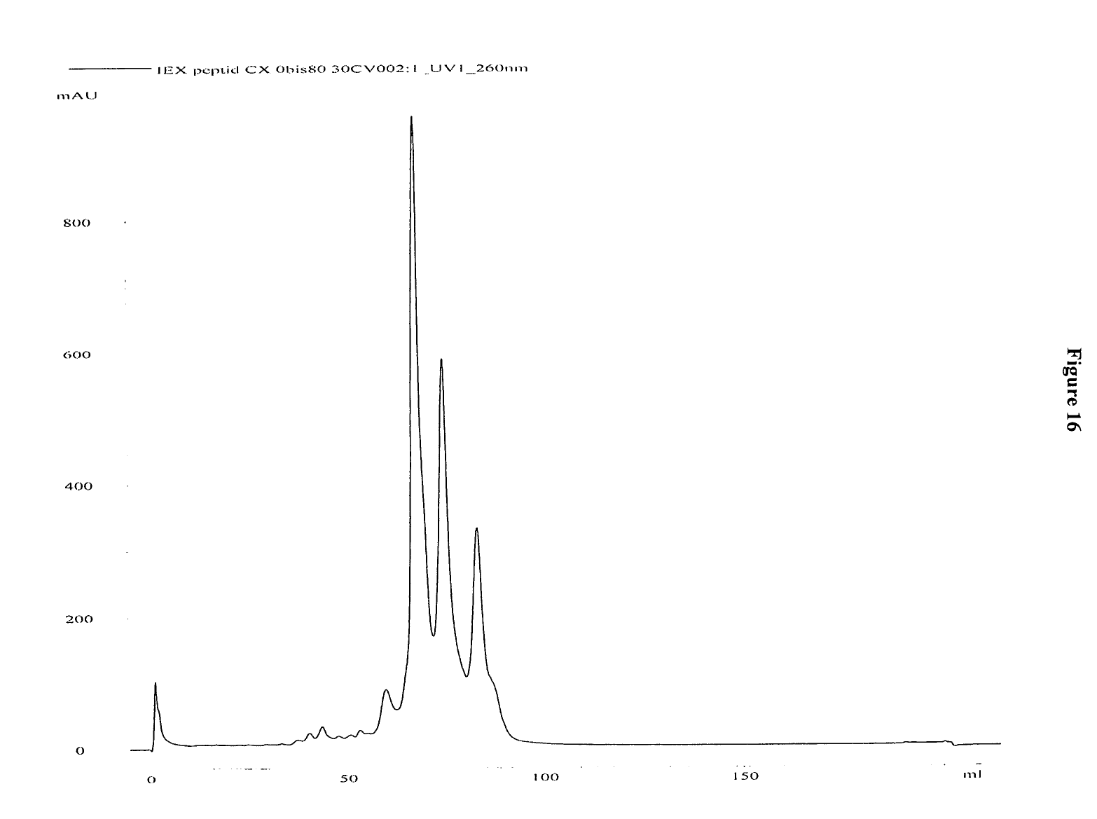

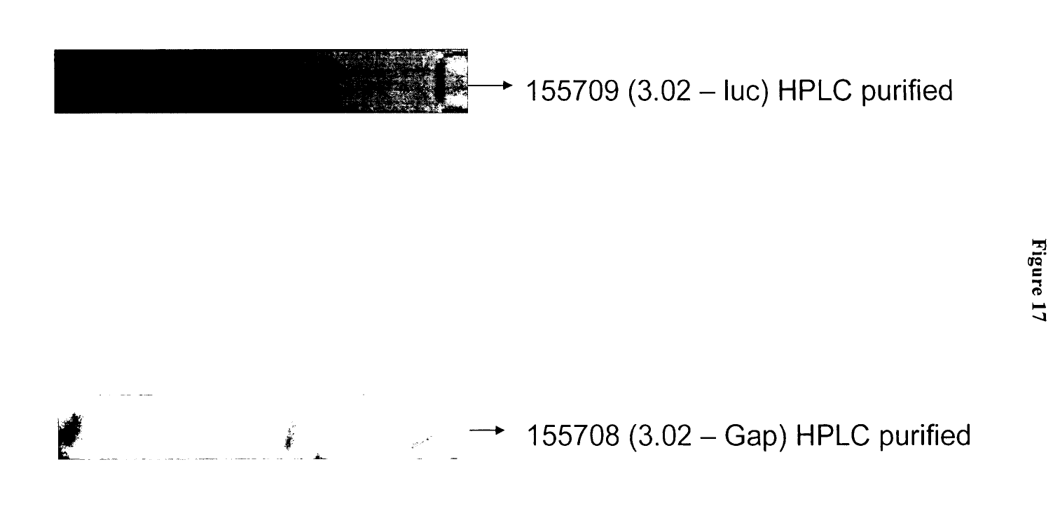

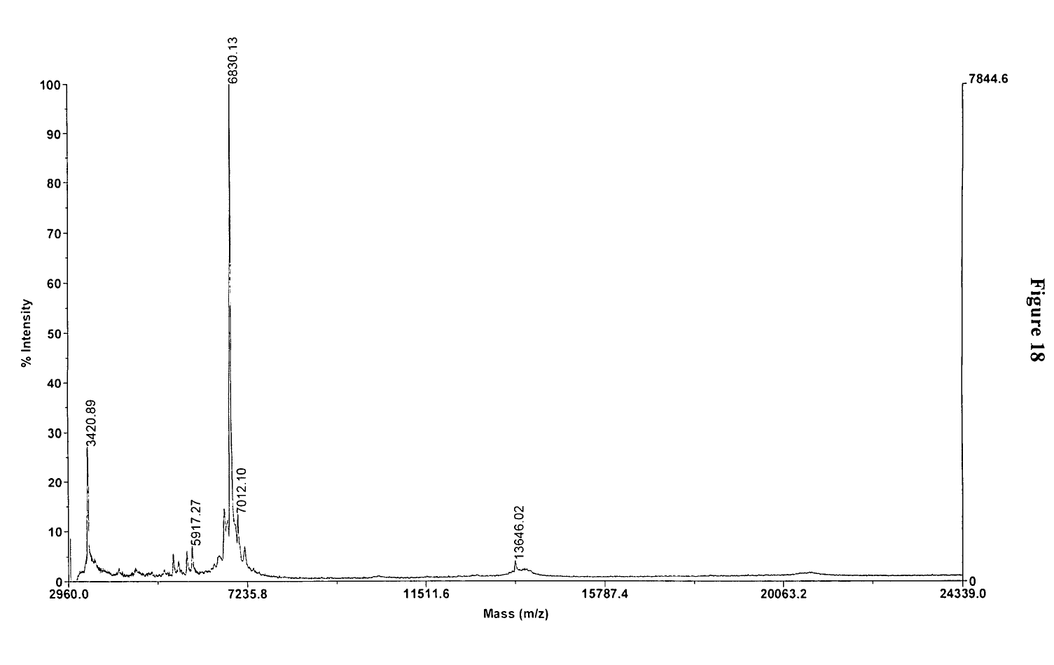

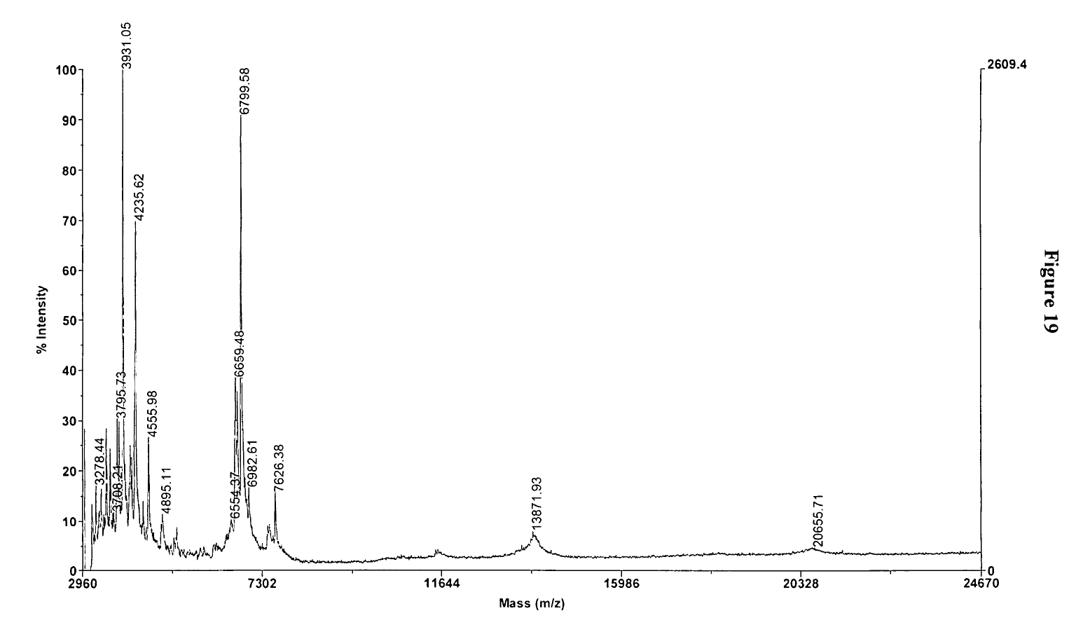

fLuc-siRNA (10 A₂₆₀ units, ˜25 nmol) from Example 20(ii) above was dissolved in 100 μL of 8 M guanidinium chloride in sterile phosphate buffered saline (PBS), pH 7.4 under argon. MTVKTEAAKGTLTYSRMRGMVAILIAFMKQ-(S-G)₃-Cys(pNPys)-(S-G)₃-THRPPMWSP VWPAKDEL (0.5 mg, ˜72 nmol) from Example 20(i) above was dissolved in 100 μl, of 8 M guanidinium chloride in degassed sterile water. The peptide solution was added to the fLuc-siRNA solution and the reaction was allowed to proceed for 17 h at 22° C. The solution was then diluted to 1 mL with sterile 50 mM ammonium acetate and loaded into a spin column (0.5 mL, Amicon Ultra with an Ultracel 10 kDa membrane). The column was washed once with 50 mM ammonium acetate followed by water. The desalted sample was removed, lyophilized and then dissolved in 0.5 mL of sterile 25 mM Tris-HCl buffer, pH 7.4 containing 6 M urea (buffer A) and loaded onto a 1 mL Resource Q anion-exchange HPLC column (GE Healthcare, part no. 17-1177-01). The column was eluted with a linear gradient from 0-80% B in 180 column volumes (CV) using a flow rate of 3 mL/min. Buffer B was 25 mM Tris-HCl, 1 M sodium bromide and 6 M urea, pH 7.4 using an Akta purifier HPLC (GE Healthcare). The column effluent was monitored at 260 nm and 550 nm (Cy3 absorbance) and three peaks were observed, the first (major) peak was identified as the desired conjugate by mass spectroscopy. The preparative anion-exchange HPLC trace is shown in FIG. 15. An identical experiment was performed for the GAPDH-siRNA, and the preparative anion-exchange HPLC trace is shown in FIG. 16. The product containing peaks were exhaustively desalted using a spin column and then lyophilized. The yield of the two purified DARE™ 3.02 constructs was in the range of 3-7 nmol. FIG. 17 shows 15% PAGE gels of the fLuc-siRNA and GAPDH-siRNA containing DARE™ 3.02 constructs, performed at 220 V and 25 mA with a running time of 1-1.5 h, using a precast 8×6.5 cm gel (Biostep, part no. 95-70-181) and standard Tris-borate running buffer containing 6 M urea. Confirmation of construct identity was performed by MALDI-TOF mass spectroscopy on a Voyager instrument, see FIGS. 18 (3.03-fLuc) and 19 (3.02-GAPDH).

### REFERENCES

1. Behlke, M A (2006). Mol Ther, 13:644-670.
2. Rozema, D. B., D. L. Lewis, et al. (2007). Proc Natl Acad Sci USA 104(32): 12982-7.
3. Sambrook et al. (1989). Molecular Cloning: A Laboratory Manual, 2d ed. Cold Spring Harbor Laboratory Press, Cold Spring Harbor, N.Y.
4. Leuenberger, H. G. W, Nagel, B. and Kolbl, H. eds. (1995). “A multilingual glossary of biotechnological terms: (IUPAC Recommendations).” Helvetica Chimica Acta, CH-4010 Base1, Switzerland.
5. Karlin and Altschul (1990). Proc. Natl. Acad. Sci. USA 87:2264-2268.
6. Karlin and Altschul (1993). Proc. Natl. Acad. Sci. USA 90:5873-5877.
7. Altschul, et al. (1990). J. Mol. Biol. 215:403-410.
8. Altschul et al. (1997). Nucleic Acids Res. 25:3389-3402.
9. Tung C-W and Ho S-Y, (2008). BMC Bioinformatics 9:310, 1-15.
10. Catic A., Collins C., Church G M, Ploegh H L (2004). Bioinformatics 20(18):3302-3307.
11. Xu P., et al. (2009). Cell 137 (1), 33-147.
12. Thrower, et al. (January 2000). EMBO 19 (1): 94-102.
13. Ivanov (Ed.) (2008). “Exocytosis and Endocytosis”, Series: Methods in Molecular Biology, Springer Vol. 440, ISBN: 978-1-58829-865-2.
14. Ghosh et al. (1994). J. Cell Science 107:2177-2189.
15. Watson et al. (2005). Adv Drug Deliv Rev 57:43-61.
16. Mallard and Johannes. (2002). Methods Mol. Med. 2002. Shiga Toxin Methods and Protocols (Edited by: D Philpott and F Ebel), 73, (17), p 209-220.
17. Bernardi, et al. (2008). Mol. Biol. Cell 19:877-884.
18. Huston et al. (1988). Proc. Nat'l Acad. Sci. 85:5879.
19. Whitlow et al. (1993). Protein Engineering 6:989.
20. Newton et al. (1996). Biochemistry 35:545.
21. Eguchi, et al. (2009). Nat Biotechnol 27(6):567-71.
22. Bevilacqua and Cech. (1996). Biochemistry 35(31):9983-94.
23. McKenna et al. (2006). J Mol Biol 358(5):1270-85.
24. Tompa et al. (2004). J. Biol. Chem. 279(20):20775-20785.
25. Waehler et al. (2007). Nat Rev Genet. 8(8):573-587.
26. Tang et al. (2007). Gene Ther 14(6):523-532.
27. Tuma and Hubbard. (2003). Physiol Rev 83(3):871-932.
28. Zito et al. (2007). EMBO J. 26(10):2443-2453.
29. Lee et al. (2001). Eur J. Biochem. 268(7):2004-2012.
30. Xia et al. (2000). J. Virol. 74(23):11359-11366.
31. Truskey et al. (2009). Transport Phenomena in Biological Systems, Chapter 11. “Cell Surface Ligand-Receptor Kinetics and Molecular Transport within Cells.” 2″ Edition Pearson Education Inc., ISBN 978-O-13-156988-1.
32. Futter, et al. (1996). J. Cell Biol. 132:1011-1023.
33. Mallard et al. (1998) J. Cell Biol. 143(4): 973-990
34. Johannes, et al. (1997). J. Biol. Chem. 272 (31):19554-19561.
35. Pelkmans, et al. (2001). Nat Cell Biol. 3(5):473-483.
36. Damm, et al. (2005). J. Cell Biol. 168(3):477-488.
37. Neu, et al. (2009). Virology. 384(2):389-399.
38. Raykhel et al. (2007). J. Cell Biol. 179(6):1193-1204.
39. Song and Smith. (1996). Arch Biochem Biophys. 334(1):67-72.
40. Johannes and Popoff (2008). Cell 135:1175-1187,
41. Sorgjerd et al. (2006). J Mol. Biol. 356(2):469-482.
42. Hirsch et al. (2009). Nature. 458(7237):453-460.
43. LaPointe et al. (2005). J Biol. Chem. 280(24):23310-23318.
44. Yue et al. (2010). Nature Chem. Biol. 6:621-629.
45. Agrawal. (1996). Trends in Biotech. 14:376-387.
46. Choi et al. (2006). Viral Immunol. 19(1):54-63.
47. Haicheur et al. (2000). J. Immunol. 165(6):3301-3308.
48. Smith et al. (2002). J. Immunol. 169(1):99-107.
49. Makkerh et al. (1996). Curr Biol. 6(8):1025-1027.
50. Mudhakir and Harashima. (2009). AAPS J. 11(1):65-77.
51. Wade and Weller. (1994). “Handbook of Pharmaceutical Excipients”, 2nd Edition.
52. Berge et al. (1977). J. Pharm. Sci. 66:1-19.
53. Oslo (Ed.) (1985). Remington's Pharmaceutical Sciences. Mace Publishing Company, Philadelphia, Pa., 17th ed.
54. Langer. (1990). Science. 249:1527-1533.
55. Szoka et al. (1980). Ann. Rev. Biophys. Bioeng. 9:467.
56. Deamer, et al. (1983). In LIPOSOMES, Marcel Dekker, New York, p. 27
57. Hope, et al. (1986). Chem. Phys. Lipids. 40:89.
58. Santel et al. (2006). Gene Ther. 13(16):1222-1234.
59. Landen et al. (2005). Cancer Res. 65(15):6910-6918.
60. Spagnou et al. (2004). Biochemistry. 43(42):13348-133456.
61. Bouxsein et al. (2007). Biochemistry. 46(16):4785-4792.
62. Castanotto and Rossi, (2009). Nature. 457(7228):426-33.
63. Dominska and Dykxhoorn. (2010). J Cell Sci. 123(Pt 8):1183-1189.
64. Amarzguioui et al. (2006). Nat. Protoc. 1(2):508-517.
65. Liu et al. (2005). Neurobiol. 19(3):407-418.
66. Park et al. (2007). J Gene Med. 9(8):691-702.
67. d'Alessandro et al. (1998). Clin. Chim. Acta 274(2):189-197.
68. Sloots et al. (2005). FEBS J. 272:4221-4236.
69. Le et al. (2000). J Cell Sci. 113(18):3227-3240.
70. Le and Nabi. (2003). J Cell Sci. 116(6):1059-1071.
71. Bolte and Cordelieres. (2006). J Microsc. 224(3):213-232.
72. Manders et al. (1992). J Cell Sci. 103(3):857-862.
73. Li et al. (2004.) J. Neurosci. 24(16):4070-4081.
74. Zinchuk and Zinchuk. (2008). Curr Protoc Cell Biol. Chapter 4: p. Unit 4 19.
75. Vandesompele et al. (2002). Genome Biol. 3(7):RESEARCH 0034.
76. Svensson et al. (2008). Mol. Ther. 16(12):1995-2001.
77. Frank-Kamenetsky et al. (2008). Proc Natl Acad Sci USA. 105(33):11915-11920.
78. Sun et al. (2008). Nat. Biotechnol. 26(12): 1379-1382.
79. Judge et al. (2009). J Clin Invest. 119(3): 661-73.
80. Marti et al. (1998). Proc Natl Acad. Sci. U.S.A. 95(26):15809-15814.
81. Akinc et al. (2009). Mol. Ther. 17(5): 872-879.
82. Judge and MacLachlan. (2008). Hum Gene Ther. 19(2):111-124.
83. Christensen et al. (2006). Immunity. 25(3):417-428.
84. Forsbach et al. (2008). J. Immunol. 180(6):3729-3738.
85. Dalal et al. (2005). Clin Cancer Res. 11(6):2364-2378.
86. Gerber et al. (2000). Cancer Res. 60(22):6253-6258.
87. Borgstrom et al. (1996). Cancer Res. 56(17):4032-4039.
88. Mordenti et al. (1999). Toxicol Pathol. 27(1):14-21.
89. Takei et al. (2004). Cancer Res. 64(10):3365-3370.
90. Kim et al. (1993). Nature. 362(6423):841-844.
91. Presta et al. (1997). Cancer Res. 57(20):4593-4599.
92. Raskopf et al. (2008). J Hepatol. 49(6):977-984.
93. Guan et al. (2005). Clin. Cancer Res. 11(7):2662-2669.
94. Kleinman et al. (2008). Nature. 452(7187):591-597.
95. Kariko et al. (2005). Immunity. 23(2):165-175.
96. Judge et al. (2006). Mol. Ther. 13(3): 494-505.
97. Geisbert et al. (2006). J Infect Dis. 193(12):1650-1657.
98. Robbins et al. (2008). Hum Gene Ther. 19(10):991-999.
99. Marques et al. (2006). Nat. Biotechnol. 24(5):559-565.
100. Gerber et al. (1999). Development. 126(6):1149-1159.
101. Mu et al. (2009). Int J Cancer. 125(12):2978-2990.
102. Makin and Dive. (2001). Trends Cell Biol. 11(11):S22-26.
103. Takei et al. (2006). Cancer. 107(4):864-873.
104. Borowsky et al. (2005). Clin Exp Metastasis. 22(1):47-59.
105. Helms et al. (2010). Cancer Immunol Immunother. 59(9):1325-1334.
106. Watson and Paxinos (Eds.). (2007). “The rat brain in stereotaxic coordinates”. 6 ed. Elsevier Academic Press, London.
107. Paxinos and Franklin (Eds.). (2007). “The mouse brain in stereotaxic coordinates”. 3rd ed. Academic Press.
108. Thakker et al. (2004). Proc Natl Acad Sci USA. 101(49):17270-17275.
109. Thakker et al. (2005). Mol. Psychiatry. 10(8):782-789.
110. Guo et al. (2002). Oncogene. 21(49):7545-7556.
111. Wesche. (2002). Int J Med. Microbiol. 291(6-7):517-521.
112. Sandvig (1991). J. Cell Biol. 115(4):971-981.
113. Khine et al. (2004). Glycobiology. 14(8):701-712.
114. Arasaki et al. (2007). Mol. Pharmacol. 71(2):454-460.
115. Drecktrah et al. (1998). J Cell Sci. 111(Pt 7):951-965.
116. Stechmann et al. (2010). Cell. 141(2):231-242.
117. Sáenz et al. (2009). Nat Chem. Biol. 5(3):157-165.

1. A conjugate for delivery of a compound into a cell comprising or consisting of:
(a) at least one module that mediates cell targeting and facilitates cellular uptake,
(b) at least one module that facilitates transport to the endoplasmic reticulum (ER),
(c) at least one module that mediates translocation from the ER to the cytosol, and
(d) at least one compound,
wherein the modules (a) to (c) and the compound (d) are linked to each other in any arrangement.

2. The conjugate of claim 1, wherein the modules and the compound are linked to each other in one of the following arrangements: (a)ₓ, (b)_{y}, (c)_{z} and (d)ₙ; (b)_{y}, (a)ₓ, (c)_{z} and (d)ₙ; (b)_{y}, (c)_{z}, (a)ₓ and (d)ₙ; (c)_{z}, (b)_{y}, (a)ₓ and (d)ₙ; (a)ₓ, (c)_{z}, (b)_{y} and (d)ₙ; (c)_{z}, (a)ₓ, (b)_{y} and (d)ₙ; (c)_{z}, (d)ₙ, (b)_{y} and (a)ₓ; (d)ₙ, (c)_{z}, (b)_{y} and (a)ₓ; (b)_{y}, (d)ₙ, (c)_{z} and (a)ₓ; (d)ₙ, (b)_{y}, (c)_{z} and (a)ₓ; (b)_{y}, (c)_{z}, (d)ₙ and (a)ₓ; (c)_{z}, (b)_{y}, (d)ₙ and (a)ₓ; (c)_{z}, (d)ₙ, (a)ₓ and (b)_{y}; (d)ₙ, (c)_{z}, (a)ₓ and (b)_{y}; (a)ₓ, (d)ₙ, (c)_{z} and (b)_{y}; (d)ₙ, (a)ₓ, (c)_{z} and (b)_{y}; (a)ₓ, (c)_{z}, (d)ₙ and (b)_{y}; (c)_{z}, (a)ₓ, (d)ₙ and (b)_{y}; (b)_{y}, (d)ₙ, (a)ₓ and (c)_{z}; (d)ₙ, (b)_{y}, (a)ₓ and (c)_{z}; (a)ₓ, (d)ₙ, (b)_{y} and (c)_{z}; (d)ₙ, (a)ₓ, (b)_{y} and (c)_{z}; (a)ₓ, (b)_{y}, (d)ₙ and (c)_{z}; or (b)_{y}, (a)ₓ, (d)ₙ and (c)_{z}, and
wherein
x is an integer of 1 to 5, preferably of 1;
y is an integer of 1 to 5; preferably of 1;
z is an integer of 1 to 5; preferably of 1; and
n is an integer of 1 to 50, preferably of 1, 2, 3, 4, 5, 6, 7, 8, 9, or 10.

3. The conjugate of claim 2, wherein the arrangements in which the modules and the compound are linked to each other are
(i) (a)ₓ, (c)_{z}, (d)ₙ, and (b)_{y}, wherein x is an integer of 1, z is an integer of 1, n is an integer of 1 and y is an integer of 1,
(ii) (a)ₓ, (c)_{z}, (d)ₙ, and (b)_{y}, wherein x is an integer of 1, z is an integer of 1, n is an integer of 2 and y is an integer of 1,
(iii) (a)ₓ, (c)_{z}, (d)ₙ, and (b)_{y}, wherein x is an integer of 1, z is an integer of 1, n is an integer of 3 and y is an integer of 1,
(iv) (a)ₓ, (d)ₙ, (c)_{z} and (b)_{y}, wherein x is an integer of 1, n is an integer of 1, z is an integer of 1 and y is an integer of 1,
(v) (a)ₓ, (d)ₙ, (c)_{z} and (b)_{y}, wherein x is an integer of 1, n is an integer of 2, z is an integer of 1 and y is an integer of 1,
(vi) (a)ₓ, (d)ₙ, (c)_{z} and (b)_{y}, wherein x is an integer of 1, n is an integer of 3, z is an integer of 1 and y is an integer of 1.

4. The conjugate of claim 1, wherein the modules and the compound are
(i) linked to each other via a covalent linkage,
(ii) linked to each other via a non-covalent linkage,
(iii) linked to each other via at least one adapter molecule, and/or
(iv) linked to each other via at least one linker molecule that optionally comprises at least one adapter molecule.

5. The conjugate of claim 2, wherein the arrangements in which the modules and the compound are linked to each other are
(i) (a)ₓ, (c)_{z}, (d)ₙ and (b)_{y}, wherein (a)ₓ is covalently linked to (c)_{z}, (c)_{z} is covalently linked to (d)ₙ, and (d)ₙ is covalently linked to (b)_{y};
(ii) (a)ₓ, (c)_{z}, (d)ₙ, and (b)_{y}, wherein (a)ₓ is covalently linked to (c)_{z}, (c)_{z} is covalently linked to (d)ₙ, and (d)ₙ is non-covalently linked to (b)_{y};
(iii) (a)ₓ, (d)ₙ, (c)_{z} and (b)_{y}, wherein (a)ₓ is covalently linked to (d)ₙ, (d)ₙ is covalently linked to (c)_{z}, and (c)_{z} is covalently linked to (b)_{y};
(iv) (a)ₓ, (d)ₙ, (c)_{z}, and (b)_{y}, wherein (a)ₓ is non-covalently linked to (d)ₙ, (d)ₙ is non-covalently linked to (c)_{z}, and (c)_{z} is covalently linked to (b)_{y};
(v) (a)ₓ, (c)_{z}, (d)ₙ, and (b)_{y}, wherein (a)ₓ is covalently linked to (c)_{z} via a linker molecule, (c)_{z} is covalently linked to (d)ₙ via a linker molecule, and (d)ₙ is covalently linked to (b)_{y} via a linker molecule;
(vi) (a)ₓ, (c)_{z}, (d)ₙ, and (b)_{y}, wherein (a)ₓ is covalently linked to (c)_{z} via a linker molecule, (c)_{z}, is covalently linked to (d)ₙ via a linker molecule, and (d)ₙ is non-covalently linked to (b)_{y};
(vii) (a)ₓ, (d)ₙ, (c)_{z}, and (b)_{y}, wherein (a)ₓ is covalently linked to (d)ₙ via a linker molecule; (d)ₙ is covalently linked to (c)_{z}, via a linker molecule and (c)_{z} is covalently linked to (b)_{y} via a linker molecule;
(viii) (a)ₓ, (d)ₙ, (c)_{z}, and (b)_{y}, wherein (a)ₓ, is non-covalently linked to (d)ₙ, (d)ₙ is non-covalently linked to (c)_{z}, and (c)_{z} is covalently linked to (b)_{y} via a linker molecule; or
(ix) (a)ₓ, (d)ₙ, (c)_{z}, and (b)_{y}, wherein (a)ₓ is non-covalently linked to (d)ₙ via an adapter molecule that is covalently linked to (a)ₓ, (d)ₙ is non-covalently linked to (c)_{z} via an adapter molecule that is covalently linked to (c)_{z}, and (c)_{z} is covalently linked to (b)_{y} via a linker molecule.

6. The conjugate of claim 30, wherein the modules and the compound are linked to each other in the following arrangement, wherein
(i) (a)ₓ is covalently linked to (c)_{z}, (c)_{z} is covalently linked to (d)ₙ, and (c)_{z} is covalently linked to (b)_{y};
(ii) (a)ₓ is covalently linked to (c)_{z}, (c)_{z} is non-covalently linked to (d)ₙ, and (c)_{z} is covalently linked to (b)_{y};
(iii) (a)ₓ is covalently linked to (d)ₙ, (a)ₓ is covalently linked to (c)_{z}, and (c)_{z} is covalently linked to (b)_{y};
(iv) (a)ₓ is non-covalently linked to (d)ₙ, (a)ₓ is covalently linked to (c)_{z}, and (c)_{z} is covalently linked to (b)_{y};
(v) (a)ₓ is covalently linked to (c)_{z} via a linker molecule, (c)_{z} is covalently linked to (d)ₙ via a linker molecule, and (c)_{z} is covalently linked to (b)_{y} via a linker molecule;
(vi) (a)ₓ is covalently linked to (c)_{z} via a linker molecule, (c)_{z} is non-covalently linked to (d)ₙ via an adapter molecule that is covalently linked to (c)_{z}, and (c)_{z} is covalently linked to (b)_{y} via a linker molecule;
(vii) (a)ₓ is covalently linked to (d)ₙ via a linker molecule, (a)ₓ is covalently linked to (c)_{z} via a linker molecule and (c)_{z} is covalently linked to (b)_{y} via a linker molecule; or
(viii) (a)ₓ is non-covalently linked to (d)ₙ via an adapter molecule that is covalently linked to (a)ₓ, (a)ₓ is covalently linked to (c)_{z} via a linker molecule, and (c)_{z} is covalently linked to (b)_{y} via a linker molecule.

7. The conjugate of claim 4, wherein the covalent linkage is a disulfide-linkage, an amide-linkage, an oxime-linkage or a hydrazone-linkage and, wherein the non-covalent linkage is an ionic linkage or a hydrophobic linkage.

8. The conjugate of claim 4, wherein the linker molecule is a peptide or a modified peptide, preferably a peptide covalently bound to polyethylene glycol (PEG) and, wherein the adapter molecule is a double stranded RNA binding protein (DRBP) or a variant thereof.

9. The conjugate of claim 4, wherein the linker molecule comprises
(i) at least one branch point, preferably a lysine side chain, a cysteine side chain, or an unnatural amino acid containing an aminoxy moiety on the side chain, and/or
(ii) at least one cleavage site, preferably a furin or a calpain cleavage site.

10. The conjugate of claim 9, wherein the cleavage site is between module (a) an module (c) or between module (a) and compound (d).

11. The conjugate of claim 9, wherein the compound is covalently linked to the branch point, preferably via an amide-linkage to the lysine side chain, via a disulfide-linkage to the cysteine side chain or via an unnatural amino acid containing an aminoxy moiety on the side chain.

12. The conjugate of claim 9, wherein the compound is non-covalently linked to the branch point via an ionic linkage or via a hydrophobic linkage to DRBD or a variant thereof that is covalently linked via a disulfide linkage to the cysteine side chain.

13. The conjugate of claim 1, wherein
(i) the module (a) comprises a cell surface receptor ligand, an antibody, a sugar, a lipid or a nanoparticle,
(ii) the module (b) comprises an oligopeptide comprising one or more of an amino acid sequence X₁X₂X₃X₄ (SEQ ID NO: 140), wherein
X₁ is E, H, K, N, P, Q, R, or S, preferably K or R,
X₂ is D, E, A, T, V, G, S, or N, preferably D, or E,
X₃ is E, or D, preferably E,
X₄ is L, or F, preferably L, and wherein optionally the N-terminus and/or C-terminus comprises 1 to 3 additional amino acid residues;
(iii) the module (c) comprises
(a) a peptide of a protein selected from the group consisting of COX2, IgM(μ), Sgk1, MATalpha2, MF(alpha)1, CPY, a toxin subunit A, a fragment thereof, or a variant thereof, or
(b) an amino acid sequence comprising CL1 (SEQ ID NO: 164), CL2 (SEQ ID NO: 165), CL6 (SEQ ID NO: 166), CL9 (SEQ ID NO: 167), CL10 (SEQ ID NO: 168), CL11 (SEQ ID NO: 169), CL12 (SEQ ID NO: 170), CL15 (SEQ ID NO: 171), CL16 (SEQ ID NO: 172) or SL17 (SEQ ID NO: 173), and
(iv) the compound (d) comprises a nucleic acid or a peptide.

14. The conjugate of claim 13, wherein
(i) the cell surface receptor ligand is selected from the group consisting of a growth factor, a lipoprotein, a transferrin, a surface binding lectin, a galectin, a c-type lectin, a toxin, a fragment thereof, and a variant thereof,
(ii) the antibody is selected from the group consisting of anti-TGN38/46, anti-transferrin receptor, and anti-growth factor receptor,
(iii) the lipid is selected from the group consisting of a phospholipid, a glycolipid, a sphingolipid, and a sterol lipid, and
(iv) the nanoparticle is selected from the group consisting of a metal, a silicate, and a polymer.

15. The conjugate of claim 14, wherein the cell surface receptor ligand is a toxin selected from the group consisting of B chain of Ricin, B chain of Abrin, B chain of Modeccin, B chain of Volkensin, B chain of Cholera toxin, B chain of Shiga toxin, B chain of Verotoxin, domains I, II and IV of Pseudomonas Exotoxin A, and B chain of Escherichia coli heat-labile enterotoxin.

16. The conjugate of claim 13, wherein the module (c) is selected from the from the group consisting of
(i) NX₁SX₂X₃X₄X₅X₆X₇X₈X₉INPTX₁₀X₁₁X₁₂X₁₃ (SEQ ID NO: 178), wherein X₁ is A, 5, or V; X₂ is 5, A, or T; X₃ is 5, or V; X₄ is R, H, or N; X₅ is 5, or T; X₆ is G, R, T, or A; X₇ is L, V, or M; X₈ is D, N, or E; X₉ is D, or N; X₁₀ is V, or L; X₁₁ is L, or V; X₁₂ is L, or I; and X₁₃ is K, or N;
(ii) GKPTLYX₁VSLX₂MSDTX₃GTX₄Y (SEQ ID NO: 190), wherein X₁ is N, or Q; X₂ is I, or V; X₃ is G, or A; and X₄ is C, or 5;
(iii) MTX₁X₂X₃X₄EX₅X₆X₇X₈X₉X₁₀X₁₁LTYSX₁₂X₁₃RGX₁₄VAX₁₅LX₁₆AFMKQRX₁₇MGLNDFIQKX₁₈X₁₉X₂₀NX₂₁YACKHX₂₂EVQSX₂₃LX₂₄X₂₅ (SEQ ID NO: 200), wherein X₁ is V, or I; X₂ is K, or Q; X₃ is A, or T; X₄ is X (X is zero amino acid) or A; X₅ is A, or T; X₆ is A, or S; X₇ is R, K, G, or V; X₈ is S, G, or P; X₉ is T, P, or A; X₁₀ is X or P; X₁₁ is X or D; X₁₂ is R, or K; X₁₃ is M, or T; X₁₄ is M, or L; X₁₅ is I, or N; X₁₆ is I, or S; X₁₇ is R, or K; X₁₈ is I, or L; X₁₉ is A, or S; X₂₀ is S, N, A, or T; X₂₁ is T, or S; X₂₂ is A, P, or T; X₂₃ is I, or Y; X₂₄ is K, or N; and X₂₅ is M, I, or L;
(iv) MRFPSIFTAVLFAASSALAAPVX₁TTTEDETAQIPAEAVIGYLDLEGDFDVAVLPFSX₁STNNGLLFIX₁TTIASIAAKEEGVSLDKREAEAWHWLQLKPGQPMYKREAEAEAWHWLQLKPGQPMYKREADAEAWHWLQLKPGQPMYKREADAEAWHWLQLKPGQPMY (SEQ ID NO: 220), wherein X₁ is N, or Q; and
(v) MNKIPIKDLLNPQITDEFKSSILDINKKLFSICCNLPKLPESVTTEEEVELRDILX₁FLSRAN (SEQ ID NO: 214), wherein X₁ is G, V, or L.

17. The conjugate of claim 16, wherein the module (c) is
(i)
(SEQ ID NO: 176)
NASSSRSGLDDINPTVLLK;
(ii)
(SEQ ID NO: 179)
NASASHSRLDDINPTVLIK;
(iii)
(SEQ ID NO: 180)
NASSSHSGLDDINPTVLLK;
(iv)
(SEQ ID NO: 184)
GKPTLYNVSLIMSDTGGTCY;
(v)
(SEQ ID NO: 185)
GKPTLYNVSLVMSDTAGTCY;
(vi)
(SEQ ID NO: 186)
GKPTLYQVSLIMSDTGGTCY;
(vii)
(SEQ ID NO: 187)
GKPTLYQVSLIMSDTGGTSY;
(viii)
(SEQ ID NO: 193)
MTVKAEAARSTLTYSRMRGMVAILIAFMKQRRMGLNDFIQKIASNTYAC
KHAEVQSILKM;
(ix)
(SEQ ID NO: 197)
MTVKTEAAKGTLTYSRMRGMVAILIAFMKQRRMGLNDFIQKIANNSYAC
KHPEVQSILKI;
(x)
(SEQ ID NO: 212)
MNKIPIKDLLNPQITDEFKSSILDINKKLFSICCNLPKLPESVTTEEEVE
LRDILGFLSRAN;
(xi)
(SEQ ID NO: 215)
MNKIPIKDLLNPQITDEFKSSILDINKKLFSICCNLPKLPESVTTEEEVE
LRDILVFLSRAN;
or
(xii)
(SEQ ID NO: 216)
MNKIPIKDLLNPQITDEFKSSILDINKKLFSICCNLPKLPESVTTEEEVE
LRDILLFLSRAN.

18. The conjugate of claim 17, wherein the module (c) is
(i)
(SEQ ID NO: 205)
MRGMVAILIAFMKQRRMGLNDFIQKIASNTYACKHAEV
QSILKM;
(ii)
(SEQ ID NO: 206)
MRGMVAILIAFMKQ;
(iii)
(SEQ ID NO: 207)
GMVAILIAF;
(iv)
(SEQ ID NO: 210)
MRGMVAILIAFMKQRRMGLNDFIQKIANNSYACKHPE
VQSILKI;
(v)
(SEQ ID NO: 217)
ITDEFKSSILDINKKLFSI;
or
(vi)
(SEQ ID NO: 218)
ITDEFKSSILDINKKLFSICCNLPKLPESV.

19. The conjugate of claim 13, wherein the nucleic acid is a single stranded DNA, a double stranded DNA, a single stranded RNA, a double stranded RNA, an siRNA, a transfer RNA (tRNA), a messenger RNA (mRNA), a micro RNA (miRNA), a small nuclear RNA (snRNA), a small hairpin RNA (shRNA) or a morpholino-modified iRNA.

20. The conjugate of claim 13, wherein the nucleic acid is chemically modified.

21. A conjugate according to claim 1 for use as a pharmaceutical.

22. A pharmaceutical composition comprising
(i) a conjugate according to claim 1, and
(ii) a pharmaceutically acceptable excipient, carrier and/or diluent.

23. A method of delivering a compound (d) to a cell comprising the steps of
(a) providing a cell,
(b) contacting a conjugate according to claim 1 comprising the compound (d) with said cell under conditions whereby the conjugate is internalized by the cell, thereby delivering the compound (d) to the cell.

24. The method according to claim 23, wherein the cell is a eukaryotic cell, an invertebrate cell, a vertebrate cell, a nematode cell, a fungal cell, an Aspergillus cell, a yeast cell, a Sacchromyces cell, a Pichia cell, an insect cell, an Sf9 cell, an animal cell, a non-human animal cell, a mammalian cell, a non-human mammalian cell, a CHO, a primate cell, a non-human primate cell, a human cell, or a plant cell.

25. A method of delivering a compound (d) to a patient comprising the step of
(a) administering a sufficient amount of a conjugate according to claim 1 to a patient, thereby delivering the compound (d) to the patient.

26. A method of modifying gene expression in a cell comprising the steps of
(a) providing a cell, and
(b) contacting the conjugate according to claim 1 comprising a compound (d) with said cell under conditions whereby the conjugate is internalized by the cell and the compound (d) of the conjugate is delivered to the cell's cytosol or nucleus, wherein the compound (d) is a nucleic acid or a peptide capable of modifying gene expression in the cell, and
(c) upon reaching the cell's cytosol or nucleus, the compound (d) modifies gene expression in the cell.

27. A method of preparing a conjugate comprising coupling at least one module (a) that mediates cell targeting and facilitates cellular uptake, at least one module (b) that facilitates transport to the endoplasmic reticulum (ER), at least one module (c) that mediates translocation from the ER to the cytosol, and at least one compound (d), wherein the modules (a), (b) and (c) and the compound (d) are linked to each other in any arrangement and in any stoichiometry.

28. A kit comprising a component to prepare the conjugate according to claim 1, wherein the kit comprises a module (a), a module (b), a module (c), and/or a compound (d) and wherein the kit comprises an optional peptide linker and/or an optional peptide comprising a cleavage site.

29. A kit comprising a delivery system comprising the conjugate according to claims 1.

30. The conjugate of claim 3, wherein the modules and the compound are
(i) linked to each other via a covalent linkage,
(ii) linked to each other via a non-covalent linkage,
(iii) linked to each other via at least one adapter molecule, and/or
(iv) linked to each other via at least one linker molecule that optionally comprises at least one adapter molecule.

31. The conjugate of claim 10, wherein the compound is covalently linked to the branch point, preferably via an amide-linkage to the lysine side chain, via a disulfide-linkage to the cysteine side chain or via an unnatural amino acid containing an aminoxy moiety on the side chain.

32. The conjugate of claim 10, wherein the compound is non-covalently linked to the branch point via an ionic linkage or via a hydrophobic linkage to DRBD or a variant thereof that is covalently linked via a disulfide linkage to the cysteine side chain.
